(19) United States
(12) Patent Application Publication
(10) Pub. No.: US 2017/0018719 A1
(43) Pub. Date: Jan. 19, 2017

(54) COMPOUND, MATERIAL FOR ORGANIC ELECTROLUMINESCENCE ELEMENT, INK COMPOSITION, ORGANIC ELECTROLUMINESCENCE ELEMENT, AND ELECTRONIC DEVICE

(71) Applicant: IDEMITSU KOSAN CO., LTD., Tokyo (JP)

(72) Inventors: Hironori KAWAKAMI, Tokyo (JP); Kiyoshi IKEDA, Sodegaura-shi (JP); Taro YAMAKI, Chiba-shi (JP); Takashi KASHIWAMURA, Ichihara-shi (JP)

(73) Assignee: IDEMITSU KOSAN CO., LTD., Tokyo (JP)

(21) Appl. No.: 15/124,523

(22) PCT Filed: Mar. 12, 2015
(86) PCT No.: PCT/JP2015/057391

§ 371 Date: Sep. 8, 2016

(30) Foreign Application Priority Data
Mar. 12, 2014 (JP) 2014-049549

(51) Int. Cl. H01L 51/00; C09K 11/06; C07D 403/14; C09D 5/24; C09K 11/02

(52) U.S. Cl. CPC H01L 51/0072; C09D 5/24; C09K 11/025; C07D 403/14; H01L 51/0067; H01L 51/0052; H01L 51/0058; H01L 51/0085; C09K 11/06; C09K 2211/1007; C09K 2211/1029; C09K 2211/185; H01L 51/5012

(57) ABSTRACT

Provided are a compound that is represented by the following general formulae (1) and (2), and has a nitrogen-containing heteroaromatic group substituted with a substituent having a particular structure containing 11 or more benzene rings in one molecule, a material for an organic electroluminescence device containing the compound, an ink composition containing a solvent and the compound, an organic electroluminescence device containing the compound, and an electronic equipment having the organic electroluminescence device mounted thereon.

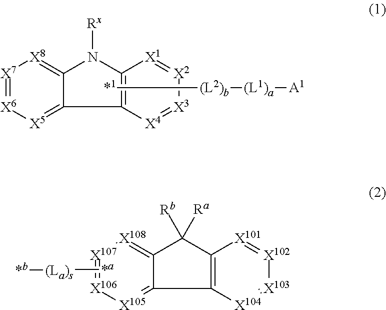

### TECHNICAL FIELD

The present invention relates to a compound, a material for an organic electroluminescence device, an ink composition, an organic electroluminescence device, and an electronic equipment.

### BACKGROUND ART

An organic electroluminescence device (which may be hereinafter referred to as an organic EL device) has been known, which has an organic thin film layer including a light emitting layer between an anode and a cathode, and provides light emission with exciton energy formed through recombination of holes and electrons injected to the light emitting layer (see PTL 1).

An organic EL device is expected as a light emitting device that has a high light emission efficiency, a high image quality and a low electric power consumption, and is excellent in low-profile design, due to the advantages thereof as a self-luminous device. It has been known that a host-dopant light emitting layer obtained by doping a host material with a light emitting material as a dopant is used as the light emitting layer of the device.

In the host-dopant light emitting layer, excitons can be efficiently formed with the charge injected to the host. The energy of the excitons thus formed is transferred to the dopant, and light emission is achieved with the dopant in a high efficiency.

In recent years, for enhancing the capability of the organic EL device, extensive studies have been made on the host-dopant system, and researches have been continuously made on a favorable host material and other materials for the organic EL device.

The method for forming the layers of the organic EL device may be roughly classified into a vapor deposition method, such as a vacuum vapor deposition method and a molecular beam vapor deposition method, and a coating method, such as a dipping method, a spin coating method, a casting method, a bar coating method and a roll coating method. Materials that are used for forming a layer by the coating method are required to have different characteristics from materials that are used for the vapor deposition method, such as temperature resistance and solubility in a solvent. Accordingly, a material that is useful for the vapor deposition method is not necessarily useful for the coating method. Furthermore, the material necessarily satisfies the various performances demanded for an organic EL device while satisfying the capability of forming a layer by the coating method.

In particular, the formation of layers by the coating method may be applied to the production of a large-size organic EL display, an illumination panel and the like, and therefore the development of a material for an organic EL device capable of being applied to the coating method is being demanded.

### CITATION LIST

### Patent Literature

PTL 1: WO 2012/086170

### SUMMARY OF INVENTION

### Technical Problem

An object of the present invention is to provide a compound that is favorable for applying to an organic EL device, layers of which are formed by a coating method. Another object thereof is to provide a material for an organic electroluminescence device containing the compound, an ink composition containing the compound, an organic electroluminescence device using the compound, and an electronic equipment having the organic electroluminescence device mounted thereon.

### Solution to Problem

As a result of earnest investigations made by the present inventors, it has been found that the problem may be solved by a compound that has a nitrogen-containing heteroaromatic group substituted with a substituent having a particular structure.

According to one embodiment of the present invention, the following items [1] to [10] may be provided.

[1] A compound that is represented by the following general formulae (1) and (2), and contains 11 or more benzene rings in one molecule:

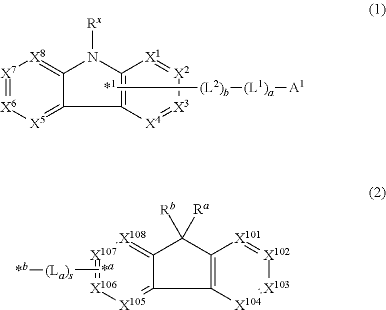

wherein

in the general formula (1),
A¹ represents a substituted or unsubstituted nitrogen-containing heteroaromatic group having from 5 to 30 ring atoms;
L¹ and L² each independently represent a substituted or unsubstituted aromatic hydrocarbon group having from 6 to 30 ring carbon atoms, a substituted or unsubstituted heterocyclic group having from 5 to 30 ring atoms, or a group containing from 2 to 4 of the groups bonded to each other;
a and b each independently represent 0 or 1, provided that when a is 0, (L¹)₀ represents a single bond, and when b is 0, (L²)₀ represents a single bond;
R^{x} represents a hydrogen atom or a substituent;
X¹ to X⁸ each represent C(R¹) to C(R⁸) or a nitrogen atom; and
R¹ to R⁸ each independently represent a hydrogen atom or a substituent, in which adjacent substituents may be bonded to each other to form a ring,
provided that any one of R¹ to R⁴ and R^{x} represents a single bond bonded to L² at the position of *¹,

in the general formula (2),
Lₐ represents a substituted or unsubstituted aromatic hydrocarbon group having from 6 to 30 ring carbon atoms or a substituted or unsubstituted heterocyclic group having from 5 to 30 ring atoms;
s represents 0 or 1, provided that when s is 0, (Lₐ)₀ represents a single bond;
X¹⁰¹ to X¹⁰⁸ each represent C(R¹⁰¹) to C(R¹⁰⁸) or a nitrogen atom, and R¹⁰¹ to R¹⁰⁸ each independently represent a hydrogen atom or a substituent, in which adjacent substituents may be bonded to each other to form a ring, provided that any one of R¹⁰⁵ to R¹⁰⁸ represents a single bond directly bonded to Lₐ at the position of *^{a}; and
R^{a} and R^{b} each independently represent a hydrogen atom, an alkyl group having from 1 to 10 carbon atoms, an aryl group having from 6 to 14 ring carbon atoms, or a heteroaryl group having from 5 to 14 ring atoms, in which R^{a} and R^{b} may be bonded to each other to form a ring, and R^{a} and R¹⁰¹, and R^{b} and R¹⁰⁸ each independently may be bonded to each other to form a ring,

the group represented by the formula (2) is bonded to at least one of L¹, L², R¹ to R⁸, and R^{x} at the position of *^{b}, in which any one of R¹ to R⁸, and R^{x} that is bonded to the group represented by the formula (2) represents a single bond, and

when plural groups each represented by the formula (2) are present, the groups may be the same as or different from each other.

[2] An ink composition containing a solvent and the compound according to the item [1].

[3] A material for an organic electroluminescence device including the compound according to the item [1].

[4] An organic electroluminescence device including a cathode, an anode, and one or more organic thin film layers between the cathode and the anode, in which

the one or more organic thin film layers includes a light emitting layer, and at least one layer of the one or more organic thin film layers contains the compound according to the item [1].

[5] An electronic equipment having the organic electroluminescence device according to the item [4] mounted thereon.

According to another embodiment of the present invention, the following items [6] to [10] may be provided.

[6] A compound that is represented by the following general formulae (3) and (4):

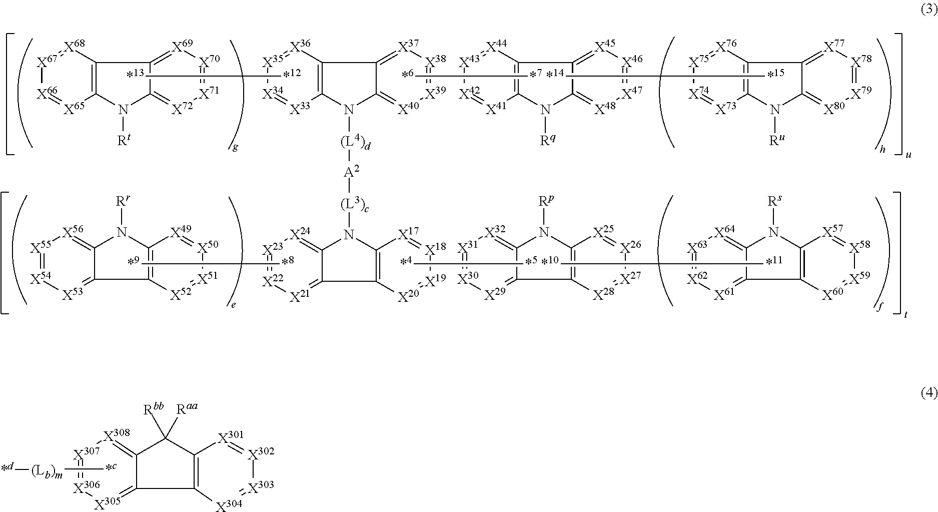

wherein

in the general formula (3),
A² represents a substituted or unsubstituted nitrogen-containing heteroaromatic group having from 5 to 30 ring atoms;
L³ and L⁴ each independently represent a substituted or unsubstituted aromatic hydrocarbon group having from 6 to 30 ring carbon atoms, a substituted or unsubstituted heterocyclic group having from 5 to 30 ring atoms, or a group containing from 2 to 4 of the groups bonded to each other;
c and d each independently represent 0 or 1, provided that when c is 0, (L³)₀ represents a single bond, and when d is 0, (L⁴)₀ represents a single bond;
R^{p} to R^{u} each independently represent a hydrogen atom or a substituent;
X¹⁷ to X⁸⁰ each represent C(R¹⁷) to C(R⁸⁰) or a nitrogen atom; and
R¹⁷ to R⁸⁰ each independently represent a hydrogen atom or a substituent, in which adjacent substituents may be bonded to each other to form a ring,
provided that any one of R¹⁷ to R²⁰ represents a single bond bonded to *⁴, any one of R^{p} and R²⁹ to R³² represents a single bond bonded to *⁵, any one of R³⁷ to R⁴⁰ represents a single bond bonded to *⁶, any one of R^{q} and R⁴¹ to R⁴⁴ represents a single bond bonded to *⁷, any one of R²¹ to R²⁴ represents a single bond bonded to *⁸, any one of R^{r} and R⁴⁹ to R⁵² represents a single bond bonded to *⁹, any one of R^{p} and R²⁵ to R²⁸ represents a single bond bonded to *¹⁰, any one of R^{s} and R⁶¹ to R⁶⁴ represents a single bond bonded to *¹¹, any one of R³³ to R³⁶ represents a single bond bonded to *¹², any one of R^{t} and R⁶⁹ to R⁷² represents a single bond bonded to *¹³, any one of R^{q} and R⁴⁵ to R⁴⁸ represents a single bond bonded to *¹⁴, and any one of R^{u} and R⁷³ to R⁷⁶ represents a single bond bonded to *¹⁵, and
when plural groups or atoms each represented by the same symbol are present for X¹⁷ to X⁸⁰, R¹⁷ to R⁸⁰, and R^{p} to R^{u}, the groups or the atoms may be the same as or different from each other;
e to h each independently represent an integer of 0 or 1, provided that when e to h are 0, the symbols in parentheses with a suffix of 0, (symbol)₀, each independently represent a hydrogen atom or a substituent; and
t and u each independently represent an integer of from 0 to 2, in which t+u=2, provided that the symbols in brackets with a suffix of 0, [symbol]₀, when t is 0, and the symbols in brackets with a suffix of 0, [symbol]₀, when u is 0 each represent a hydrogen atom,

in the general formula (4),
L_{b} represents a substituted or unsubstituted aromatic hydrocarbon group having from 6 to 30 ring carbon atoms or a substituted or unsubstituted heterocyclic group having from 5 to 30 ring atoms;
m represents 0 or 1, provided that when m is 0, (L_{b})₀ represents a single bond;
X³⁰¹ to X³⁰⁸ each represent C(R³⁰¹) to C(R³⁰⁸) or a nitrogen atom, and R³⁰¹ to R³⁰⁸ each independently represent a hydrogen atom or a substituent, in which adjacent substituents may be bonded to each other to form a ring, provided that any one of R³⁰⁵ to R³⁰⁸ represents a single bond directly bonded to L_{b} at the position of *^{c}; and
R^{aa} and R^{bb} each independently represent a hydrogen atom, an alkyl group having from 1 to 10 carbon atoms, an aryl group having from 6 to 14 ring carbon atoms, or a heteroaryl group having from 5 to 14 ring atoms, in which R^{aa} and R^{bb} may be bonded to each other to form a ring, and R^{aa} and R³⁰¹, and R^{bb} and R³⁰⁸ each independently may be bonded to each other to form a ring,

the group represented by the formula (4) is bonded to at least one of L³, L⁴, X¹⁷ to X⁸⁰, and R^{p} to R^{u} at the position of *^{d}, in which any one of R¹⁷ to R⁸⁰ and R^{p} to R^{u} that is bonded to the group represented by the formula (4) represents a single bond, and

when plural groups each represented by the formula (4) are present, the groups may be the same as or different from each other.

[7] An ink composition containing a solvent and the compound according to the item [6].

[8] A material for an organic electroluminescence device, containing the compound according to the item [6].

[9] An organic electroluminescence device containing a cathode, an anode, and one or more organic thin film layers between the cathode and the anode, in which

the one or more organic thin film layers includes a light emitting layer, and at least one layer of the one or more organic thin film layers contains the compound according to the item [6].

[10] An electronic equipment having the organic electroluminescence device according to the item [9] mounted thereon.

### Advantageous Effects of Invention

According to the present invention, a compound that is favorable for applying to an organic EL device, layers of which are formed by a coating method, can be provided.

### BRIEF DESCRIPTION OF DRAWING

FIG. 1 is a diagram showing a schematic configuration of an organic EL device according to one embodiment of the present invention.

### DESCRIPTION OF EMBODIMENT

In the description herein, in the expression “a substituted or unsubstituted ZZ group having from XX to YY carbon atoms”, “from XX to YY carbon atoms” means the number of carbon atoms of the ZZ group that is unsubstituted, and the number of carbon atoms of the substituent of the ZZ group that is substituted is not included. The number “YY” is larger than the number “XX”, and the numbers “XX” and “YY” each represent an integer of 1 or more.

In the description herein, in the expression “a substituted or unsubstituted ZZ group having from XX to YY atoms”, “from XX to YY atoms” means the number of atoms of the ZZ group that is unsubstituted, and the number of atoms of the substituent of the ZZ group that is substituted is not included. The number “YY” is larger than the number “XX”, and the numbers “XX” and “YY” each represent an integer of 1 or more.

In the description herein, the number of ring carbon atoms of a compound having a structure containing atoms that are bonded to form a ring (such as a monocyclic compound, a condensed ring compound, a crosslinked compound, a carbocyclic compound and a heterocyclic compound) means the number of carbon atoms in the atoms constituting the ring itself. In the case where the ring is substituted with a substituent, the carbon atom contained in the substituent is not included in the number of ring carbon atoms. The numbers of ring carbon atoms described hereinbelow are the same unless otherwise indicated. For example, a benzene ring has a number of ring carbon atoms of 6, a naphthalene ring has a number of ring carbon atoms of 10, a pyridinyl group has a number of ring carbon atoms of 5, and a furanyl group has a number of ring carbon atoms of 4. In the case where a benzene ring or a naphthalene ring is substituted, for example, with an alkyl group, the number of carbon atoms of the alkyl group is not included in the number of ring carbon atoms. In the case where a fluorene ring is substituted, for example, with a fluorene ring, (which includes a spirofluorene ring) the number of carbon atoms of the fluorene ring as the substituent is not included in the number of ring carbon atoms.

In the description herein, the number of ring atoms of a compound having a structure containing atoms that are bonded to form a ring (such as a monocyclic ring, a condensed ring and a group of rings) means the number of atoms constituting the ring itself (such as a monocyclic compound, a condensed ring compound, a crosslinked compound, a carbocyclic compound, and a heterocyclic compound). An atom that does not constitute the ring (for example, a hydrogen atom that terminates the bond of the atom constituting the ring) and an atom that is contained in a substituent when the ring is substituted with the substituent are not included in the number of ring atoms. The numbers of ring atoms described hereinbelow are the same unless otherwise indicated. For example, a pyridine ring has a number of ring atoms of 6, a quinazoline ring has a number of ring atoms of 10, and a furan group has a number of ring atoms of 5. A hydrogen atom that is bonded to a carbon atom of a pyridine ring or a quinazoline ring, or an atom forming a substituent is not included in the number of ring atoms. In the case where a fluorene ring is bonded to, for example, a fluorine ring as a substituent (which includes a spirofluorene ring), the number of atoms of the fluorene ring as the substituent is not included in the number of ring atoms.

In the description herein, “a hydrogen atom” encompasses isotopes with different numbers of neutrons, i.e., a protium, a deuterium, and a tritium.

In the description herein, “a heteroaryl group”, “a heteroarylene group”, and “a heterocyclic group” each mean a group that contains at least one hetero atom as a ring atom, and the hetero atom is preferably selected from a nitrogen atom, an oxygen atom, a sulfur atom, a silicon atom, and a selenium atom.

In the description herein, “a substituted or unsubstituted carbazolyl group” means the following groups, and a substituted carbazolyl group containing one of the following groups having an arbitrary substituent.

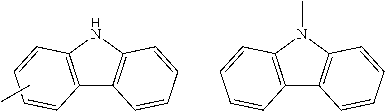

The substituted carbazolyl groups may be ring-condensed by bonding the arbitrary substituents to each other, may contain a hetero atom, such as a nitrogen atom, an oxygen atom, a silicon atom, and a selenium atom, and may be bonded at any of the 1- to 9-positions. Specific examples of the substituted carbazolyl group include the following groups.

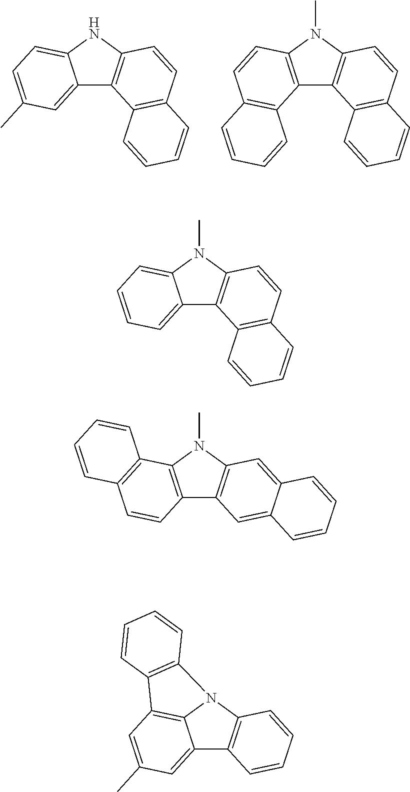

In the description herein, “a substituted or unsubstituted dibenzofuranyl group” and “a substituted or unsubstituted dibenzothiophenyl group” mean the following groups, and a substituted dibenzofuranyl group and a substituted dibenzothiophenyl group containing the following groups respectively having an arbitrary substituent.

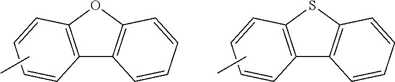

The substituted dibenzofuranyl groups and the substituted dibenzothiophenyl groups each may be ring-condensed by bonding the arbitrary substituents to each other, may contain a hetero atom, such as a nitrogen atom, an oxygen atom, a silicon atom, and a selenium atom, and may be bonded at any of the 1- to 8-positions.

Specific examples of the substituted dibenzofuranyl group and the substituted dibenzothiophenyl group include the following groups.

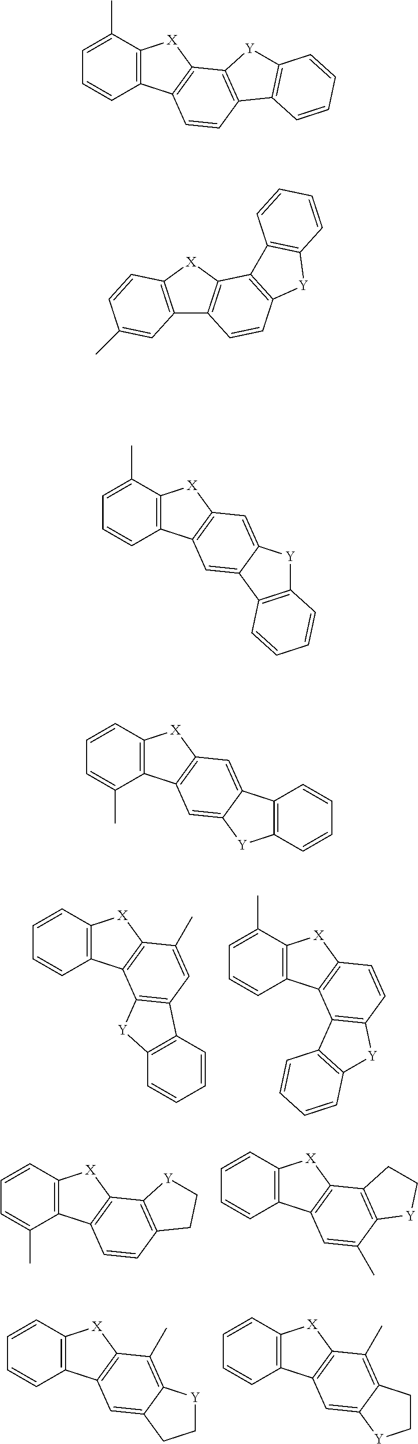

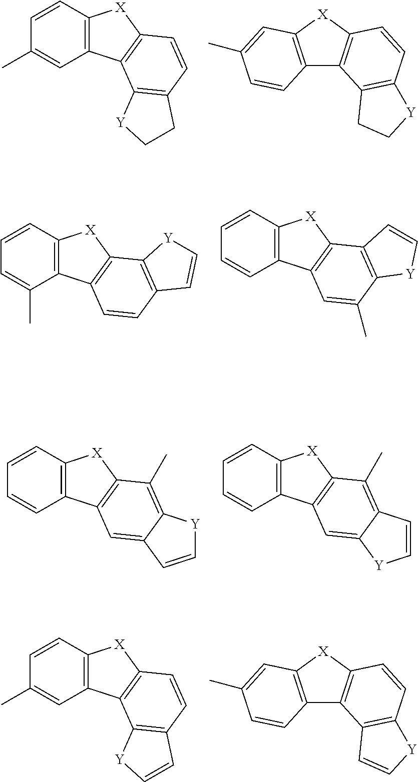

wherein in the above formulae, X represents an oxygen atom or a sulfur atom, and Y represents an oxygen atom, a sulfur atom, NH, NR^{ab} (wherein R^{ab} represents an alkyl group or an aryl group), CH₂, or CR^{cd}₂ (wherein Red represents an alkyl group or an aryl group).

“A substituent”, or the substituent in the expression “substituted or unsubstituted” is preferably at least one selected from the group consisting of an alkyl group having from 1 to 50 carbon atoms (preferably from 1 to 18, and more preferably from 1 to 8 carbon atoms); a cycloalkyl group having from 3 to 50 ring carbon atoms (preferably from 3 to 10, more preferably from 3 to 8, and further preferably 5 or 6 ring carbon atoms); an aryl group having from 6 to 50 ring carbon atoms (preferably from 6 to 25, and more preferably from 6 to 18 ring carbon atoms); an aralkyl group having from 7 to 51 carbon atoms (preferably from 7 to 30, and more preferably 7 to 20 carbon atoms) having an aryl group having from 6 to 50 ring carbon atoms (preferably from 6 to 25, and more preferably from 6 to 18 ring carbon atoms); an amino group; a monosubstituted or disubstituted amino group having a substituent selected from an alkyl group having from 1 to 50 carbon atoms (preferably from 1 to 18, and more preferably from 1 to 8 carbon atoms) and an aryl group having from 6 to 50 ring carbon atoms (preferably from 6 to 25, and more preferably from 6 to 18 ring carbon atoms); an alkoxy group having an alkyl group having from 1 to 50 carbon atoms (preferably from 1 to 18, and more preferably from 1 to 8 carbon atoms); an aryloxy group having an aryl group having from 6 to 50 ring carbon atoms (preferably from 6 to 25, and more preferably from 6 to 18 ring carbon atoms); a monosubstituted, disubstituted or trisubstituted silyl group having a substituent selected from an alkyl group having from 1 to 50 carbon atoms (preferably from 1 to 18, and more preferably from 1 to 8 carbon atoms) and an aryl group having from 6 to 50 ring carbon atoms (preferably from 6 to 25, and more preferably from 6 to 18 ring carbon atoms); a heteroaryl group having from 5 to 50 ring atoms (preferably from 5 to 24, and more preferably from 5 to 13 ring atoms); a haloalkyl group having from 1 to 50 carbon atoms (preferably from 1 to 18, and more preferably from 1 to 8 carbon atoms); a halogen atom (such as a fluorine atom, a chlorine atom, a bromine atom, and an iodine atom); a cyano group; a nitro group; a sulfonyl group having a substituent selected from an alkyl group having from 1 to 50 carbon atoms (preferably from 1 to 18, and more preferably from 1 to 8 carbon atoms) and an aryl group having from 6 to 50 ring carbon atoms (preferably from 6 to 25, and more preferably from 6 to 18 ring carbon atoms); a disubstituted phosphoryl group having a substituent selected from an alkyl group having from 1 to 50 carbon atoms (preferably from 1 to 18, and more preferably from 1 to 8 carbon atoms) and an aryl group having from 6 to 50 ring carbon atoms (preferably from 6 to 25, and more preferably from 6 to 18 ring carbon atoms); an alkylsulfonyloxy group; an arylsulfonyloxy group; an alkylcarbonyloxy group; an arylcarbonyloxy group; a boron-containing group; a zinc-containing group; a tin-containing group; a silicon-containing group; a magnesium-containing group; a lithium-containing group; a hydroxyl group; an alkyl-substituted or aryl-substituted carbonyl group; a carboxyl group; a vinyl group; a (meth)acryloyl group; an epoxy group; and an oxetanyl group.

The substituents may be further substituted with the aforementioned arbitrary substituent. The substituents may form a ring by bonding plural substituents to each other.

In the expression “substituted or unsubstituted”, “unsubstituted” means that the group is not substituted with the substituent, but a hydrogen atom is bonded thereto.

In the aforementioned substituents, more preferred examples include a substituted or unsubstituted alkyl group having from 1 to 50 carbon atoms (preferably from 1 to 18, and more preferably from 1 to 8 carbon atoms), a substituted or unsubstituted cycloalkyl group having from 3 to 50 ring carbon atoms (preferably from 3 to 10, more preferably from 3 to 8, and further preferably 5 or 6 ring carbon atoms), a substituted or unsubstituted aryl group having from 6 to 50 ring carbon atoms (preferably from 6 to 25, and more preferably from 6 to 18 ring carbon atoms), a monosubstituted or disubstituted amino group having a substituent selected from a substituted or unsubstituted alkyl group having from 1 to 50 carbon atoms (preferably from 1 to 18, and more preferably from 1 to 8 carbon atoms) and a substituted or unsubstituted aryl group having from 6 to 50 ring carbon atoms (preferably from 6 to 25, and more preferably from 6 to 18 ring carbon atoms), a substituted or unsubstituted heteroaryl group having from 5 to 50 ring atoms (preferably from 5 to 24, and more preferably from 5 to 13 ring atoms), a halogen atom, and a cyano group.

In the description herein, the preferred definitions may be arbitrarily selected, and a combination of the preferred definitions may be more preferred.

### Compound (A)

In one embodiment of the present invention, a compound that is represented by the following general formulae (1) and (2), and contains 11 or more benzene rings in one molecule (which may be hereinafter abbreviated simply as a compound (A)) is provided.

The compound may have a glass transition temperature (Tg) that tends to be higher than a compound having 10 or less benzene rings, due to the presence of 11 or more benzene rings.

In an organic EL device, the device performance may be deteriorated due to heat generated on driving the device by applying an electric current thereto, and therefore the stability of the device on driving the device is expected to be enhanced by increasing the thermal stability of the compound (A) by increasing the glass transition temperature thereof through the presence of 11 or more benzene rings.

After coating to form a layer containing the compound (A), the layer may be dried by heating in the process of drying and removing an organic solvent used for coating. For retaining a stable thin film in the drying and heating process, the compound (A) preferably has high Tg.

In the case where the material for forming the film has low Tg, a phase transition from a solid phase to a liquid phase may occur on heating, and the shape of the thin film may not be retained due to flowage of the film. In this point of view, the number of benzene rings is preferably 12 or more, and more preferably 13 or more.

In the description herein, the number of benzene rings in the compound means the number of the aromatic 6-membered rings formed only of carbon and hydrogen present in one molecule of the compound. The similar structures contained in such structures as a condensed ring compound and a heterocyclic compound are also counted as the number of benzene rings. For example, naphthalene has a number of benzene rings of 2, carbazole has a number of benzene rings of 2, and quinazoline has a number of benzene rings of 1.

The compound (A) has the structure, in which the structure represented by the following general formula (2) is bonded to the structure represented by the following general formula (1), and therefore the compound (A) is preferred for applying to an organic EL device, layers of which are formed by a coating method, and is useful as a material for an organic electroluminescence device.

The structure represented by the general formula (2) having a larger molecular weight due to the substituents, such as the phenyl groups, is bonded to the structure represented by the general formula (1), and thereby the compound (A) has high Tg. Accordingly, in the case where a layer containing the compound (A) is coated and then dried under heating, the thin film can be stably formed.

One of the features of the structure represented by the general formula (2) is that R^{a} and R^{b} are bonded to the sp₃ carbon. An organic semiconductor material as represented by an organic EL material is formed of a molecular structure having a high planarity formed mainly of sp₂ carbon for achieving the charge transporting function. However, the group of compounds having such a molecular skeleton tends to be stable in a solid state due to the intermolecular interaction strongly acting, and in the case where a film is to be formed by coating, the material has considerably low solubility in an organic solvent and is difficult to dissolve in a desired organic solvent.

In one embodiment of the present invention, it has been found that the planarity of the molecule can be disturbed by introducing sp₃ carbon into the molecular skeleton, and selecting suitable substituents R^{a} and R^{b}. The disturbing the planarity may diminish the intermolecular interaction and thus enhances the solubility in an organic solvent.

According to the constitution, the compound can be dissolved in a desired organic solvent, and a thin film can be formed by an appropriate coating method.

In the case where the highest occupied molecular orbital (HOMO) and the lowest unoccupied molecular orbital (LUMO) of the structure represented by the general formula (2) have energy levels that are close to the HOMO and the LUMO of the structure represented by the general formula (1), there is a possibility that the molecular orbitals are largely spread to vary the ionization potential (Ip) and the electron affinity (Ea) of the structure represented by the general formula (1). By combining the structure represented by the general formula (2) to the structure represented by the general formula (1) in a manner avoiding the overlap of the HOMO and the LUMO, the spreading the molecular orbitals due to the structure represented by the general formula (2) bonded can be prevented, and the intermolecular interaction can be diminished while maintaining the intended properties of the structure represented by the general formula (1):

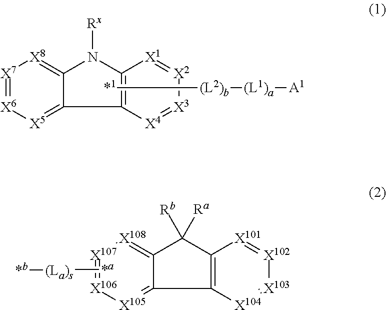

wherein

in the general formula (1),
A¹ represents a substituted or unsubstituted nitrogen-containing heteroaromatic group having from 5 to 30 ring atoms;
L¹ and L² each independently represent a substituted or unsubstituted aromatic hydrocarbon group having from 6 to 30 ring carbon atoms, a substituted or unsubstituted heterocyclic group having from 5 to 30 ring atoms, or a group containing from 2 to 4 of the groups bonded to each other;
a and b each independently represent 0 or 1, provided that when a is 0, (L¹)₀ represents a single bond, and when b is 0, (L²)₀ represents a single bond;
R^{x} represents a hydrogen atom or a substituent;
X¹ to X⁸ each represent C(R¹) to C(R⁸) or a nitrogen atom; and
R¹ to R⁸ each independently represent a hydrogen atom or a substituent, in which adjacent substituents may be bonded to each other to form a ring,
provided that any one of R¹ to R⁴ and R^{x} represents a single bond bonded to L² at the position of *¹,

in the general formula (2),
Lₐ represents a substituted or unsubstituted aromatic hydrocarbon group having from 6 to 30 ring carbon atoms or a substituted or unsubstituted heterocyclic group having from 5 to 30 ring atoms;
s represents 0 or 1, provided that when s is 0, (Lₐ)₀ represents a single bond;
X¹⁰¹ to X¹⁰⁸ each represent C(R¹⁰¹) to C(R¹⁰⁸) or a nitrogen atom, and R¹⁰¹ to R¹⁰⁸ each independently represent a hydrogen atom or a substituent, in which adjacent substituents may be bonded to each other to form a ring, provided that any one of R¹⁰⁵ to R¹⁰⁸ represents a single bond directly bonded to Lₐ at the position of *^{a}; and
R^{a} and R^{b} each independently represent a hydrogen atom, an alkyl group having from 1 to 10 carbon atoms, an aryl group having from 6 to 14 ring carbon atoms, or a heteroaryl group having from 5 to 14 ring atoms, in which R^{a} and R^{b} may be bonded to each other to form a ring, and R^{a} and R¹⁰¹ and R^{b} and R¹⁰⁸ each independently may be bonded to each other to form a ring,

the group represented by the formula (2) is bonded to at least one of L¹, L², R¹ to R⁸, and R^{x} at the position of *^{b}, in which any one of R¹ to R⁸, and R^{x} that is bonded to the group represented by the formula (2) represents a single bond, and

when plural groups each represented by the formula (2) are present, the groups may be the same as or different from each other.

### Description of Groups in General Formula (1)

In the general formula (1), the nitrogen-containing heteroaromatic group represented by A¹ has from 5 to 30 ring atoms, and preferably from 6 to 20, and more preferably from 6 to 14 ring atoms. The nitrogen-containing heteroaromatic group is preferably any one of a monocyclic ring, a condensed ring constituted by two rings, and a condensed ring constituted by three rings.

The number of a nitrogen atom contained in the nitrogen-containing heteroaromatic group is preferably from 1 to 3, and more preferably 2 or 3. In particular, in the case where the nitrogen-containing heteroaromatic group is a monocyclic ring, the number of a nitrogen atom contained is preferably 2 or 3, and more preferably 3, and in the case where the nitrogen-containing heteroaromatic group is a condensed ring constituted by two rings or three rings, the number of a nitrogen atom contained is preferably 2. Further, the nitrogen-containing heteroaromatic group preferably contains only a nitrogen atom as a hetero atom. On the other hand, the nitrogen-containing heteroaromatic group may contain a hetero atom other than a nitrogen atom, such as an oxygen atom, a sulfur atom, a silicon atom, and a selenium atom.

Specific examples of the nitrogen-containing heteroaromatic group represented by A¹ include residual groups derived from compounds selected from pyrrole, pyridine, pyridazine, pyrimidine, pyrazine, triazine, imidazole, pyrazole, oxadiazole, thiadiazole, triazole, tetrazole, indole, isoindole, indolizine, quinolizine, quinoline, isoquinoline, naphthyridine, cinnoline, phthalazine, quinazoline, benzo[f]quinazoline, benzo[h]quinazoline, azafluoranthene, diazafluoranthene, quinoxaline, benzimidazole, indazole, carbazole, biscarbazole, phenanthridine, acridine, phenanthroline, phenazine, azatriphenylene, diazatriphenylene, hexaazatriphenylene, azacarbazole, azadibenzofuran, azadibenzothiophene, and dinaphtho[2′,3′:2,3:2′,3′:6,7]carbazole. The residual group referred herein means a mon- or poly-valent group obtained by eliminating one hydrogen atom from the compound.

The nitrogen-containing heteroaromatic group is preferably residual groups of the compounds from the following groups among the aforementioned compounds.

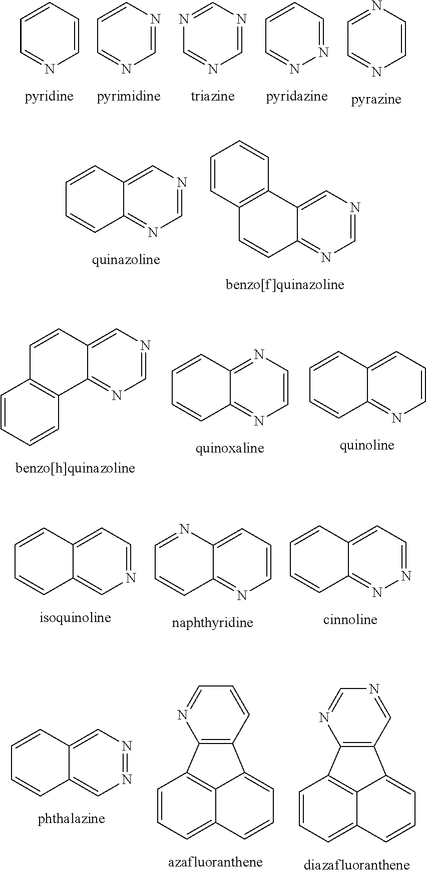

In one embodiment of the present invention, the nitrogen-containing heteroaromatic group represented by A¹ is preferably a residual group derived from a nitrogen-containing heterocyclic ring represented by the following general formula (N1).

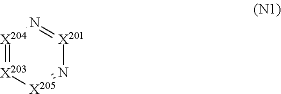

wherein in the general formula (N1), X²⁰¹ to X²⁰⁴ each represent C(R²⁰¹) to C(R²⁰⁴) or a nitrogen atom. R²⁰¹ to R²⁰⁴ each independently represent a hydrogen atom or a substituent, in which two selected from R²⁰² to R²⁰⁴ may be bonded to each other to form a ring.

In one embodiment of the present invention, the nitrogen-containing heteroaromatic hydrocarbon group represented by A¹ is preferably a residual group derived from a nitrogen-containing heterocyclic ring represented by any one of the following general formulae (N2) to (N6).

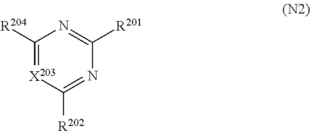

wherein in the general formula (N2), X²⁰³ represents C(R²⁰³) or a nitrogen atom. R²⁰¹ to R²⁰⁴ each independently represent a hydrogen atom or a substituent, in which two selected from R²⁰² to R²⁰⁴ may be bonded to each other to form a ring.

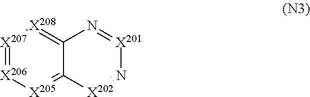

wherein in the general formula (N3), X²⁰¹, X²⁰², and X²⁰⁵ to X²⁰⁸ each represent C(R²⁰¹), C(R²⁰²), C(R²⁰⁵) to C(R²⁰⁸), or a nitrogen atom. R²⁰¹, R²⁰², and R²⁰⁵ to R²⁰⁸ each independently represent a hydrogen atom or a substituent, in which two selected from R²⁰², and R²⁰⁵ to R²⁰⁸ may be bonded to each other to form a ring.

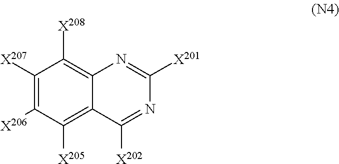

wherein in the general formula (N4), R²⁰¹, R²⁰², and R²⁰⁵ to R²⁰⁸ each independently represent a hydrogen atom or a substituent, and two selected from R²⁰², and R²⁰⁵ to R²⁰⁸ may be bonded to each other to form a ring.

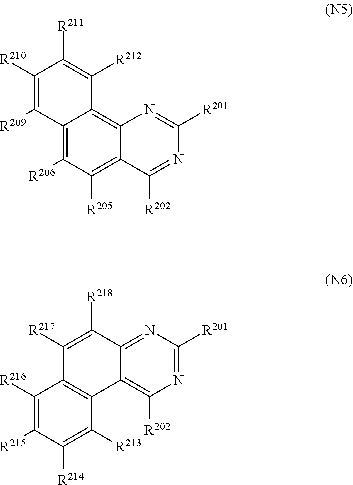

wherein in the general formulae (N5) and (N6), R²⁰¹, R²⁰², and R²⁰⁵ to R²¹⁶ each independently represent a hydrogen atom or a substituent.

In the general formula (1), the aromatic hydrocarbon group represented by L¹ and L² has from 6 to 30 ring carbon atoms, and preferably from 6 to 18, more preferably from 6 to 13, further preferably from 6 to 12, and particularly preferably from 6 to 10 ring carbon atoms.

The aromatic hydrocarbon group represented by L¹ and L² is preferably a divalent to tetravalent residual group derived from any one of compounds represented by the following formulae, and in one embodiment of the present invention, at least one (preferably all) of L¹ and L² is preferably a divalent to tetravalent residual group derived from any one of compounds represented by the following formulae.

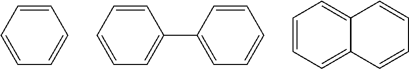

wherein in the formulae, the carbon atoms each may have a substituent.

The aromatic hydrocarbon group represented by L¹ and L² is preferably any one of groups represented by the following formulae, and in one embodiment of the present invention, at least one (preferably all) of L¹ and L² is preferably any one of groups represented by the following formulae.

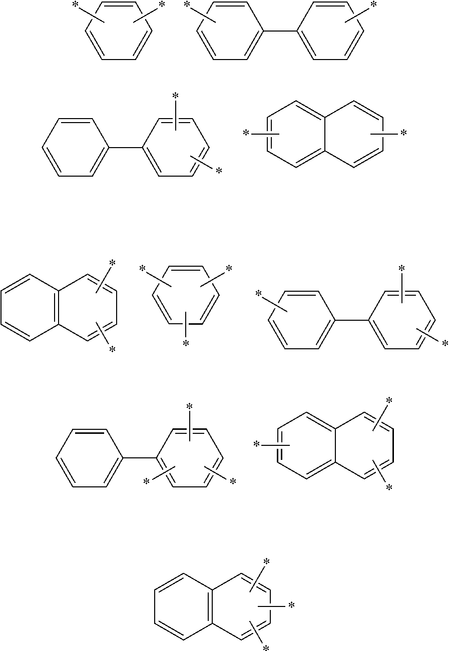

wherein in the formulae, * represents the bonding position, and the carbon atoms at the positions other than the bonding positions may have a substituent.

Among the above groups, the aromatic hydrocarbon group represented by L¹ and L² is more preferably any one of groups represented by the following formulae, and at least one (preferably all) of L¹ and L² is more preferably any one of groups represented by the following formulae.

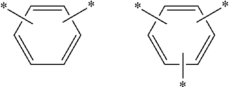

wherein in the formulae, * represents the bonding position, and the carbon atoms at the positions other than the bonding positions may have a substituent.

Among the above groups, the aromatic hydrocarbon group represented by L¹ and L² is further preferably any one of groups represented by the following formulae, and at least one (preferably all) of L¹ and L² is further preferably any one of groups represented by the following formulae.

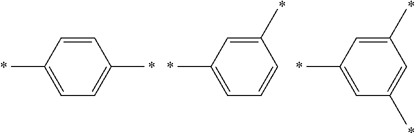

wherein in the formulae, * represents the bonding position, and the carbon atoms at the positions other than the bonding positions may have a substituent.

Examples of the aromatic hydrocarbon group represented by L¹ and L² also include groups represented by the following formulae, in addition to the groups described above.

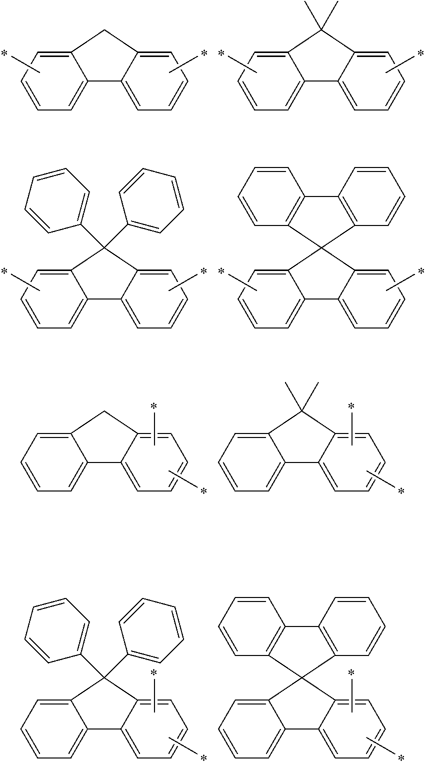

wherein in the formulae, * represents the bonding position, and the carbon atoms at the positions other than the bonding positions may have a substituent.

Specific examples of the case where the aromatic hydrocarbon group represented by L¹ and L² is a divalent group include groups represented by the following formulae.

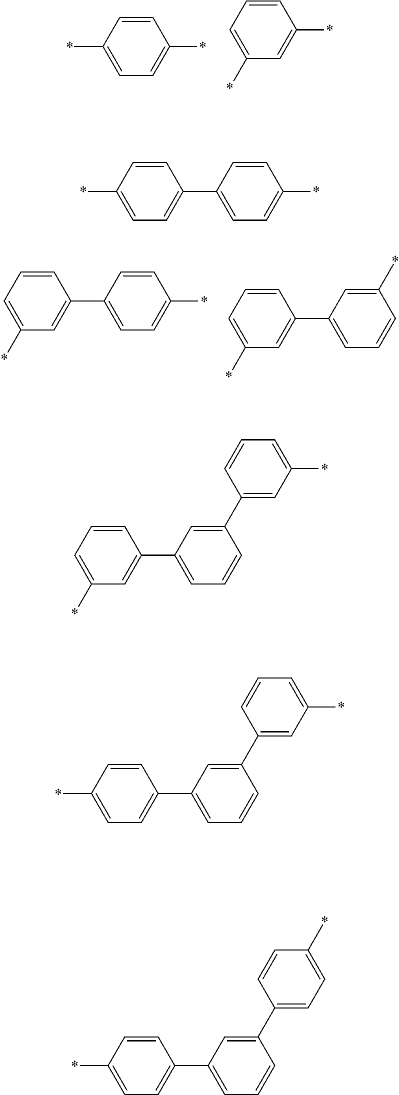

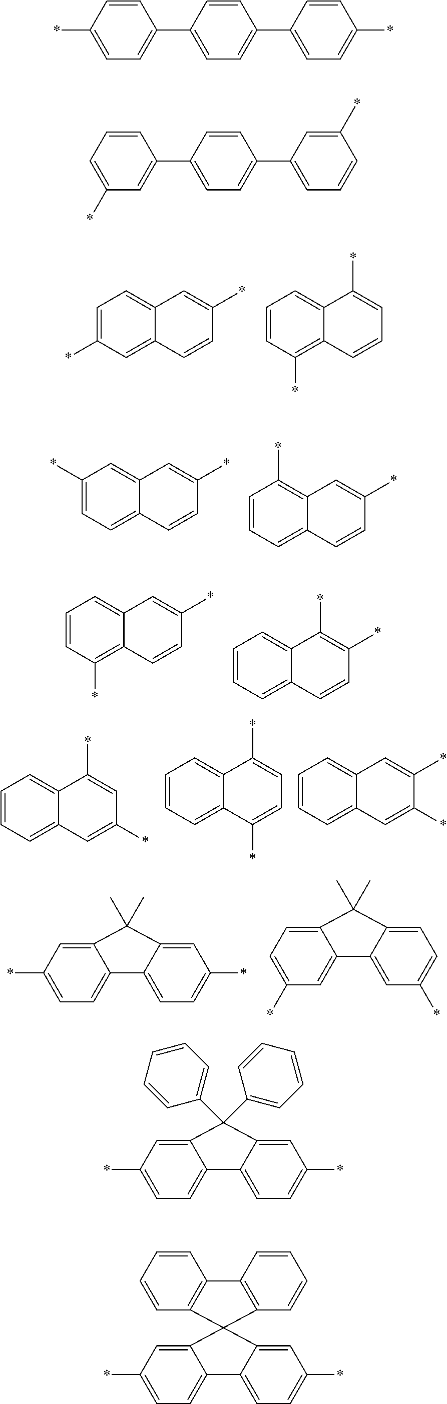

wherein in the formulae, * represents the bonding position, and the carbon atoms at the positions other than the bonding positions may have a substituent.

The heterocyclic group represented by L¹ and L² has from 5 to 30 ring atoms, and preferably from 5 to 18, more preferably from 5 to 13, and particularly preferably from 5 to 10 ring atoms. Examples of the heterocyclic group include a residual group of a nitrogen-containing heterocyclic compound, such as pyrrole, pyridine, imidazopyridine, pyrazole, triazole, tetrazole, indole, isoindole and carbazole; a residual group of an oxygen-containing heterocyclic compound, such as furan, benzofuran, isobenzofuran, dibenzofuran, oxazole, oxadiazole, benzoxazole, benzonaphthofuran and dinaphthofuran; and a residual group of a sulfur-containing heterocyclic compound, such as thiophene, benzothiophene, dibenzothiophene, thiazole, thiadiazole, benzothiazole, benzonaphthothiophene and dinaphthothiophene.

The group containing from 2 to 4 of the groups bonded to each other represented by L¹ and L² is a group that contains from 2 to 4 groups of a substituted or unsubstituted aromatic hydrocarbon group having from 6 to 30 ring carbon atoms and a substituted or unsubstituted heterocyclic group having from 5 to 30 ring atoms, which are bonded to each other. In this case, the order of bonding is not particularly limited.

Among the aforementioned groups, L¹ and L² each preferably represent a substituted or unsubstituted aromatic hydrocarbon group having from 6 to 30 ring carbon atoms. The preferred ones of the aromatic hydrocarbon group are described above.

In the general formula (1), a and b each independently represent 0 or 1. When a is 0, (L¹)₀ represents a single bond. When b is 0, (L²)₀ represents a single bond.

In the general formula (1), R^{x} represents a hydrogen atom or a substituent.

In the general formula (1), X¹ to X⁸ each represent C(R¹) to C(R⁸) or a nitrogen atom, and R¹ to R⁸ each independently represent a hydrogen atom or a substituent, in which adjacent substituents may be bonded to each other to form a ring.

Any one of R¹ to R⁴ and R^{x} represents a single bond bonded to L² at the position of *¹.

X¹ to X⁸ are preferably C(R¹) to C(R⁸), and R¹ to R⁸ that are not bonded to the group represented by the general formula (2) described later each preferably represent a hydrogen atom.

In the case where R^{x} is not bonded to L² at the position of *¹, and the case where R^{x} is not bonded to the group represented by the general formula (2), R^{x} more preferably represents a substituent, and further preferably represents a substituted or unsubstituted aryl group having from 6 to 30 ring carbon atoms or a substituted or unsubstituted heteroaryl group having from 5 to 30 ring atoms.

In the case where R^{x} represents an aryl group, the aryl group preferably has from 6 to 30 ring carbon atoms, and more preferably from 6 to 18, further preferably from 6 to 13, still further preferably from 6 to 12, and particularly preferably from 6 to 10 ring carbon atoms.

Examples of the aryl group include a phenyl group, a naphthyl group (such as a 1-naphthyl group and a 2-naphthyl group), a naphthylphenyl group, a biphenylyl group, a terphenylyl group, a quarterphenylyl group, a quinquephenylyl group, an acenaphthylenyl group, an anthryl group, a benzoanthryl group, an aceanthryl group, a phenanthryl group, a benzophenanthryl group, a phenalenyl group, a fluorenyl group (such as a 9,9-dimethylfluorenyl group, a 9,9-diphenylfluorenyl group and a 9,9′-spirobifluorenyl group), a benzofluorenyl group, a dibenzofluorenyl group, a picenyl group, a pentaphenyl group, a pentacenyl group, a pyrenyl group, a chrysenyl group, a benzochrysenyl group, a s-indanyl group, an as-indanyl group, a fluoranthenyl group, a benzofluoranthenyl group, a tetracenyl group, a triphenylenyl group, a benzotriphenylenyl group, a perylenyl group, a coronyl group, and a dibenzoanthryl group. These groups include isomers thereof when the isomers are present.

In one embodiment of the present invention, R^{x} preferably represents a condensed ring group having from 10 to 30 (preferably from 10 to 20, and more preferably from 10 to 14) ring carbon atoms in the aryl group. Examples of the condensed ring group include a naphthyl group (such as a 1-naphthyl group and a 2-naphthyl group), an acenaphthylenyl group, an anthryl group, a benzoanthryl group, an aceanthryl group, a phenanthryl group, a benzophenanthryl group, a phenalenyl group, a fluorenyl group (such as a 9,9-dimethylfluorenyl group, a 9,9-diphenylfluorenyl group and a 9,9′-spirobifluorenyl group), a benzofluorenyl group, a dibenzofluorenyl group, a picenyl group, a pentacenyl group, a pyrenyl group, a chrysenyl group, a benzochrysenyl group, a fluoranthenyl group, a benzofluoranthenyl group, a tetracenyl group, a perylenyl group, a coronyl group, and a dibenzoanthryl group.

More specifically, the aryl group is preferably an aryl group selected from the following groups.

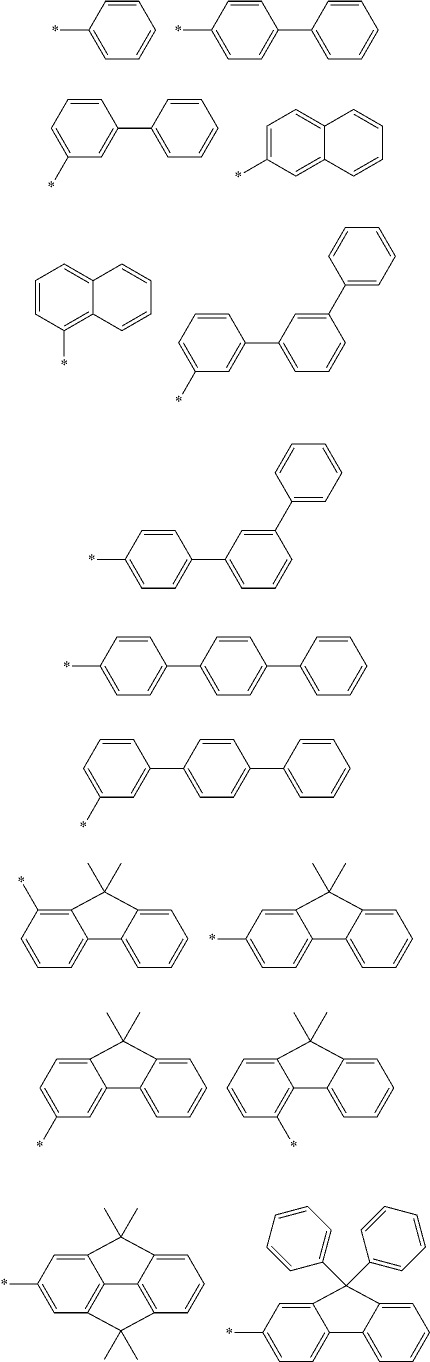

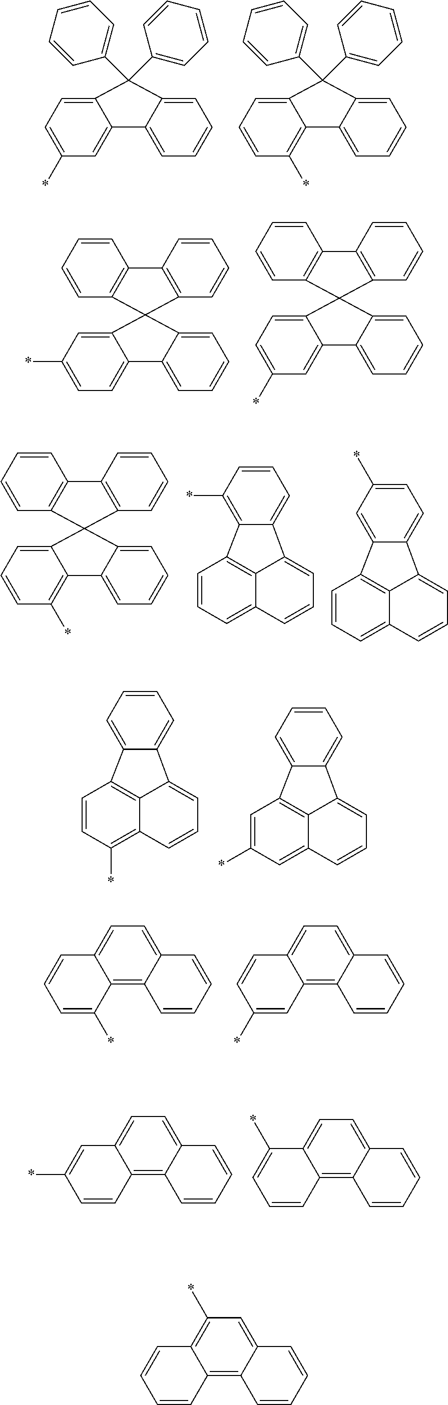

wherein in the formulae, * represents the bonding position, and the carbon atoms at the positions other than the bonding positions may have a substituent.

In the case where R^{x} represents a heteroaryl group, the heteroaryl group preferably has from 5 to 30 ring atoms, and more preferably from 5 to 20, further preferably from 5 to 14, and still further preferably from 5 to 10 ring atoms.

The heteroaryl group contains at least one, preferably from 1 to 5, more preferably from 1 to 3, and further preferably 1 to 2, the same or different hetero atoms. Examples of the hetero atom include a nitrogen atom, a sulfur atom, an oxygen atom, and a phosphorus atom, from which the hetero atom is preferably selected.

Examples of the heteroaryl group include a pyrrolyl group, a furyl group, a thienyl group, a pyridyl group, a pyridazinyl group, a pyrimidinyl group, a pyrazinyl group, a triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, a tetrazolyl group, an indolyl group, an isoindolyl group, a benzofuranyl group, an isobenzofuranyl group, a benzothiophenyl group, an isobenzothiophenyl group, an indolizinyl group, a quinolizinyl group, a quinolyl group, an isoquinolyl group, a cinnolinyl group, a phthalazinyl group, a quinazolinyl group, a quinoxalinyl group, a benzimidazolyl group, a benzoxazolyl group, a benzothiazolyl group, an indazolyl group, a benzoisoxazolyl group, a benzoisothiazolyl group, a dibenzofuranyl group, a dibenzothiophenyl group, a carbazolyl group, a biscarbazolyl group, a phenanthridinyl group, an acridinyl group, a phenanthrolinyl group, a phenazinyl group, a phenothiazinyl group, a phenoxazinyl group, an azatriphenylenyl group, a diazatriphenylenyl group, a xanthenyl group, an azacarbazolyl group, an azadibenzofuranyl group, an azadibenzothiophenyl group, a benzofuranobenzothiophenyl group, a benzothienobenzothiophenyl group, a dibenzofuranonaphthyl group, a dibenzothienonaphthyl group, a dinaphthothienothiophenyl group, and a dinaphtho[2′,3′:2,3:2′,3′:6,7]carbazolyl group.

R^{x} preferably represents a substituted or unsubstituted aryl group having from 6 to 30 ring carbon atoms. The preferred ones of the aryl group are described above.

### Description of Groups in General Formula (2)

In the general formula (2), Lₐ represents a substituted or unsubstituted aromatic hydrocarbon group having from 6 to 30 ring carbon atoms or a substituted or unsubstituted heterocyclic group having from 5 to 30 ring atoms. The descriptions of the aromatic hydrocarbon group and the heterocyclic group represented by Lₐ are the same as the case of L¹ and L², and preferred ones thereof are also the same. Among these, the aromatic hydrocarbon group represented by Lₐ is preferably a phenylene group or a naphthylene group, and more preferably a phenylene group.

In the general formula (2), s represents 0 or 1, and preferably 0, provided that when s is 0, (Lₐ)₀ represents a single bond.

Preferred examples of the case where Lₐ represents a substituted or unsubstituted aromatic hydrocarbon group having from 6 to 30 ring carbon atoms or a substituted or unsubstituted heterocyclic group having from 5 to 30 ring atoms are the same as above, and in the general formula (2), the case where s is 0, and (Lₐ)₀ represents a single bond is further preferred.

X¹⁰¹ to X¹⁰⁸ each represent C(R¹⁰¹) to C(R¹⁰⁸) or a nitrogen atom, and R¹⁰¹ to R¹⁰⁸ each independently represent a hydrogen atom or a substituent, in which adjacent substituents may be bonded to each other to form a ring, provided that any one of R¹⁰⁵ to R¹⁰⁸ represents a single bond directly bonded to Lₐ at the position of *^{a}.

R^{a} and R^{b} each independently represent a hydrogen atom, an alkyl group having from 1 to 10 carbon atoms, an aryl group having from 6 to 14 ring carbon atoms, or a heteroaryl group having from 5 to 14 ring atoms, in which R^{a} and R^{b} may be bonded to each other to form a ring, and R^{a} and R¹⁰¹, and R^{b} and R¹⁰⁸ each independently may be bonded to each other to form a ring.

The group represented by the formula (2) is bonded to at least one of L¹, L², R¹ to R⁸, and R^{x} at the position of *^{b}, in which any one of R¹ to R⁸, and R^{x} that is bonded to the group represented by the formula (2) represents a single bond.

When plural groups each represented by the formula (2) are present, the groups may be the same as or different from each other.

More specifically, the structure represented by the general formula (2) is preferably an aryl group selected from the following groups.

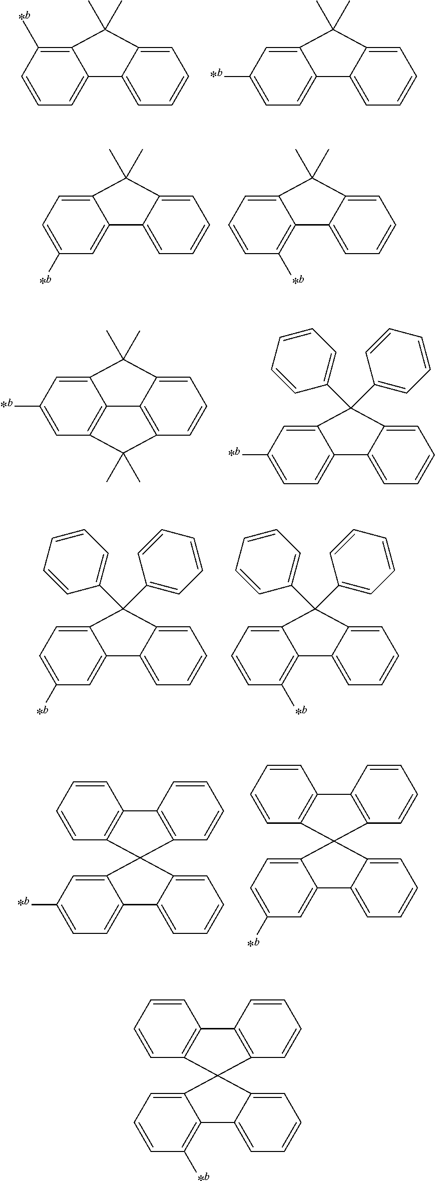

wherein in the formulae, *^{b} is *^{b} in the general formula (2) and represents the bonding position. In the formulae, the carbon atoms at the positions other than the bonding positions may have a substituent.

### General Formulae of Preferred Compounds in Compound (A)

General formulae of preferred compounds in the compound (A) of the present invention are described below. The definitions of the groups are the same as in the general formulae (1) and (2) unless otherwise expressly indicated, and preferred ones thereof are also the same.

In one embodiment of the compound (A) of the present invention, the structure represented by the general formulae (1) and (2) is preferably a structure represented by the following general formulae (1a) and (2a):

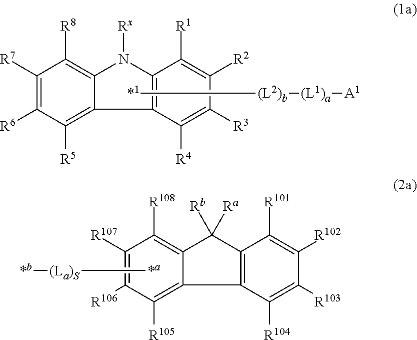

wherein

in the general formula (1a), A¹, L¹, L², a, b, R¹ to R⁸, and R^{x} have the same meanings as in the formula (1), and preferred ones thereof are also the same, and

in the general formula (2a), Lₐ, s, R¹⁰¹ to R¹⁰⁸, R^{a}, and R^{b} have the same meanings as in the formula (2), and preferred ones thereof are also the same.

In one embodiment of the compound (A) of the present invention, the structure represented by the general formulae (1) and (2) is preferably a structure represented by the following general formulae (1a-1) and (2a):

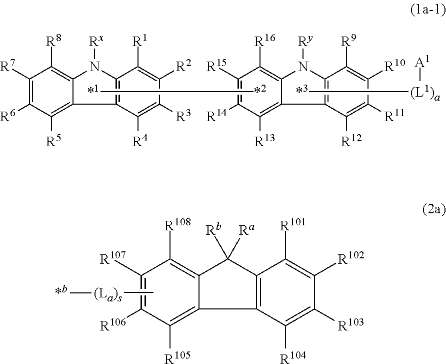

wherein

in the general formula (1a-1),
A¹, L¹, and a have the same meanings as in the formula (1a), and preferred ones thereof are also the same;
R^{x} and R^{y} each independently represent a hydrogen atom or a substituent; and
R¹ to R¹⁶ each independently represent a hydrogen atom or a substituent, in which adjacent substituents may be bonded to each other to form a ring,
provided that any one of R¹ to R⁴, and R^{x} represents a single bond bonded to *¹, any one of R¹³ to R¹⁶ represents a single bond bonded to *², and any one of R⁹ to R¹², and R^{y} represents a single bond bonded to L¹ at the position of *³, and

in the general formula (2a),
Lₐ, s, R¹⁰¹ to R¹⁰⁸, R^{a}, and R^{b} have the same meanings as above, and preferred ones thereof are also the same,

provided that the group represented by the formula (2a) is bonded to at least one of L¹, R¹ to R¹⁶, R^{x}, and R^{y} at the position of *^{b}.

In one embodiment of the compound (A) of the present invention, the structure represented by the general formulae (1a-1) and (2) is preferably a structure represented by the following general formulae (1a-2) and (2a):

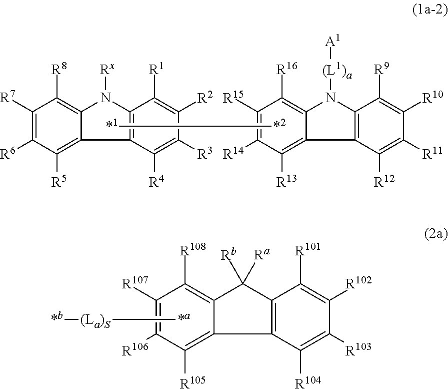

wherein

in the general formula (1a-2), A¹, L¹, a, R¹ to R¹⁶, and R^{x} have the same meanings as in the formula (1a-1), and preferred ones thereof are also the same, and

in the general formula (2a), Lₐ, s, R¹⁰¹ to R¹⁰⁸, R^{a}, and R^{b} have the same meanings as above, and preferred ones thereof are also the same,

provided that the group represented by the formula (2a) is bonded to at least one of L¹, R¹ to R¹⁶, and R^{x} at the position of *^{b}.

In one embodiment of the compound (A) of the present invention, the structure represented by the general formulae (1a-1) and (2) is more preferably a structure represented by the following general formulae (1a-3) and (2a):

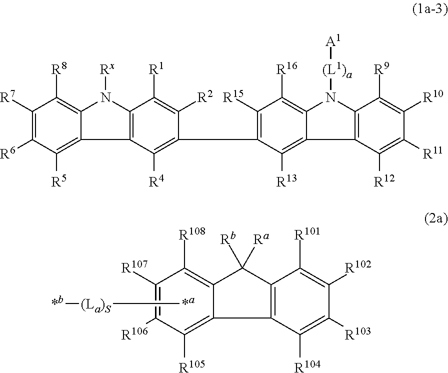

wherein

in the general formula (1a-3), A¹, L¹, a, R¹ to R¹⁶, and R^{x} have the same meanings as in the formula (1a-1), and preferred ones thereof are also the same, and

in the general formula (2a), Lₐ, s, R¹⁰¹ to R¹⁰⁸, R^{a}, and R^{b} have the same meanings as above, and preferred ones thereof are also the same,

provided that the group represented by the formula (2a) is bonded to at least one of L¹, R¹ to R¹⁶, and R^{x} at the position of *^{b}.

In one embodiment of the compound (A) of the present invention, the structure represented by the general formulae (1) and (2) bonded to each other is preferably a structure represented by the following general formula (1b):

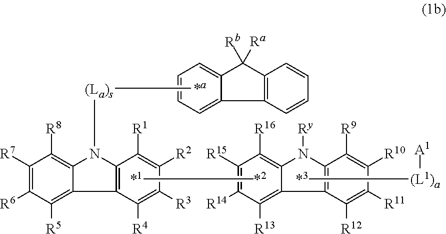

wherein in the general formula (1b), A¹, Lₐ, L¹, a, s, R¹ to R¹⁶, R^{y}, R^{a}, and R^{b} have the same meanings as in the formulae (1a-1) and (2a), and preferred ones thereof are also the same.

In one embodiment of the compound (A) of the present invention, the structure represented by the general formula (1b) is preferably a structure represented by the following general formula (1b-1):

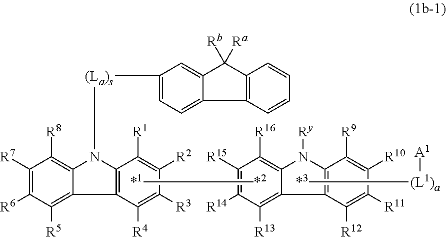

wherein in the general formula (1b-1), A¹, Lₐ, L¹, a, s, R¹ to R¹⁶, R^{y}, R^{a}, and R^{b} have the same meanings as in the formulae (1a-1) and (2a), and preferred ones thereof are also the same.

In one embodiment of the compound (A) of the present invention, the structure represented by the general formula (1b) is more preferably a structure represented by the following general formula (1b-2):

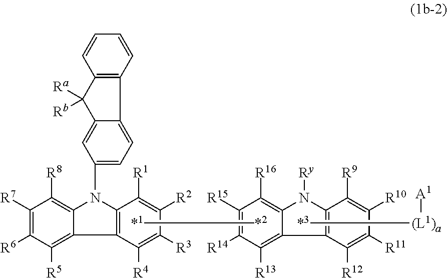

wherein in the general formula (1b-2), A¹, L¹, a, R¹ to R¹⁶, R^{y}, R^{a}, and R^{b} have the same meanings as in the formulae (1a-1) and (2a), and preferred ones thereof are also the same.

In one embodiment of the compound (A) of the present invention, the structure represented by the general formula (1b) is further preferably a structure represented by the following general formula (1b-3):

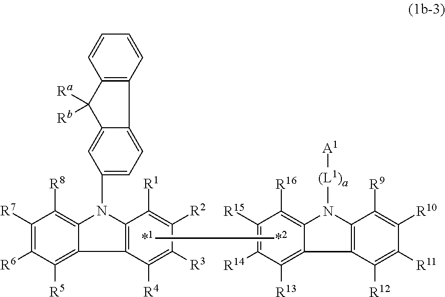

wherein in the general formula (1b-3), A¹, L¹, a, R¹ to R¹⁶, R^{a}, and R^{b} have the same meanings as in the formulae (1a-1) and (2a), and preferred ones thereof are also the same.

In one embodiment of the compound (A) of the present invention, the structure represented by the general formula (1b) is still further preferably a structure represented by the following general formula (1b-4):

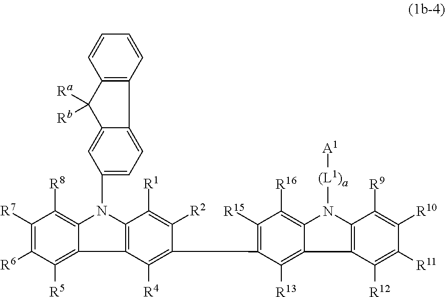

wherein in the general formula (1b-4), A¹, L¹, a, R¹, R², R⁴ to R¹³, R¹⁵, R¹⁶, R^{a}, and R^{b} have the same meanings as in the formulae (1a-1) and (2a), and preferred ones thereof are also the same.

In one embodiment of the compound (A) of the present invention, the structure represented by the general formulae (1) and (2) is preferably a structure represented by the following general formulae (1a-a) and (2a):

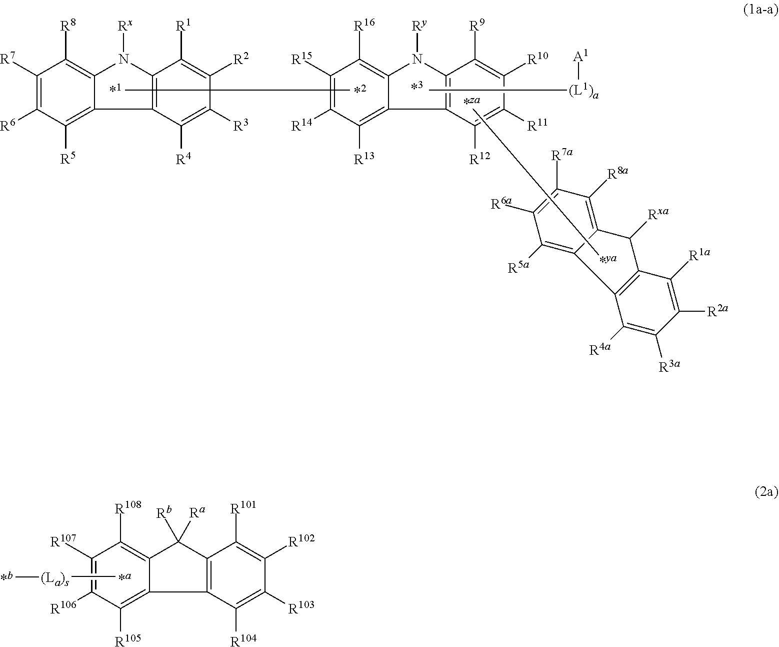

wherein

in the general formula (1a-a),
A¹, L¹, a, R¹ to R¹⁶, R^{x}, and R^{y} have the same meanings as in the formula (1a-1);
R^{xa} represents a hydrogen atom or a substituent; and
R^{1a} to R^{8a} each independently represent a hydrogen atom or a substituent, in which adjacent substituents may be bonded to each other,
provided that any one of R^{5a} to R^{8a}, and R^{xa} represents a single bond bonded to *^{ya}, and any one of R⁹ to R¹² represents a single bond bonded to *^{za}, and

in the general formula (2a),
Lₐ, s, R¹⁰¹ to R¹⁰⁸, R^{a}, and R^{b} have the same meanings as above, and preferred ones thereof are also the same,

provided that the group represented by the formula (2a) is bonded to at least one of L¹, R¹ to R¹⁶, R^{1a} to R^{8a}, R^{x}, R^{y} and R^{xa} at the position of *^{b}.

In one embodiment of the compound (A) of the present invention, the structure represented by the general formulae (1) and (2) is preferably a structure represented by the following general formulae (1-i) and (2a):

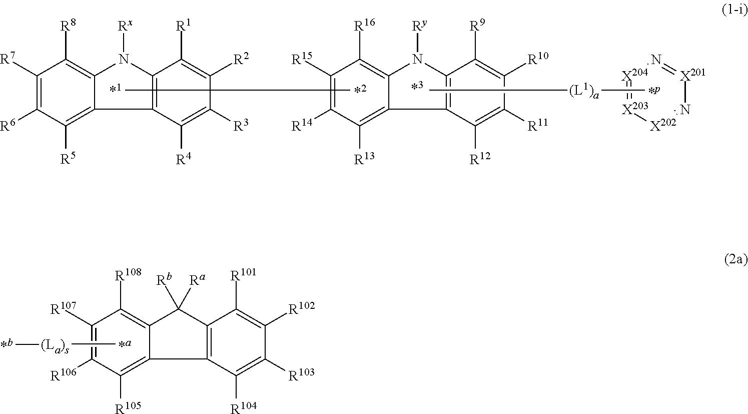

wherein

in the general formula (1-i),
L¹, a, R¹ to R¹⁶, R^{x}, and R^{y} have the same meanings as in the formula (1a-1), and preferred ones thereof are also the same; and
X²⁰¹ to X²⁰⁴ each represent C(R²⁰¹) to C(R²⁰⁴) or a nitrogen atom, and R²⁰¹ to R²⁰⁴ each independently represent a hydrogen atom or a substituent, in which adjacent substituents may be bonded to each other, provided that any one of R²⁰¹ to R²⁰⁴ represents a single bond directly bonded to L¹ at the position of *^{p}, and

in the general formula (2a),
Lₐ, s, R¹⁰¹ to R¹⁰⁸, R^{a}, and R^{b} have the same meanings as above, and preferred ones thereof are also the same.

In one embodiment of the compound (A) of the present invention, the structure represented by the general formulae (1) and (2) is preferably a structure represented by the following general formulae (1-ii) and (2a):

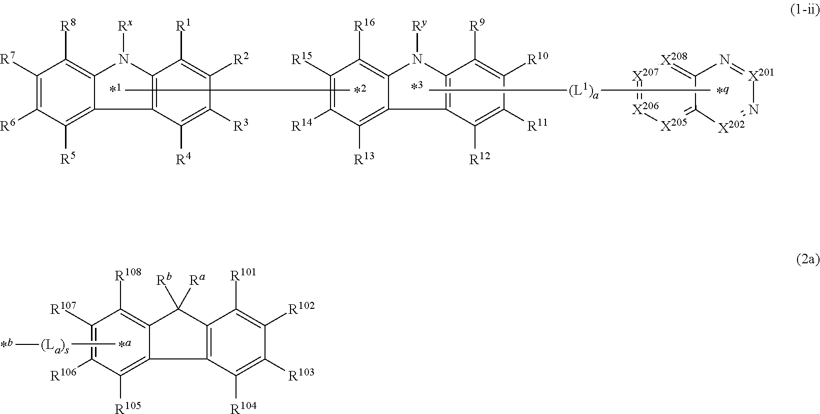

wherein

in the general formula (1-ii),
L¹, a, R¹ to R¹⁶, R^{x}, and R^{y} have the same meanings as in the formula (1a-1), and preferred ones thereof are also the same; and
X²⁰¹, X²⁰², and X²⁰⁵ to X²⁰⁸ each represent C(R²⁰¹), C(R²⁰²), and C(R²⁰⁵) to C(R²⁰⁸) or a nitrogen atom, and R²⁰¹, R²⁰², and R²⁰⁵ to R²⁰⁸ each independently represent a hydrogen atom or a substituent, in which adjacent substituents may be bonded to each other, provided that any one of R²⁰¹, R²⁰², and R²⁰⁵ to R²⁰⁸ represents a single bond directly bonded to L¹ at the position of *^{q}, and

in the general formula (2a),
Lₐ, s, R¹⁰¹ to R¹⁰⁸, R^{a}, and R^{b} have the same meanings as above, and preferred ones thereof are also the same.

In one embodiment of the compound (A) of the present invention, the structure represented by the general formulae (1) and (2) is preferably a structure represented by the following general formulae (1-iii) and (2a):

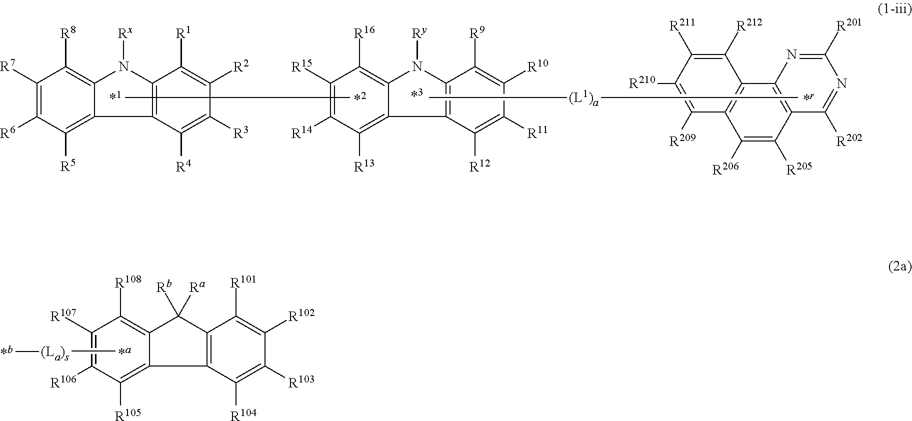

wherein

in the general formula (1-iii),
L¹, a, R¹ to R¹⁶, R^{x}, and R^{y} have the same meanings as in the formula (1a-1), and preferred ones thereof are also the same; and
R²⁰¹, R²⁰², R²⁰⁵, R²⁰⁶, and R²⁰⁹ to R²¹² each independently represent a hydrogen atom or a substituent, in which adjacent substituents may be bonded to each other, provided that any one of R²⁰¹, R²⁰², R²⁰⁵, R²⁰⁶, and R²⁰⁹ to R²¹² represents a single bond directly bonded to L¹ at the position of *^{r}, and

in the general formula (2a),
Lₐ, s, R¹⁰¹ to R¹⁰⁸, R^{a}, and R^{b} have the same meanings as above, and preferred ones thereof are also the same.

In one embodiment of the compound (A) of the present invention, the structure represented by the general formulae (1) and (2) is preferably a structure represented by the following general formulae (1-iv) and (2a):

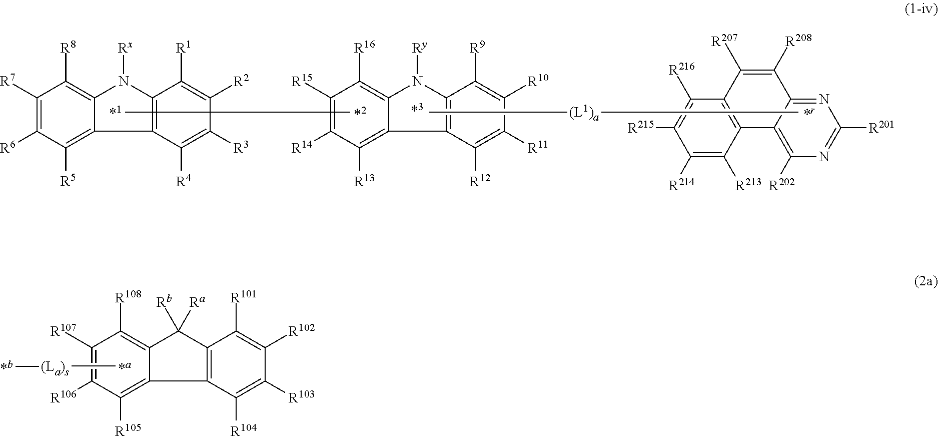

wherein

in the general formula (1-iv),
L¹, a, R¹ to R¹⁶, R^{x}, and R^{y} have the same meanings as in the formula (1a-1), and preferred ones thereof are also the same; and
R²⁰¹, R²⁰², R²⁰⁷, R²⁰⁸, and R²¹³ to R²¹⁶ each independently represent a hydrogen atom or a substituent, in which adjacent substituents may be bonded to each other, provided that any one of R²⁰¹, R²⁰², R²⁰⁷, R²⁰⁸, and R²¹³ to R²¹⁶ represents a single bond directly bonded to L¹ at the position of *^{r}, and

in the general formula (2a),
Lₐ, s, R¹⁰¹ to R¹⁰⁸, R^{a}, and R^{b} have the same meanings as above, and preferred ones thereof are also the same.

### Compound (B)

In one embodiment of the present invention, a compound that is represented by the following general formulae (3) and (4) (which may be hereinafter abbreviated simply as a compound (B)) is provided. The compound (B) has a structure containing the structure represented by the following general formula (4) that is bonded to the structure represented by the following general formula (3), and therefore the compound (B) can be formed into a layer by a coating method, is a compound that is capable of addressing the demand of the enhancement of various characteristics (such as the electron transporting capability, the hole transporting capability, the light emission efficiency and the lifetime) of an organic EL device, and is useful as a material for an organic electroluminescence device.

The compound (B) may include a compound that has the same structure as the compound (A). In one embodiment of the present invention, the characteristics of the structure represented by the following general formula (4) and the reason why the structure represented by the following general formula (4) is bonded to the following general formula (3) are the same as the characteristics of the structure represented by the general formula (2) and the reason why the structure represented by the general formula (2) is bonded to the general formula (1) described above.

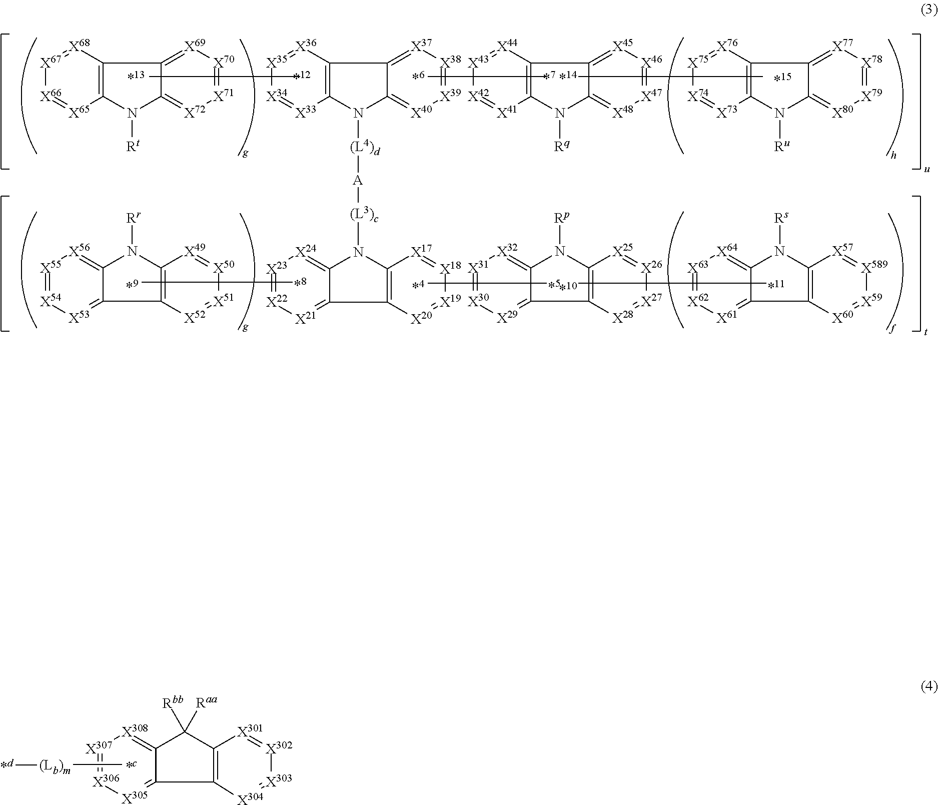

wherein

in the general formula (3),
A² represents a substituted or unsubstituted nitrogen-containing heteroaromatic group having from 5 to 30 ring atoms;
L³ and L⁴ each independently represent a substituted or unsubstituted aromatic hydrocarbon group having from 6 to 30 ring carbon atoms, a substituted or unsubstituted heterocyclic group having from 5 to 30 ring atoms, or a group containing from 2 to 4 of the groups bonded to each other;
c and d each independently represent 0 or 1, provided that when c is 0, (L³)₀ represents a single bond, and when d is 0, (L⁴)₀ represents a single bond;
R^{p} to R^{u} each independently represent a hydrogen atom or a substituent;
X¹⁷ to X⁸⁰ each represent C(R¹⁷) to C(R⁸⁰) or a nitrogen atom; and
R¹⁷ to R⁸⁰ each independently represent a hydrogen atom or a substituent, in which adjacent substituents may be bonded to each other to form a ring,
provided that any one of R¹⁷ to R²⁰ represents a single bond bonded to *⁴, any one of R^{p} and R²⁹ to R³² represents a single bond bonded to *⁵, any one of R³⁷ to R⁴⁰ represents a single bond bonded to *⁶, any one of R^{q} and R⁴¹ to R⁴⁴ represents a single bond bonded to *⁷, any one of R²¹ to R²⁴ represents a single bond bonded to *⁸, any one of R^{r} and R⁴⁹ to R⁵² represents a single bond bonded to *⁹, any one of R^{p} and R²⁵ to R²⁸ represents a single bond bonded to *¹⁰, any one of R^{s} and R⁶¹ to R⁶⁴ represents a single bond bonded to *¹¹, any one of R³³ to R³⁶ represents a single bond bonded to *¹², any one of R^{t} and R⁶⁹ to R⁷² represents a single bond bonded to *¹³, any one of R^{q} and R⁴⁵ to R⁴⁸ represents a single bond bonded to *¹⁴, and any one of R^{u} and R⁷³ to R⁷⁶ represents a single bond bonded to *¹⁵, and
when plural groups or atoms each represented by the same symbol are present for X¹⁷ to X⁸⁰, R¹⁷ to R⁸⁰, and R^{p} to R^{u}, the groups or the atoms may be the same as or different from each other;
e to h each independently represent an integer of 0 or 1, provided that when e to h are 0, the symbols in parentheses with a suffix of 0, (symbol)₀, each independently represent a hydrogen atom or a substituent; and
t and u each independently represent an integer of from 0 to 2, in which t+u=2, provided that the symbols in brackets with a suffix of 0, [symbol]₀, when t is 0, and the symbols in brackets with a suffix of 0, [symbol]₀, when u is 0 each represent a hydrogen atom,

in the general formula (4),
L_{b} represents a substituted or unsubstituted aromatic hydrocarbon group having from 6 to 30 ring carbon atoms or a substituted or unsubstituted heterocyclic group having from 5 to 30 ring atoms;
m represents 0 or 1, provided that when m is 0, (L^{b})₀ represents a single bond;
X³⁰¹ to X³⁰⁸ each represent C(R³⁰¹) to C(R³⁰⁸) or a nitrogen atom, and R³⁰¹ to R³⁰⁸ each independently represent a hydrogen atom or a substituent, in which adjacent substituents may be bonded to each other to form a ring, provided that any one of R³⁰⁵ to R³⁰⁸ represents a single bond directly bonded to L_{b} at the position of *^{c}; and
R^{aa} and R^{bb} each independently represent a hydrogen atom, an alkyl group having from 1 to 10 carbon atoms, an aryl group having from 6 to 14 ring carbon atoms, or a heteroaryl group having from 5 to 14 ring atoms, in which R^{aa} and R^{bb} may be bonded to each other to form a ring, and R^{aa} and R³⁰¹, and R^{bb} and R³⁰⁸ each independently may be bonded to each other to form a ring,

the group represented by the formula (4) is bonded to at least one of L³, L⁴, X¹⁷ to X⁸⁰, and R^{p} to R^{u} at the position of *^{d}, in which any one of R¹⁷ to R⁸⁰ and R^{p} to R^{u} that is bonded to the group represented by the formula (4) represents a single bond, and

when plural groups each represented by the formula (4) are present, the groups may be the same as or different from each other.

### Description of Groups in General Formula (3)

In the general formula (3), A² is the same as the group represented by A¹ in the general formula (1), and the preferred ones thereof are also the same.

In the preferred general formulae of the compound (B) described later, X⁵⁰¹ to X⁵⁰⁸ and R⁵⁰¹ to R⁵¹⁶ in the structure of A² are the same as X²⁰¹ to X²⁰⁸ and R²⁰¹ to R²¹⁶ in the general formulae (N1) to (N6), and the preferred ones thereof are also the same.

In the general formula (3), L³ and L⁴ are the same as the groups represented by L¹ and L² in the general formula (1), and the preferred ones thereof are also the same.

In the general formula (3), c and d each independently represent 0 or 1, provided that when c is 0, (L³)₀ represents a single bond, and when d is 0, (L⁴)₀ represents a single bond.

In the general formula (3), R^{p} to R^{u} each independently represent a hydrogen atom or a substituent, and are the same as the group represented by R^{x} in the general formula (1), and the preferred ones thereof are also the same.

In the general formula (3), X¹⁷ to X⁸⁰ each represent C(R¹⁷) to C(R⁸⁰) or a nitrogen atom. R¹⁷ to R⁸⁰ each independently represent a hydrogen atom or a substituent, in which adjacent substituents may be bonded to each other to form a ring, and are the same as the group represented by R^{x} in the general formula (1), and the preferred ones thereof are also the same.

Any one of R¹⁷ to R²⁰ represents a single bond bonded to *⁴, any one of R^{p} and R²⁹ to R³² represents a single bond bonded to *⁵, any one of R³⁷ to R⁴⁰ represents a single bond bonded to *⁶, any one of R^{q} and R⁴¹ to R⁴⁴ represents a single bond bonded to *⁷, any one of R²¹ to R²⁴ represents a single bond bonded to *⁸, any one of R^{r} and R⁴⁹ to R⁵² represents a single bond bonded to *⁹, any one of R^{p} and R²⁵ to R²⁸ represents a single bond bonded to *¹⁰, any one of R^{s} and R⁶¹ to R⁶⁴ represents a single bond bonded to *¹¹, any one of R³³ to R³⁶ represents a single bond bonded to *¹², any one of R^{t} and R⁶⁹ to R⁷² represents a single bond bonded to *¹³, any one of R^{q} and R⁴⁵ to R⁴⁸ represents a single bond bonded to *¹⁴, and any one of R^{u} and R⁷³ to R⁷⁶ represents a single bond bonded to *¹⁵.

In the general formula (3), when plural groups or atoms each represented by the same symbol are present for X¹⁷ to X⁸⁰, R¹⁷ to R⁸⁰, and R^{p} to R^{u}, the groups or the atoms may be the same as or different from each other.

In the general formula (3), e to h each independently represent an integer of 0 or 1, provided that when e to h are 0, the symbols in parentheses with a suffix of 0, (symbol)₀, each independently represent a hydrogen atom or a substituent.

In the general formula (3), t and u each independently represent an integer of from 0 to 2, in which t+u=2, provided that the symbols in brackets with a suffix of 0, [symbol]₀, when t is 0, and the symbols in brackets with a suffix of 0, [symbol]₀, when u is 0 each represent a hydrogen atom.

### Description of Groups in General Formula (4)

In the general formula (4), L_{b} is the same as the group represented by Lₐ in the general formula (2), and the preferred ones thereof are also the same.

In the general formula (4), m represents 0 or 1, and preferably 0, provided that when m is 0, (L_{b})₀ represents a single bond.

Preferred examples of the case where L_{b} represents a substituted or unsubstituted aromatic hydrocarbon group having from 6 to 30 ring carbon atoms or a substituted or unsubstituted heterocyclic group having from 5 to 30 ring atoms are as described above, and in the general formula (4), the case where m=0, and (L_{b})₀ represents a single bond is further preferred.

In the general formula (4), X³⁰¹ to X³⁰⁸ each represent C(R³⁰¹) to C(R³⁰⁸) or a nitrogen atom. R³⁰¹ to R³⁰⁸ each independently represent a hydrogen atom or a substituent, in which adjacent substituents may be bonded to each other to form a ring. Any one of R³⁰⁵ to R³⁰⁸ represents a single bond directly bonded to L_{b} at the position of *^{c}.

In the general formula (4), R^{aa} and R^{bb} each independently represent a hydrogen atom, an alkyl group having from 1 to 10 carbon atoms, an aryl group having from 6 to 14 ring carbon atoms, or a heteroaryl group having from 5 to 14 ring atoms. R^{aa} and R^{bb} may be bonded to each other to form a ring. R^{aa} and R³⁰¹, and R^{bb} and R³⁰⁸ each independently may be bonded to each other to form a ring.

The group represented by the formula (4) is bonded to at least one of L³, L⁴, X¹⁷ to X⁸⁰, and R^{p} to R^{u} at the position of *^{d}, in which any one of R¹⁷ to R⁸⁰ and R^{p} to R^{u} that is bonded to the group represented by the formula (4) represents a single bond.

When plural groups each represented by the formula (4) are present, the groups may be the same as or different from each other.

More specifically, the structure represented by the general formula (4) is preferably an aryl group selected from the following groups.

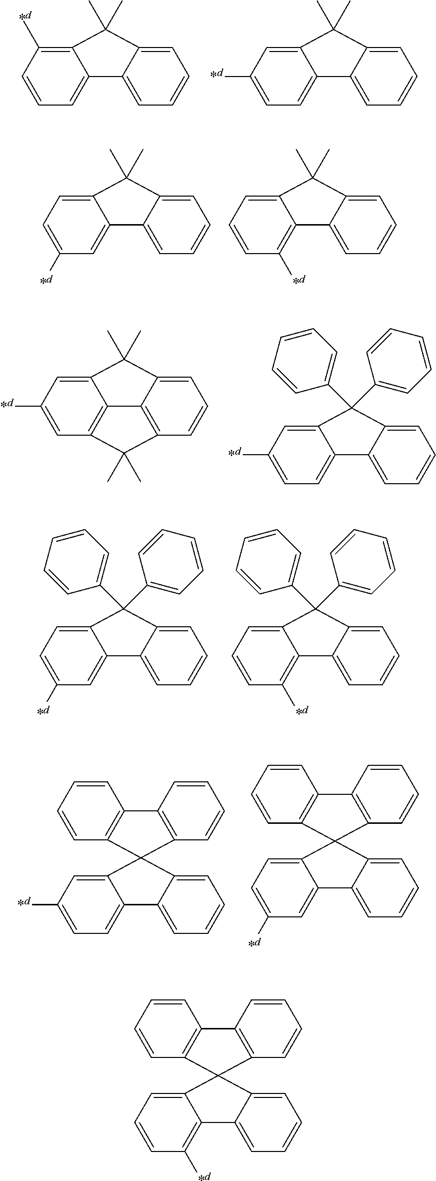

wherein in the formulae, *^{d} is *^{d} in the general formula (4) and represents the bonding position, and the carbon atoms at the positions other than the bonding positions may have a substituent.

### General Formulae of Preferred Compounds in Compound (B)

General formulae of preferred compounds in the compound (B) of the present invention are described below. The definitions of the groups are the same as in the general formulae (3) and (4) unless otherwise expressly indicated, and preferred ones thereof are also the same.

In one embodiment of the compound (B) of the present invention, the structure represented by the general formulae (3) and (4) is preferably a structure represented by the following general formulae (3a) and (4a):

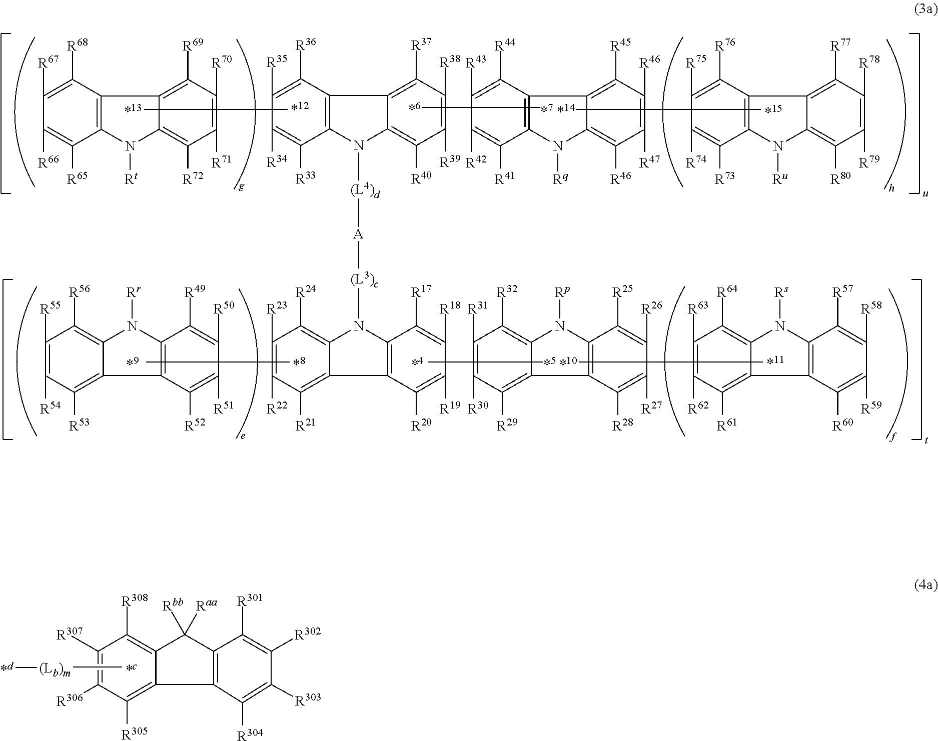

wherein

in the general formula (3a), A², L³, L⁴, c to h, t, u, R¹⁷ to R⁸⁰, and R^{p} to R^{u} have the same meanings as in the formula (3), and preferred ones thereof are also the same, and

in the general formula (4a), L_{b}, m, R³⁰¹ to R³⁰⁸, R^{aa}, and R^{bb} have the same meanings as in the formula (4), and preferred ones thereof are also the same.

In one embodiment of the compound (B) of the present invention, the structure represented by the general formulae (3) and (4) is preferably a structure represented by the following general formulae (3a-1) and (4a):

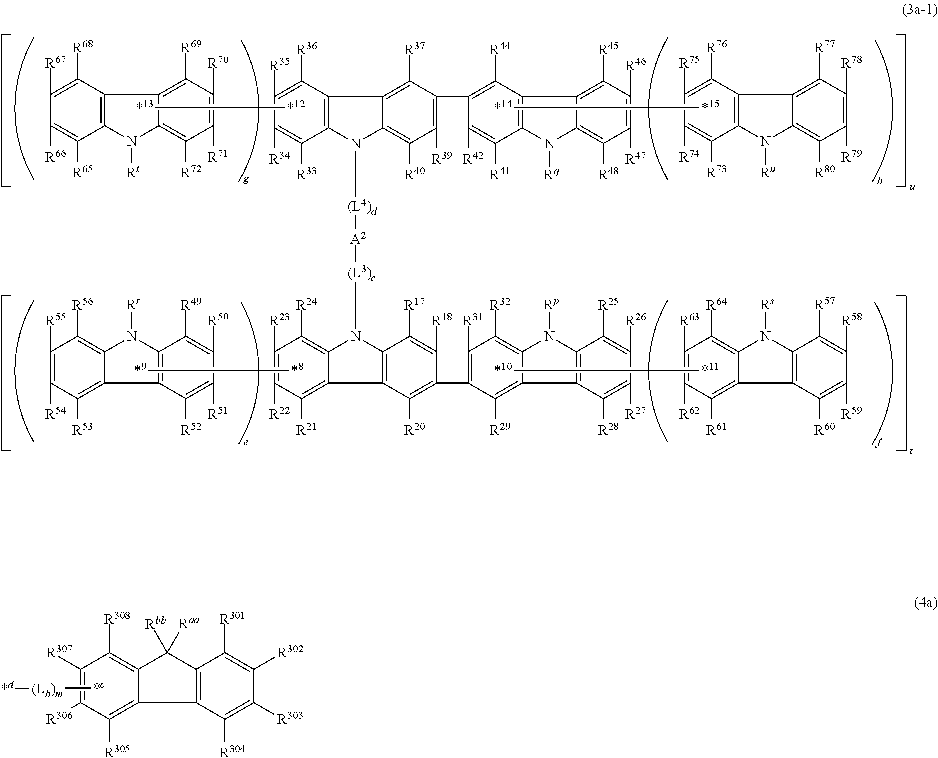

wherein

in the general formula (3a-1), A², L³, L⁴, c to h, t, u, R¹⁷, R¹⁸, R²⁰ to R²⁹, R³¹ to R³⁷, R³⁹ to R⁴², R⁴⁴ to R⁸⁰, and R^{p} to R^{u} have the same meanings as in the formula (3a), and preferred ones thereof are also the same, and

in the general formula (4a), L_{b}, m, R³⁰¹ to R³⁰⁸, R^{aa}, and R^{bb} have the same meanings as above, and preferred ones thereof are also the same.

In one embodiment of the compound (B) of the present invention, the structure represented by the general formulae (3) and (4) bonded to each other is preferably a structure represented by the following general formula (3b):

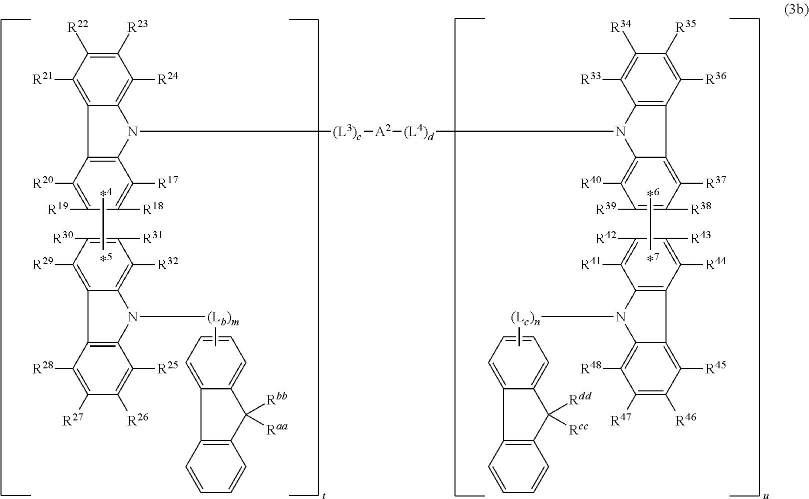

wherein

in the general formula (3b),
A², L³, L⁴, c, d, t, u, R¹⁷ to R⁴⁸, L_{b}, m, R^{aa} and R^{bb} have the same meanings as in the formula (3a), and preferred ones thereof are also the same;
L_{c} represents a substituted or unsubstituted aromatic hydrocarbon group having from 6 to 30 ring carbon atoms or a substituted or unsubstituted heterocyclic group having from 5 to 30 ring atoms;
n represents 0 or 1, provided that when n is 0, (L_{c})₀ represents a single bond; and
R^{cc} and R^{dd} each independently represent a hydrogen atom, an alkyl group having from 1 to 10 carbon atoms, an aryl group having from 6 to 14 ring carbon atoms, or a heteroaryl group having from 5 to 14 ring atoms, in which R^{cc} and R^{dd} may be bonded to each other to form a ring. R^{cc} and R^{dd} have the same meaning as R^{aa} and R^{bb}, and preferred ones thereof are also the same.

In one embodiment of the compound (B) of the present invention, the structure represented by the general formula (3b) is preferably a structure represented by the following general formula (3b-1):

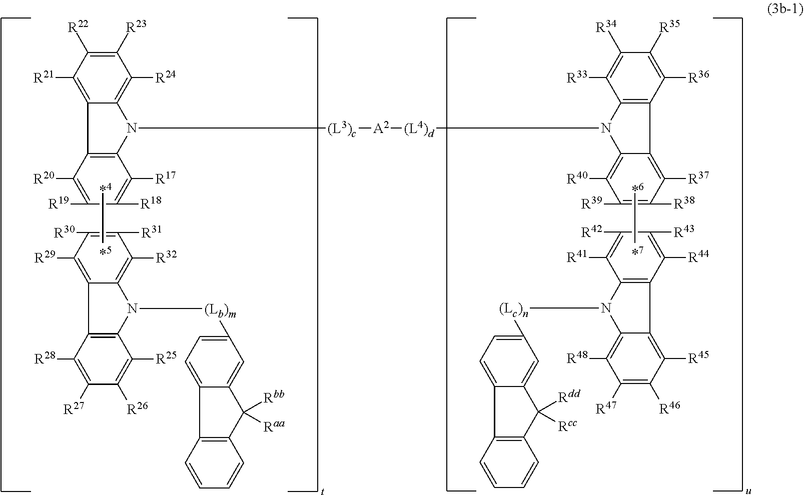

wherein in the general formula (3b-1), A², L³, L⁴, c, d, t, u, R¹⁷ to R⁴⁸, L_{b}, L_{c}, m, n, and R^{aa} to R^{dd} have the same meanings as in the formula (3b), and preferred ones thereof are also the same.

In one embodiment of the compound (B) of the present invention, the structure represented by the general formula (3b) is more preferably a structure represented by the following general formula (3b-2):

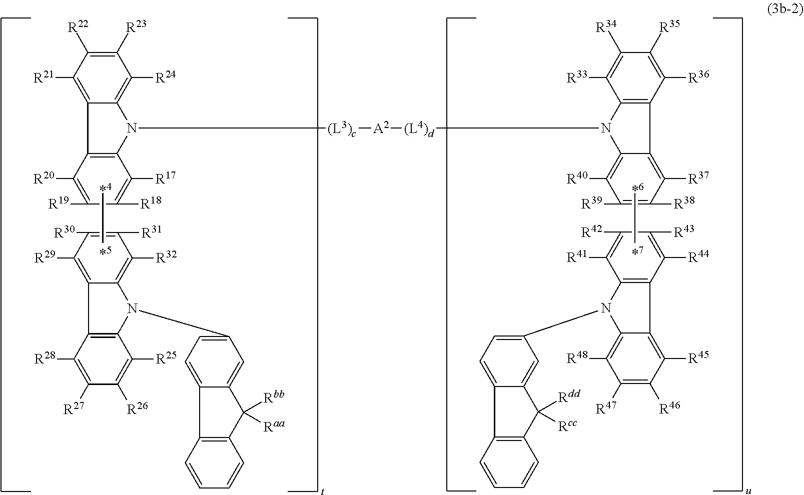

wherein in the general formula (3b-2), A², L³, L⁴, c, d, t, u, R¹⁷ to R⁴⁸, and R^{aa} to R^{dd} have the same meanings as in the formula (3b), and preferred ones thereof are also the same.

In one embodiment of the compound (B) of the present invention, the structure represented by the general formula (3b) is further preferably a structure represented by the following general formula (3b-3):

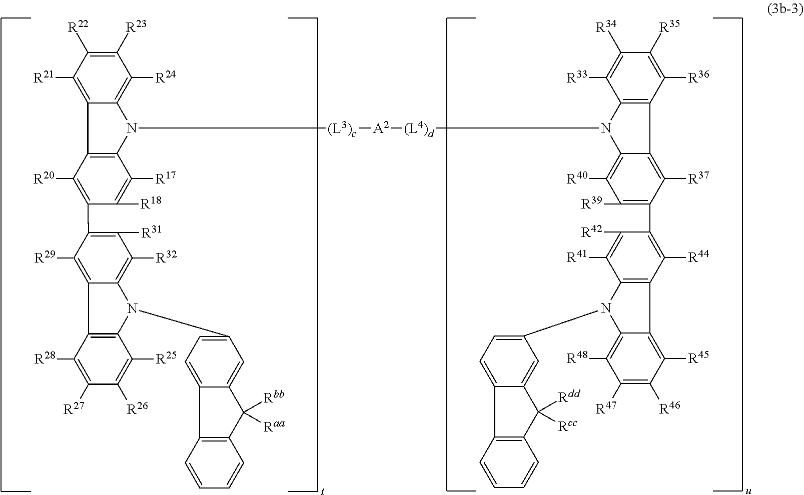

wherein in the general formula (3b-3), A², L³, L⁴, c, d, t, u, R¹⁷, R¹⁸, R²⁰ to R²⁹, R³¹ to R³⁷, R³⁹ to R⁴², R⁴⁴ to R⁴⁸, and R^{aa} to R^{dd} have the same meanings as in the formula (3b), and preferred ones thereof are also the same.

In one embodiment of the compound (B) of the present invention, the structure represented by the general formulae (3) and (4) may be a structure represented by the following general formulae (3c) and (4a):

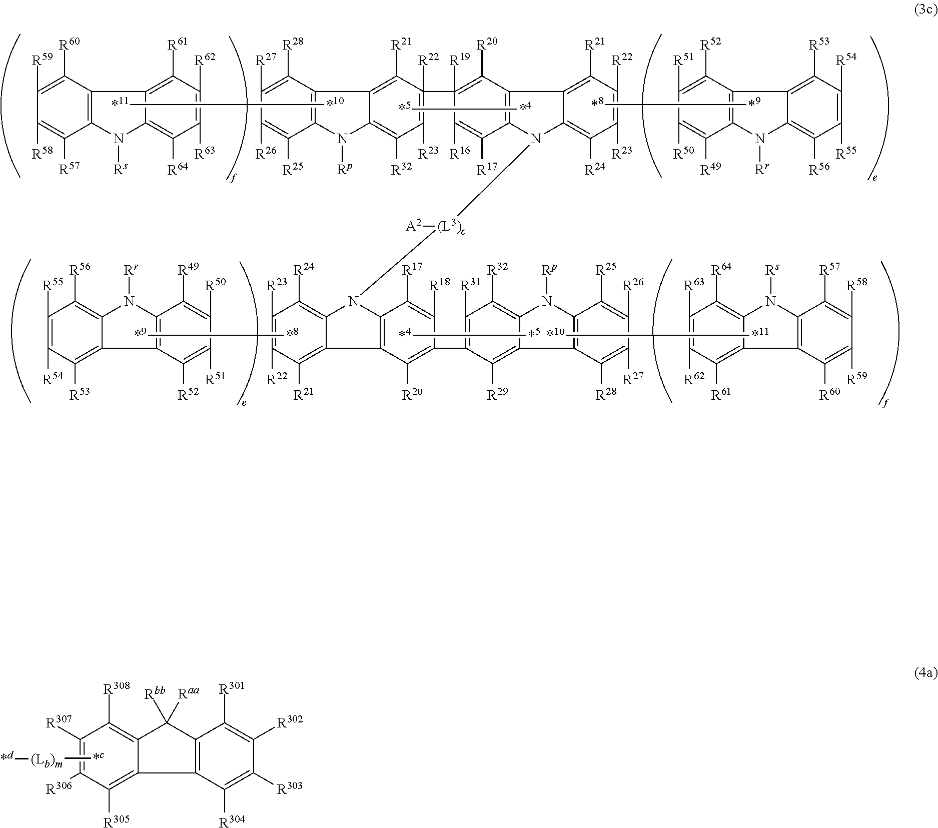

wherein

in the general formula (3c), A², L³, c, e, f, R¹⁷ to R³², R⁴⁹ to R⁶⁴, R^{p}, R^{r}, and R^{s} have the same meanings as in the formula (3a), and preferred ones thereof are also the same, and

in the general formula (4a), L_{b}, m, R³⁰¹ to R³⁹⁸, R^{aa}, and R^{bb} have the same meanings as above, and preferred ones thereof are also the same.

In one embodiment of the compound (B) of the present invention, the structure represented by the general formulae (3) and (4) may be a structure represented by the following general formulae (3d) and (4):

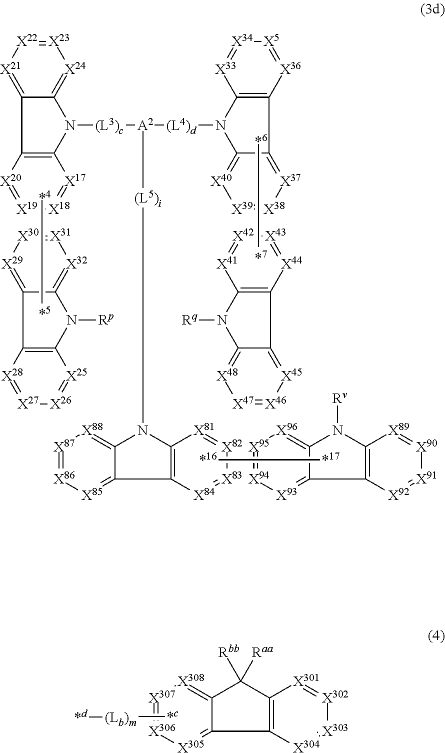

wherein

in the general formula (3d),
A², L³, L⁴, c, d, X¹⁷ to X⁴⁸, R^{p}, and R^{q} have the same meanings as in the formula (3a), and preferred ones thereof are also the same;
L⁵ represents a substituted or unsubstituted aromatic hydrocarbon group having from 6 to 30 ring carbon atoms, a substituted or unsubstituted heterocyclic group having from 5 to 30 ring atoms, or a group containing from 2 to 4 of the groups bonded to each other;
i represents 0 or 1, provided that when i is 0, (L⁵)₀ represents a single bond;
R^{v} represents a hydrogen atom or a substituent;
X⁸¹ to X⁹⁶ each represent C(R⁸¹) to C(R⁹⁶) or a nitrogen atom; and
R⁸¹ to R⁹⁶ each independently represent a hydrogen atom or a substituent, in which adjacent substituents may be bonded to each other to form a ring,
provided that any one of R⁸¹ to R⁸⁴ represents a single bond bonded to *¹⁶, and any one of R^{v}, and R⁹³ to R⁹⁶ represents a single bond bonded to *¹⁷, and

in the general formula (4),
L_{b}, m, X³⁰¹ to X³⁰⁸, R^{aa}, and R^{bb} have the same meanings as above, and preferred ones thereof are also the same.

In one embodiment of the compound (B) of the present invention, the structure represented by the general formulae (3d) and (4) may be a structure represented by the following general formulae (3d-1) and (4a-1);

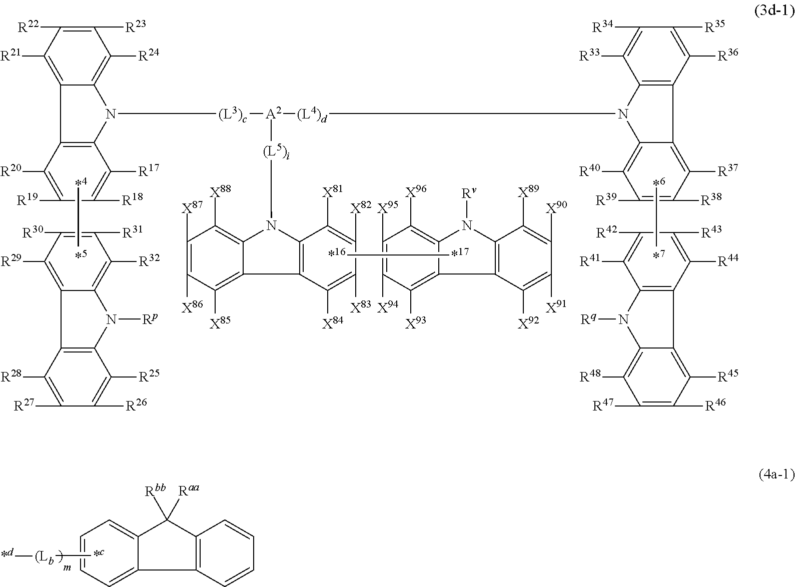

wherein

in the general formula (3d-1), A², L³ to L⁵, c, d, i, R¹⁷ to R⁴⁸, R⁸¹ to R⁹⁶, R^{p}, R^{q}, and R^{v} have the same meanings as in the formula (3d), and preferred ones thereof are also the same, and

in the general formula (4a-1), L_{b}, m, R^{aa}, and R^{bb} have the same meanings as in the formula (4), and preferred ones thereof are also the same.

In one embodiment of the compound (B) of the present invention, the structure represented by the general formulae (3) and (4) bonded to each other may be a structure represented by the following general formula (3e):

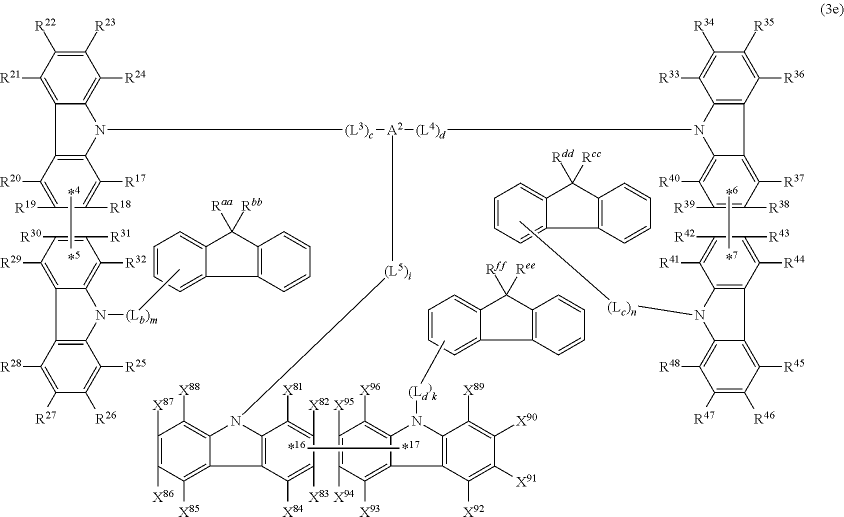

wherein

in the general formula (3e), A², L³ to L⁵, c, d, i, R¹⁷ to R⁴⁸, R⁸¹ to R⁹⁶, L_{b}, m, R^{aa}, and R^{bb} have the same meanings as in the formulae (3d) and (4), and preferred ones thereof are also the same;

L_{c} and L_{d} each independently represent a substituted or unsubstituted aromatic hydrocarbon group having from 6 to 30 ring carbon atoms or a substituted or unsubstituted heterocyclic group having from 5 to 30 ring atoms;

k and n each independently represent 0 or 1, provided that when k is 0, (L_{d})₀ represents a single bond, and when n is 0, (L_{c})₀ represents a single bond; and

R^{cc} to R^{ff} each independently represent a hydrogen atom, an alkyl group having from 1 to 10 carbon atoms, an aryl group having from 6 to 14 ring carbon atoms, or a heteroaryl group having from 5 to 14 ring atoms, in which R^{cc} and R^{dd}, and R^{ee} and R^{ff} each independently may be bonded to each other to form a ring.

In one embodiment of the compound (B) of the present invention, the structure represented by the general formulae (3) and (4) may be a structure represented by the following general formulae (3-i) and (4a):

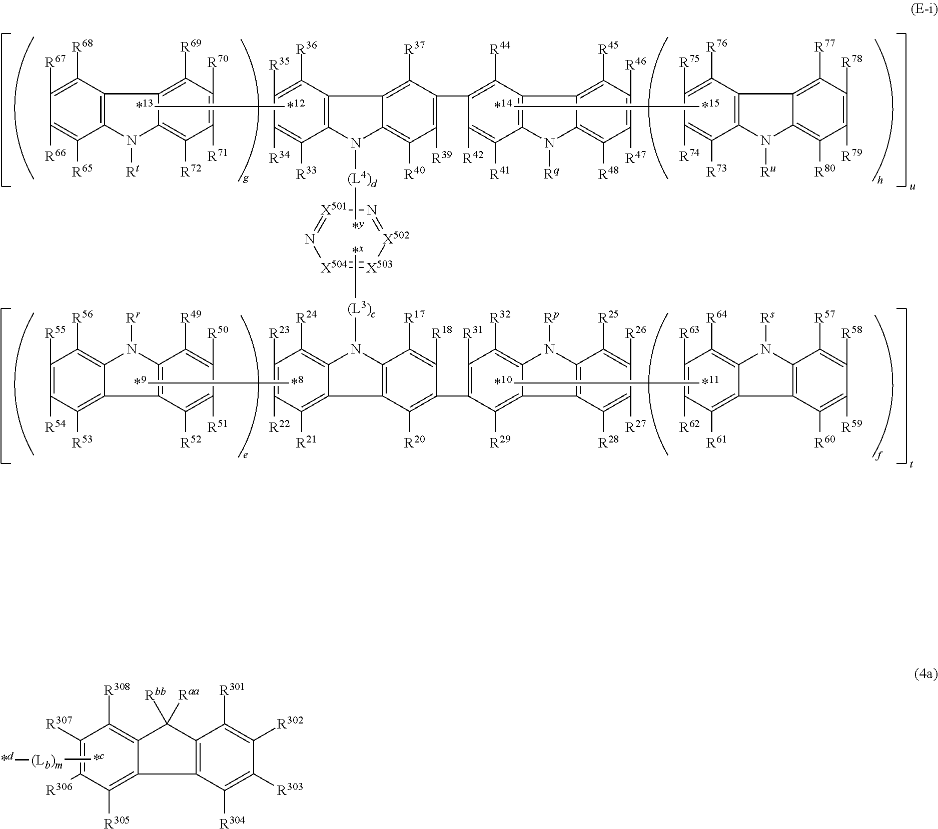

wherein

in the general formula (3-i),
L³, L⁴, c to h, t, u, R¹⁷ to R⁸⁰, and R^{p} to R^{u} have the same meanings as in the formula (3), and preferred ones thereof are also the same;
one of X⁵⁰¹ to X⁵⁰⁴ represents a carbon atom bonded to *^{x}, one of the other three represents a carbon atom bonded to *^{y}, and the balance thereof represents C(R₂), C(R₃), or a nitrogen atom; and

R₂ and R₃ each represent a hydrogen atom or a substituent, and

in the general formula (4a),
L_{b}, m, R³⁰¹ to R³⁰⁸, R^{aa}, and R^{bb} have the same meanings as above, and preferred ones thereof are also the same.

In one embodiment of the compound (B) of the present invention, the structure represented by the general formulae (3) and (4) may be a structure represented by the following general formulae (3-ii) and (4a):

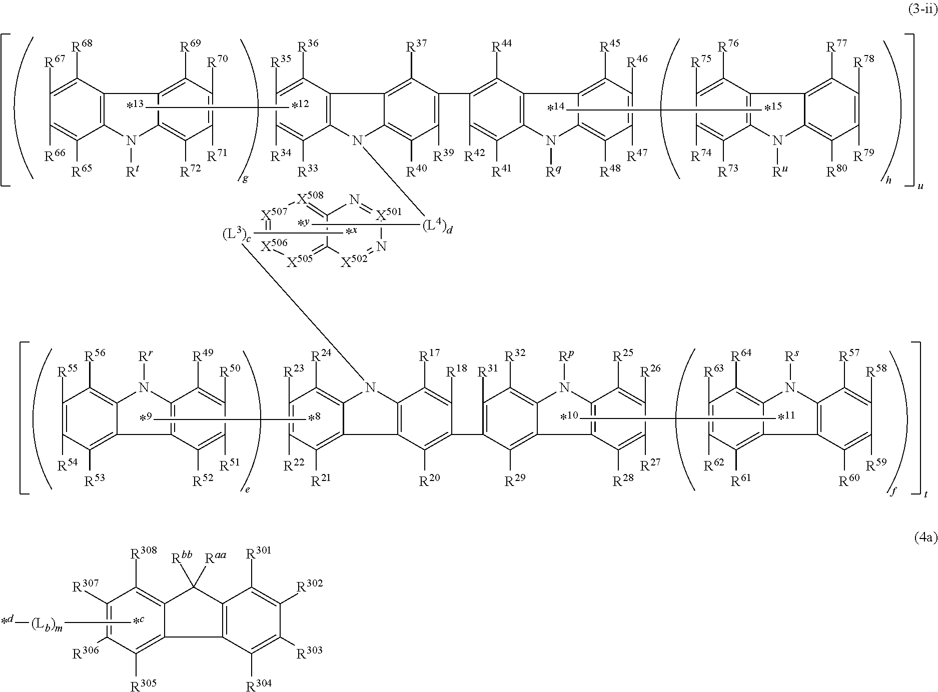

wherein

in the general formula (3-ii),
L³, L⁴, c to h, t, u, R¹⁷ to R⁸⁰, and R^{p} to R^{u} have the same meanings as in the formula (3), and preferred ones thereof are also the same;
one of X⁵⁰¹, X⁵⁰², and X⁵⁰⁵ to X⁵⁰⁸ represents a carbon atom bonded to *^{x}, one of the other five represents a carbon atom bonded to *^{y}, and the balance thereof represents C(R₂) to C(R₅), or a nitrogen atom; and

R₂ to R₅ each represent a hydrogen atom or a substituent, and

in the general formula (4a),
L_{b}, m, R³⁰¹ to R³⁰⁸, R^{aa}, and R^{bb} have the same meanings as above, and preferred ones thereof are also the same.

In one embodiment of the compound (B) of the present invention, the structure represented by the general formulae (3) and (4) may be a structure represented by the following general formulae (3-iii) and (4a):

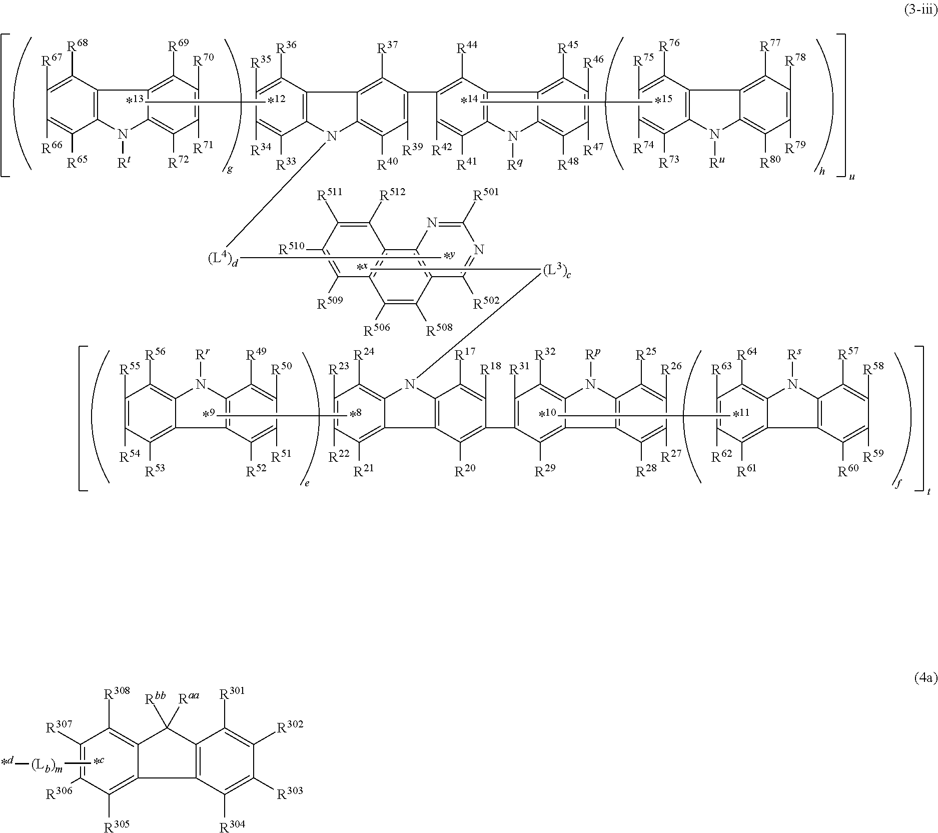

wherein

in the general formula (3-iii),
L³, L⁴, c to h, t, u, R¹⁷ to R⁸⁰, and R^{p} to R^{u} have the same meanings as in the formula (3), and preferred ones thereof are also the same; and
one of R⁵⁰¹, R⁵⁰², R⁵⁰⁵, R⁵⁰⁶, and R⁵⁰⁹ to R⁵¹² represents a single bond bonded to *^{x}, one of the other seven represents a single bond bonded to *^{y}, and the balance thereof represents a hydrogen atom or a substituent, and

in the general formula (4a),
L_{b}, m, R³⁰¹ to R³⁰⁸, R^{aa}, and R^{bb} have the same meanings as above, and preferred ones thereof are also the same.

In one embodiment of the compound (B) of the present invention, the structure represented by the general formulae (3) and (4) may be a structure represented by the following general formulae (3-iv) and (4a):

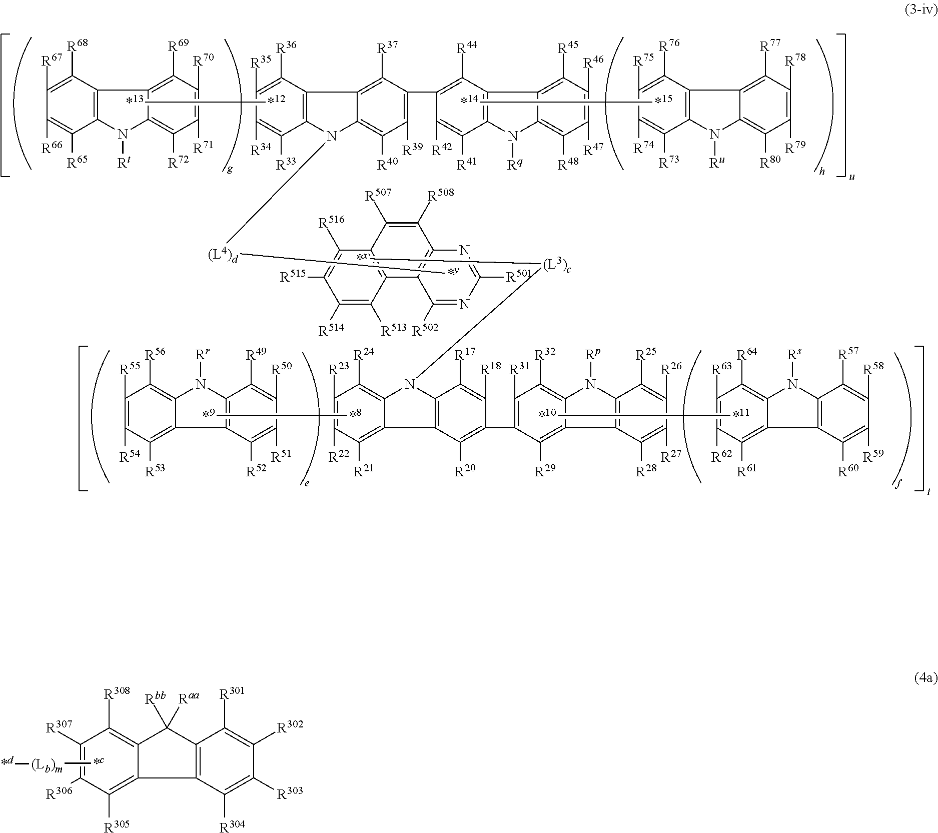

wherein

in the general formula (3-iv),
L³, L⁴, c to h, t, u, R¹⁷ to R⁸⁰, and R^{p} to R^{u} have the same meanings as in the formula (3), and preferred ones thereof are also the same; and
one of R⁵⁰¹, R⁵⁰², R⁵⁰⁷, R⁵⁰⁸, and R⁵¹³ to R⁵¹⁶ represents a single bond bonded to *^{x}, one of the other seven represents a single bond bonded to *^{y}, and the balance thereof represents a hydrogen atom or a substituent, and

in the general formula (4a),
L_{b}, m, R³⁰¹ to R³⁰⁸, R^{aa}, and R^{bb} have the same meanings as above, and preferred ones thereof are also the same.

Specific examples of the compounds (A) and (B) are shown below, but the present invention is not limited to the examples.

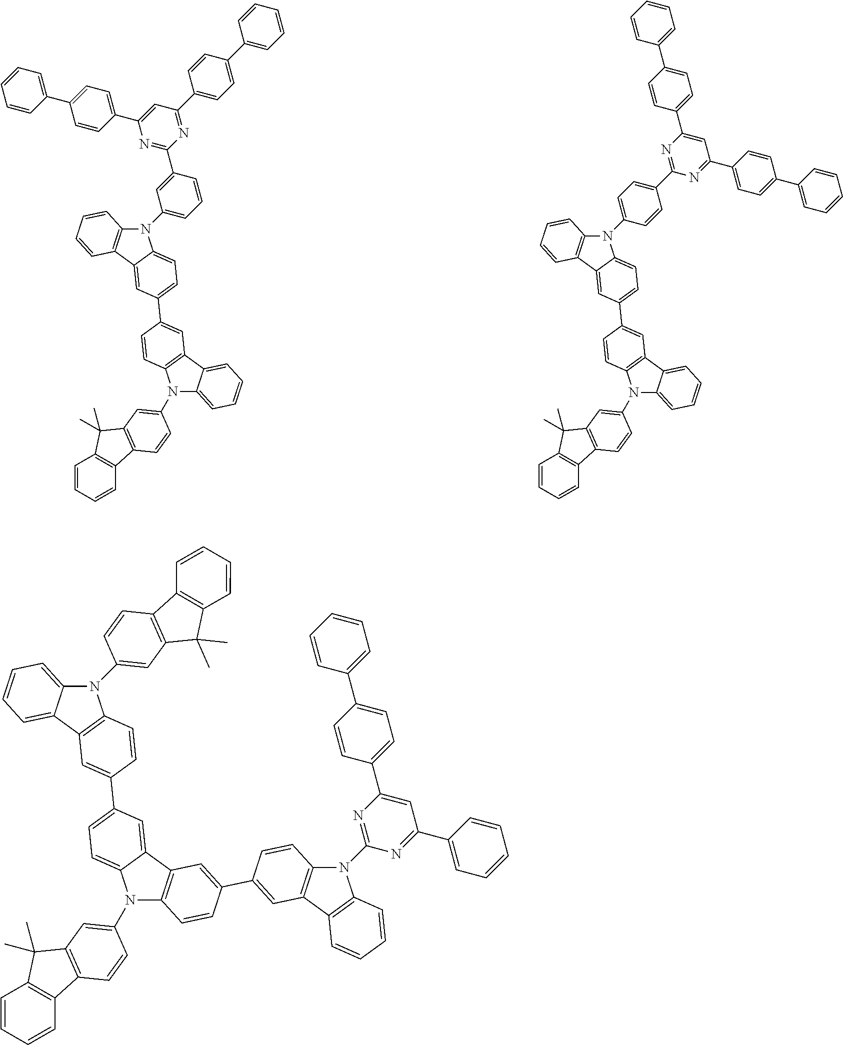 [SMILES: CC1(C)c2ccccc2-c2ccc(-n3c4ccccc4c4cc(-c5ccc6c(c5)c5ccccc5n6-c5cccc(-c6nc(-c7ccc(-c8ccccc8)cc7)cc(-c7ccc(-c8ccccc8)cc7)n6)c5)ccc43)cc21 ; CC1(C)c2ccccc2-c2ccc(-n3c4ccccc4c4cc(-c5ccc6c(c5)c5ccccc5n6-c5ccc(-c6nc(-c7ccc(-c8ccccc8)cc7)cc(-c7ccc(-c8ccccc8)cc7)n6)cc5)ccc43)cc21 ; CC1(C)c2ccccc2-c2ccc(-n3c4ccccc4c4cc(-c5ccc6c(c5)c5cc(-c7ccc8c(c7)c7ccccc7n8-c7nc(-c8ccccc8)cc(-c8ccc(-c9ccccc9)cc8)n7)ccc5n6-c5ccc6c(c5)C(C)(C)c5ccccc5-6)ccc43)cc21]

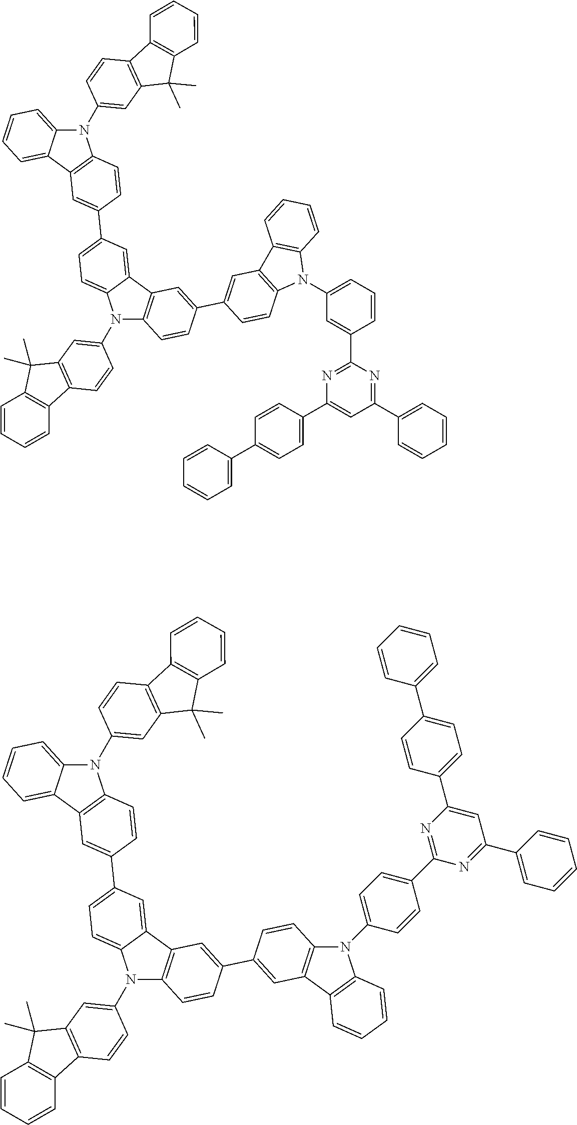 [SMILES: CC1(C)c2ccccc2-c2ccc(-n3c4ccccc4c4cc(-c5ccc6c(c5)c5cc(-c7ccc8c(c7)c7ccccc7n8-c7cccc(-c8nc(-c9ccccc9)cc(-c9ccc(-c%10ccccc%10)cc9)n8)c7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccccc9)cc(-c9ccc(-c%10ccccc%10)cc9)n8)cc7)ccc5n6-c5ccc6c(c5)C(C)(C)c5ccccc5-6)ccc43)cc21]

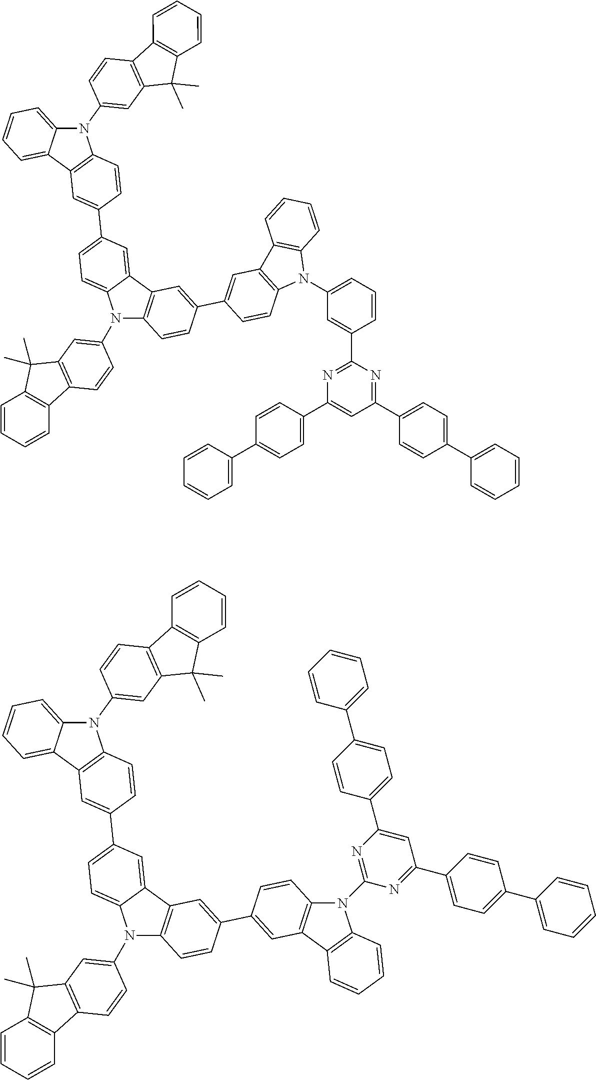 [SMILES: CC1(C)c2ccccc2-c2ccc(-n3c4ccccc4c4cc(-c5ccc6c(c5)c5cc(-c7ccc8c(c7)c7ccccc7n8-c7cccc(-c8nc(-c9ccc(-c%10ccccc%10)cc9)cc(-c9ccc(-c%10ccccc%10)cc9)n8)c7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7nc(-c8ccc(-c9ccccc9)cc8)cc(-c8ccc(-c9ccccc9)cc8)n7)ccc5n6-c5ccc6c(c5)C(C)(C)c5ccccc5-6)ccc43)cc21]

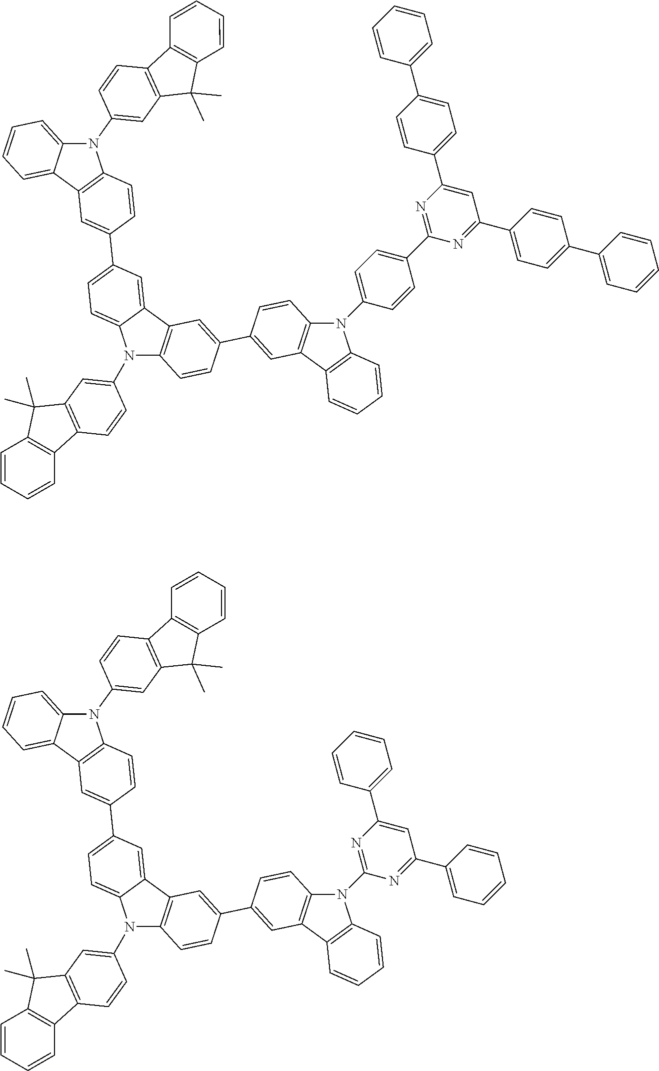 [SMILES: CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccc(-c%10ccccc%10)cc9)cc(-c9ccc(-c%10ccccc%10)cc9)n8)cc7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7nc(-c8ccccc8)cc(-c8ccccc8)n7)ccc5n6-c5ccc6c(c5)C(C)(C)c5ccccc5-6)ccc43)cc21]

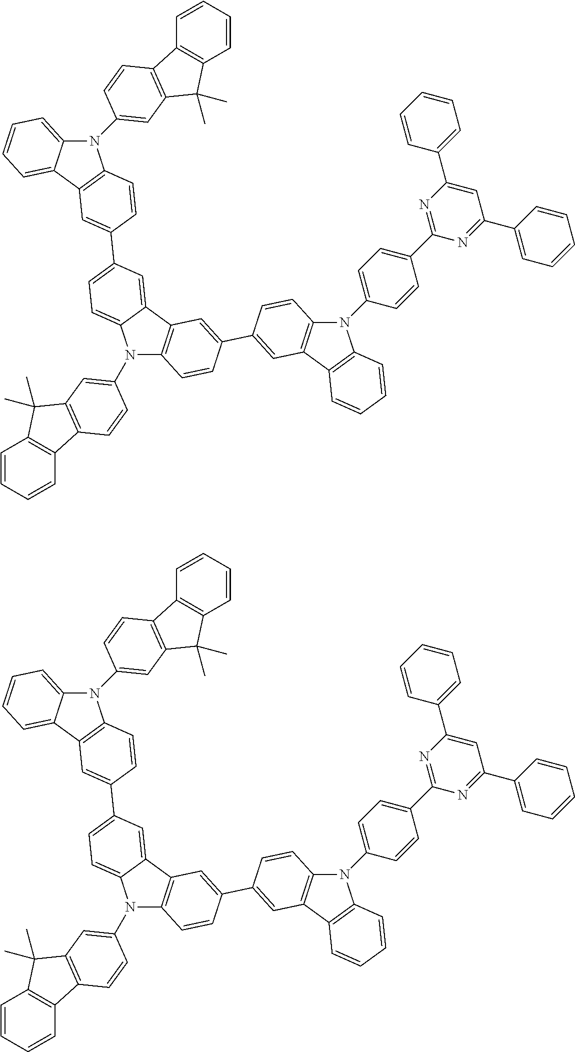 [SMILES: CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccccc9)cc(-c9ccccc9)n8)cc7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccccc9)cc(-c9ccccc9)n8)cc7)ccc5n6-c5ccc6c(c5)C(C)(C)c5ccccc5-6)ccc43)cc21]

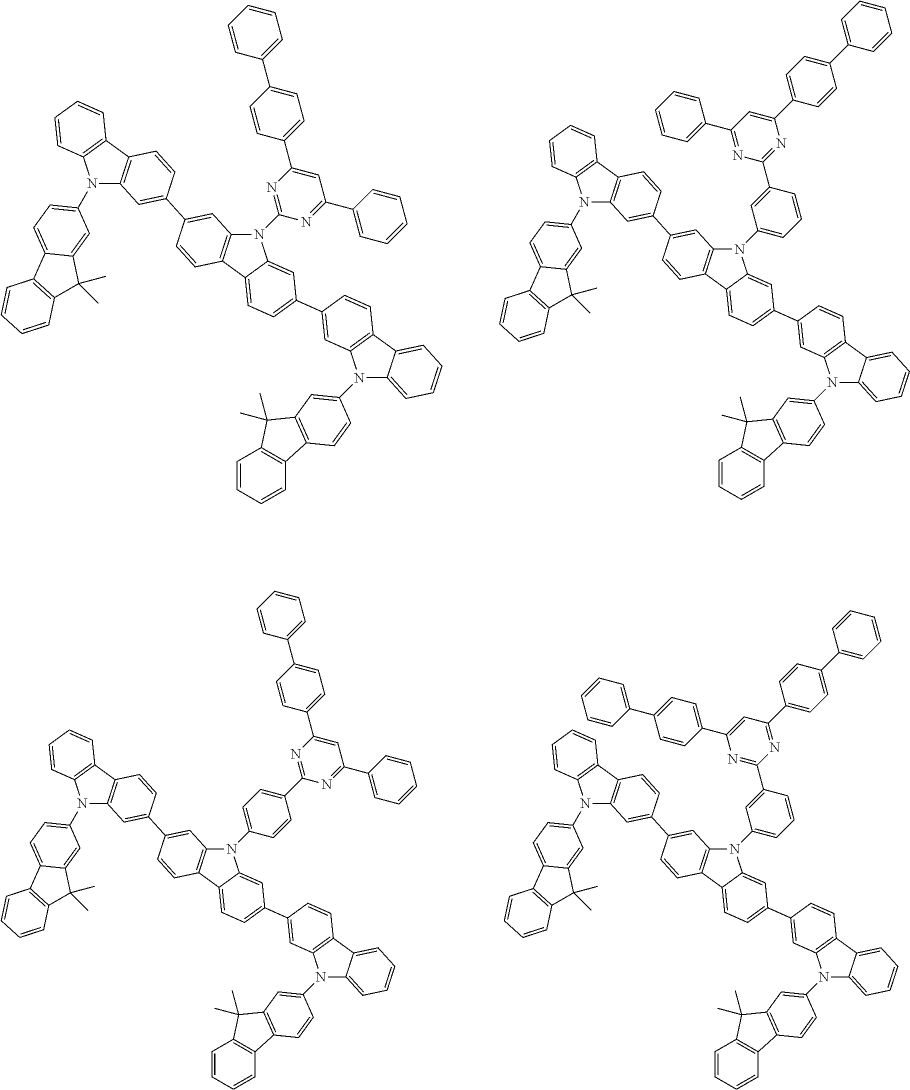

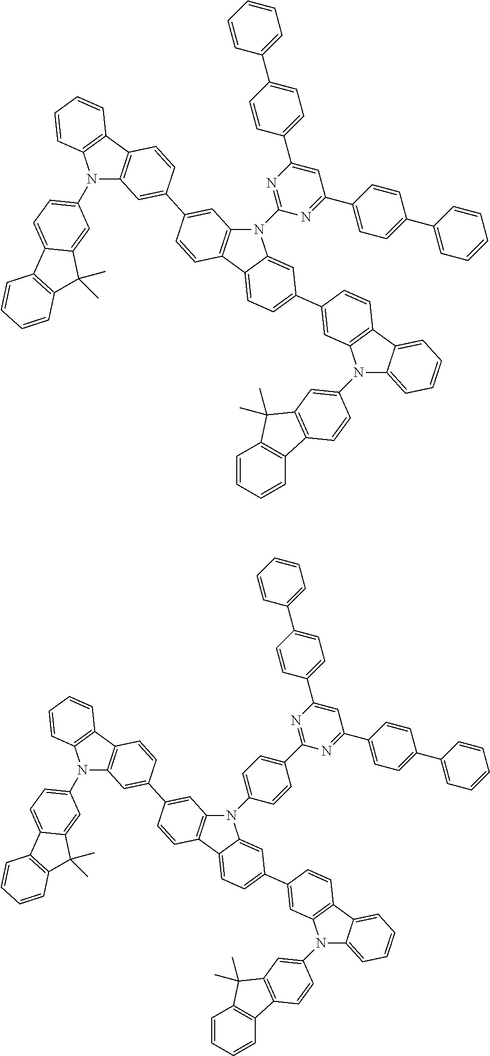 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7nc(-c8ccc(-c9ccccc9)cc8)cc(-c8ccc(-c9ccccc9)cc8)n7)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7ccc(-c8nc(-c9ccc(-c%10ccccc%10)cc9)cc(-c9ccc(-c%10ccccc%10)cc9)n8)cc7)c6c5)cc43)cc21]

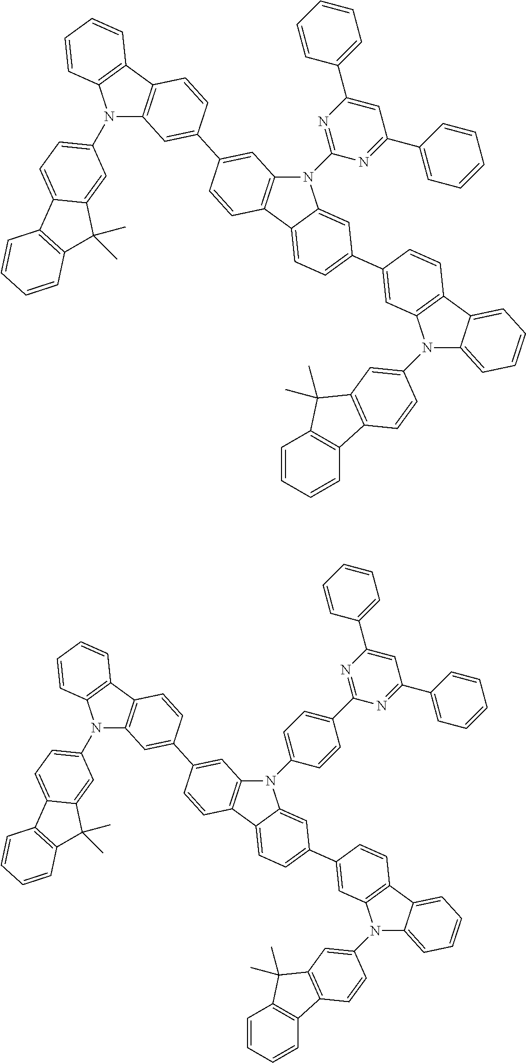 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7nc(-c8ccccc8)cc(-c8ccccc8)n7)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7ccc(-c8nc(-c9ccccc9)cc(-c9ccccc9)n8)cc7)c6c5)cc43)cc21]

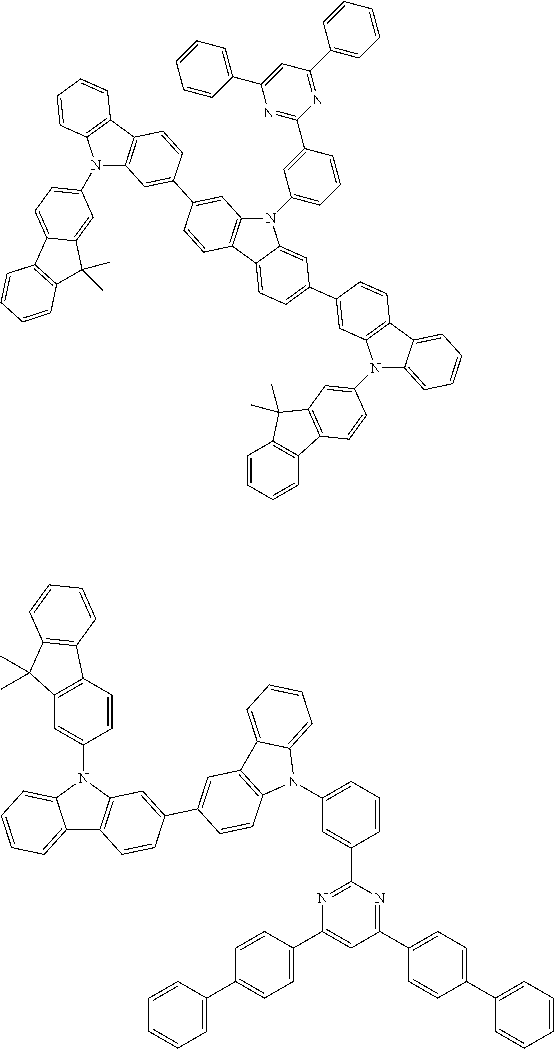 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7cccc(-c8nc(-c9ccccc9)cc(-c9ccccc9)n8)c7)c6c5)cc43)cc21 ; CC1(C)c2ccccc2-c2ccc(-n3c4ccccc4c4ccc(-c5ccc6c(c5)c5ccccc5n6-c5cccc(-c6nc(-c7ccc(-c8ccccc8)cc7)cc(-c7ccc(-c8ccccc8)cc7)n6)c5)cc43)cc21]

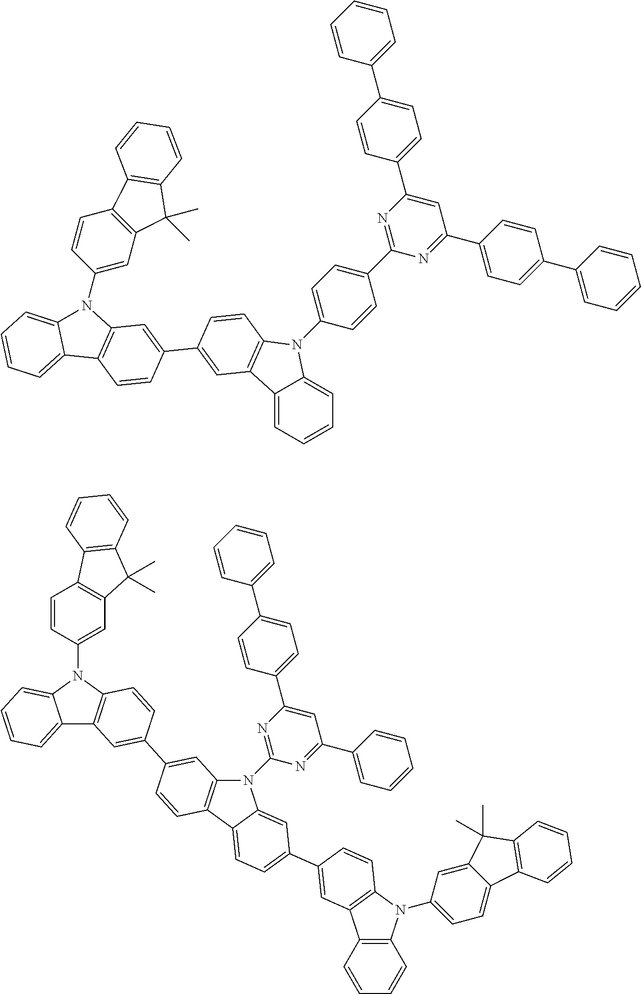 [SMILES: CC1(C)c2ccccc2-c2ccc(-n3c4ccccc4c4ccc(-c5ccc6c(c5)c5ccccc5n6-c5ccc(-c6nc(-c7ccc(-c8ccccc8)cc7)cc(-c7ccc(-c8ccccc8)cc7)n6)cc5)cc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7nc(-c8ccccc8)cc(-c8ccc(-c9ccccc9)cc8)n7)c6c5)ccc43)cc21]

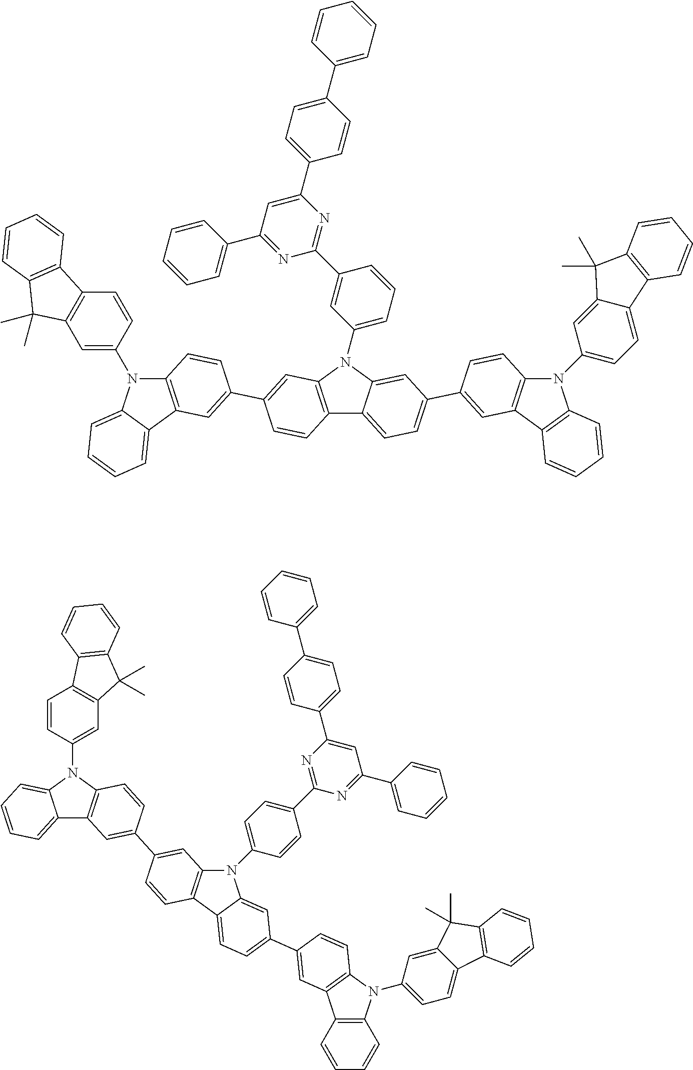 [SMILES: CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7cccc(-c8nc(-c9ccccc9)cc(-c9ccc(-c%10ccccc%10)cc9)n8)c7)c6c5)ccc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7ccc(-c8nc(-c9ccccc9)cc(-c9ccc(-c%10ccccc%10)cc9)n8)cc7)c6c5)ccc43)cc21]

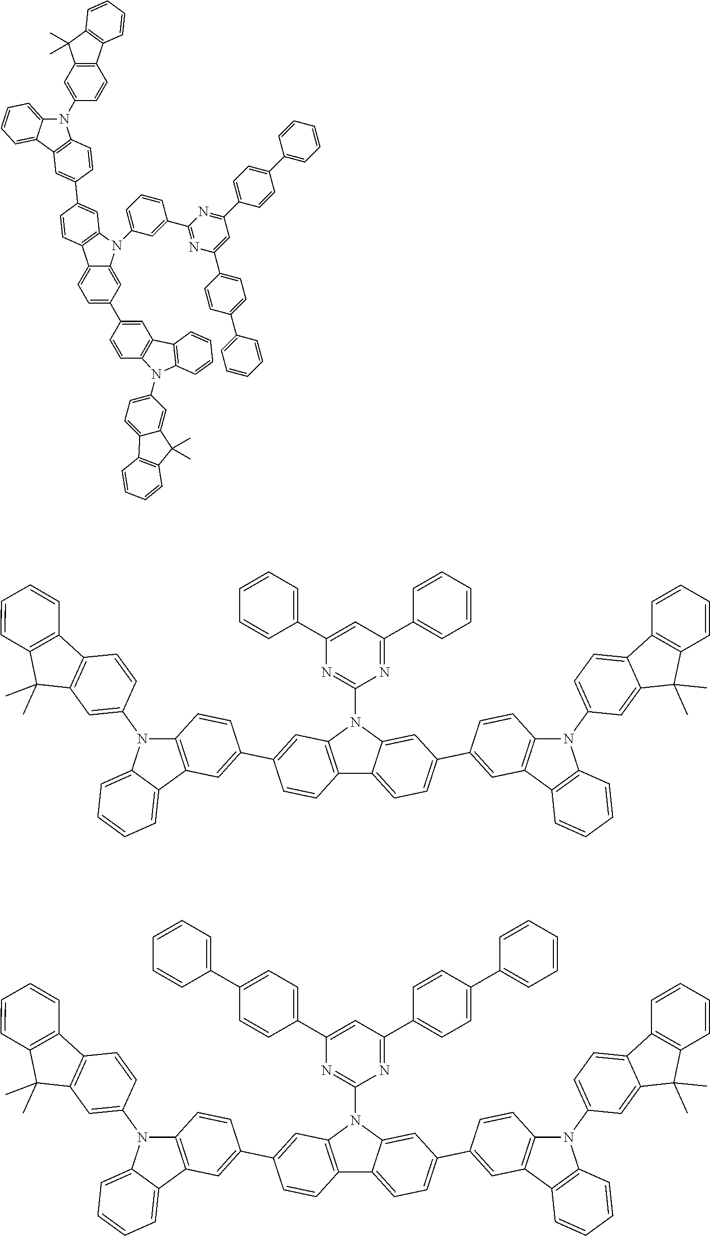

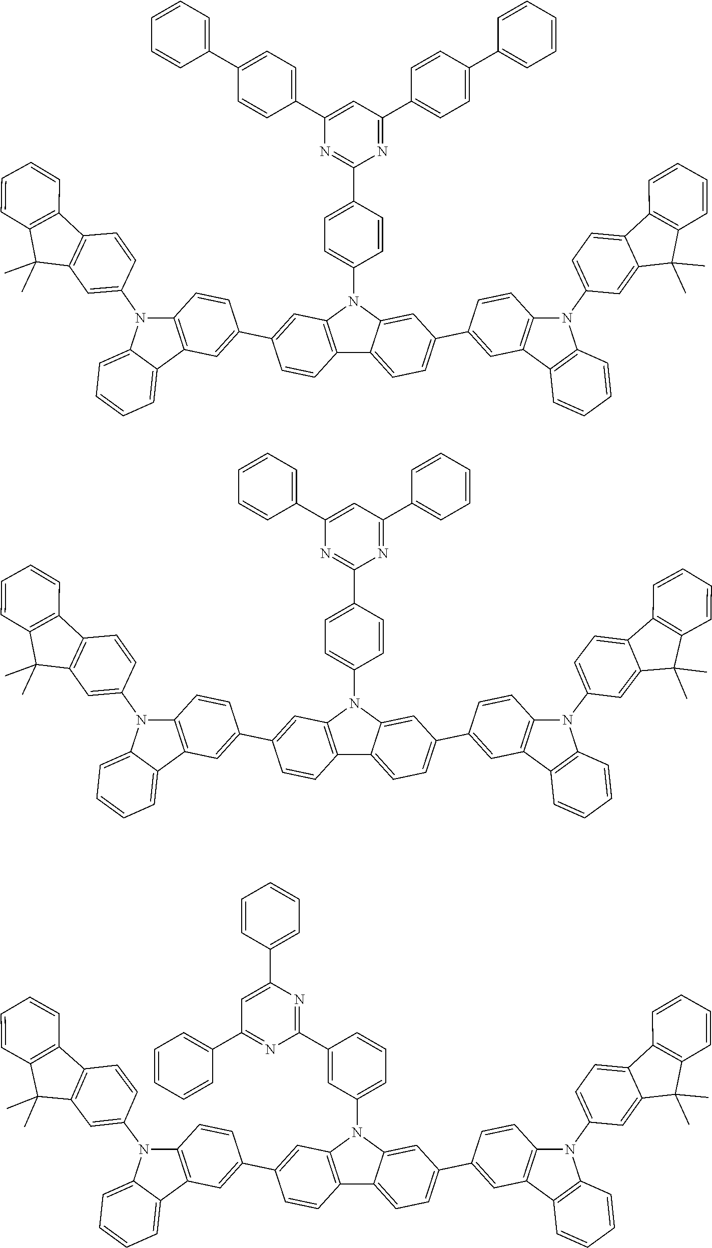

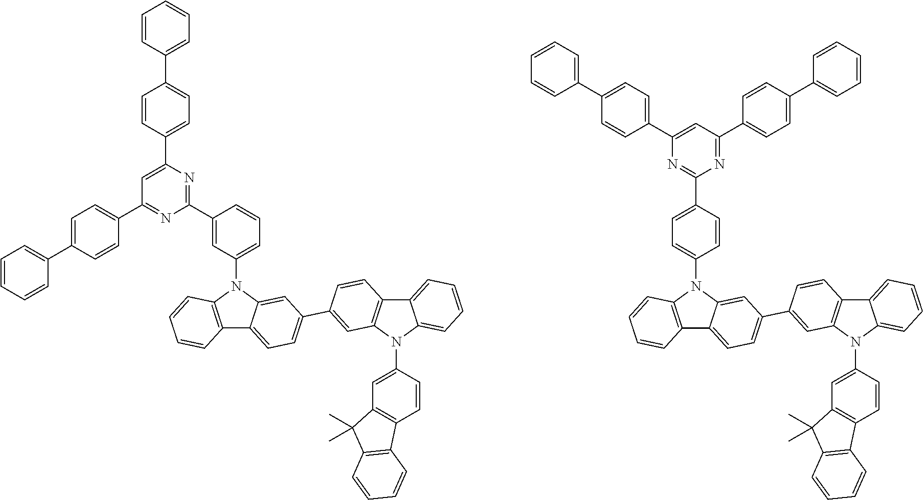 [SMILES: CC1(C)c2ccccc2-c2ccc(-n3c4ccccc4c4ccc(-c5ccc6c7ccccc7n(-c7cccc(-c8nc(-c9ccc(-c%10ccccc%10)cc9)cc(-c9ccc(-c%10ccccc%10)cc9)n8)c7)c6c5)cc43)cc21 ; CC1(C)c2ccccc2-c2ccc(-n3c4ccccc4c4ccc(-c5ccc6c7ccccc7n(-c7ccc(-c8nc(-c9ccc(-c%10ccccc%10)cc9)cc(-c9ccc(-c%10ccccc%10)cc9)n8)cc7)c6c5)cc43)cc21]

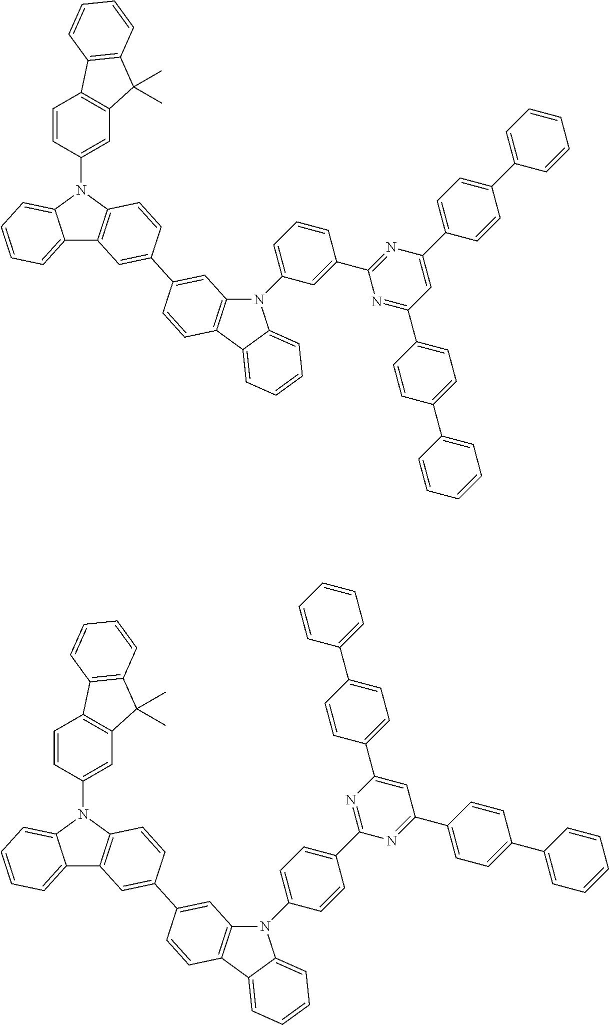 [SMILES: CC1(C)c2ccccc2-c2ccc(-n3c4ccccc4c4cc(-c5ccc6c7ccccc7n(-c7cccc(-c8nc(-c9ccc(-c%10ccccc%10)cc9)cc(-c9ccc(-c%10ccccc%10)cc9)n8)c7)c6c5)ccc43)cc21 ; CC1(C)c2ccccc2-c2ccc(-n3c4ccccc4c4cc(-c5ccc6c7ccccc7n(-c7ccc(-c8nc(-c9ccc(-c%10ccccc%10)cc9)cc(-c9ccc(-c%10ccccc%10)cc9)n8)cc7)c6c5)ccc43)cc21]

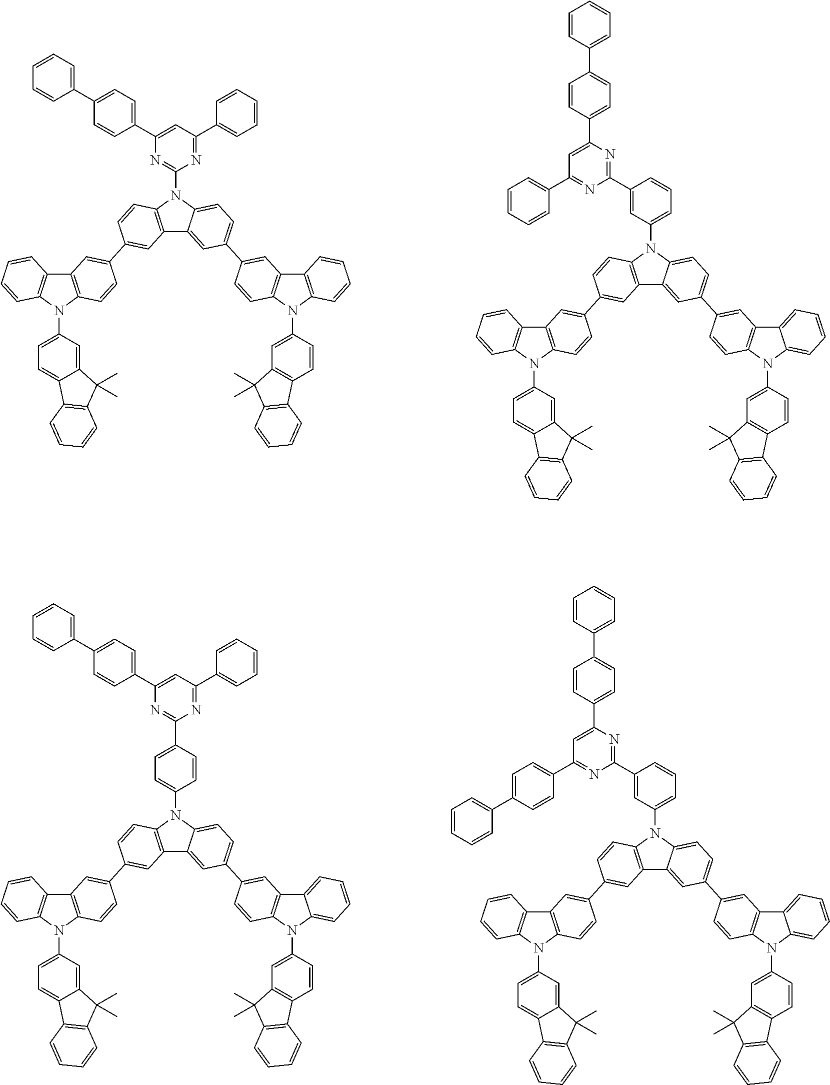

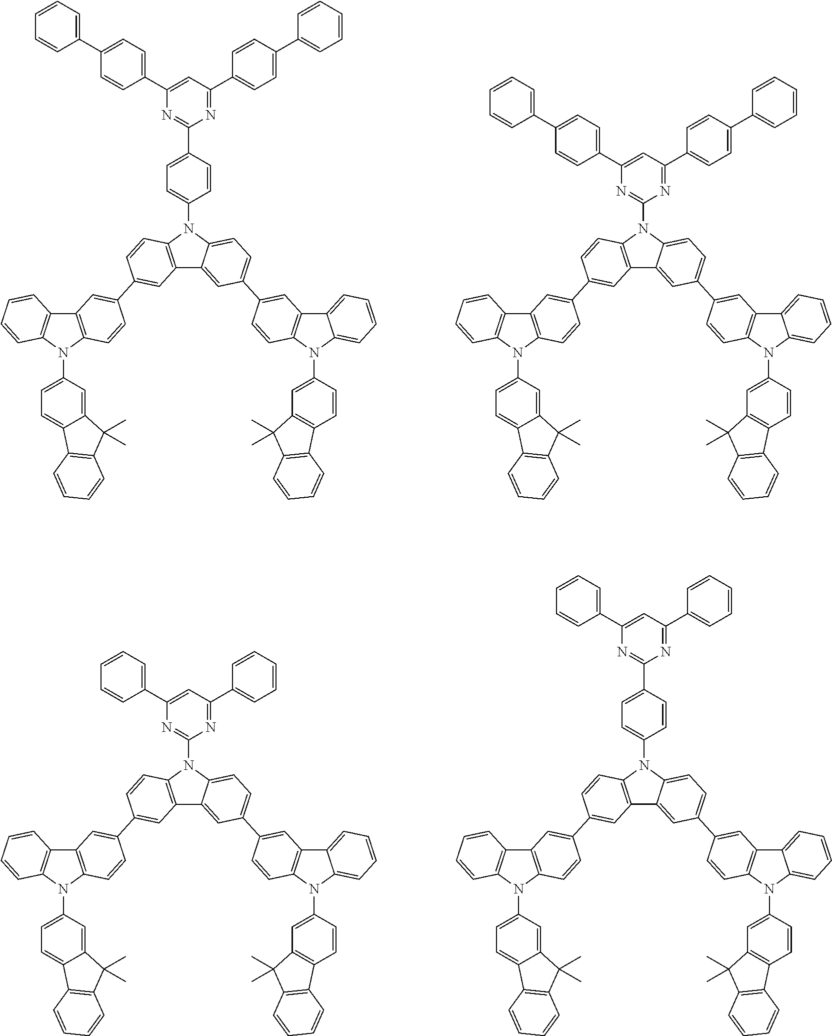

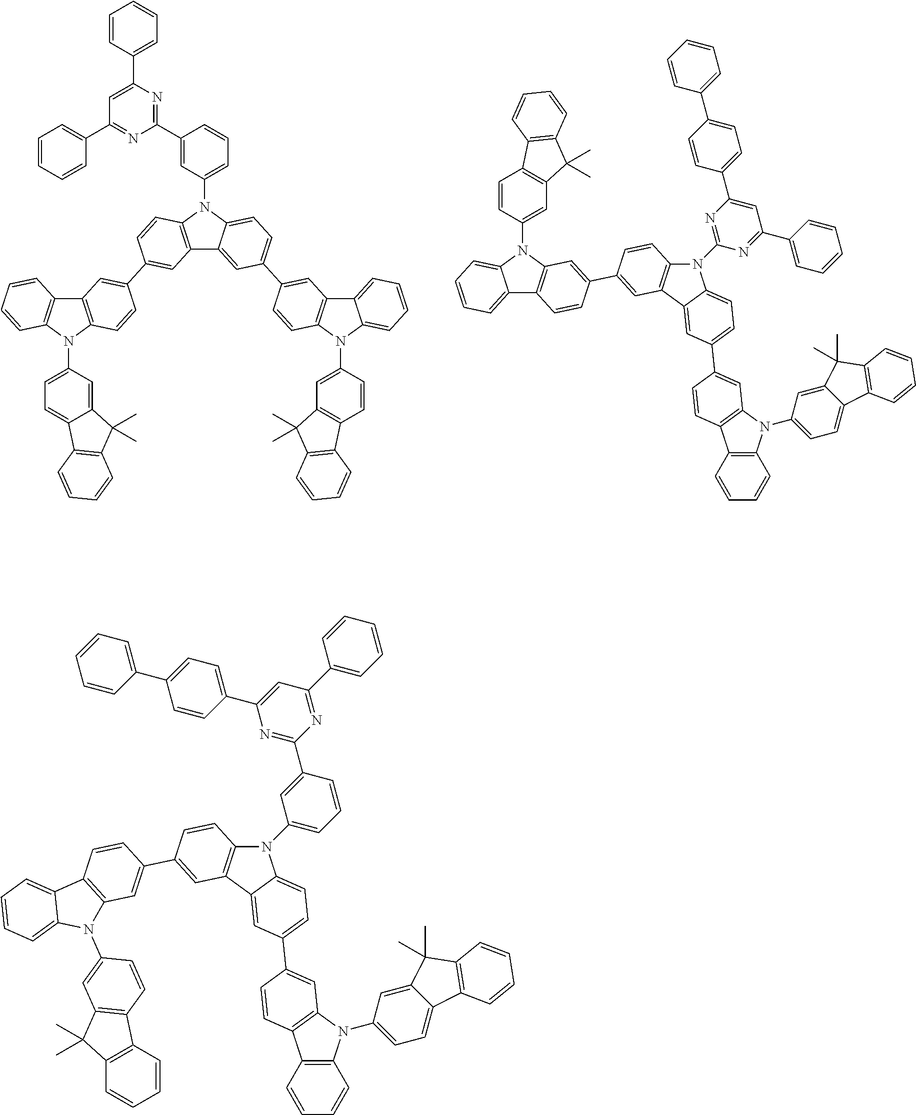

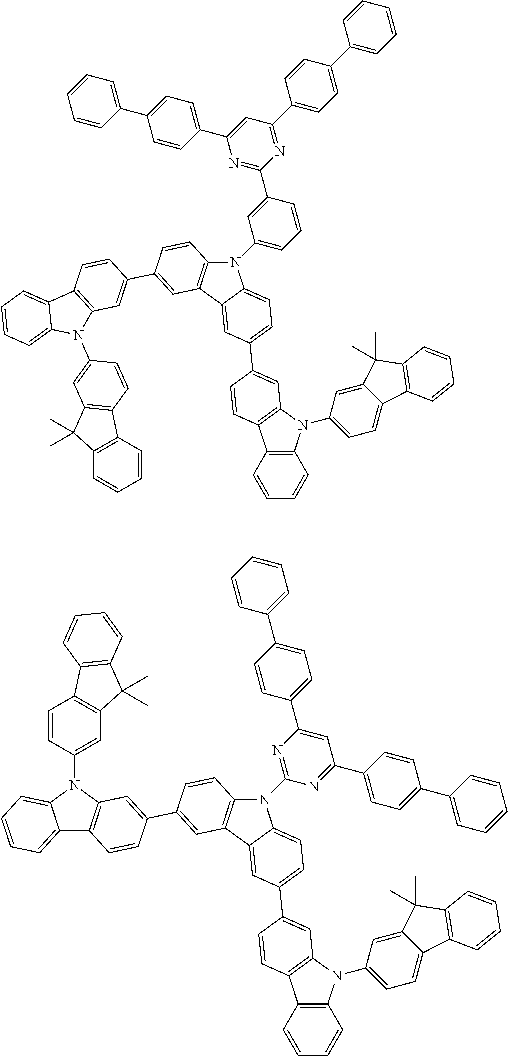 [SMILES: CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5cccc(-c6nc(-c7ccc(-c8ccccc8)cc7)cc(-c7ccc(-c8ccccc8)cc7)n6)c5)cc43)cc21 ; CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5nc(-c6ccc(-c7ccccc7)cc6)cc(-c6ccc(-c7ccccc7)cc6)n5)cc43)cc21]

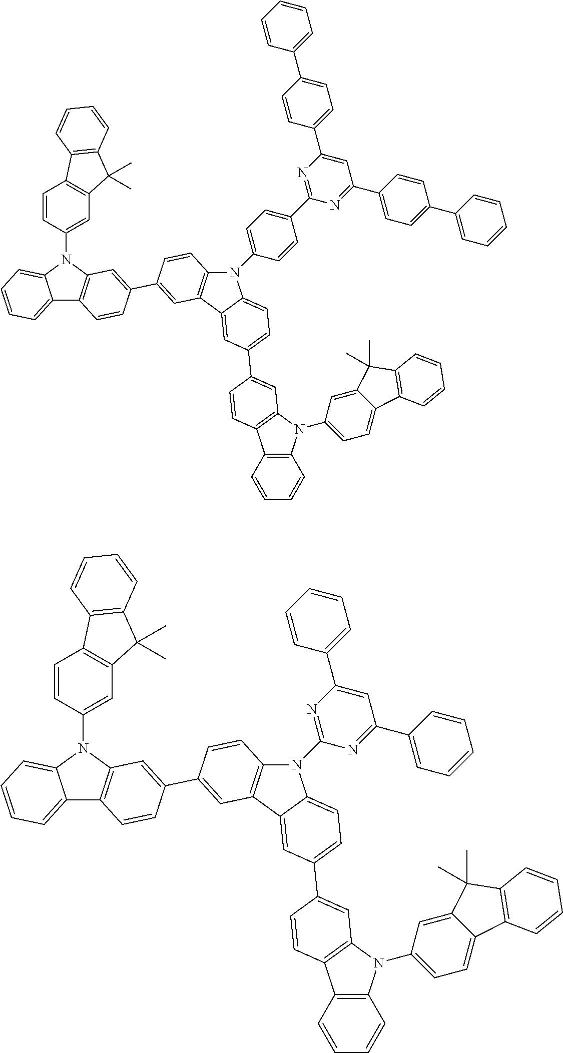 [SMILES: CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5ccc(-c6nc(-c7ccc(-c8ccccc8)cc7)cc(-c7ccc(-c8ccccc8)cc7)n6)cc5)cc43)cc21 ; CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5nc(-c6ccccc6)cc(-c6ccccc6)n5)cc43)cc21]

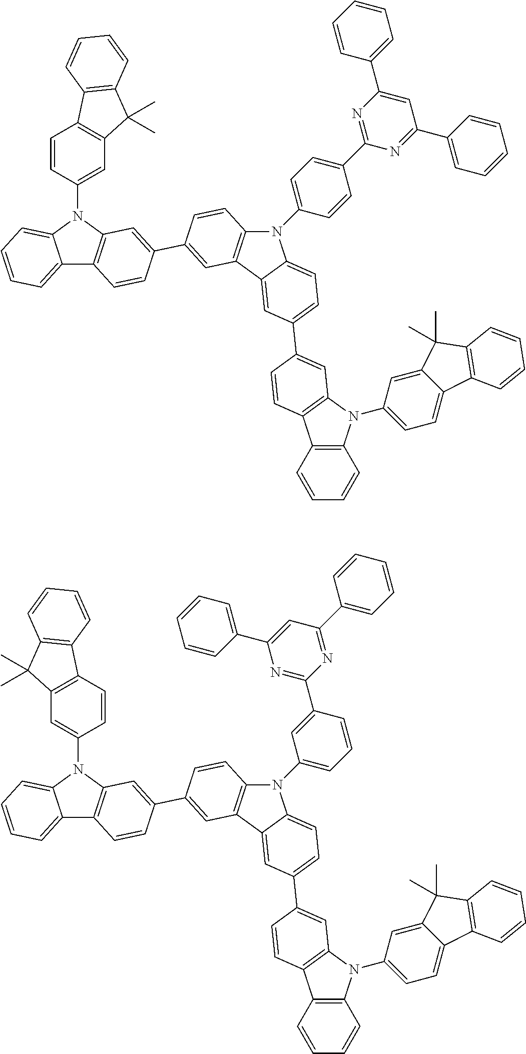 [SMILES: CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5ccc(-c6nc(-c7ccccc7)cc(-c7ccccc7)n6)cc5)cc43)cc21 ; CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5cccc(-c6nc(-c7ccccc7)cc(-c7ccccc7)n6)c5)cc43)cc21]

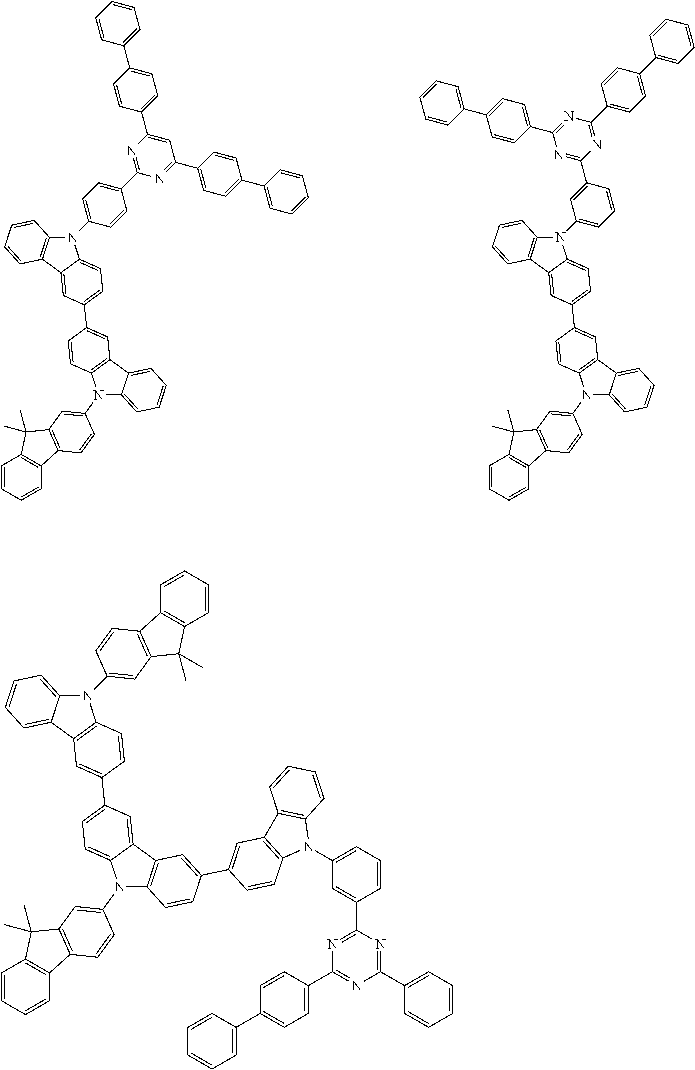 [SMILES: CC1(C)c2ccccc2-c2ccc(-n3c4ccccc4c4cc(-c5ccc6c(c5)c5ccccc5n6-c5ccc(-c6nc(-c7ccc(-c8ccccc8)cc7)cc(-c7ccc(-c8ccccc8)cc7)n6)cc5)ccc43)cc21 ; CC1(C)c2ccccc2-c2ccc(-n3c4ccccc4c4cc(-c5ccc6c(c5)c5ccccc5n6-c5cccc(-c6nc(-c7ccc(-c8ccccc8)cc7)nc(-c7ccc(-c8ccccc8)cc7)n6)c5)ccc43)cc21 ; CC1(C)c2ccccc2-c2ccc(-n3c4ccccc4c4cc(-c5ccc6c(c5)c5cc(-c7ccc8c(c7)c7ccccc7n8-c7cccc(-c8nc(-c9ccccc9)nc(-c9ccc(-c%10ccccc%10)cc9)n8)c7)ccc5n6-c5ccc6c(c5)C(C)(C)c5ccccc5-6)ccc43)cc21]

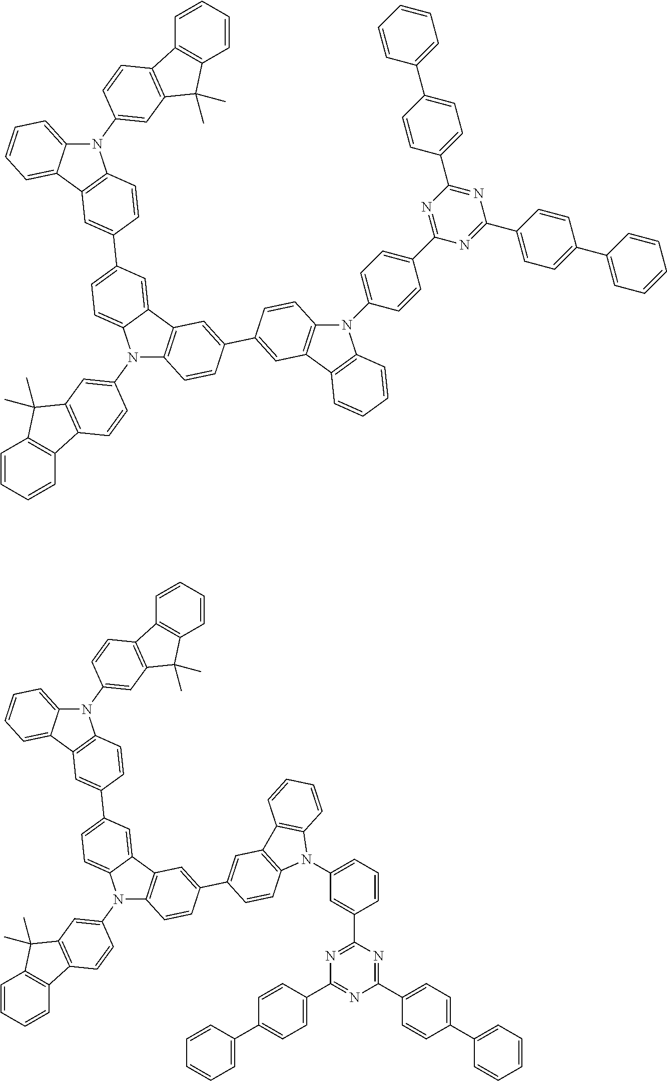 [SMILES: CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccc(-c%10ccccc%10)cc9)nc(-c9ccc(-c%10ccccc%10)cc9)n8)cc7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7cccc(-c8nc(-c9ccc(-c%10ccccc%10)cc9)nc(-c9ccc(-c%10ccccc%10)cc9)n8)c7)ccc5n6-c5ccc6c(c5)C(C)(C)c5ccccc5-6)ccc43)cc21]

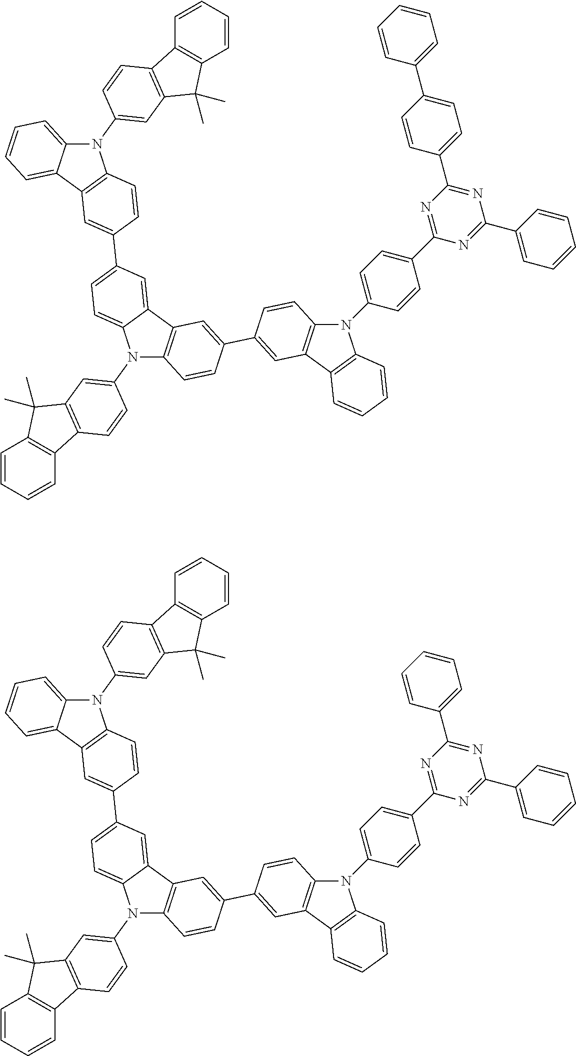 [SMILES: CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccccc9)nc(-c9ccc(-c%10ccccc%10)cc9)n8)cc7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccccc9)nc(-c9ccccc9)n8)cc7)ccc5n6-c5ccc6c(c5)C(C)(C)c5ccccc5-6)ccc43)cc21]

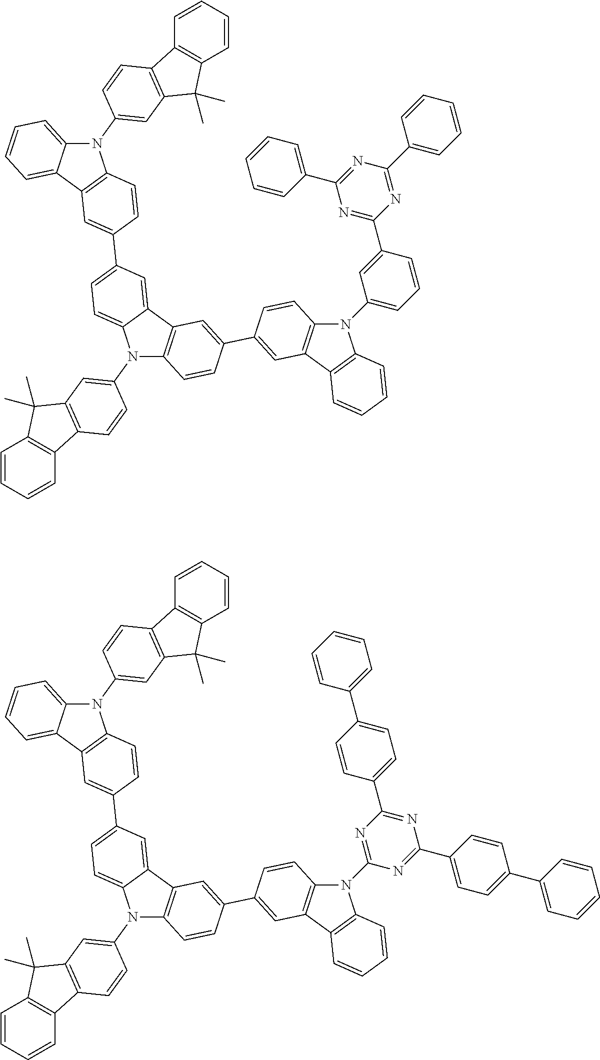 [SMILES: CC1(C)c2ccccc2-c2ccc(-n3c4ccccc4c4cc(-c5ccc6c(c5)c5cc(-c7ccc8c(c7)c7ccccc7n8-c7cccc(-c8nc(-c9ccccc9)nc(-c9ccccc9)n8)c7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7nc(-c8ccc(-c9ccccc9)cc8)nc(-c8ccc(-c9ccccc9)cc8)n7)ccc5n6-c5ccc6c(c5)C(C)(C)c5ccccc5-6)ccc43)cc21]

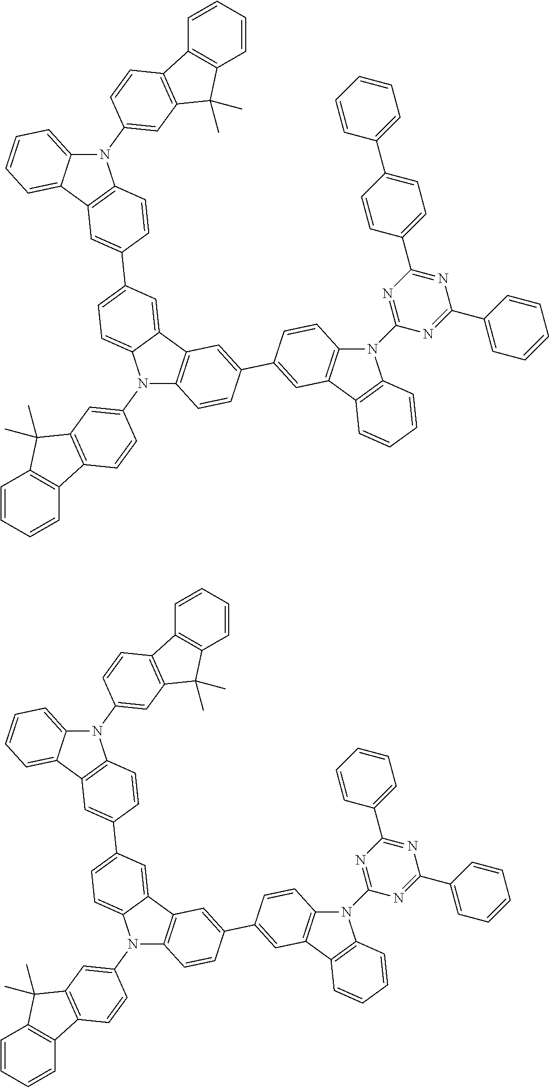 [SMILES: CC1(C)c2ccccc2-c2ccc(-n3c4ccccc4c4cc(-c5ccc6c(c5)c5cc(-c7ccc8c(c7)c7ccccc7n8-c7nc(-c8ccccc8)nc(-c8ccc(-c9ccccc9)cc8)n7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7nc(-c8ccccc8)nc(-c8ccccc8)n7)ccc5n6-c5ccc6c(c5)C(C)(C)c5ccccc5-6)ccc43)cc21]

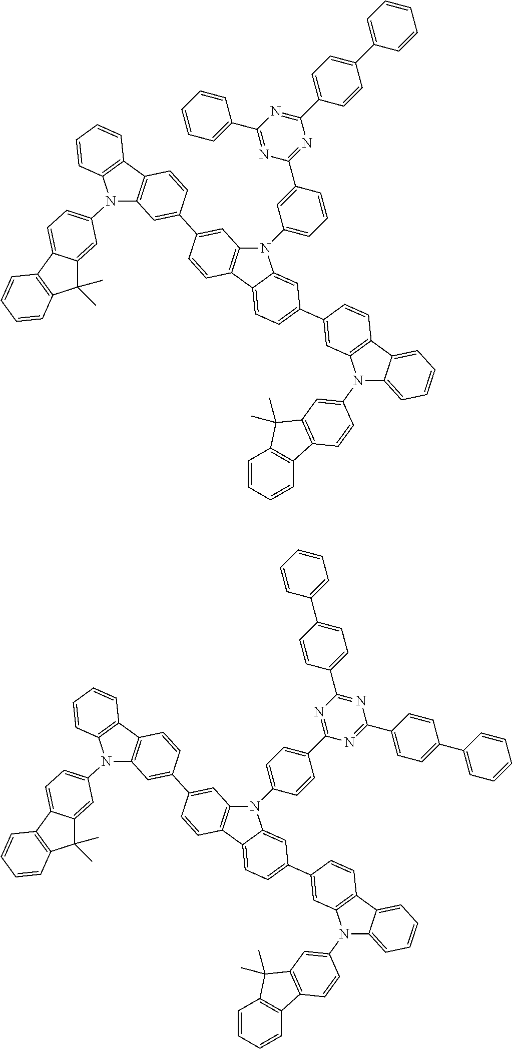 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7cccc(-c8nc(-c9ccccc9)nc(-c9ccc(-c%10ccccc%10)cc9)n8)c7)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7ccc(-c8nc(-c9ccc(-c%10ccccc%10)cc9)nc(-c9ccc(-c%10ccccc%10)cc9)n8)cc7)c6c5)cc43)cc21]

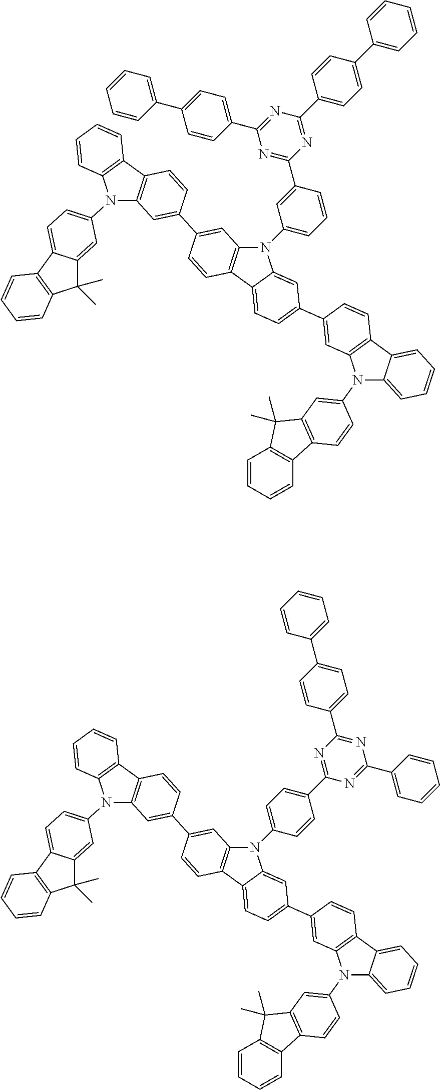 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7cccc(-c8nc(-c9ccc(-c%10ccccc%10)cc9)nc(-c9ccc(-c%10ccccc%10)cc9)n8)c7)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7ccc(-c8nc(-c9ccccc9)nc(-c9ccc(-c%10ccccc%10)cc9)n8)cc7)c6c5)cc43)cc21]

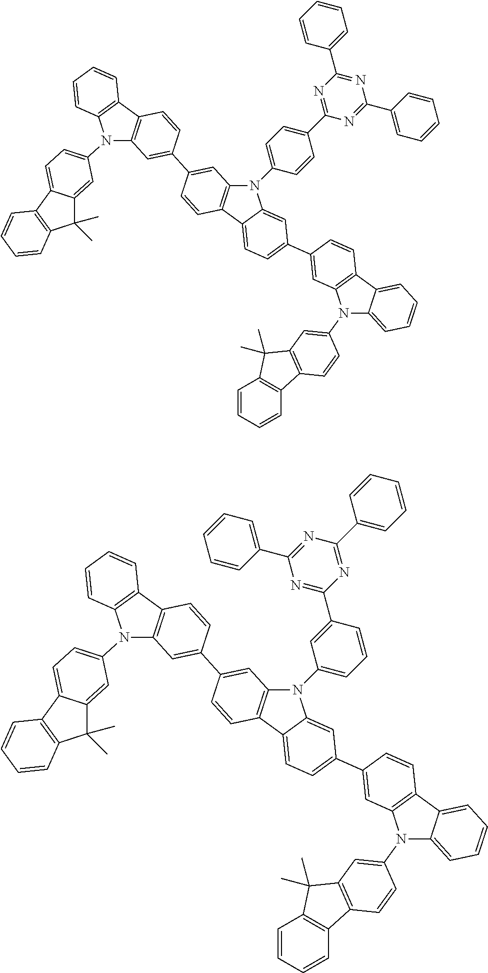 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7ccc(-c8nc(-c9ccccc9)nc(-c9ccccc9)n8)cc7)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7cccc(-c8nc(-c9ccccc9)nc(-c9ccccc9)n8)c7)c6c5)cc43)cc21]

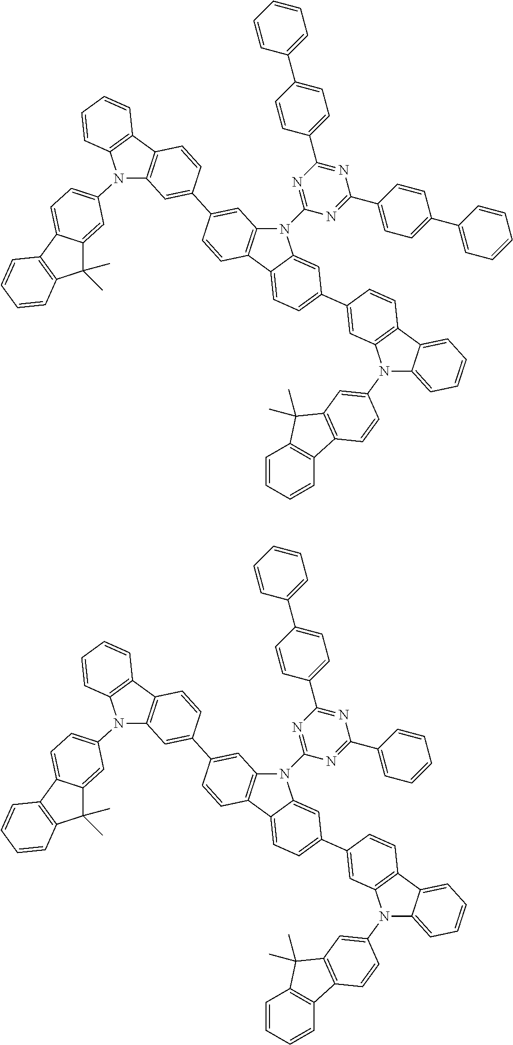 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7nc(-c8ccc(-c9ccccc9)cc8)nc(-c8ccc(-c9ccccc9)cc8)n7)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7nc(-c8ccccc8)nc(-c8ccc(-c9ccccc9)cc8)n7)c6c5)cc43)cc21]

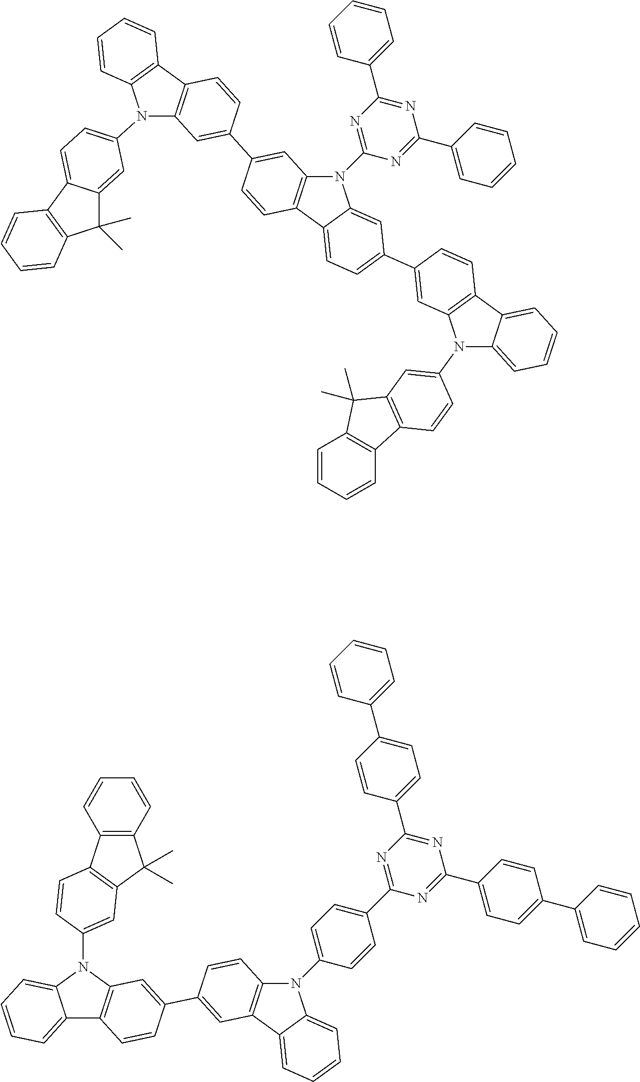 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7nc(-c8ccccc8)nc(-c8ccccc8)n7)c6c5)cc43)cc21 ; CC1(C)c2ccccc2-c2ccc(-n3c4ccccc4c4ccc(-c5ccc6c(c5)c5ccccc5n6-c5ccc(-c6nc(-c7ccc(-c8ccccc8)cc7)nc(-c7ccc(-c8ccccc8)cc7)n6)cc5)cc43)cc21]

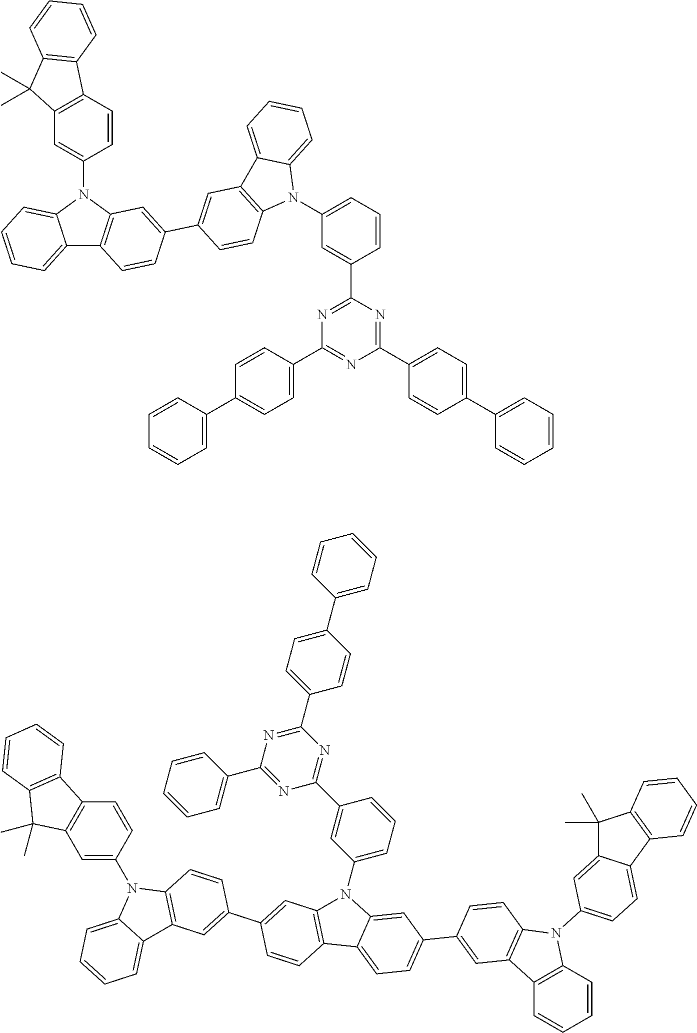 [SMILES: CC1(C)c2ccccc2-c2ccc(-n3c4ccccc4c4ccc(-c5ccc6c(c5)c5ccccc5n6-c5cccc(-c6nc(-c7ccc(-c8ccccc8)cc7)nc(-c7ccc(-c8ccccc8)cc7)n6)c5)cc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7cccc(-c8nc(-c9ccccc9)nc(-c9ccc(-c%10ccccc%10)cc9)n8)c7)c6c5)ccc43)cc21]

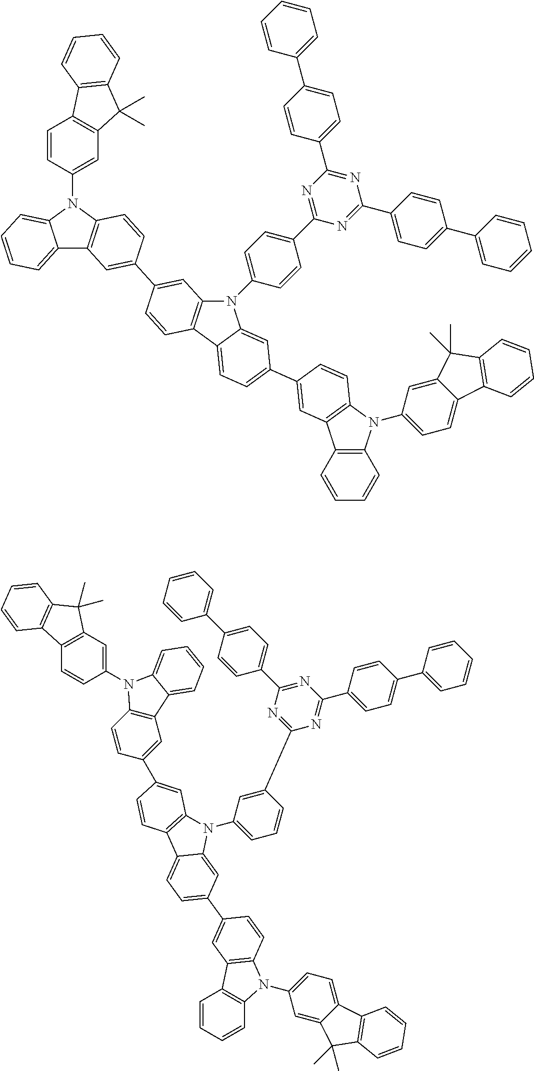 [SMILES: CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7ccc(-c8nc(-c9ccc(-c%10ccccc%10)cc9)nc(-c9ccc(-c%10ccccc%10)cc9)n8)cc7)c6c5)ccc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7cccc(-c8nc(-c9ccc(-c%10ccccc%10)cc9)nc(-c9ccc(-c%10ccccc%10)cc9)n8)c7)c6c5)ccc43)cc21]

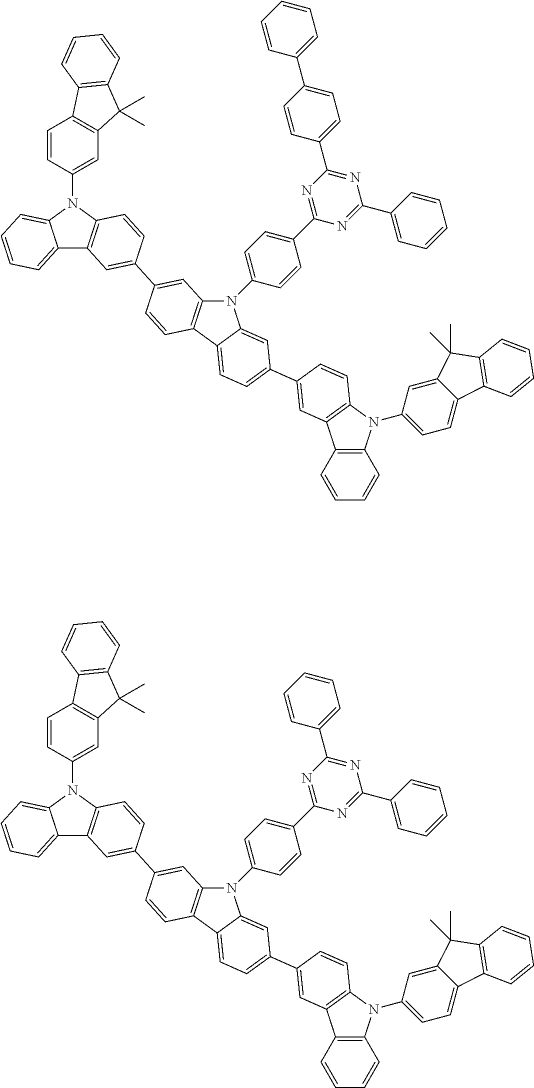 [SMILES: CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7ccc(-c8nc(-c9ccccc9)nc(-c9ccc(-c%10ccccc%10)cc9)n8)cc7)c6c5)ccc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7ccc(-c8nc(-c9ccccc9)nc(-c9ccccc9)n8)cc7)c6c5)ccc43)cc21]

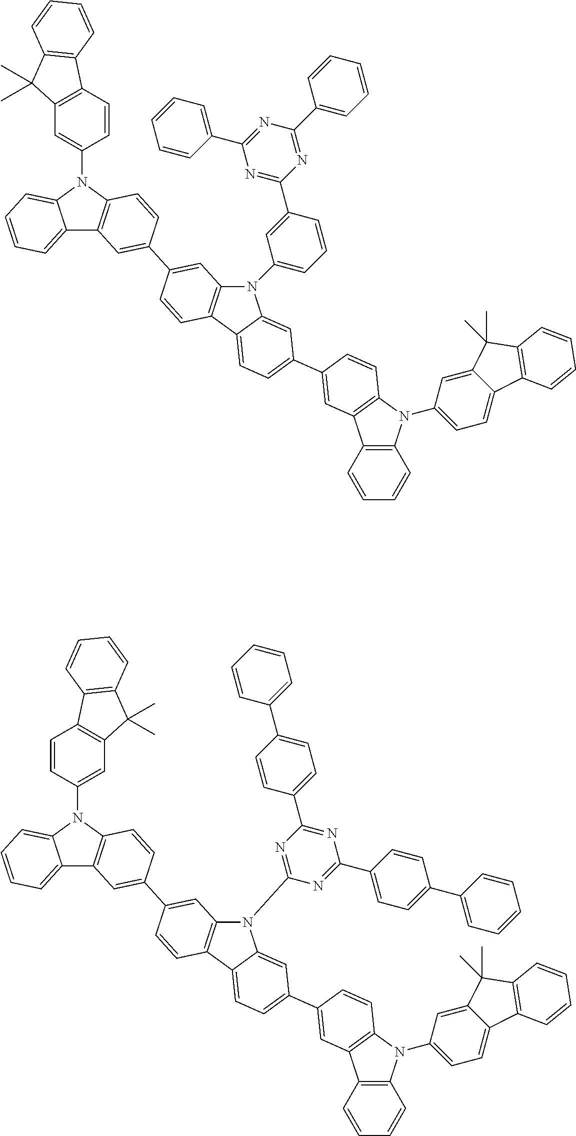 [SMILES: CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7cccc(-c8nc(-c9ccccc9)nc(-c9ccccc9)n8)c7)c6c5)ccc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7nc(-c8ccc(-c9ccccc9)cc8)nc(-c8ccc(-c9ccccc9)cc8)n7)c6c5)ccc43)cc21]

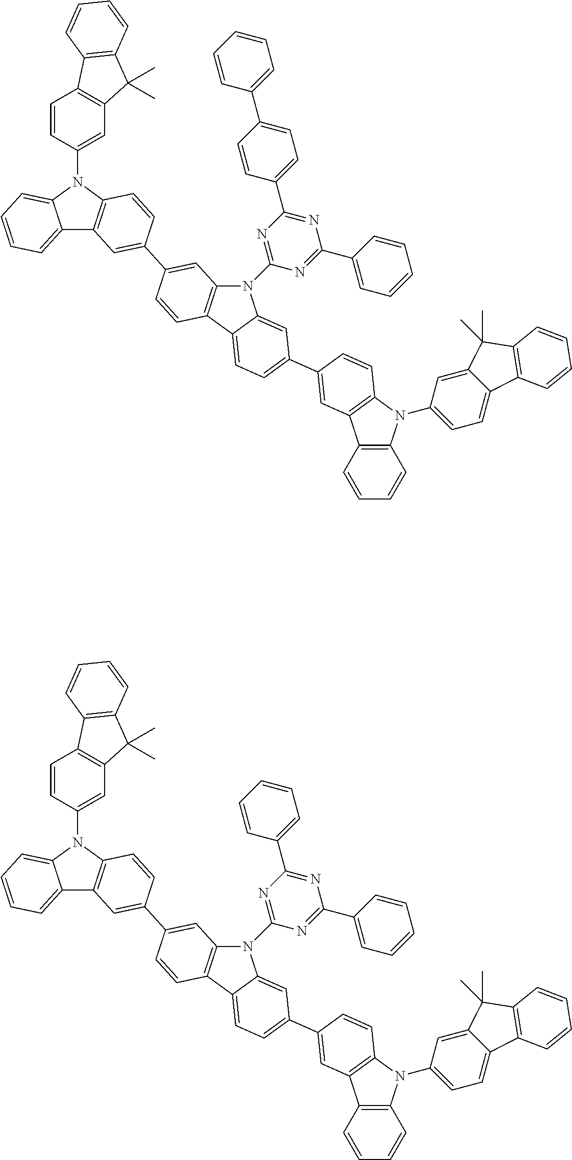 [SMILES: CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7nc(-c8ccccc8)nc(-c8ccc(-c9ccccc9)cc8)n7)c6c5)ccc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7nc(-c8ccccc8)nc(-c8ccccc8)n7)c6c5)ccc43)cc21]

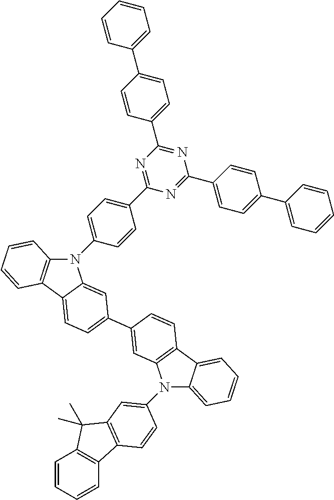 [SMILES: CC1(C)c2ccccc2-c2ccc(-n3c4ccccc4c4ccc(-c5ccc6c7ccccc7n(-c7ccc(-c8nc(-c9ccc(-c%10ccccc%10)cc9)nc(-c9ccc(-c%10ccccc%10)cc9)n8)cc7)c6c5)cc43)cc21]

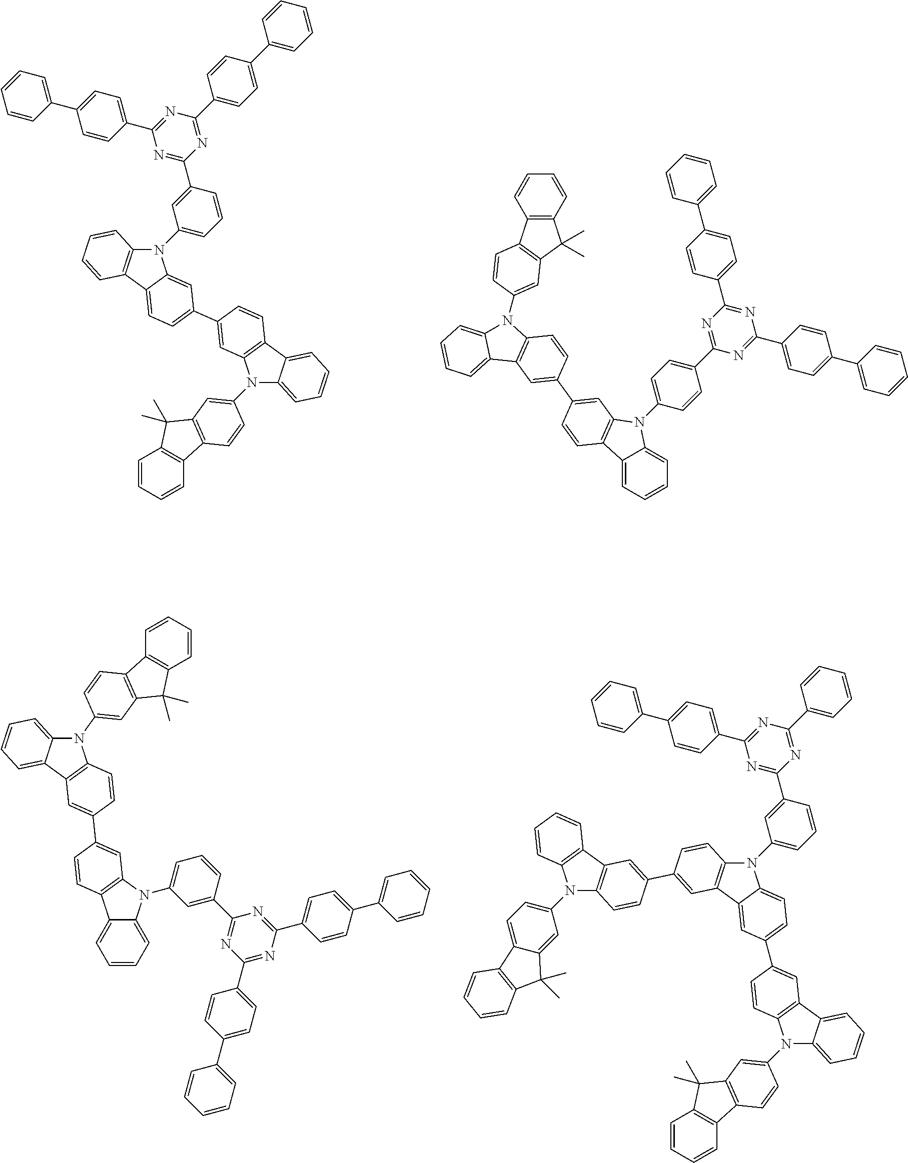

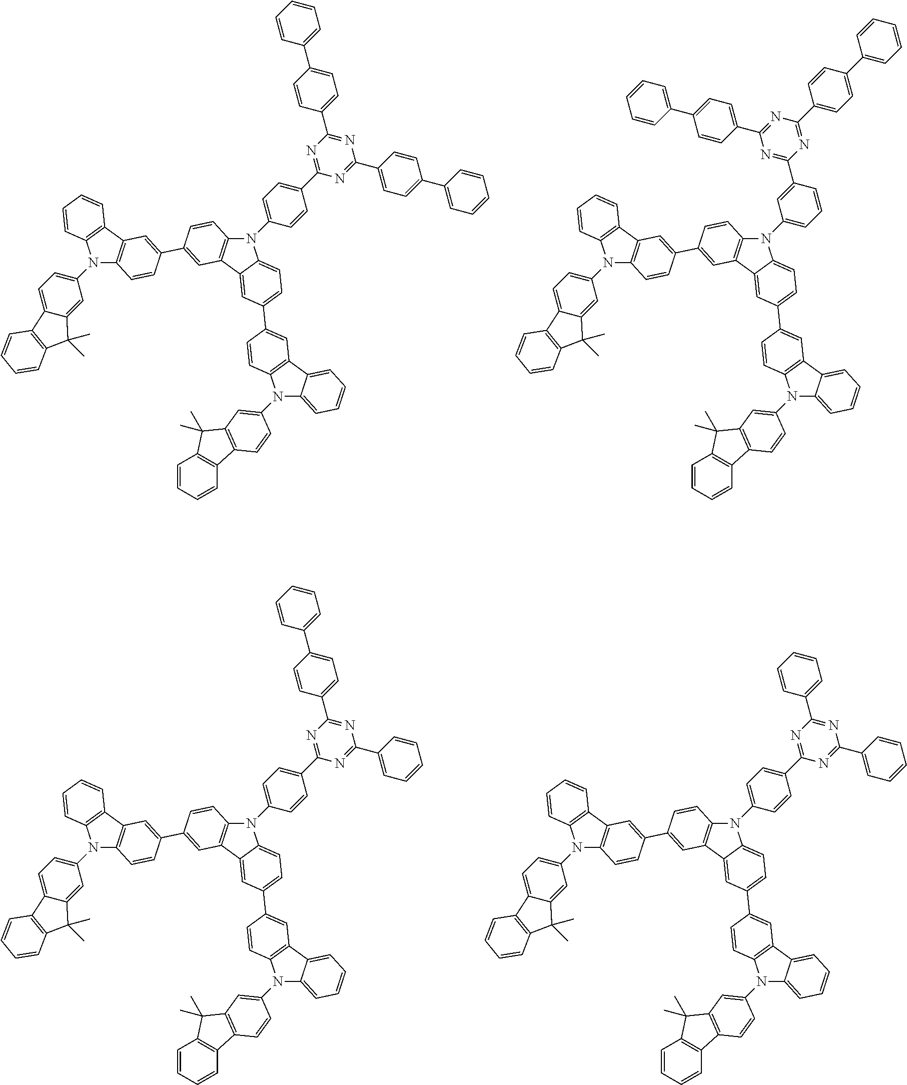

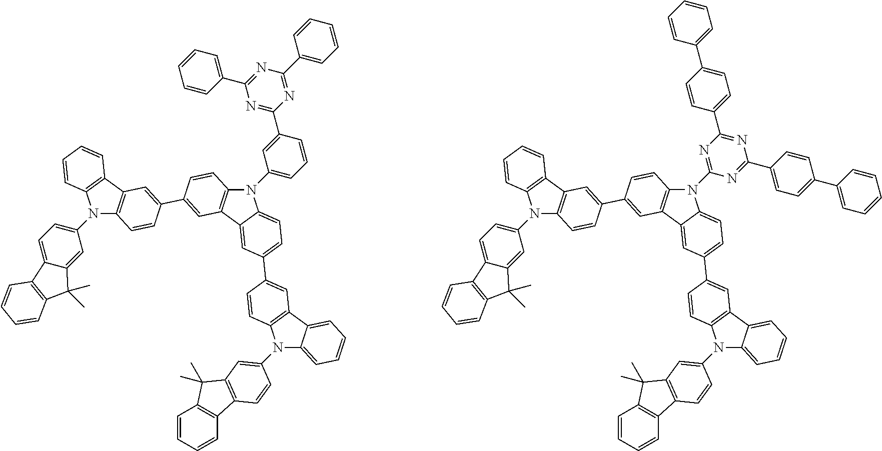 [SMILES: CC1(C)c2ccccc2-c2ccc(-n3c4ccccc4c4cc(-c5ccc6c(c5)c5cc(-c7ccc8c(c7)c7ccccc7n8-c7ccc8c(c7)C(C)(C)c7ccccc7-8)ccc5n6-c5cccc(-c6nc(-c7ccccc7)nc(-c7ccccc7)n6)c5)ccc43)cc21 ; CC1(C)c2ccccc2-c2ccc(-n3c4ccccc4c4cc(-c5ccc6c(c5)c5cc(-c7ccc8c(c7)c7ccccc7n8-c7ccc8c(c7)C(C)(C)c7ccccc7-8)ccc5n6-c5nc(-c6ccc(-c7ccccc7)cc6)nc(-c6ccc(-c7ccccc7)cc6)n5)ccc43)cc21]

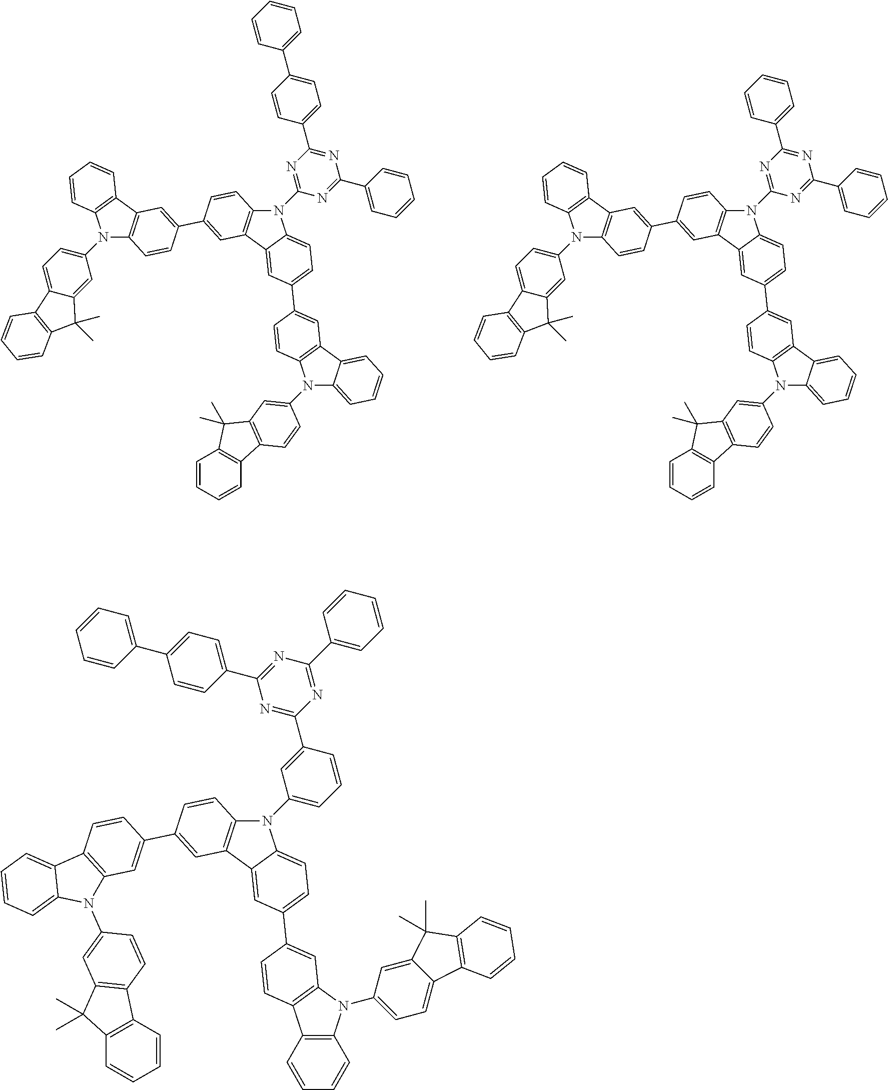

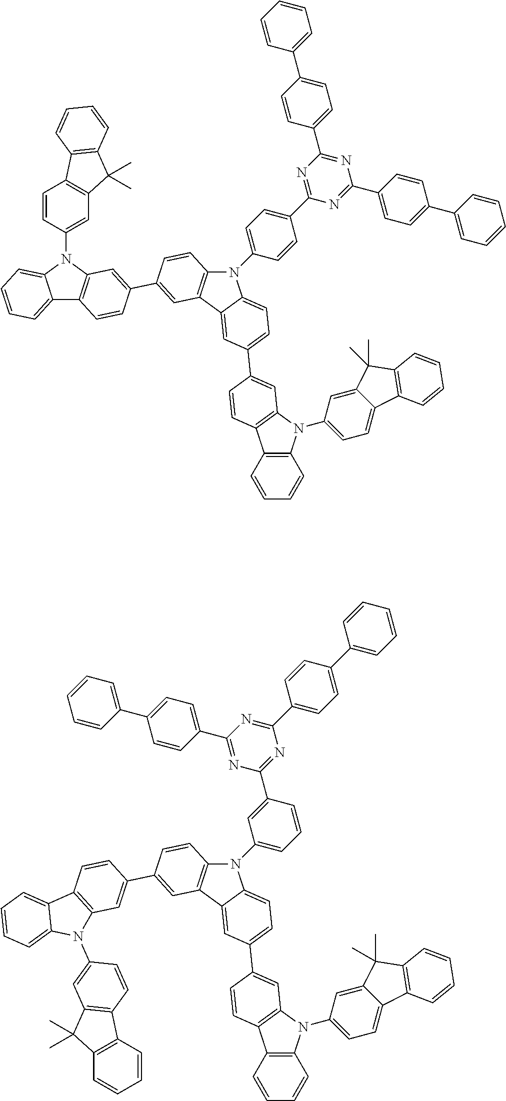 [SMILES: CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5ccc(-c6nc(-c7ccc(-c8ccccc8)cc7)nc(-c7ccc(-c8ccccc8)cc7)n6)cc5)cc43)cc21 ; CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5cccc(-c6nc(-c7ccc(-c8ccccc8)cc7)nc(-c7ccc(-c8ccccc8)cc7)n6)c5)cc43)cc21]

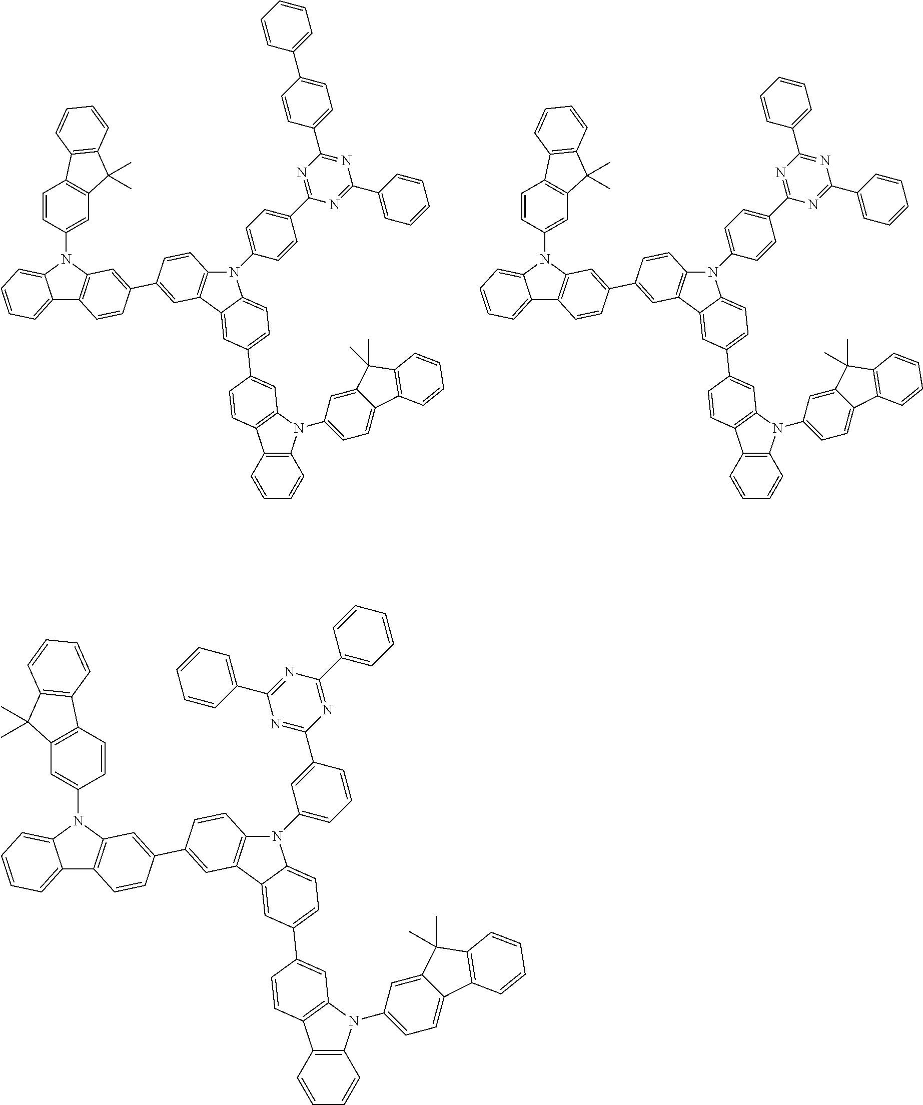

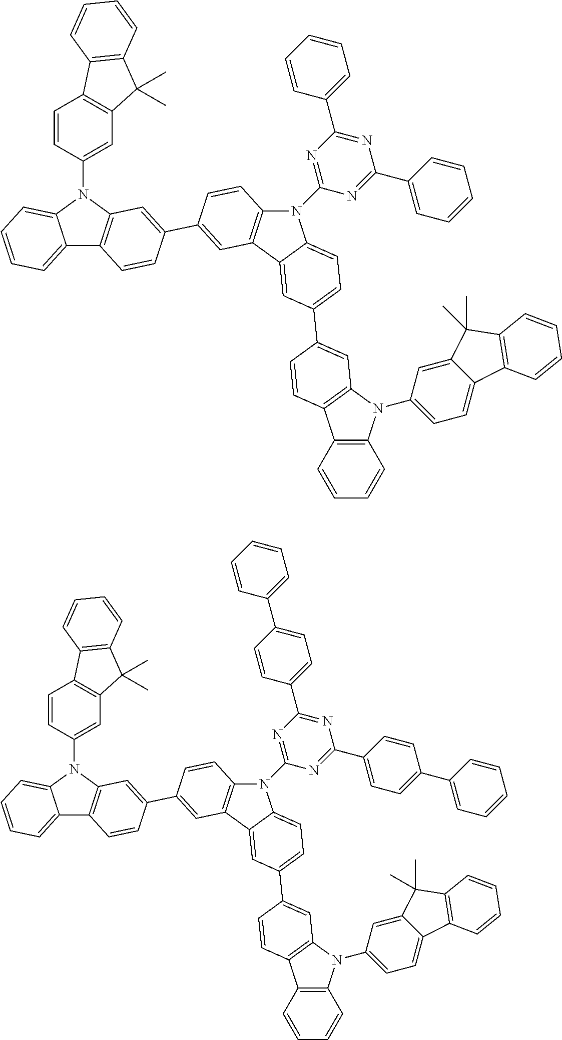 [SMILES: CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5nc(-c6ccccc6)nc(-c6ccccc6)n5)cc43)cc21 ; CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5nc(-c6ccc(-c7ccccc7)cc6)nc(-c6ccc(-c7ccccc7)cc6)n5)cc43)cc21]

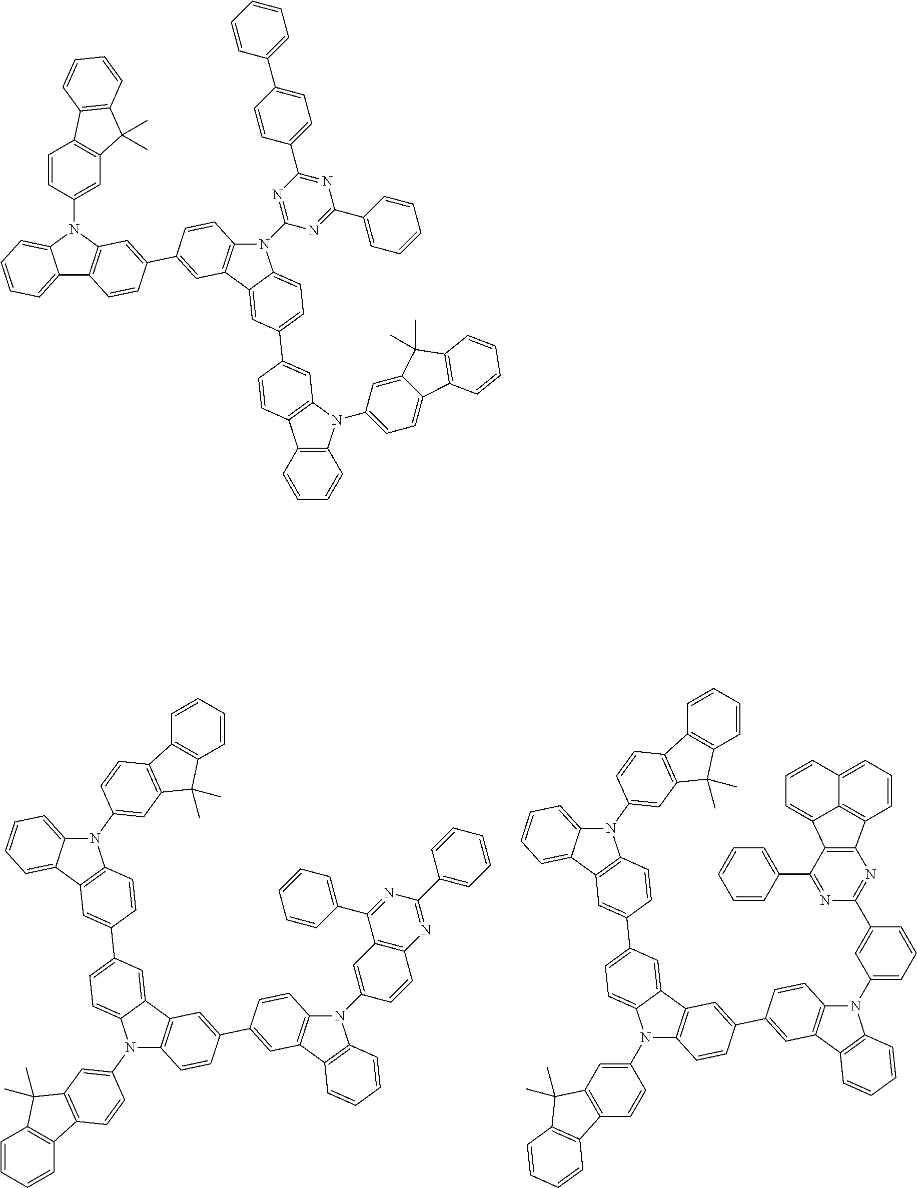

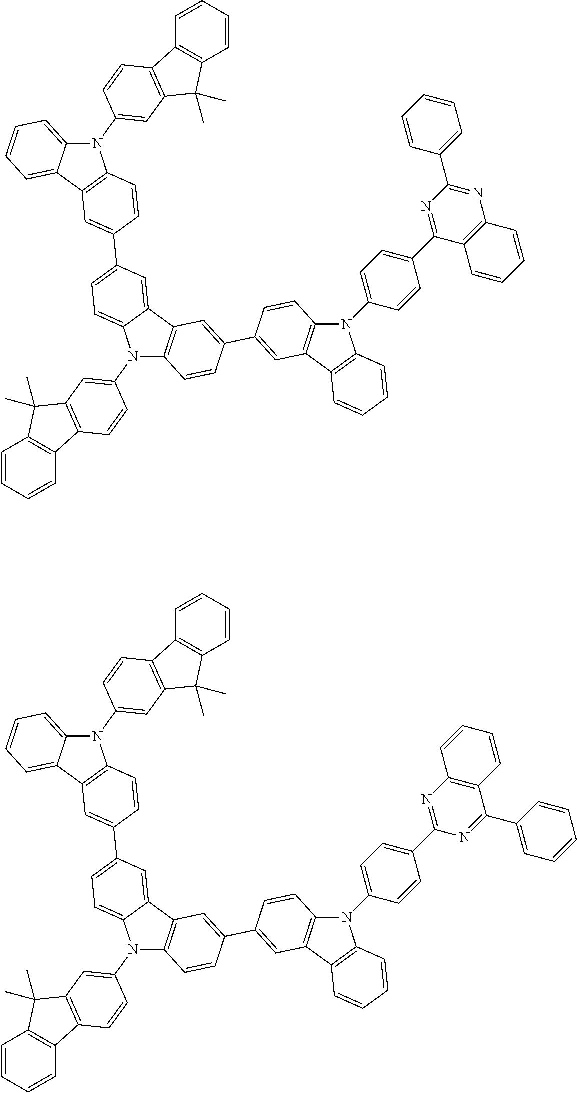 [SMILES: CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccccc9)nc9ccccc89)cc7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7ccc(-c8nc(-c9ccccc9)c9ccccc9n8)cc7)ccc5n6-c5ccc6c(c5)C(C)(C)c5ccccc5-6)ccc43)cc21]

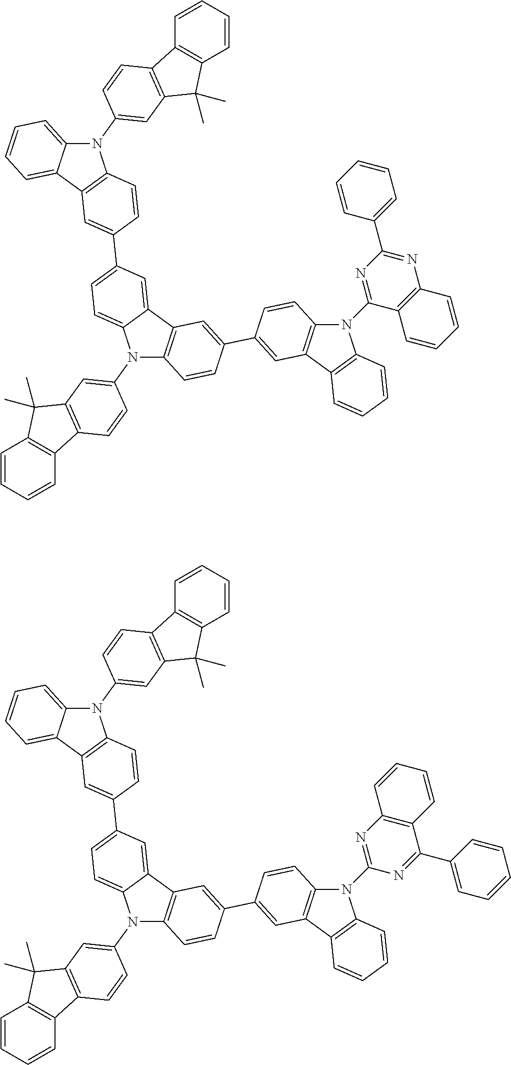 [SMILES: CC1(C)c2ccccc2-c2ccc(-n3c4ccccc4c4cc(-c5ccc6c(c5)c5cc(-c7ccc8c(c7)c7ccccc7n8-c7nc(-c8ccccc8)nc8ccccc78)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7nc(-c8ccccc8)c8ccccc8n7)ccc5n6-c5ccc6c(c5)C(C)(C)c5ccccc5-6)ccc43)cc21]

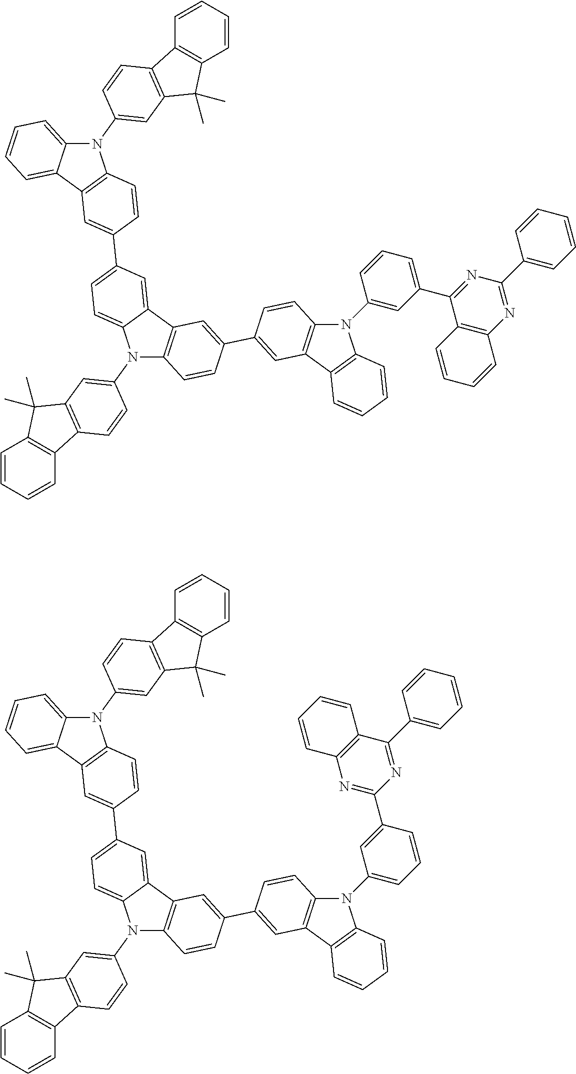 [SMILES: CC1(C)c2ccccc2-c2ccc(-n3c4ccccc4c4cc(-c5ccc6c(c5)c5cc(-c7ccc8c(c7)c7ccccc7n8-c7cccc(-c8nc(-c9ccccc9)nc9ccccc89)c7)ccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5cc(-c7ccc8c(c7)c7ccccc7n8-c7cccc(-c8nc(-c9ccccc9)c9ccccc9n8)c7)ccc5n6-c5ccc6c(c5)C(C)(C)c5ccccc5-6)ccc43)cc21]

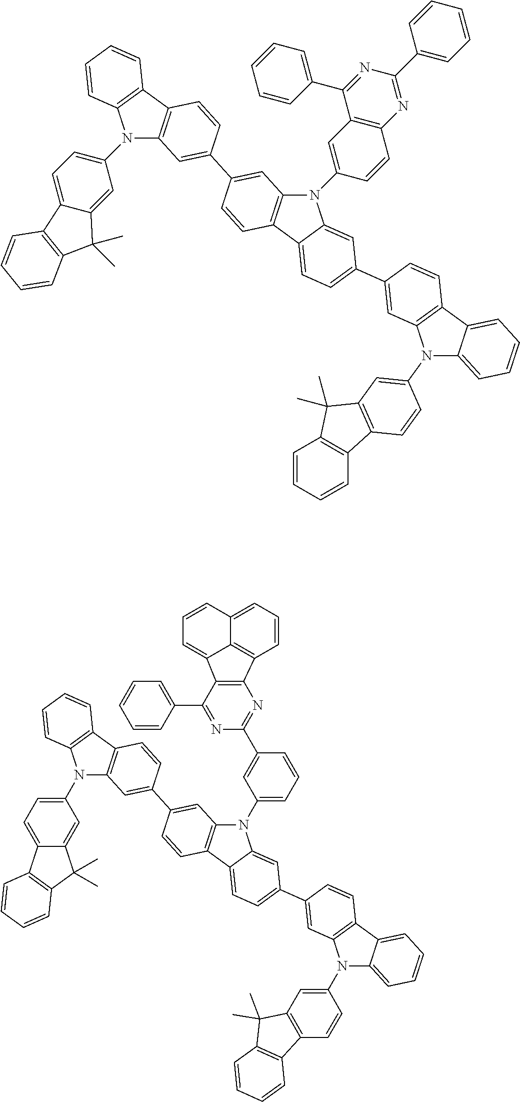 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7ccc8nc(-c9ccccc9)nc(-c9ccccc9)c8c7)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7cccc(-c8nc(-c9ccccc9)c9c(n8)-c8cccc%10cccc-9c8%10)c7)c6c5)cc43)cc21]

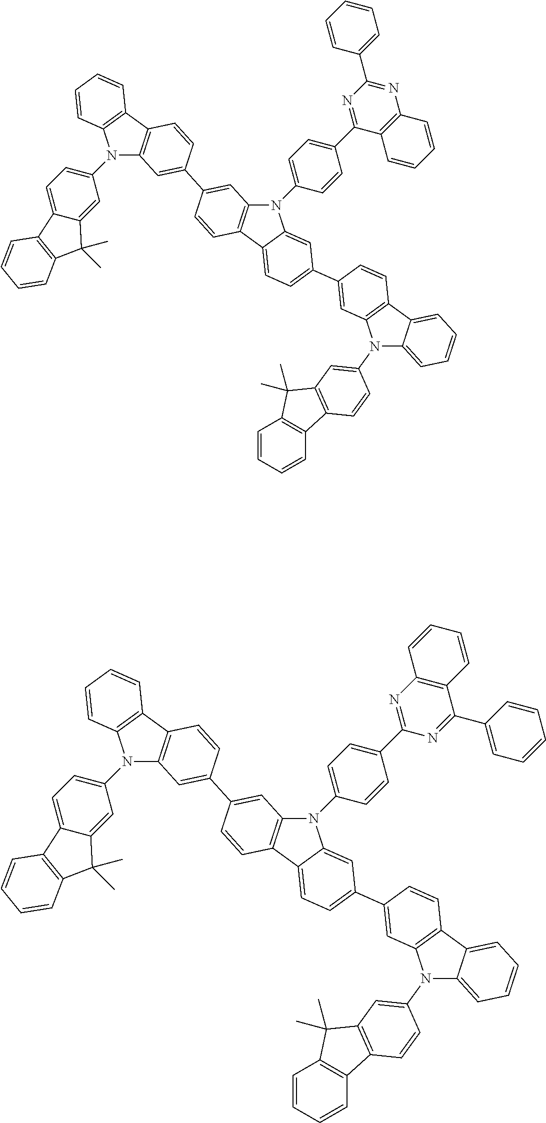 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7ccc(-c8nc(-c9ccccc9)nc9ccccc89)cc7)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7ccc(-c8nc(-c9ccccc9)c9ccccc9n8)cc7)c6c5)cc43)cc21]

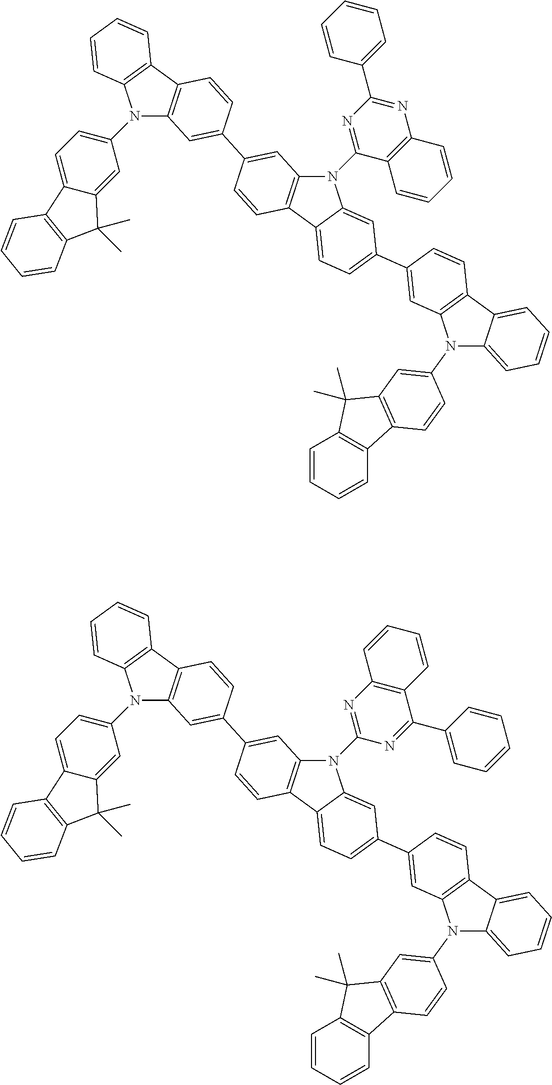 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7nc(-c8ccccc8)nc8ccccc78)c6c5)cc43)cc21 ; CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7nc(-c8ccccc8)c8ccccc8n7)c6c5)cc43)cc21]

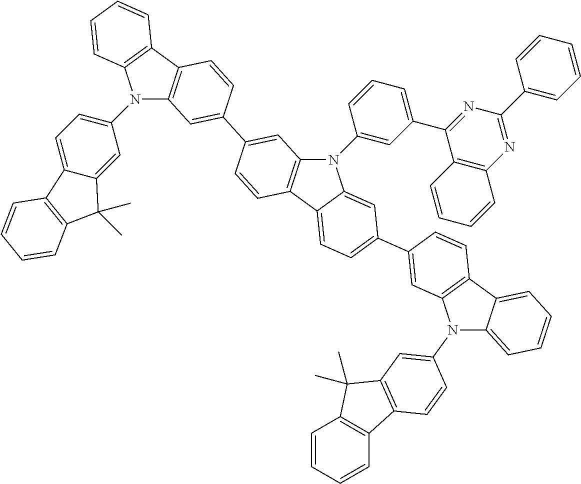 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7cccc(-c8nc(-c9ccccc9)nc9ccccc89)c7)c6c5)cc43)cc21]

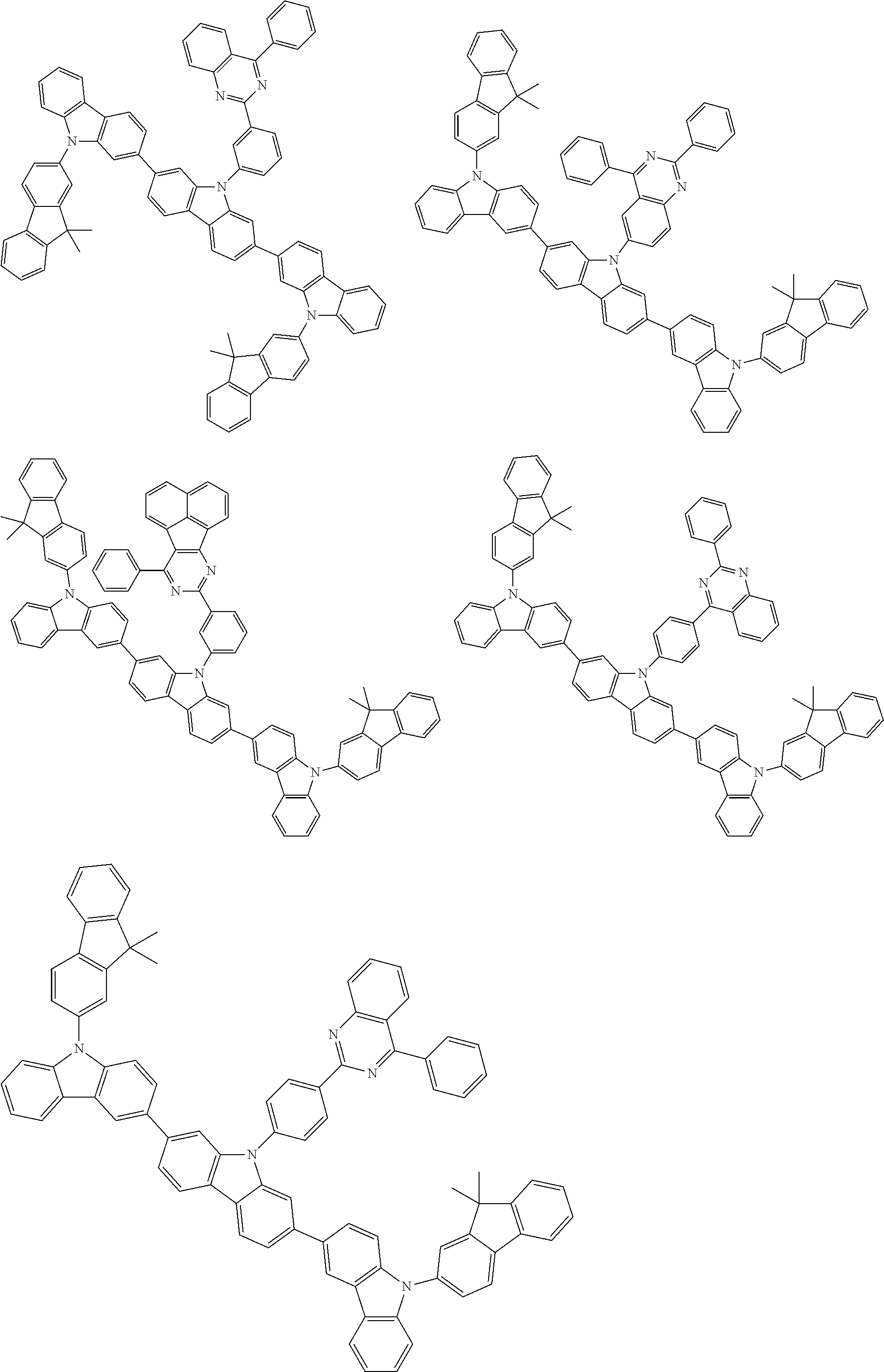

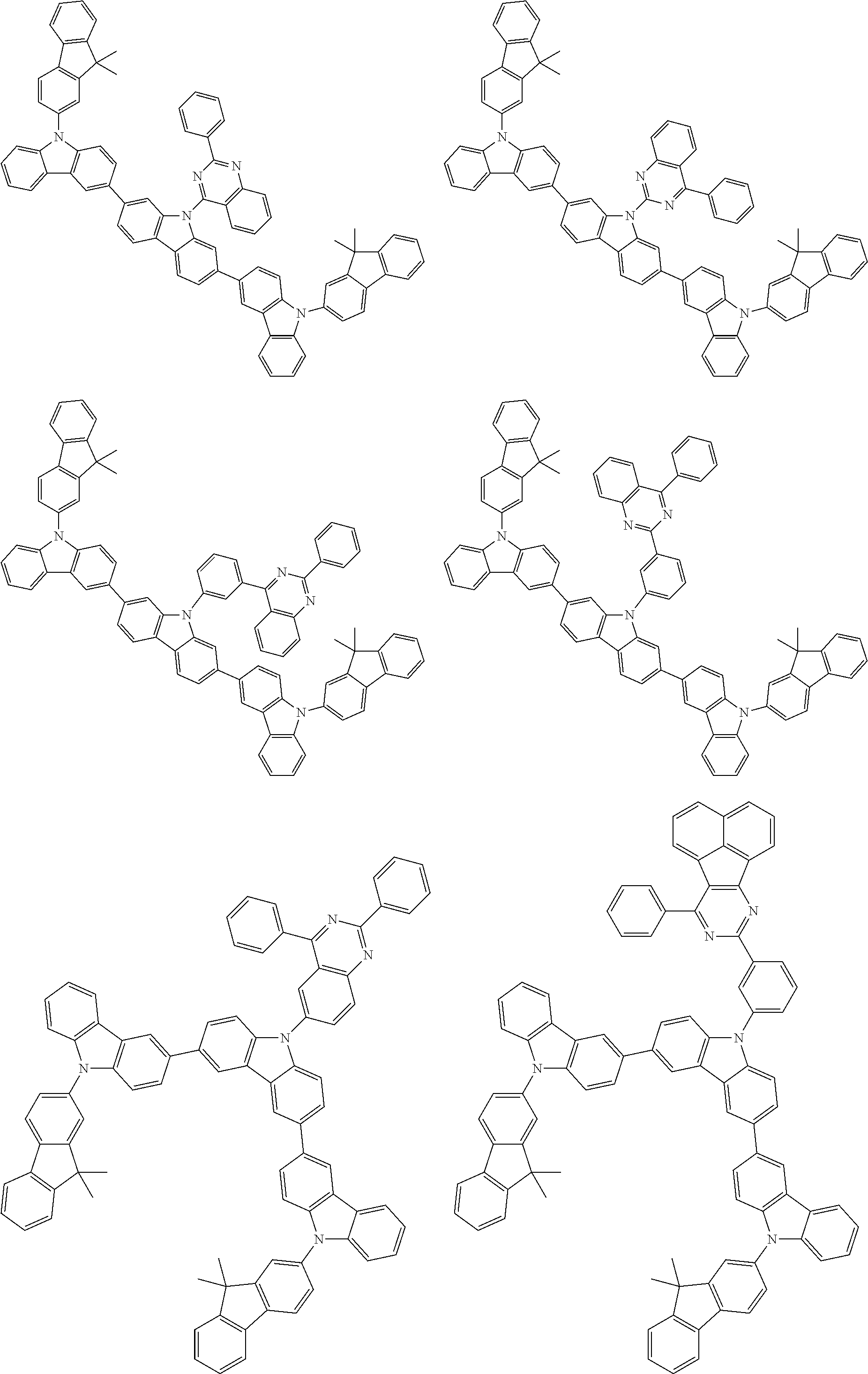

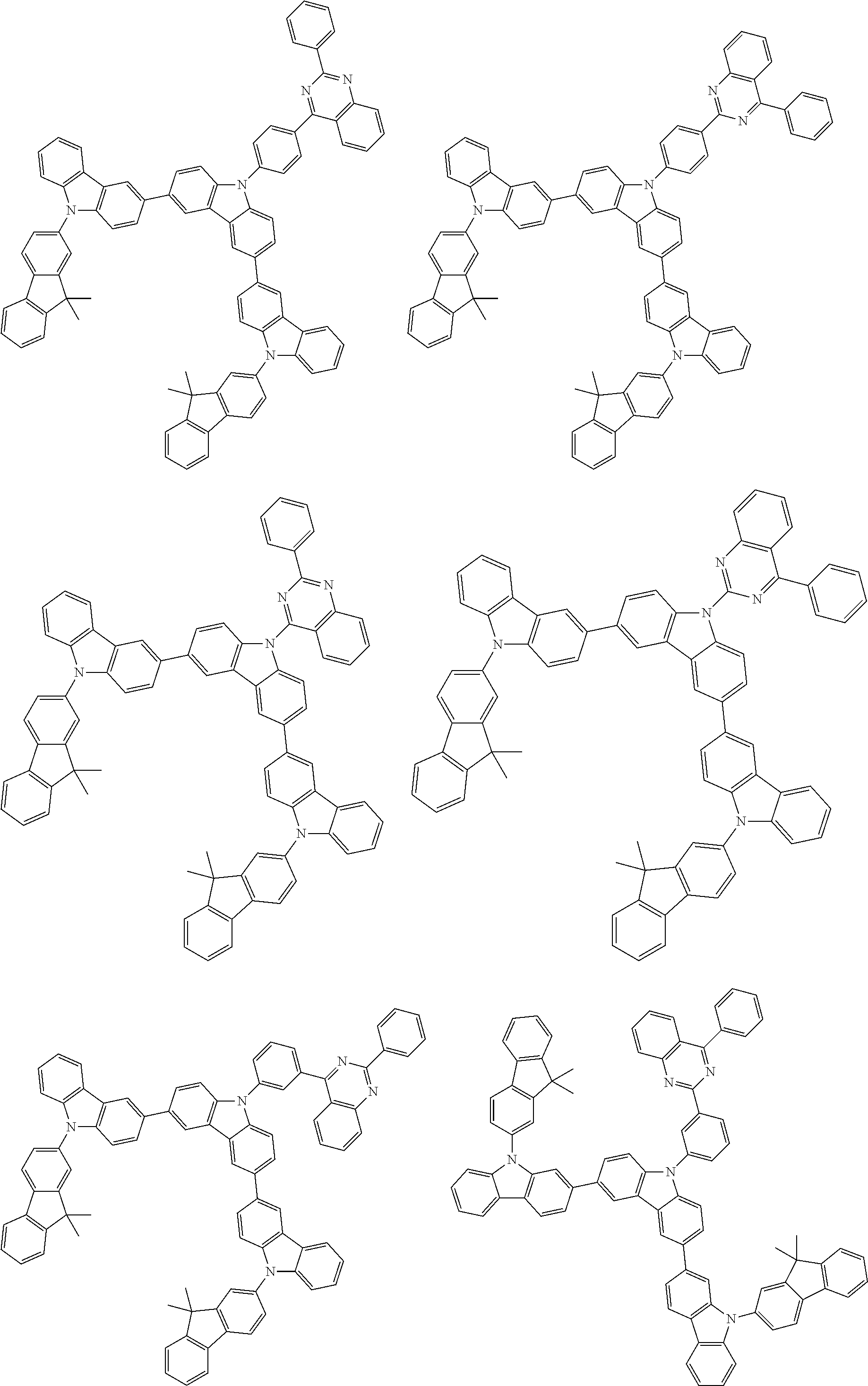

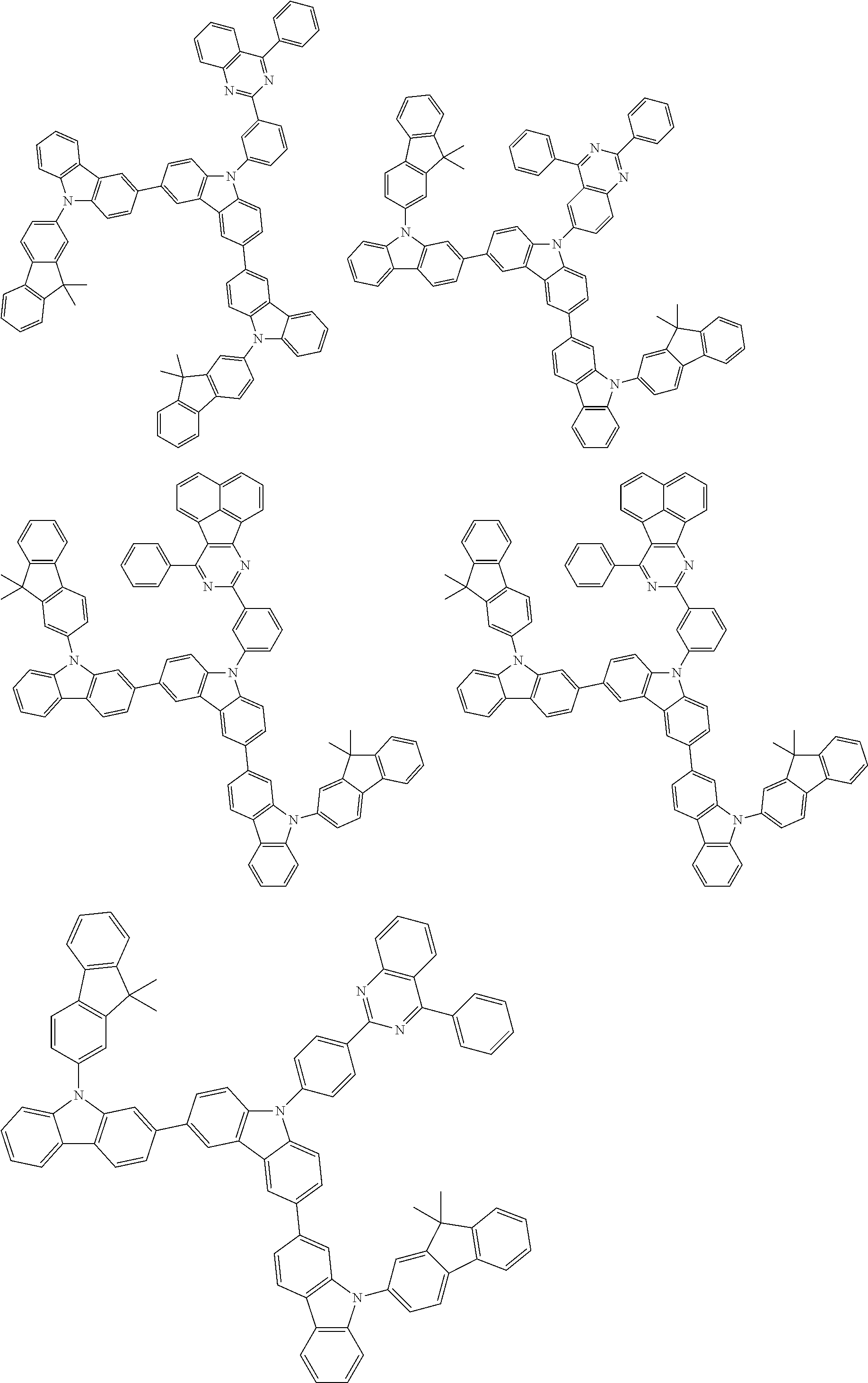

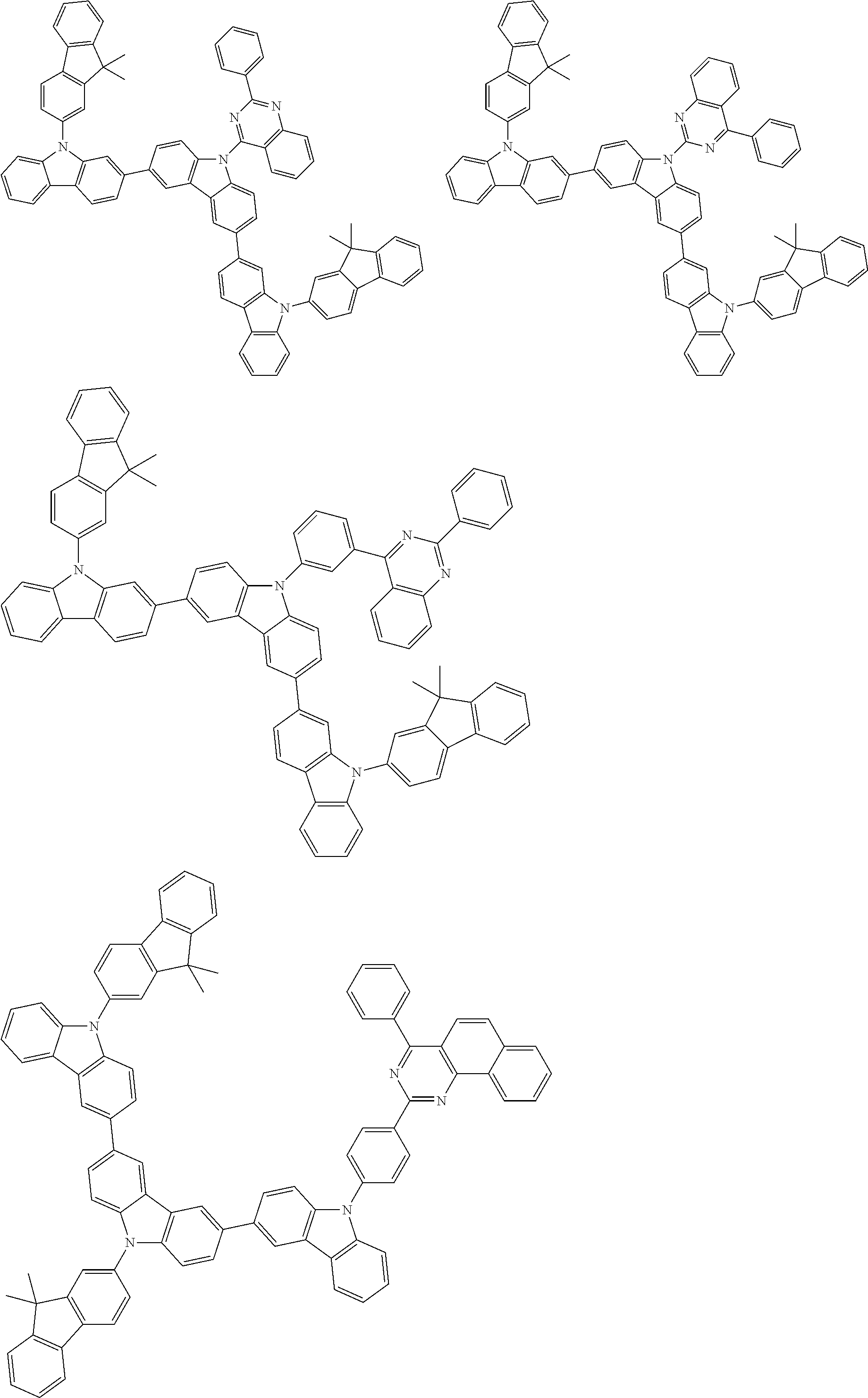

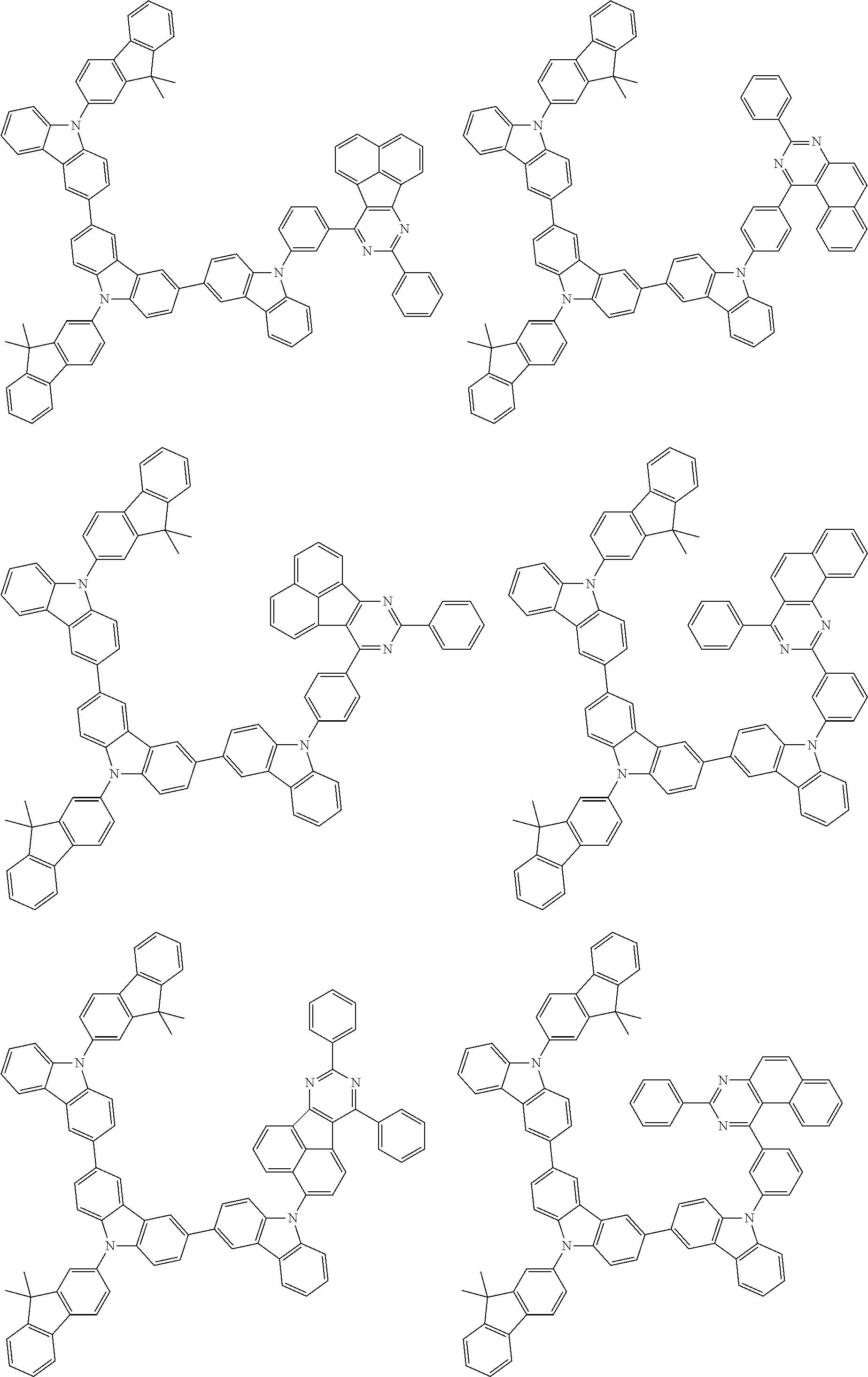

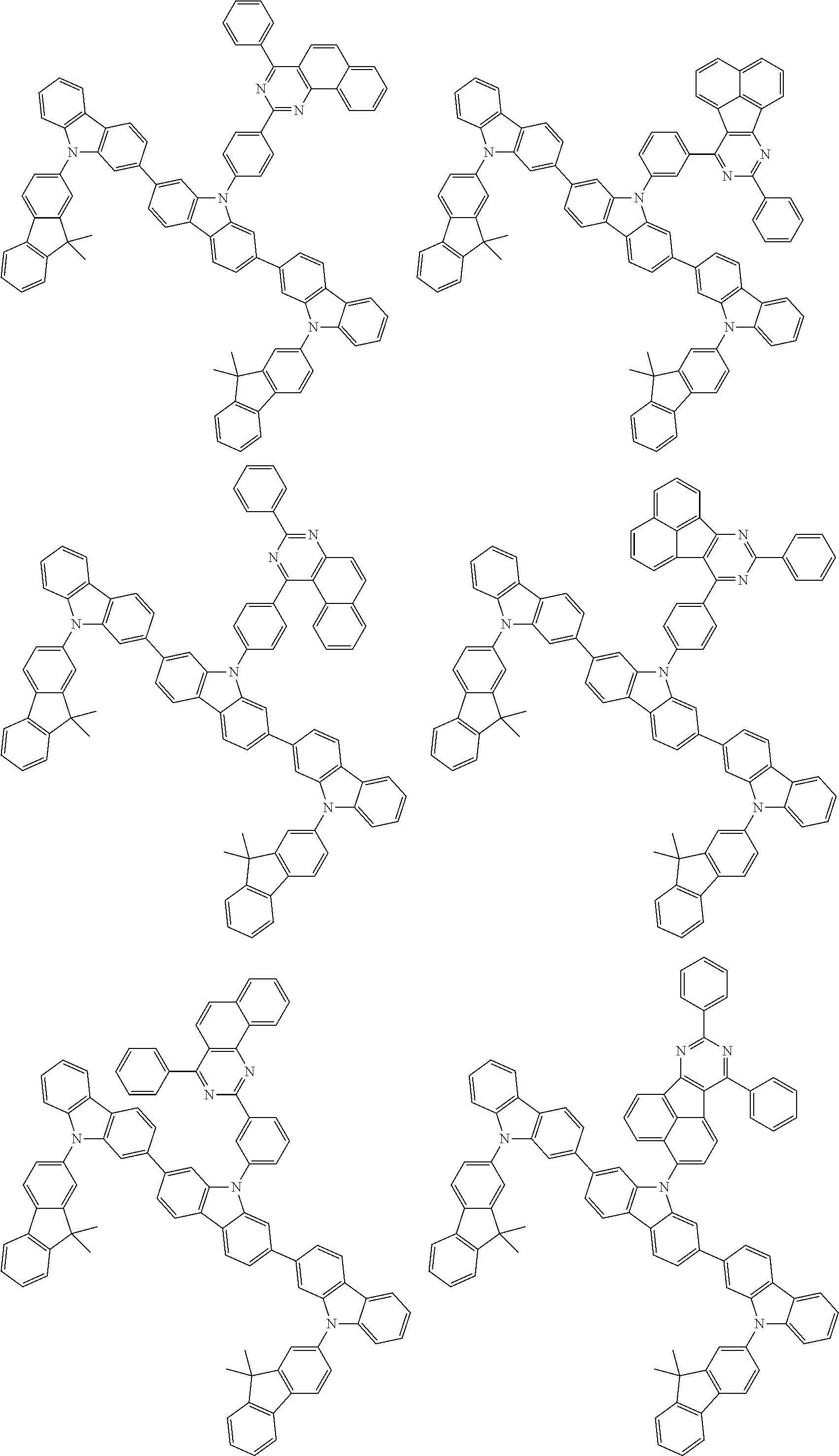

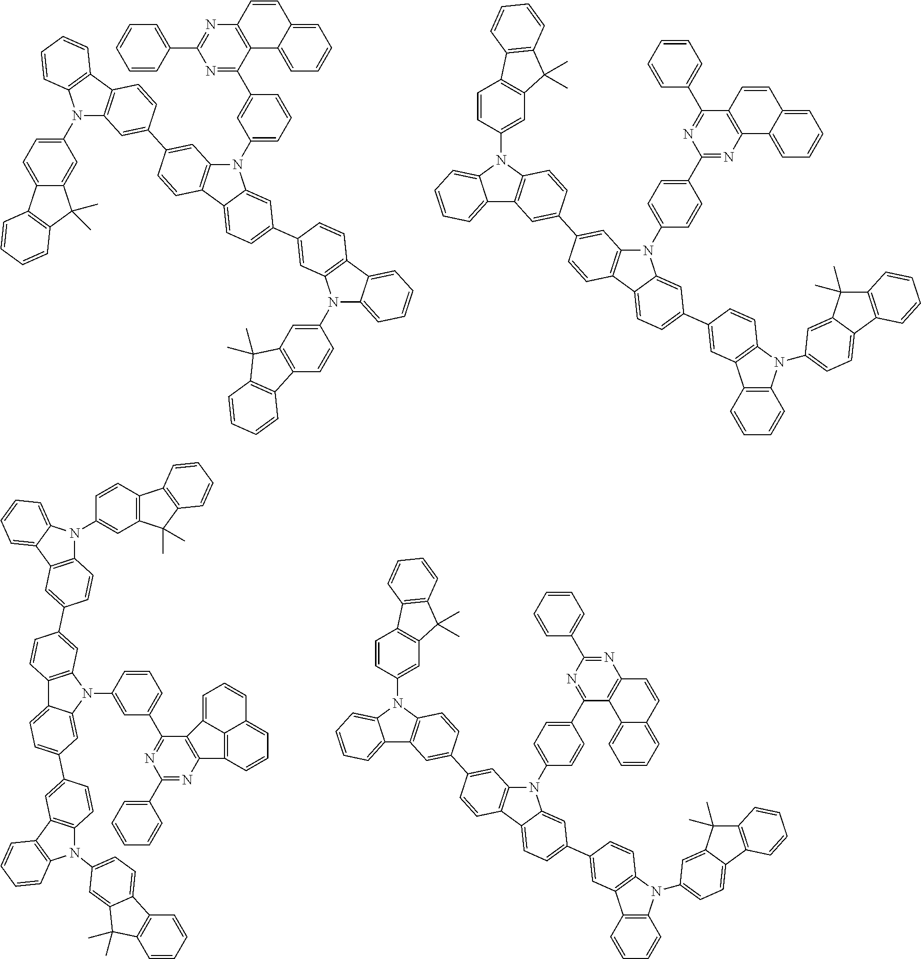

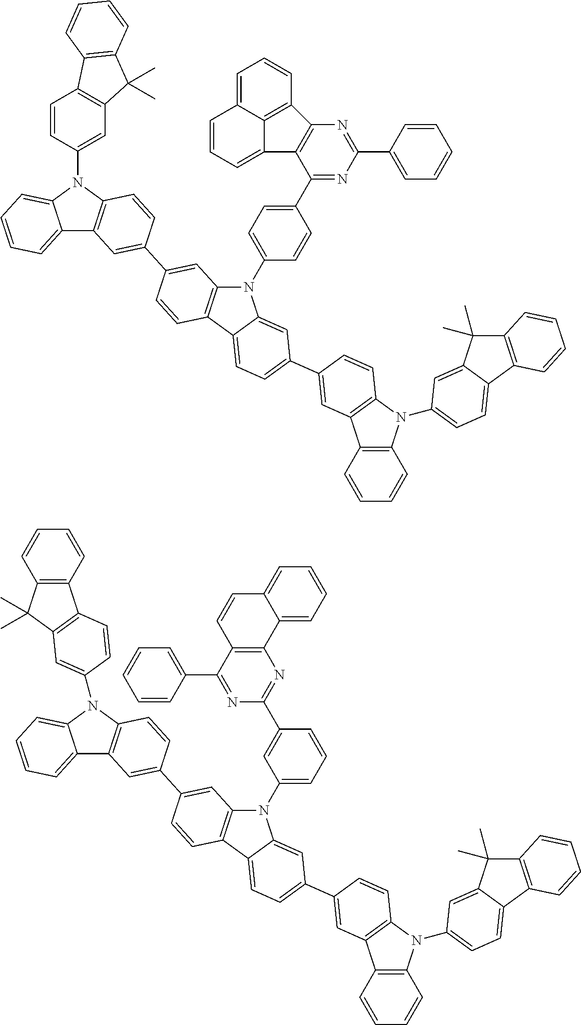 [SMILES: CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7ccc(-c8nc(-c9ccccc9)nc9c8-c8cccc%10cccc-9c8%10)cc7)c6c5)ccc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7cccc(-c8nc(-c9ccccc9)c9ccc%10ccccc%10c9n8)c7)c6c5)ccc43)cc21]

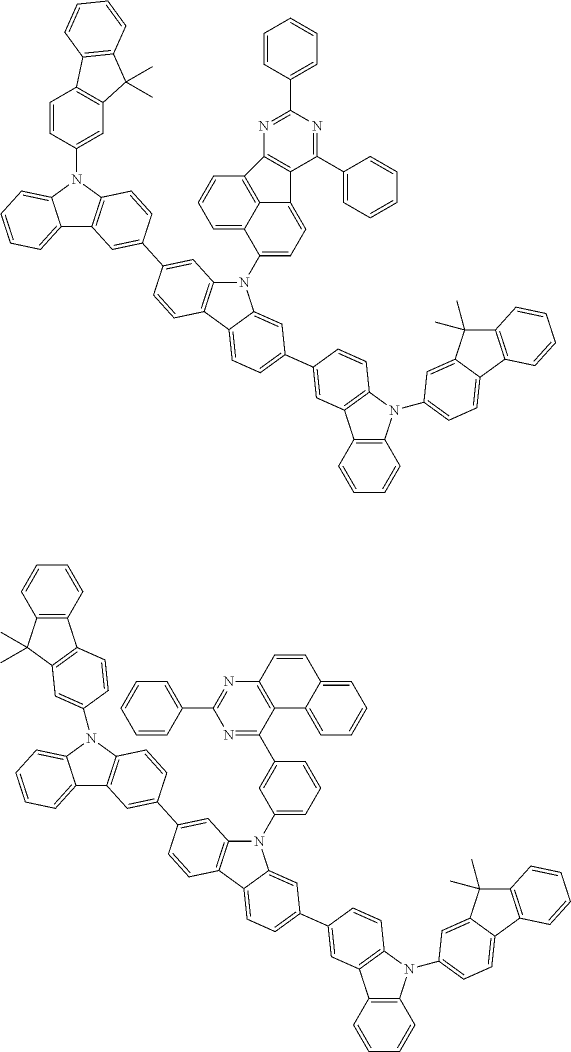 [SMILES: CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7ccc8c9c(cccc79)-c7nc(-c9ccccc9)nc(-c9ccccc9)c7-8)c6c5)ccc43)cc21 ; CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7cccc(-c8nc(-c9ccccc9)nc9ccc%10ccccc%10c89)c7)c6c5)ccc43)cc21]

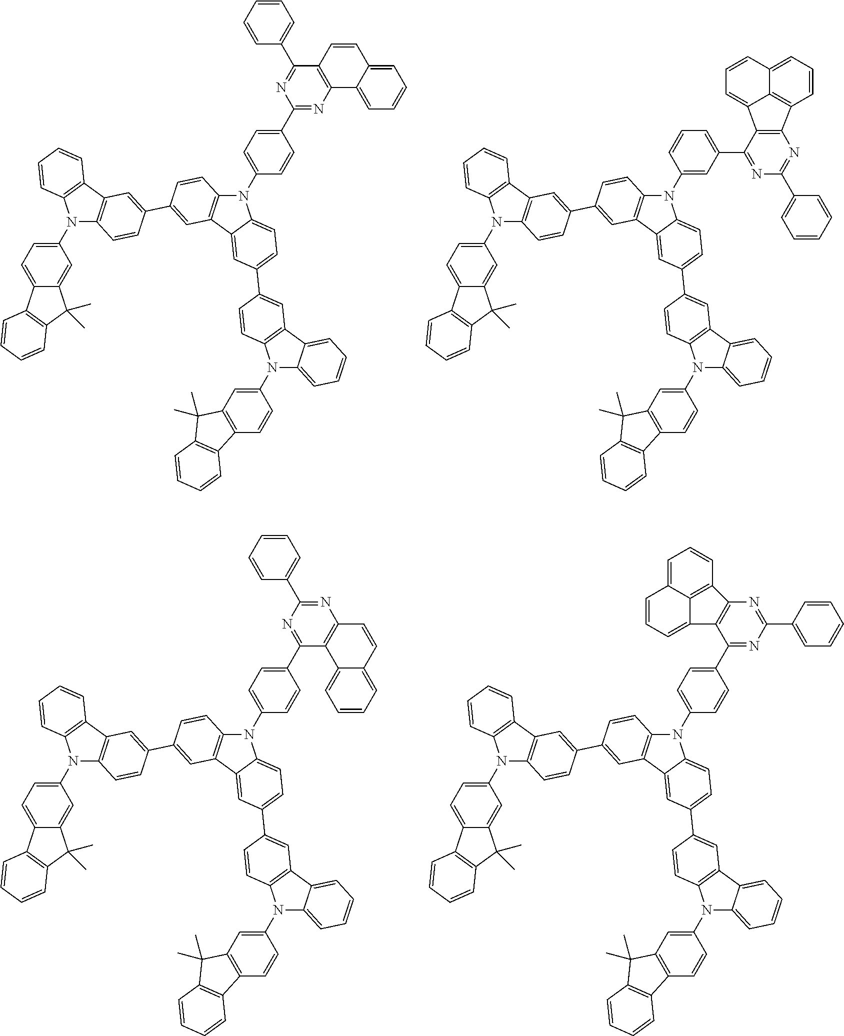

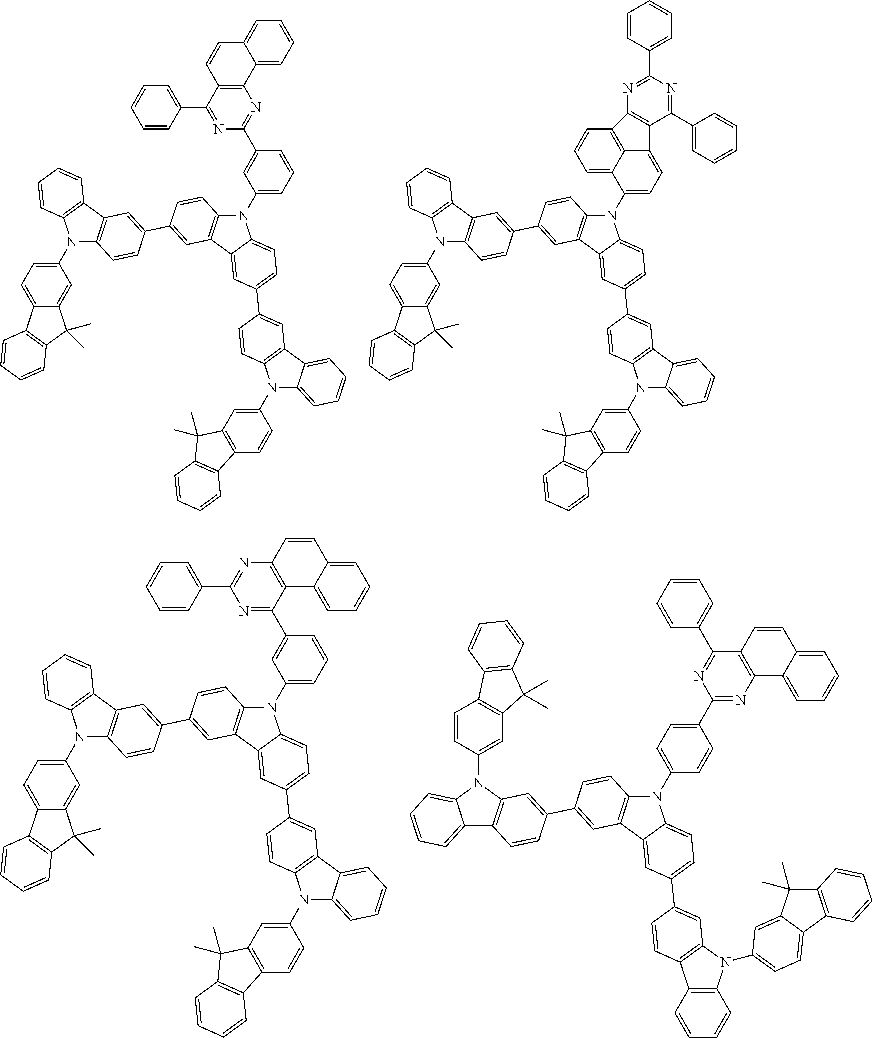

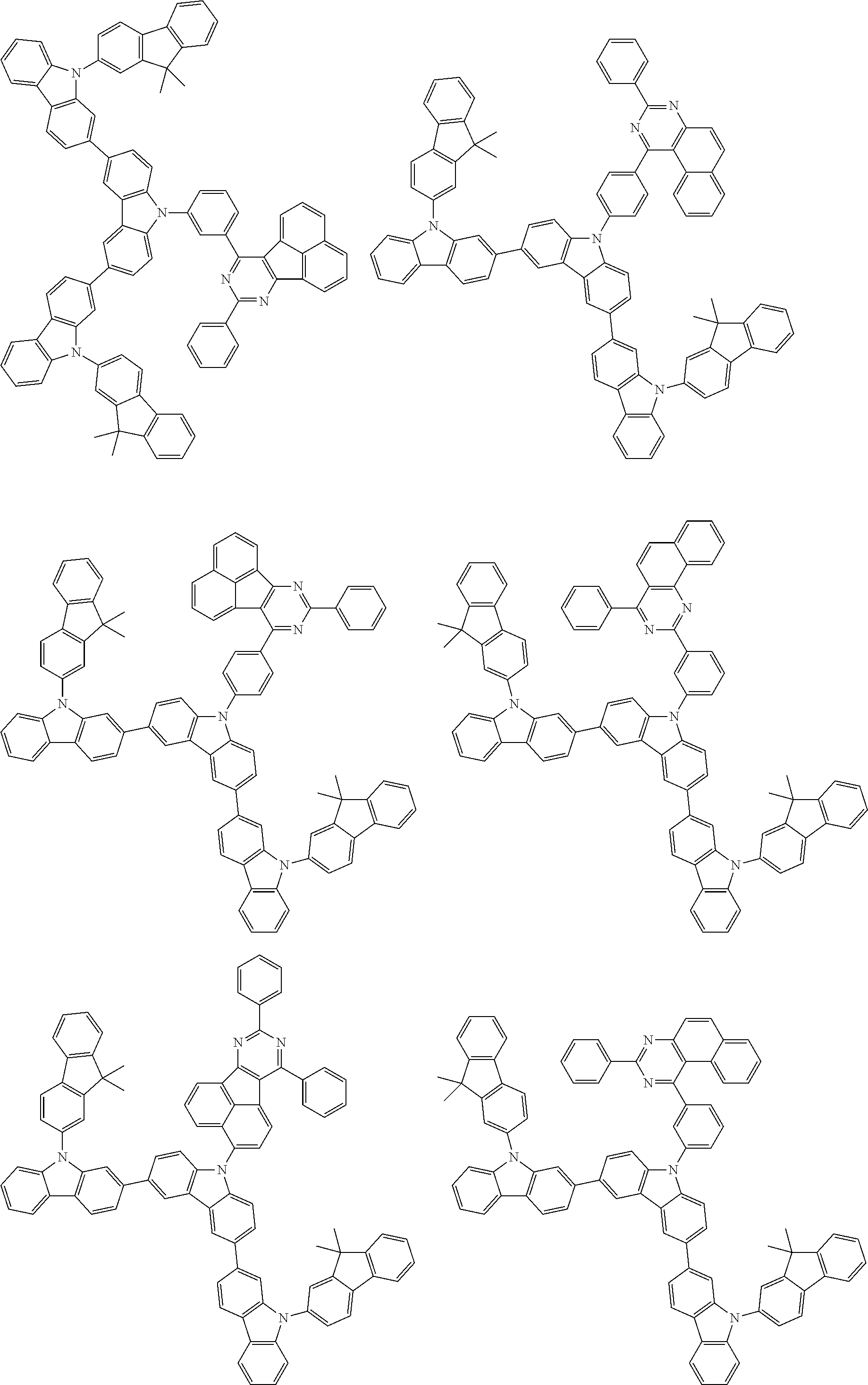

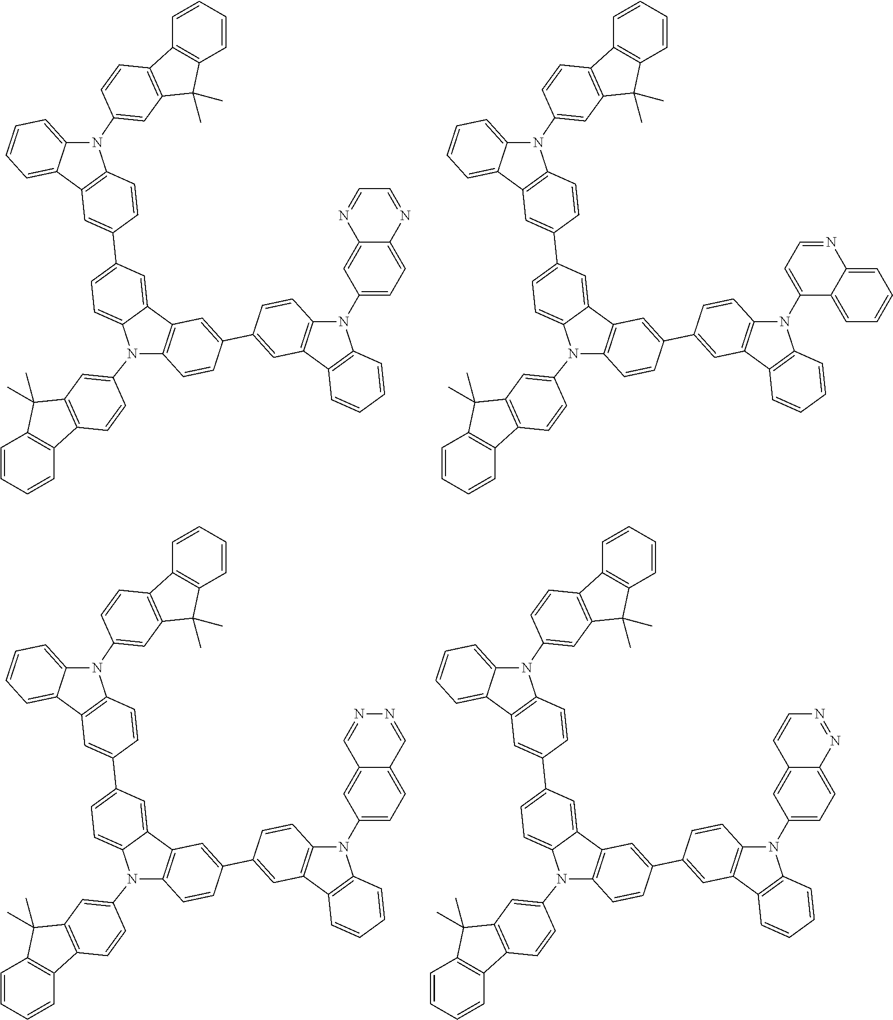

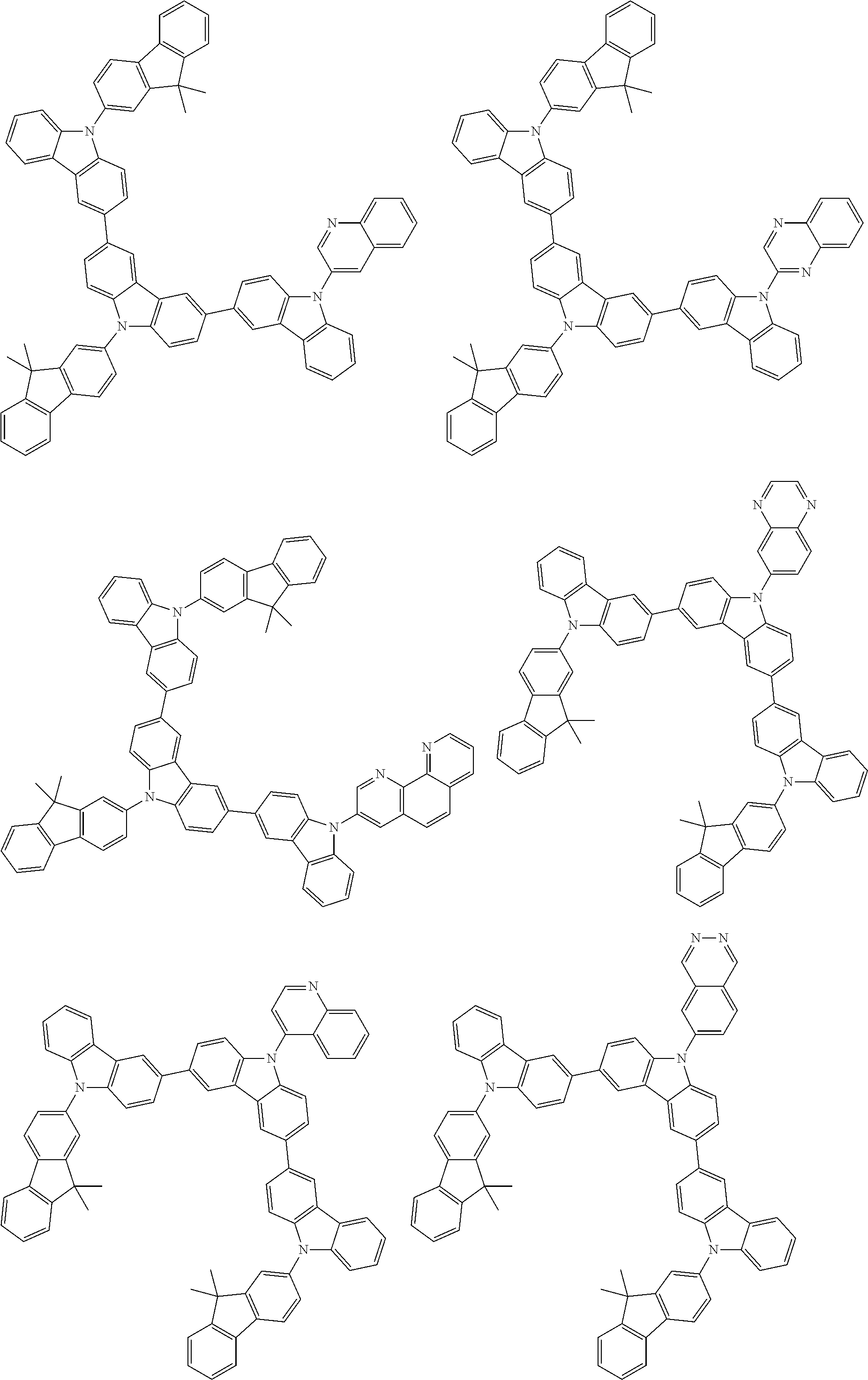

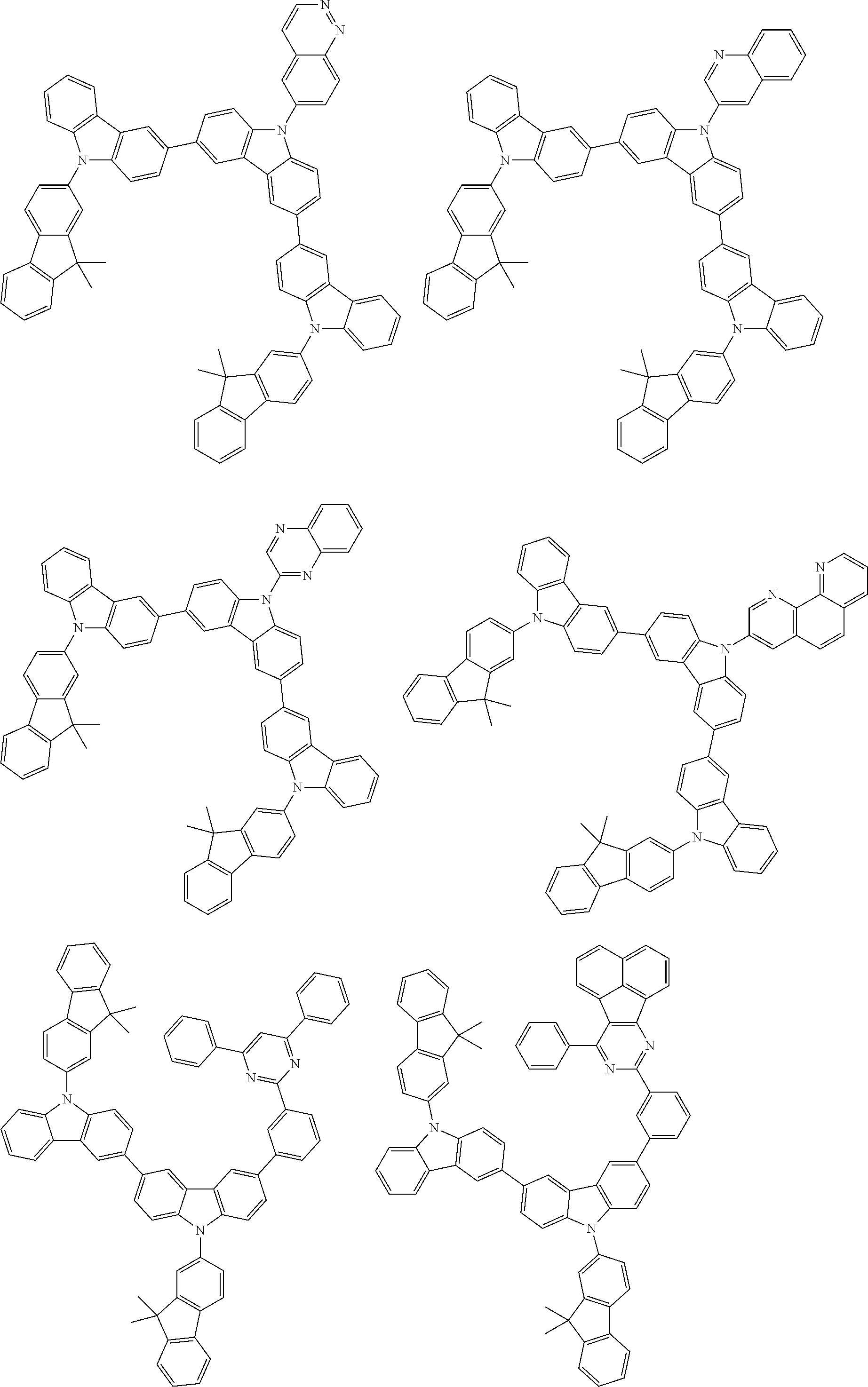

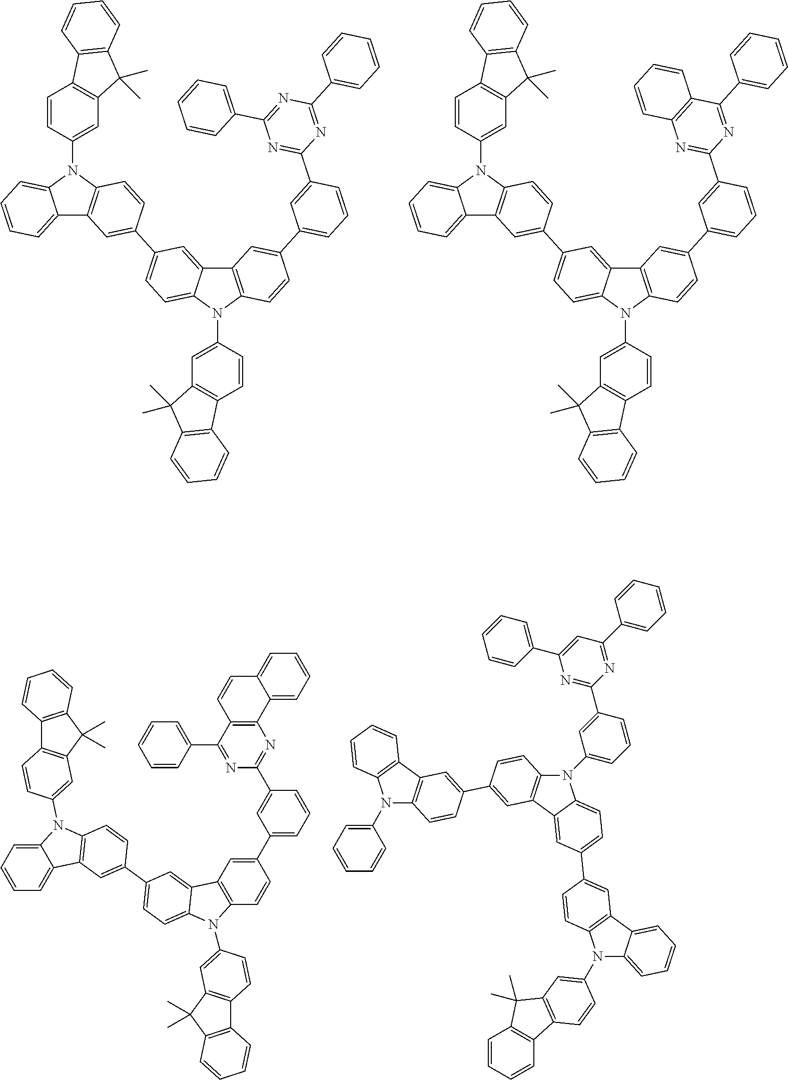

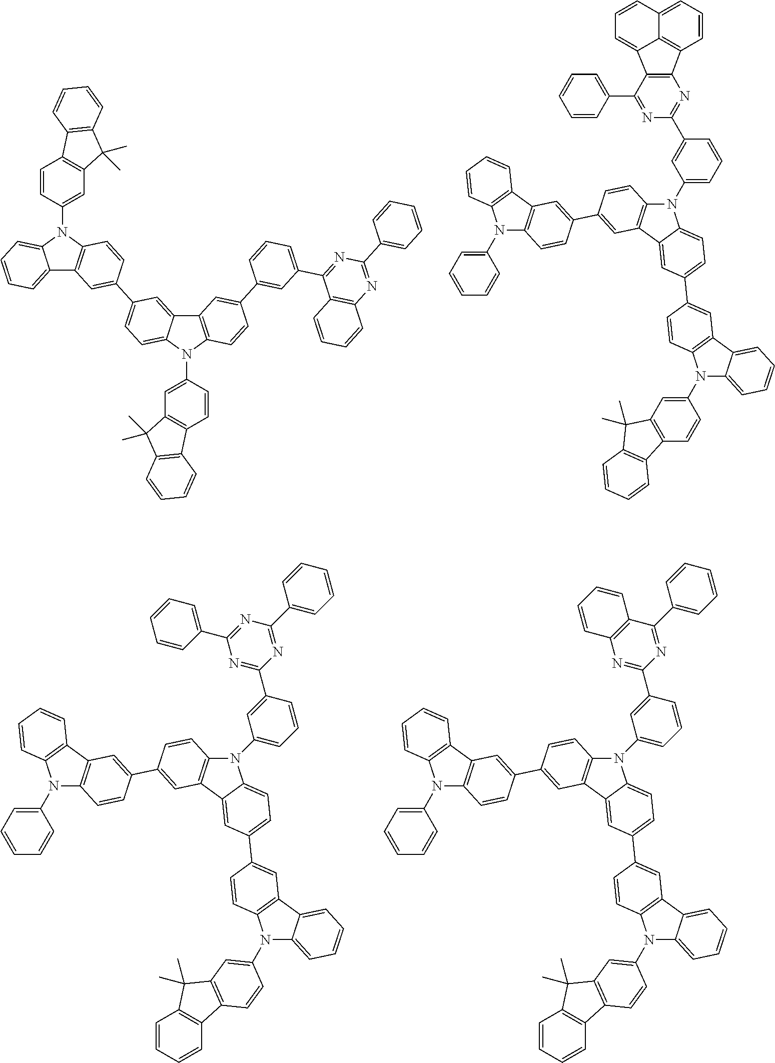

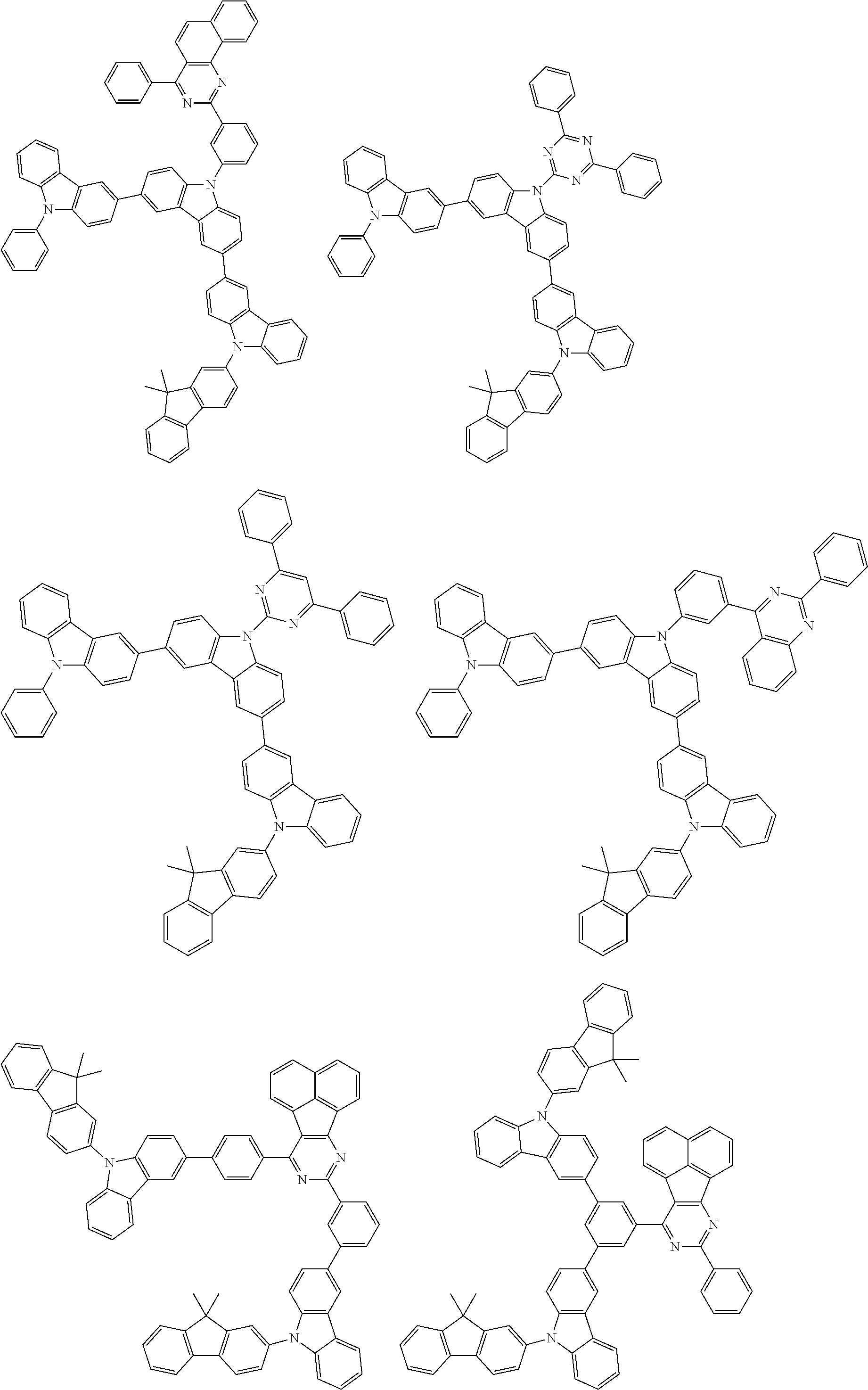

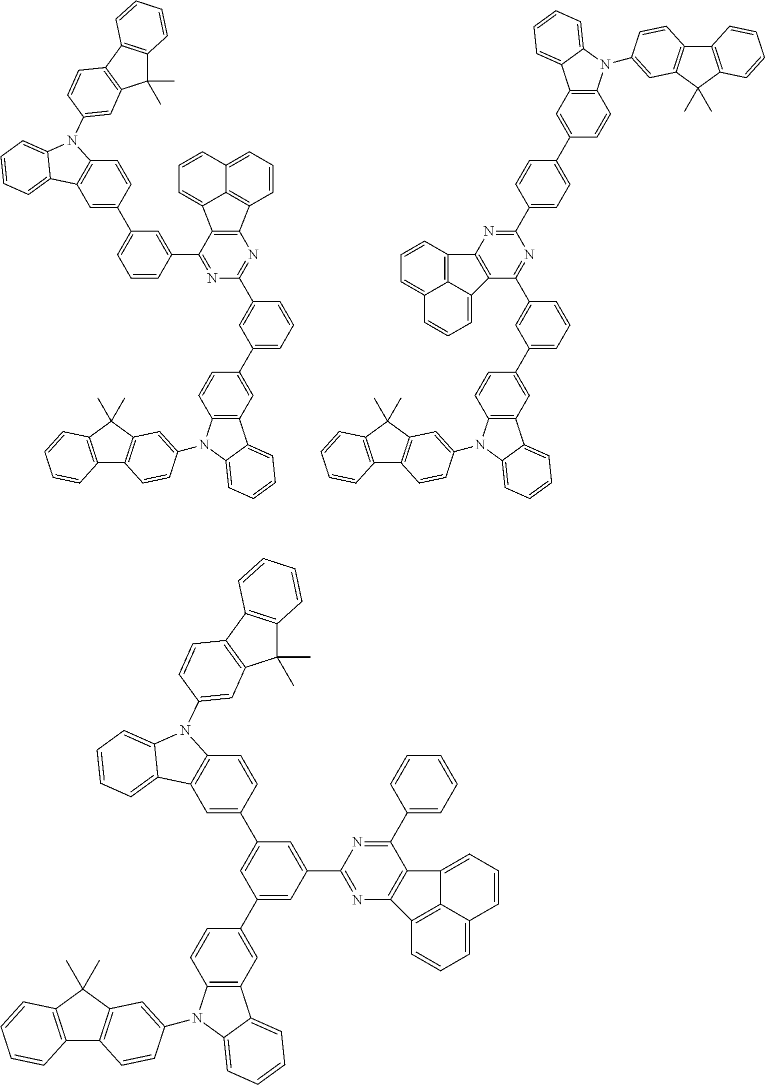

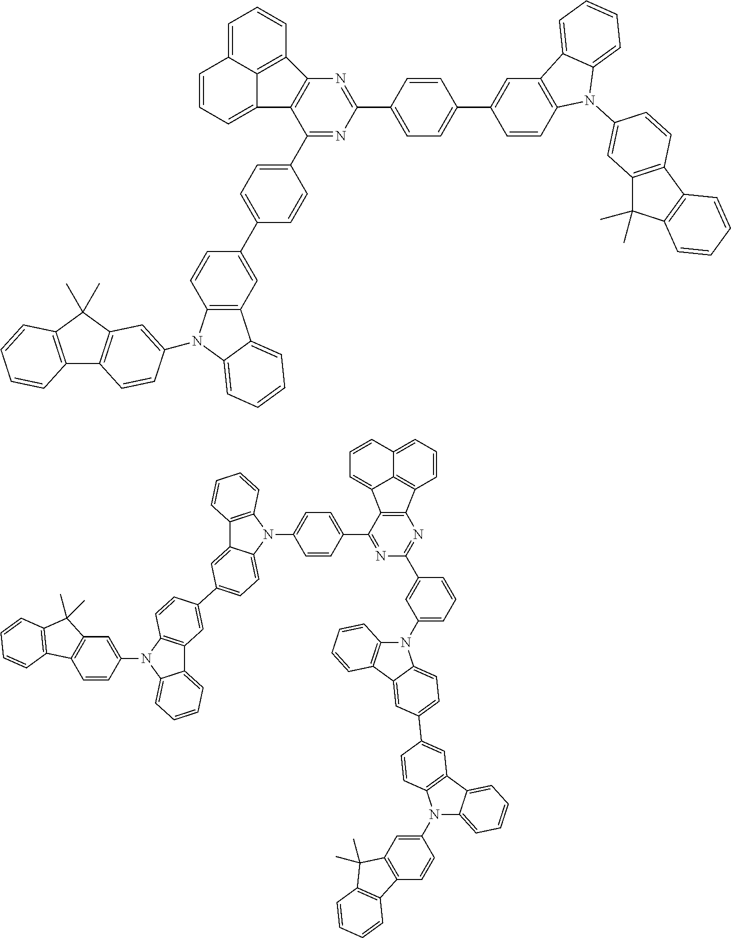 [SMILES: CC1(C)c2ccccc2-c2ccc(-n3c4ccccc4c4cc(-c5ccc(-c6nc(-c7ccc(-c8ccc9c(c8)c8ccccc8n9-c8ccc9c(c8)C(C)(C)c8ccccc8-9)cc7)c7c(n6)-c6cccc8cccc-7c68)cc5)ccc43)cc21 ; CC1(C)c2ccccc2-c2ccc(-n3c4ccccc4c4cc(-c5ccc6c(c5)c5ccccc5n6-c5ccc(-c6nc(-c7cccc(-n8c9ccccc9c9cc(-c%10ccc%11c(c%10)c%10ccccc%10n%11-c%10ccc%11c(c%10)C(C)(C)c%10ccccc%10-%11)ccc98)c7)nc7c6-c6cccc8cccc-7c68)cc5)ccc43)cc21]

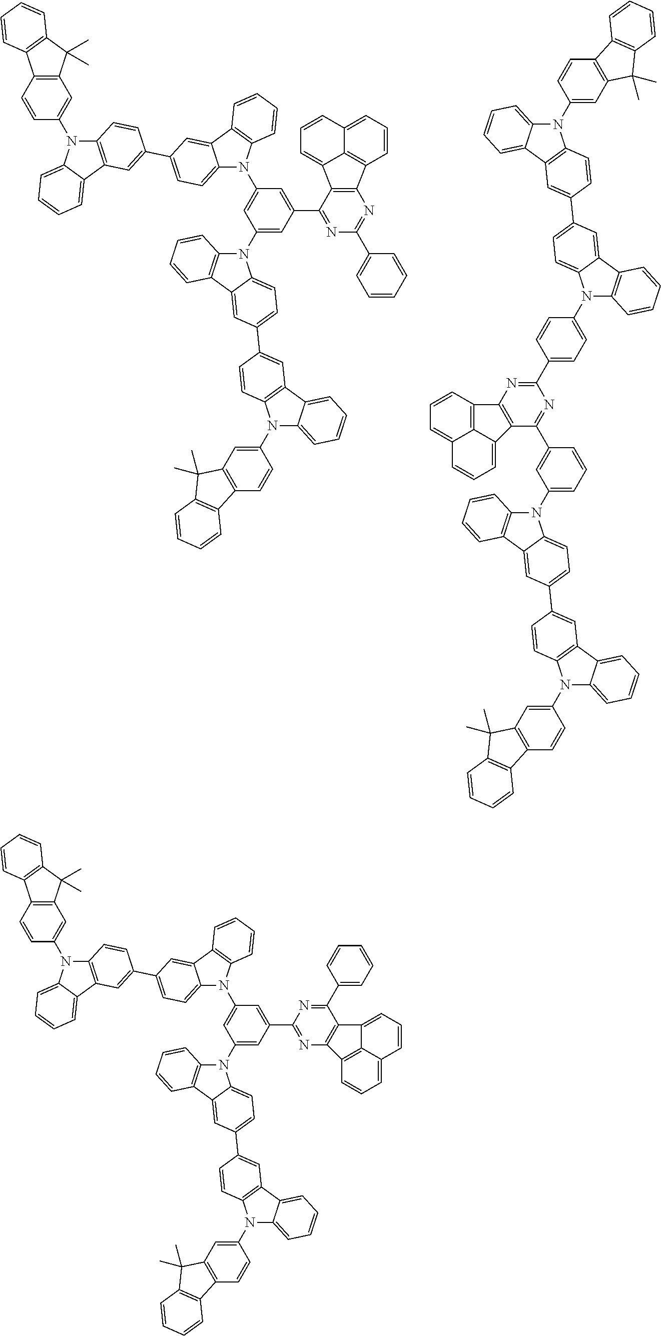

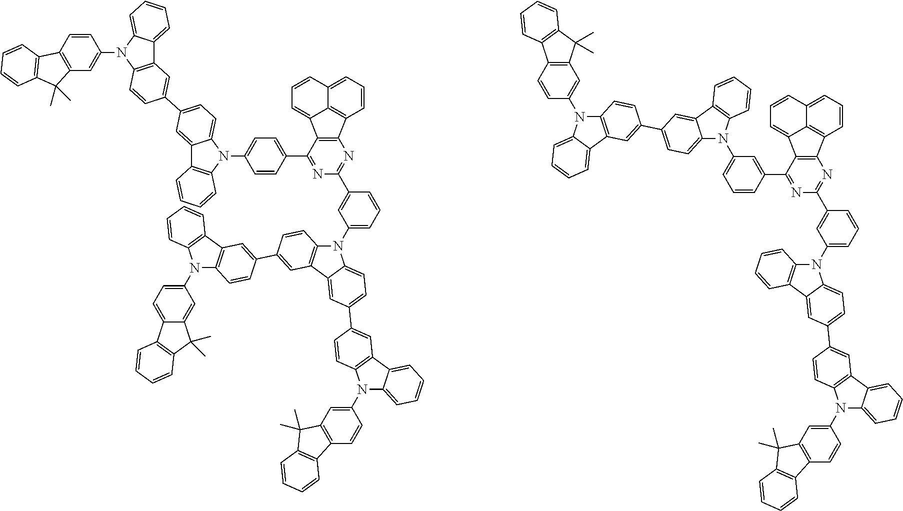

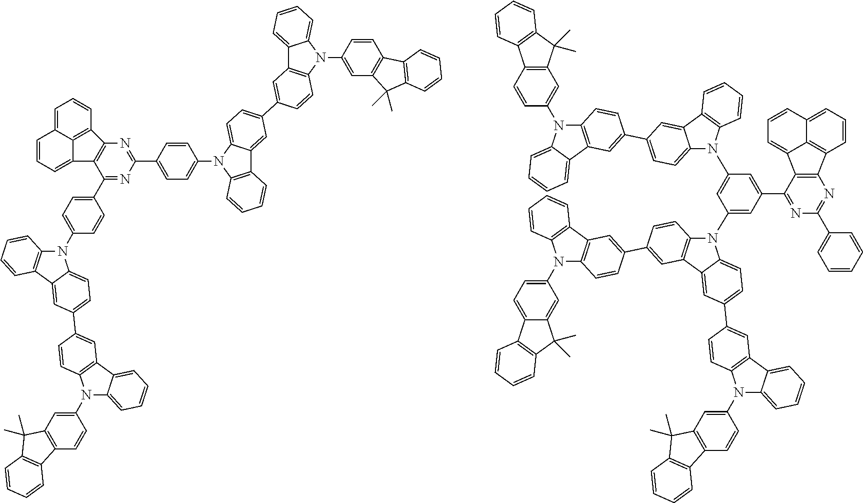 [SMILES: CC1(C)c2ccccc2-c2ccc(-n3c4ccccc4c4cc(-c5ccc6c(c5)c5ccccc5n6-c5ccc(-c6nc(-c7ccc(-n8c9ccccc9c9cc(-c%10ccc%11c(c%10)c%10ccccc%10n%11-c%10ccc%11c(c%10)C(C)(C)c%10ccccc%10-%11)ccc98)cc7)c7c(n6)-c6cccc8cccc-7c68)cc5)ccc43)cc21 ; CC1(C)c2ccccc2-c2ccc(-n3c4ccccc4c4cc(-c5ccc6c(c5)c5ccccc5n6-c5cc(-c6nc(-c7ccccc7)nc7c6-c6cccc8cccc-7c68)cc(-n6c7ccc(-c8ccc9c(c8)c8ccccc8n9-c8ccc9c(c8)C(C)(C)c8ccccc8-9)cc7c7cc(-c8ccc9c(c8)c8ccccc8n9-c8ccc9c(c8)C(C)(C)c8ccccc8-9)ccc76)c5)ccc43)cc21]

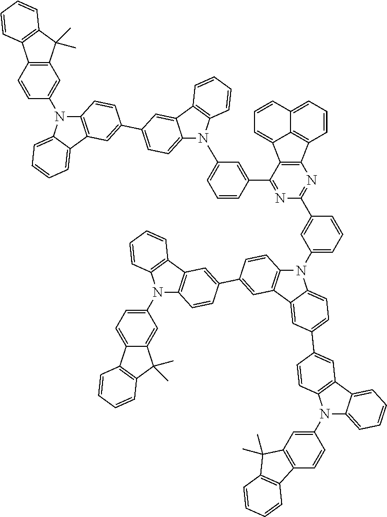 [SMILES: CC1(C)c2ccccc2-c2ccc(-n3c4ccccc4c4cc(-c5ccc6c(c5)c5ccccc5n6-c5cccc(-c6nc(-c7cccc(-n8c9ccc(-c%10ccc%11c(c%10)c%10ccccc%10n%11-c%10ccc%11c(c%10)C(C)(C)c%10ccccc%10-%11)cc9c9cc(-c%10ccc%11c(c%10)c%10ccccc%10n%11-c%10ccc%11c(c%10)C(C)(C)c%10ccccc%10-%11)ccc98)c7)nc7c6-c6cccc8cccc-7c68)c5)ccc43)cc21]

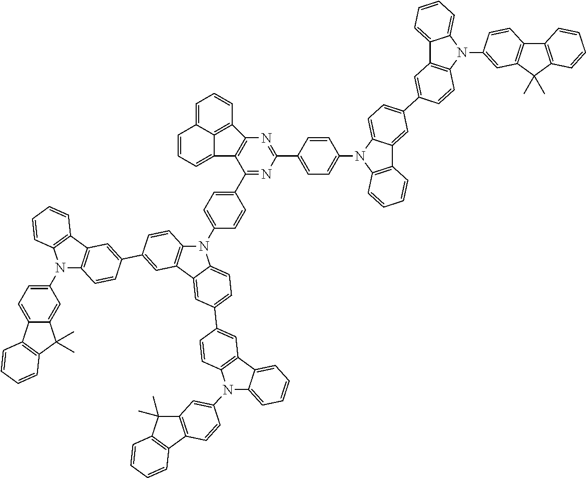 [SMILES: CC1(C)c2ccccc2-c2ccc(-n3c4ccccc4c4cc(-c5ccc6c(c5)c5ccccc5n6-c5ccc(-c6nc(-c7ccc(-n8c9ccc(-c%10ccc%11c(c%10)c%10ccccc%10n%11-c%10ccc%11c(c%10)C(C)(C)c%10ccccc%10-%11)cc9c9cc(-c%10ccc%11c(c%10)c%10ccccc%10n%11-c%10ccc%11c(c%10)C(C)(C)c%10ccccc%10-%11)ccc98)cc7)c7c(n6)-c6cccc8cccc-7c68)cc5)ccc43)cc21]

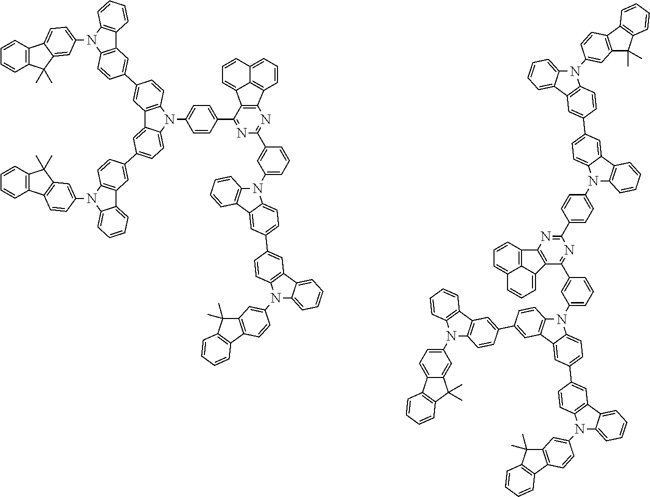

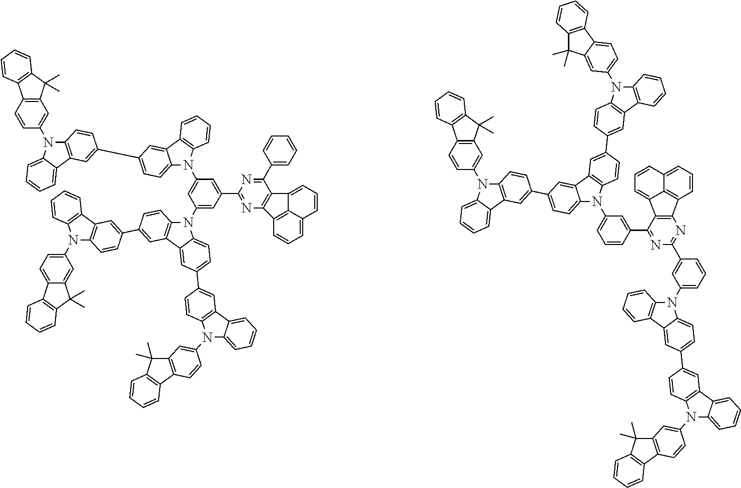

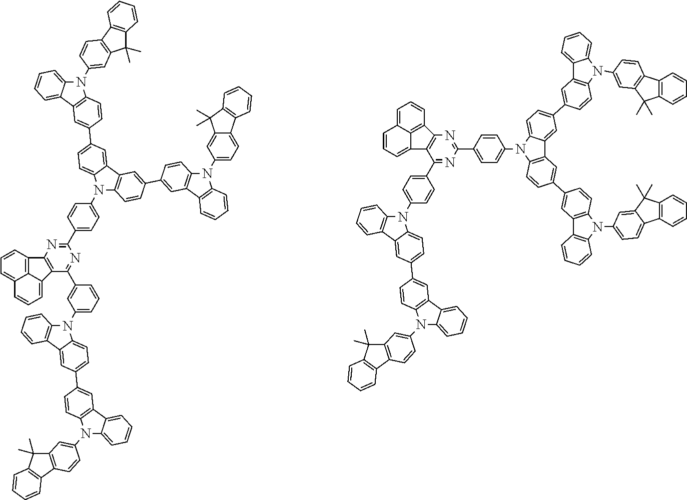

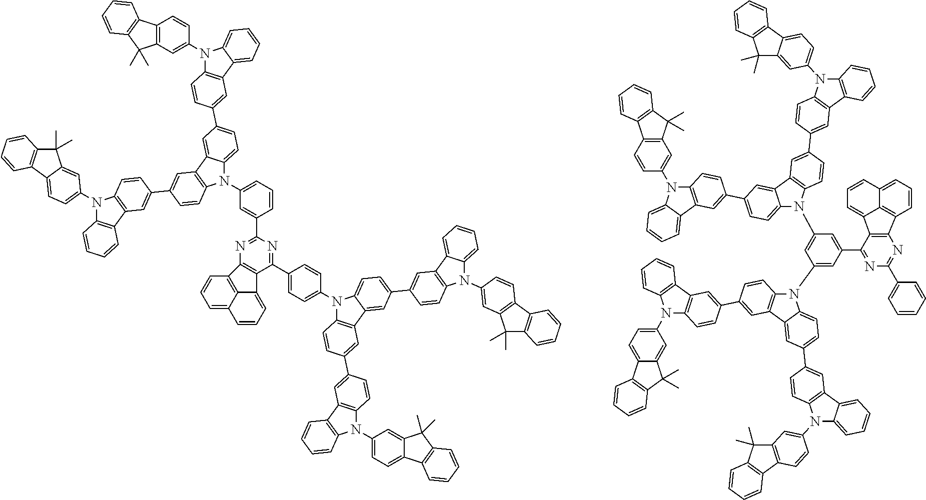

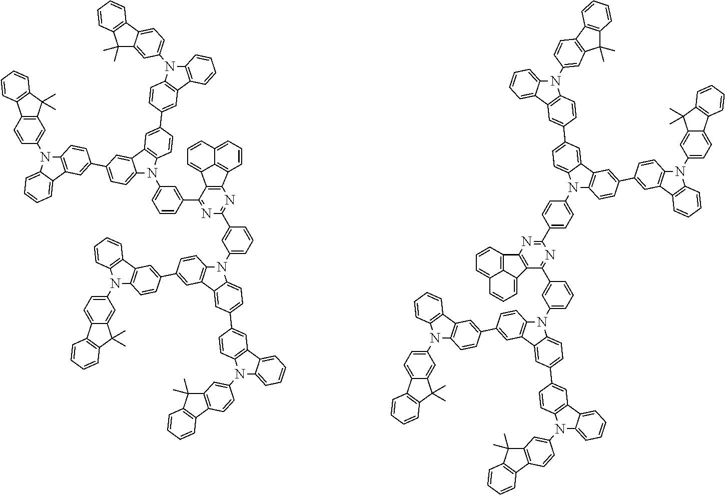

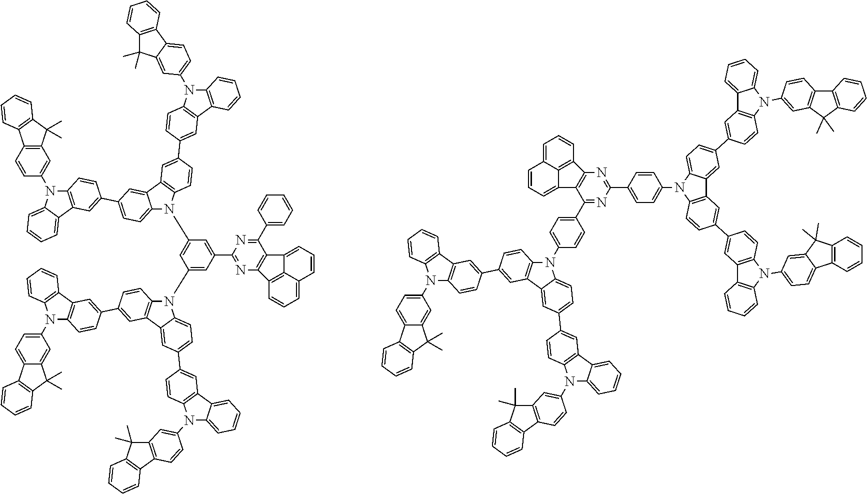

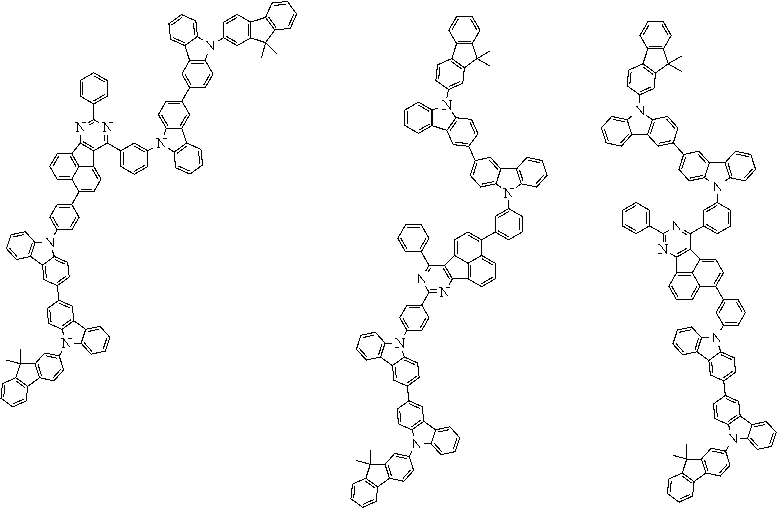

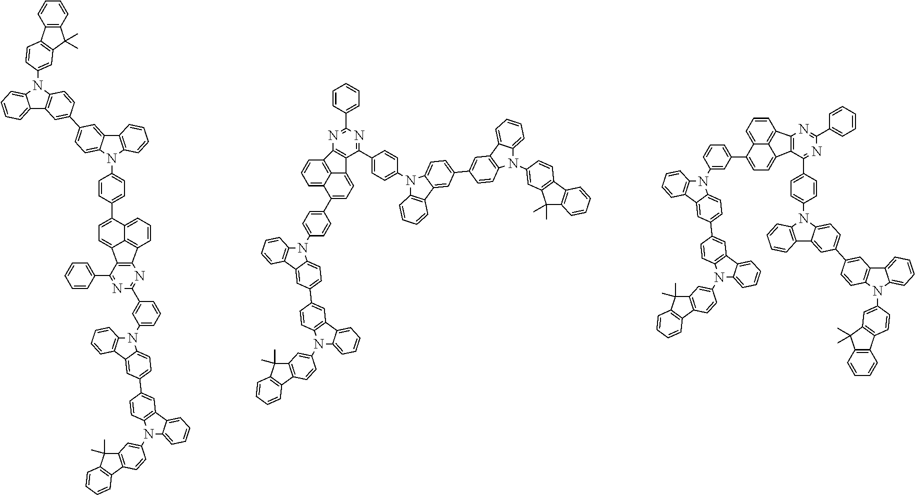

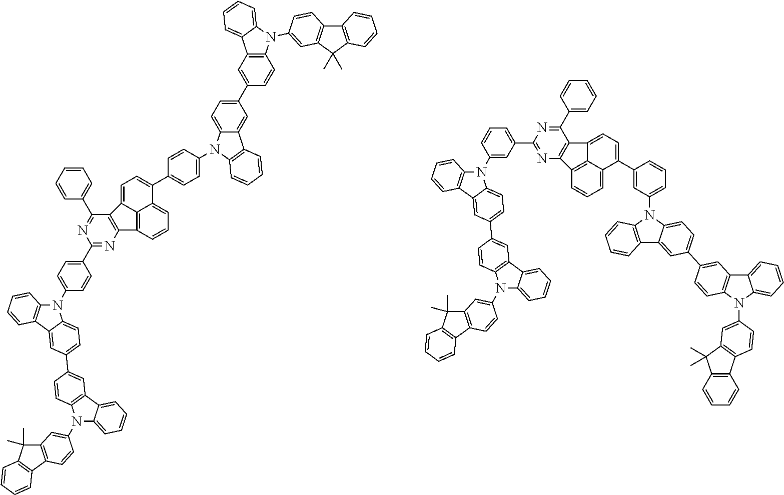 [SMILES: CC1(C)c2ccccc2-c2ccc(-n3c4ccccc4c4cc(-c5ccc6c(c5)c5ccccc5n6-c5ccc(-c6nc(-c7ccccc7)c7c(n6)-c6cccc8c(-c9ccc(-n%10c%11ccccc%11c%11cc(-c%12ccc%13c(c%12)c%12ccccc%12n%13-c%12ccc%13c(c%12)C(C)(C)c%12ccccc%12-%13)ccc%11%10)cc9)ccc-7c68)cc5)ccc43)cc21 ; CC1(C)c2ccccc2-c2ccc(-n3c4ccccc4c4cc(-c5ccc6c(c5)c5ccccc5n6-c5cccc(-c6nc(-c7ccccc7)c7c(n6)-c6cccc8c(-c9cccc(-n%10c%11ccccc%11c%11cc(-c%12ccc%13c(c%12)c%12ccccc%12n%13-c%12ccc%13c(c%12)C(C)(C)c%12ccccc%12-%13)ccc%11%10)c9)ccc-7c68)c5)ccc43)cc21]

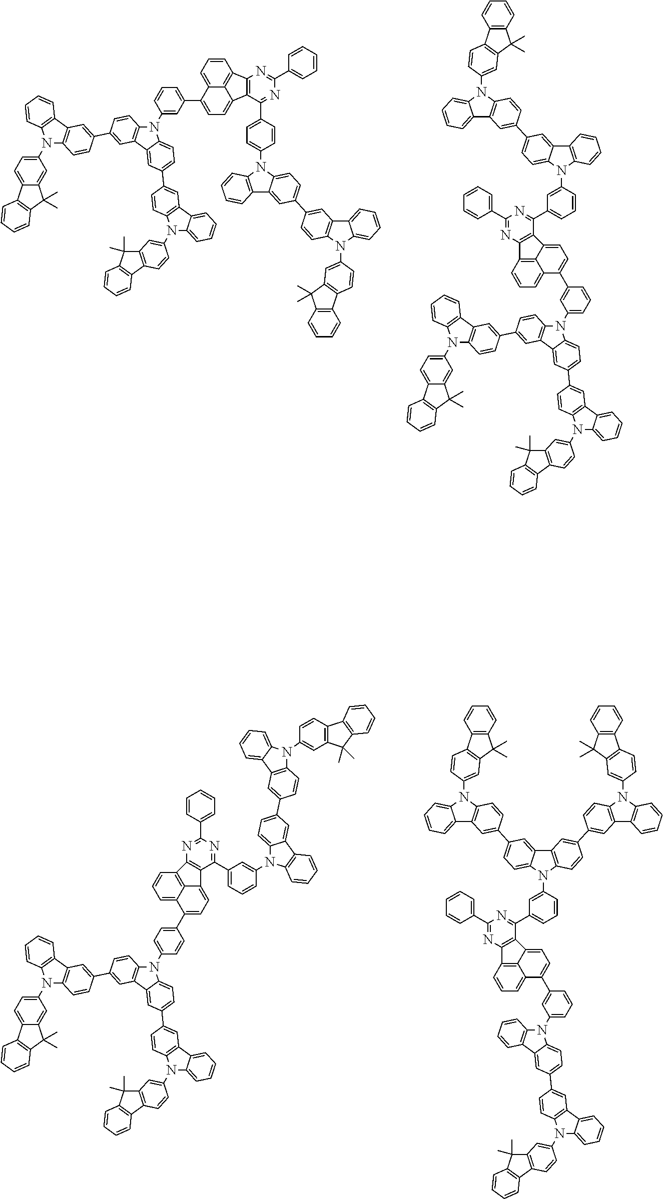

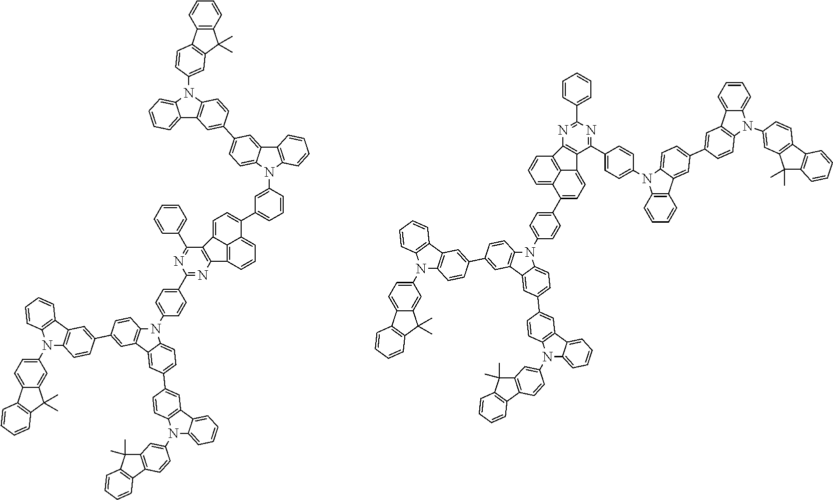

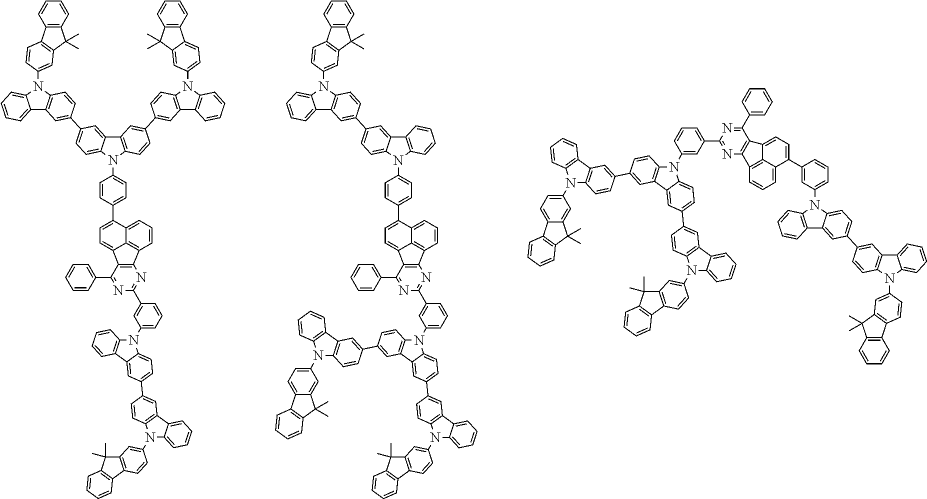

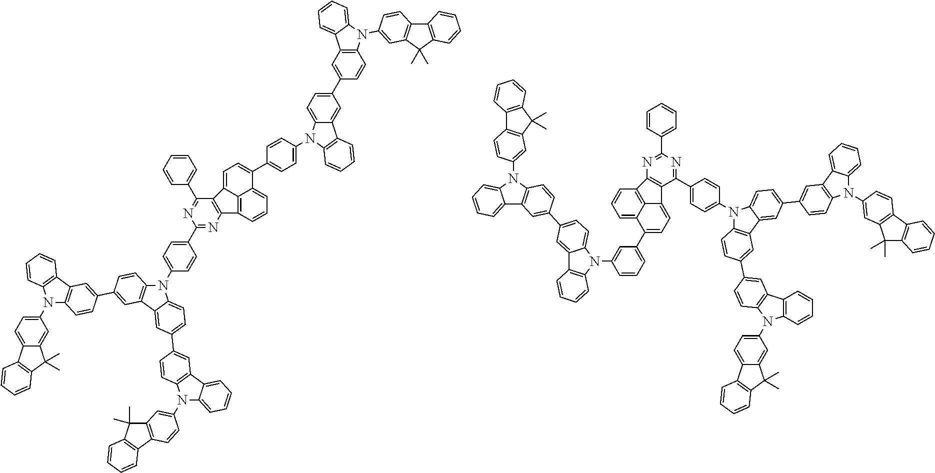

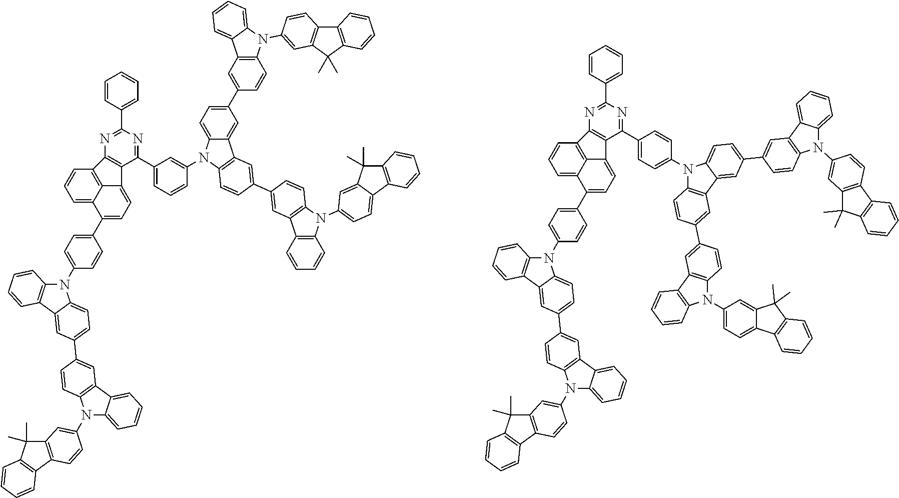

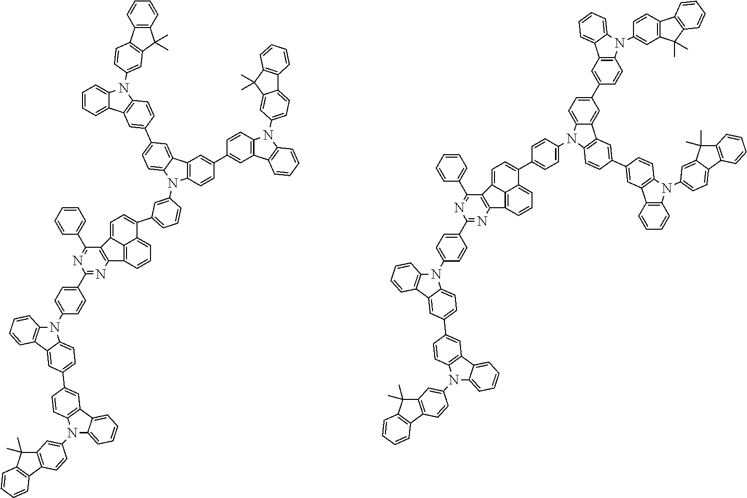

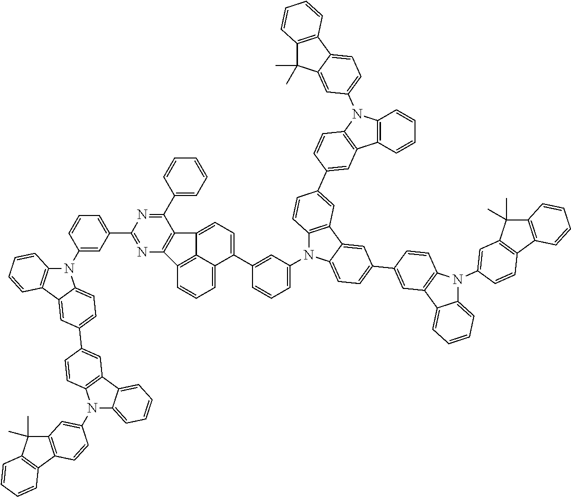 [SMILES: CC1(C)c2ccccc2-c2ccc(-n3c4ccccc4c4cc(-c5ccc6c(c5)c5ccccc5n6-c5cccc(-c6nc(-c7ccccc7)c7c(n6)-c6cccc8c(-c9cccc(-n%10c%11ccc(-c%12ccc%13c(c%12)c%12ccccc%12n%13-c%12ccc%13c(c%12)C(C)(C)c%12ccccc%12-%13)cc%11c%11cc(-c%12ccc%13c(c%12)c%12ccccc%12n%13-c%12ccc%13c(c%12)C(C)(C)c%12ccccc%12-%13)ccc%11%10)c9)ccc-7c68)c5)ccc43)cc21]

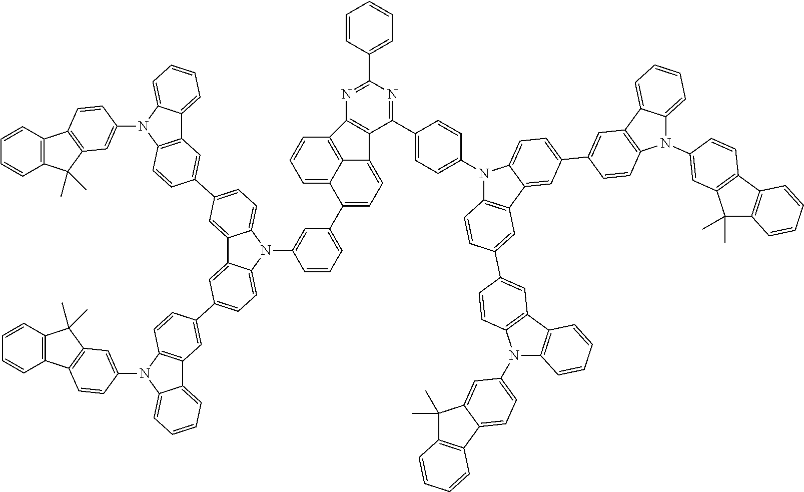 [SMILES: CC1(C)c2ccccc2-c2ccc(-n3c4ccccc4c4cc(-c5ccc6c(c5)c5cc(-c7ccc8c(c7)c7ccccc7n8-c7ccc8c(c7)C(C)(C)c7ccccc7-8)ccc5n6-c5ccc(-c6nc(-c7ccccc7)nc7c6-c6ccc(-c8cccc(-n9c%10ccc(-c%11ccc%12c(c%11)c%11ccccc%11n%12-c%11ccc%12c(c%11)C(C)(C)c%11ccccc%11-%12)cc%10c%10cc(-c%11ccc%12c(c%11)c%11ccccc%11n%12-c%11ccc%12c(c%11)C(C)(C)c%11ccccc%11-%12)ccc%109)c8)c8cccc-7c68)cc5)ccc43)cc21]

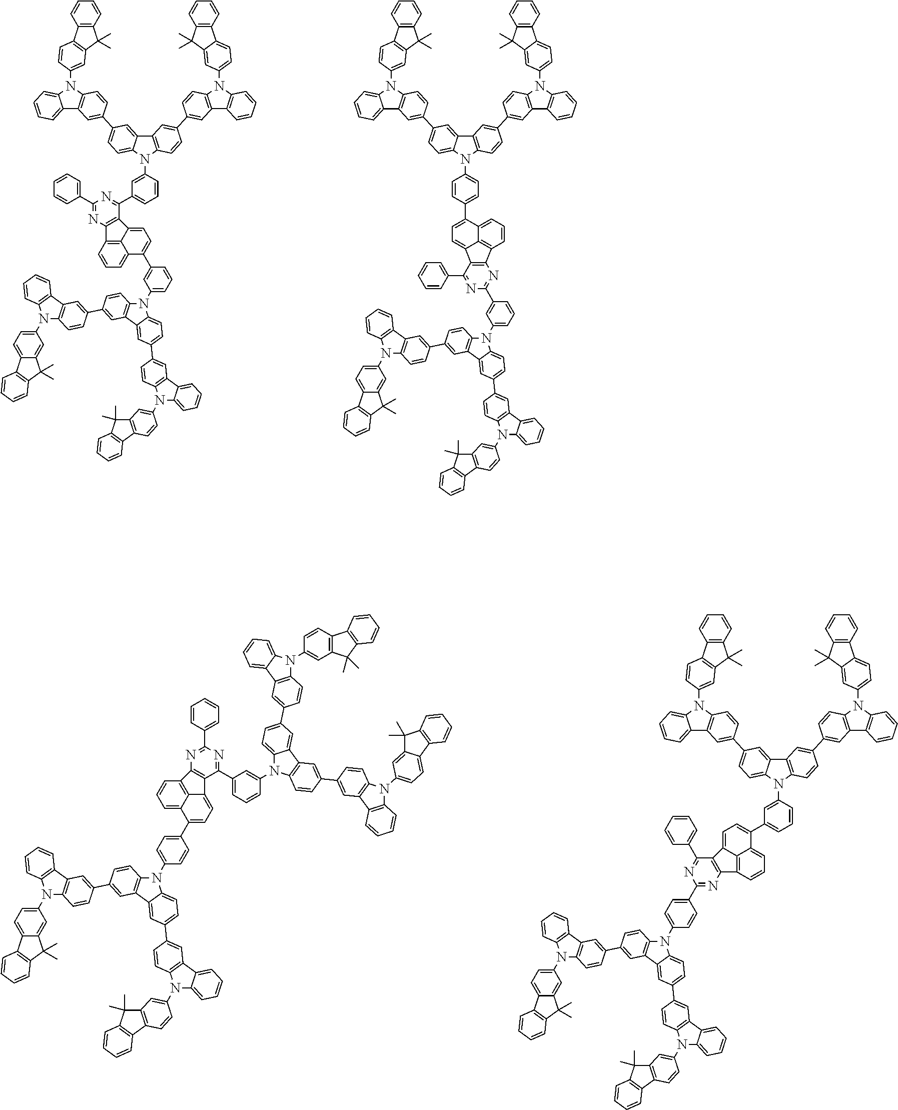

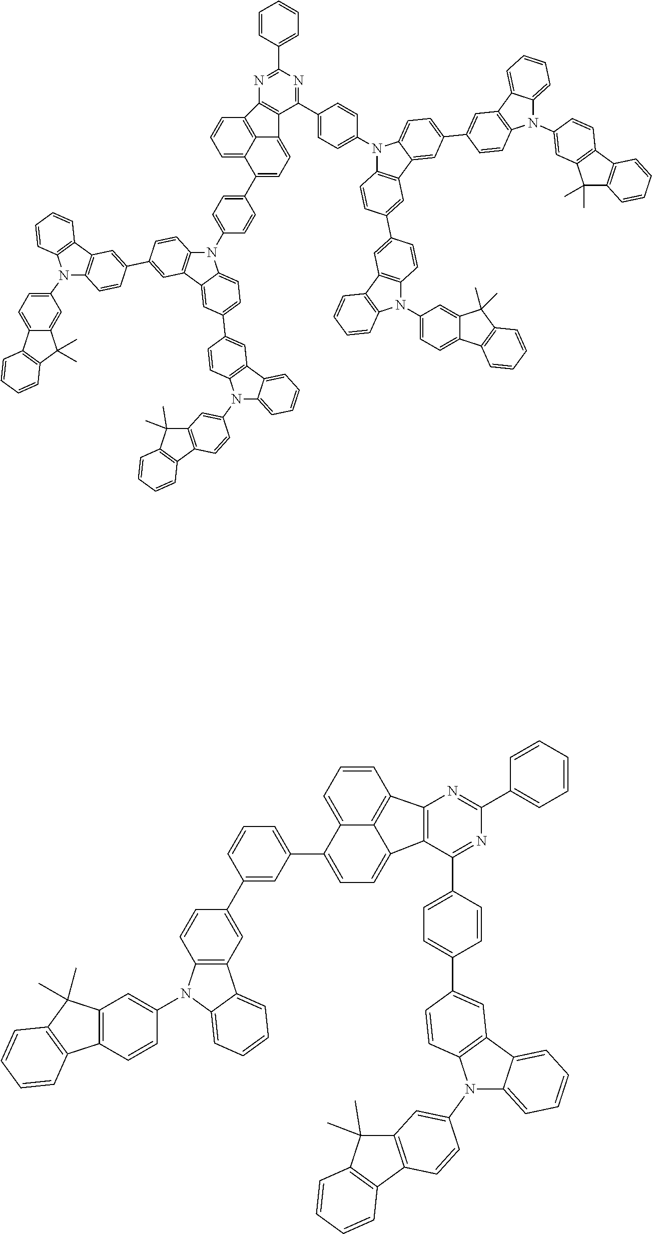

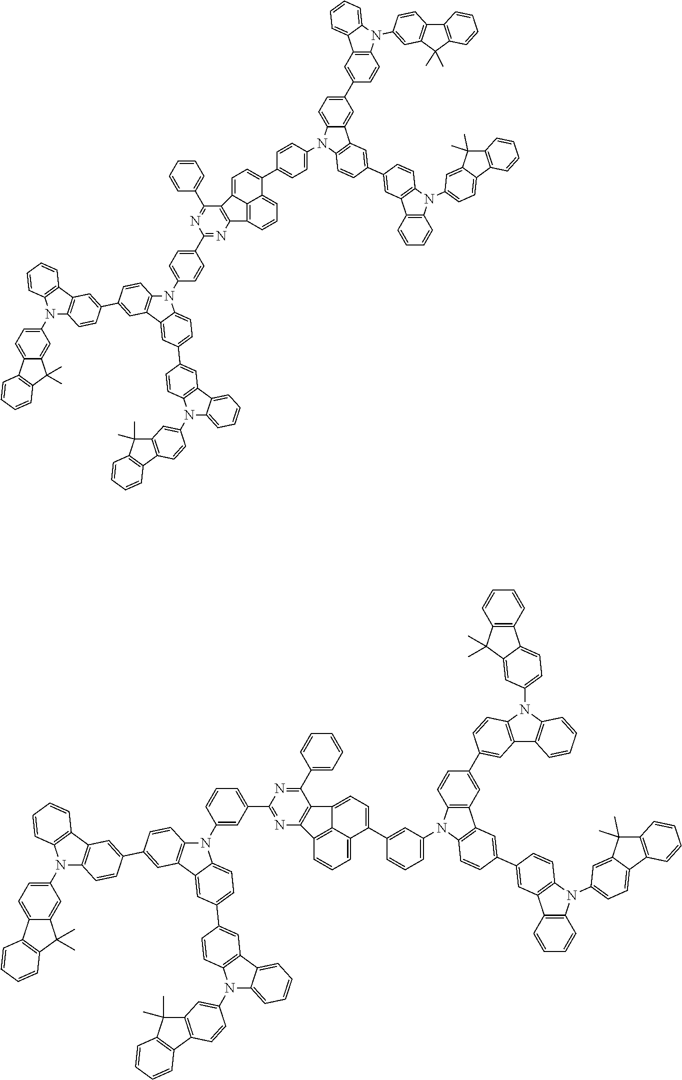

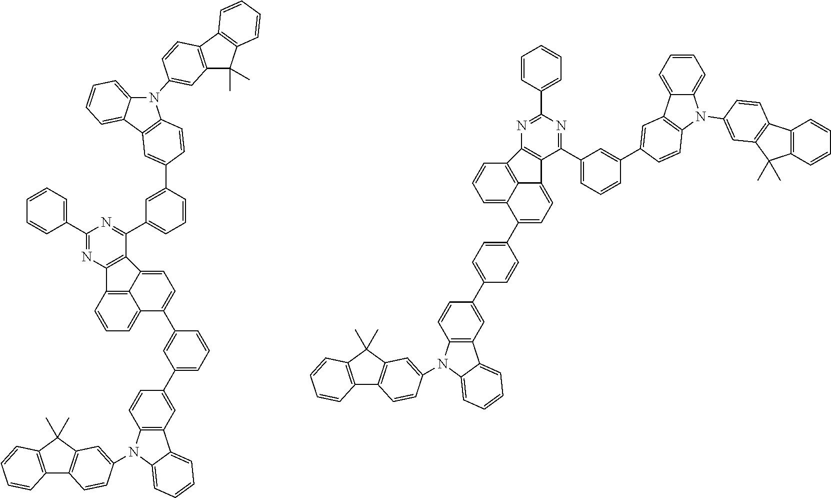 [SMILES: CC1(C)c2ccccc2-c2ccc(-n3c4ccccc4c4cc(-c5cccc(-c6nc(-c7ccccc7)nc7c6-c6ccc(-c8cccc(-c9ccc%10c(c9)c9ccccc9n%10-c9ccc%10c(c9)C(C)(C)c9ccccc9-%10)c8)c8cccc-7c68)c5)ccc43)cc21 ; CC1(C)c2ccccc2-c2ccc(-n3c4ccccc4c4cc(-c5ccc(-c6ccc7c8c(cccc68)-c6nc(-c8ccccc8)nc(-c8cccc(-c9ccc%10c(c9)c9ccccc9n%10-c9ccc%10c(c9)C(C)(C)c9ccccc9-%10)c8)c6-7)cc5)ccc43)cc21]

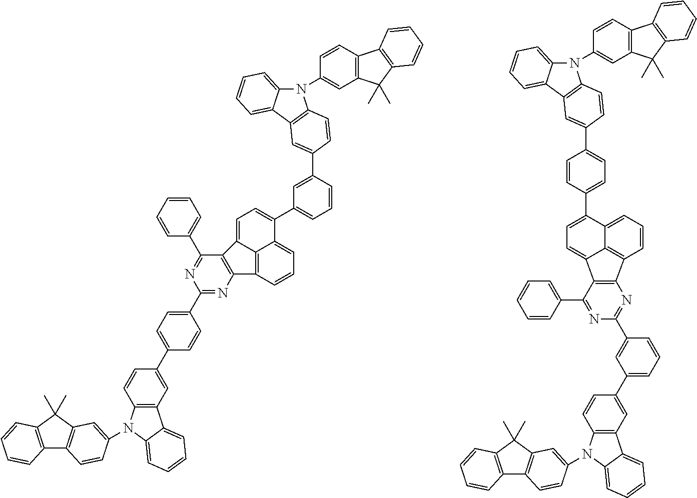 [SMILES: CC1(C)c2ccccc2-c2ccc(-n3c4ccccc4c4cc(-c5ccc(-c6nc(-c7ccccc7)c7c(n6)-c6cccc8c(-c9cccc(-c%10ccc%11c(c%10)c%10ccccc%10n%11-c%10ccc%11c(c%10)C(C)(C)c%10ccccc%10-%11)c9)ccc-7c68)cc5)ccc43)cc21 ; CC1(C)c2ccccc2-c2ccc(-n3c4ccccc4c4cc(-c5ccc(-c6ccc7c8c(cccc68)-c6nc(-c8cccc(-c9ccc%10c(c9)c9ccccc9n%10-c9ccc%10c(c9)C(C)(C)c9ccccc9-%10)c8)nc(-c8ccccc8)c6-7)cc5)ccc43)cc21]

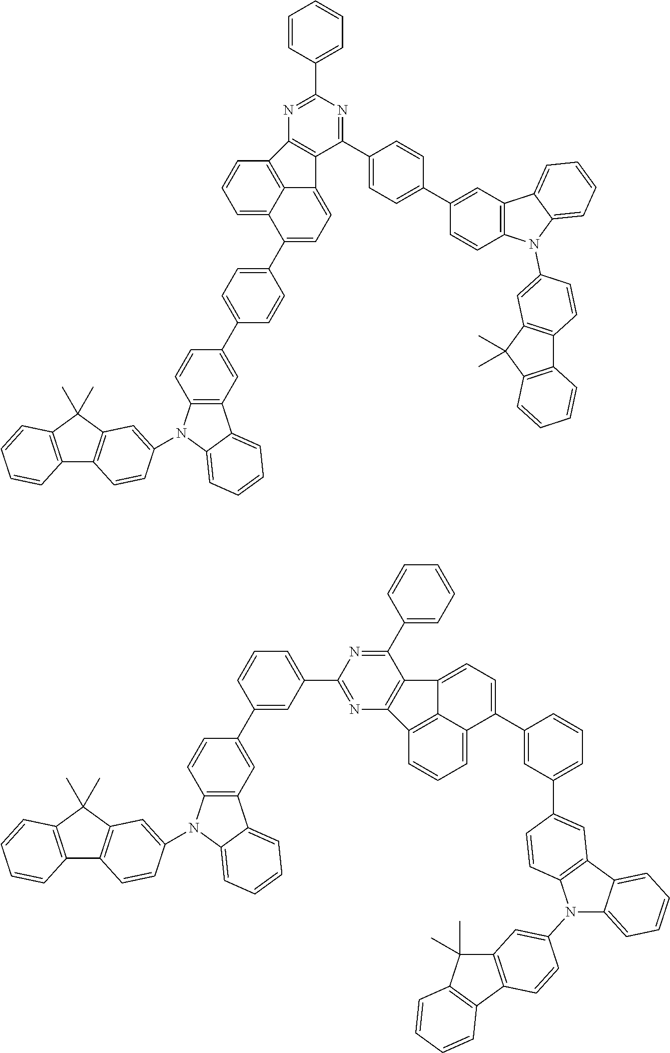 [SMILES: CC1(C)c2ccccc2-c2ccc(-n3c4ccccc4c4cc(-c5ccc(-c6nc(-c7ccccc7)nc7c6-c6ccc(-c8ccc(-c9ccc%10c(c9)c9ccccc9n%10-c9ccc%10c(c9)C(C)(C)c9ccccc9-%10)cc8)c8cccc-7c68)cc5)ccc43)cc21 ; CC1(C)c2ccccc2-c2ccc(-n3c4ccccc4c4cc(-c5cccc(-c6nc(-c7ccccc7)c7c(n6)-c6cccc8c(-c9cccc(-c%10ccc%11c(c%10)c%10ccccc%10n%11-c%10ccc%11c(c%10)C(C)(C)c%10ccccc%10-%11)c9)ccc-7c68)c5)ccc43)cc21]

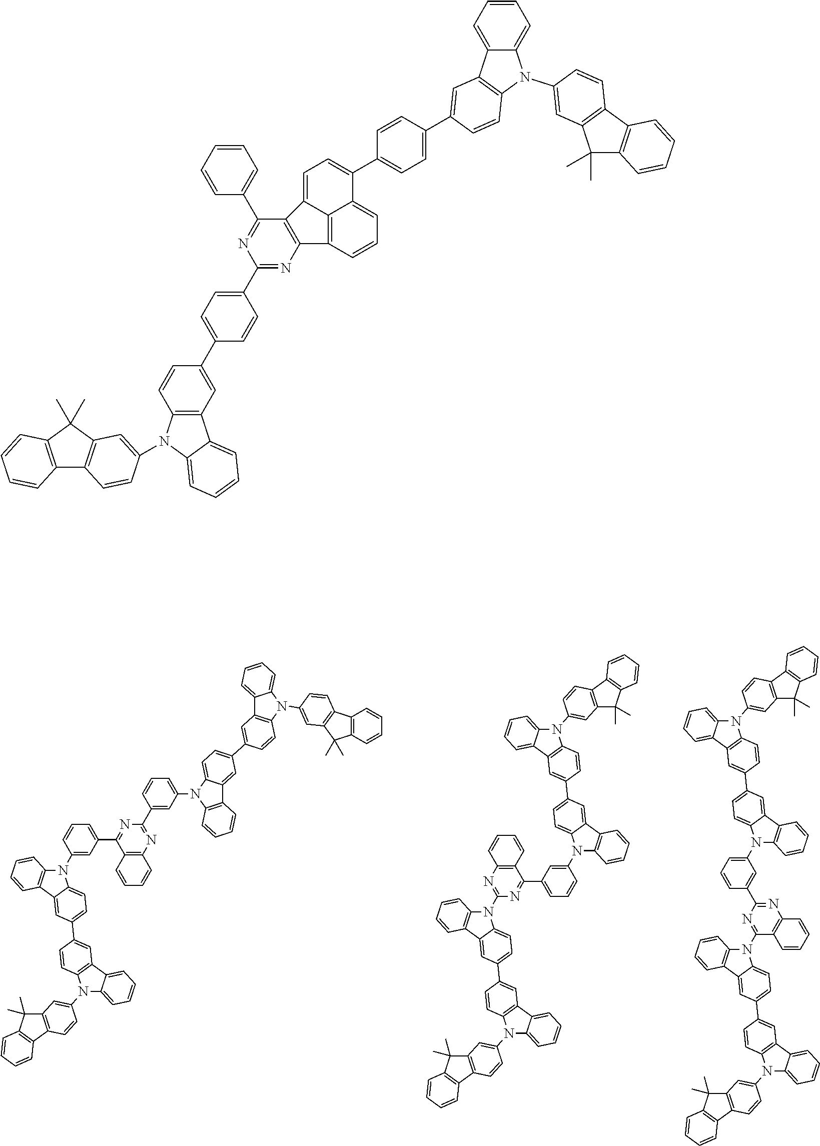

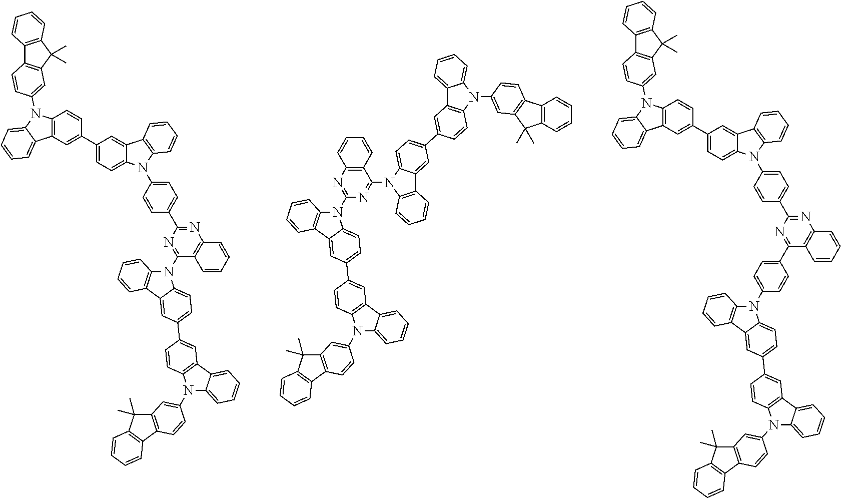

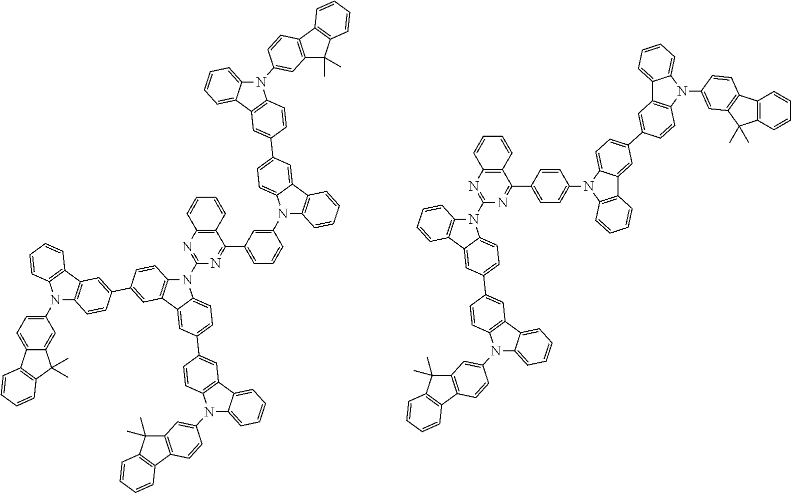 [SMILES: CC1(C)c2ccccc2-c2ccc(-n3c4ccccc4c4cc(-c5ccc6c(c5)c5ccccc5n6-c5cccc(-c6nc(-n7c8ccc(-c9ccc%10c(c9)c9ccccc9n%10-c9ccc%10c(c9)C(C)(C)c9ccccc9-%10)cc8c8cc(-c9ccc%10c(c9)c9ccccc9n%10-c9ccc%10c(c9)C(C)(C)c9ccccc9-%10)ccc87)nc7ccccc67)c5)ccc43)cc21 ; CC1(C)c2ccccc2-c2ccc(-n3c4ccccc4c4cc(-c5ccc6c(c5)c5ccccc5n6-c5ccc(-c6nc(-n7c8ccccc8c8cc(-c9ccc%10c(c9)c9ccccc9n%10-c9ccc%10c(c9)C(C)(C)c9ccccc9-%10)ccc87)nc7ccccc67)cc5)ccc43)cc21]

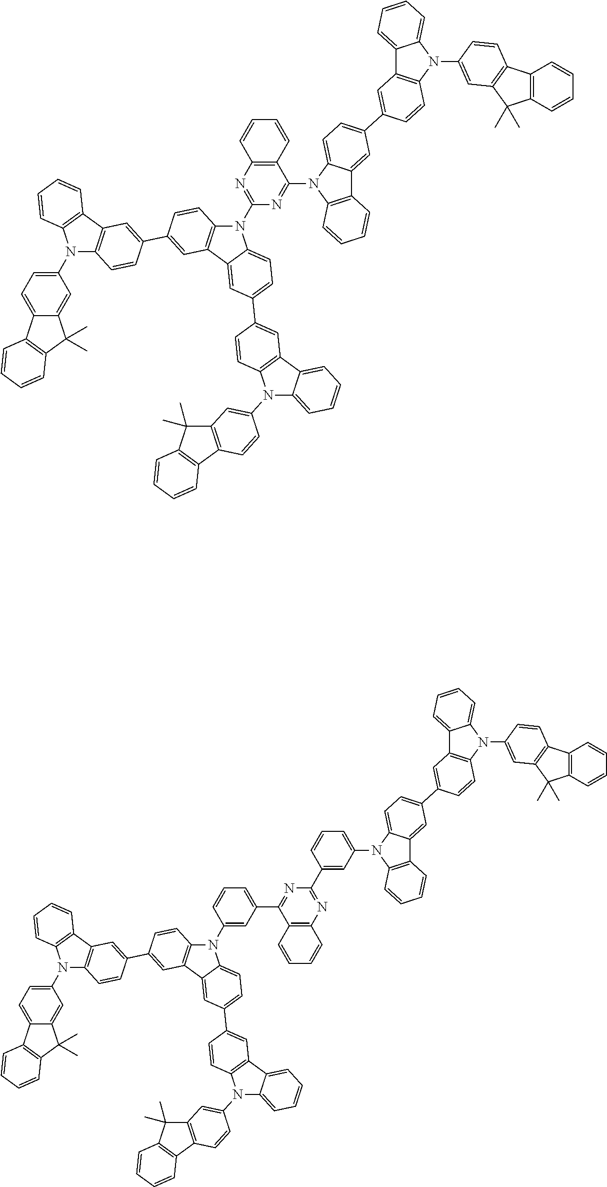 [SMILES: CC1(C)c2ccccc2-c2ccc(-n3c4ccccc4c4cc(-c5ccc6c(c5)c5cc(-c7ccc8c(c7)c7ccccc7n8-c7ccc8c(c7)C(C)(C)c7ccccc7-8)ccc5n6-c5nc(-n6c7ccccc7c7cc(-c8ccc9c(c8)c8ccccc8n9-c8ccc9c(c8)C(C)(C)c8ccccc8-9)ccc76)c6ccccc6n5)ccc43)cc21 ; CC1(C)c2ccccc2-c2ccc(-n3c4ccccc4c4cc(-c5ccc6c(c5)c5ccccc5n6-c5cccc(-c6nc(-c7cccc(-n8c9ccc(-c%10ccc%11c(c%10)c%10ccccc%10n%11-c%10ccc%11c(c%10)C(C)(C)c%10ccccc%10-%11)cc9c9cc(-c%10ccc%11c(c%10)c%10ccccc%10n%11-c%10ccc%11c(c%10)C(C)(C)c%10ccccc%10-%11)ccc98)c7)c7ccccc7n6)c5)ccc43)cc21]

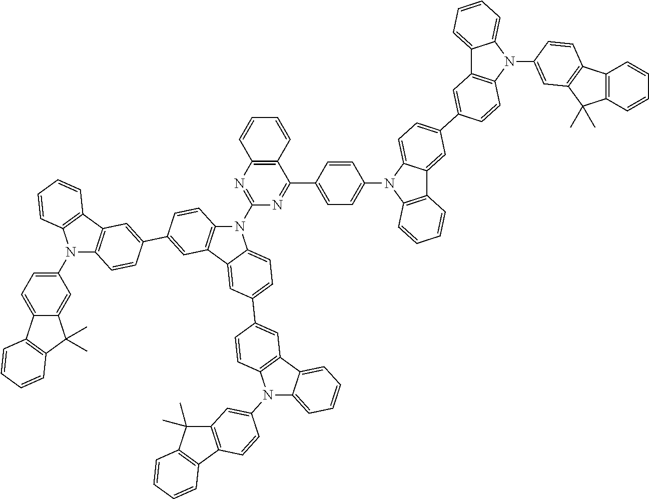 [SMILES: CC1(C)c2ccccc2-c2ccc(-n3c4ccccc4c4cc(-c5ccc6c(c5)c5ccccc5n6-c5ccc(-c6nc(-n7c8ccc(-c9ccc%10c(c9)c9ccccc9n%10-c9ccc%10c(c9)C(C)(C)c9ccccc9-%10)cc8c8cc(-c9ccc%10c(c9)c9ccccc9n%10-c9ccc%10c(c9)C(C)(C)c9ccccc9-%10)ccc87)nc7ccccc67)cc5)ccc43)cc21]

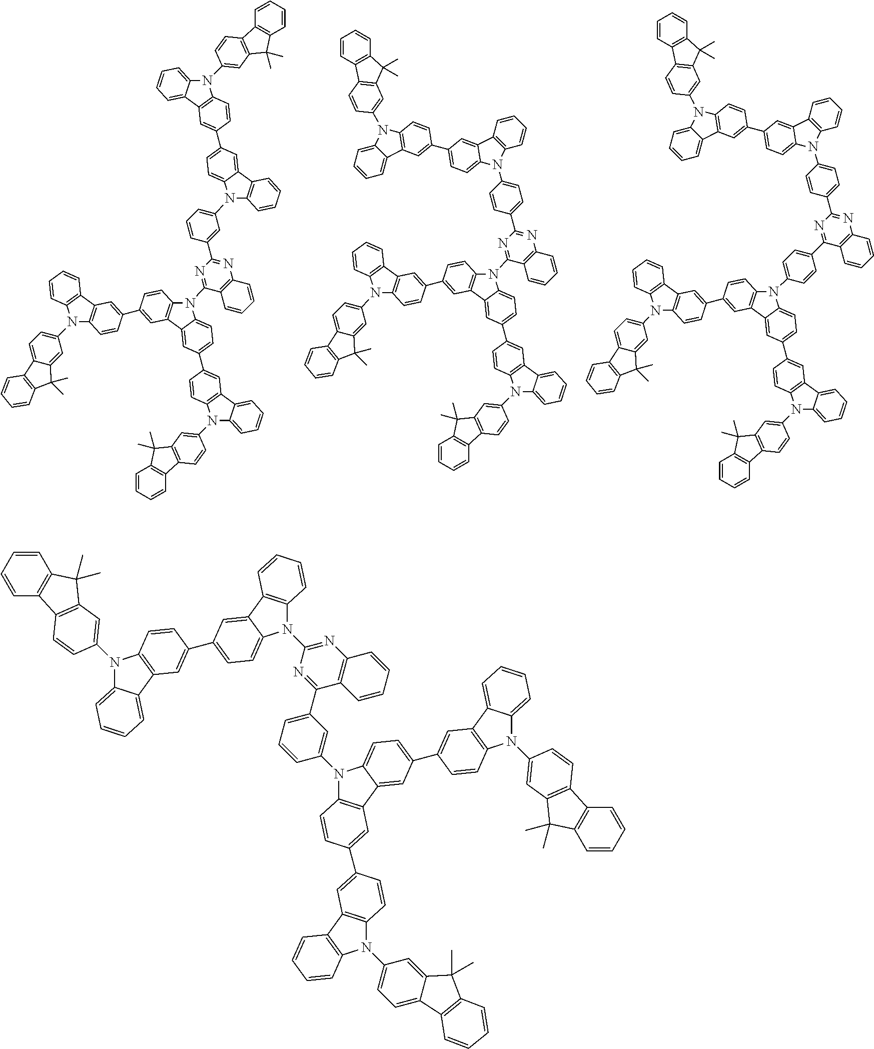

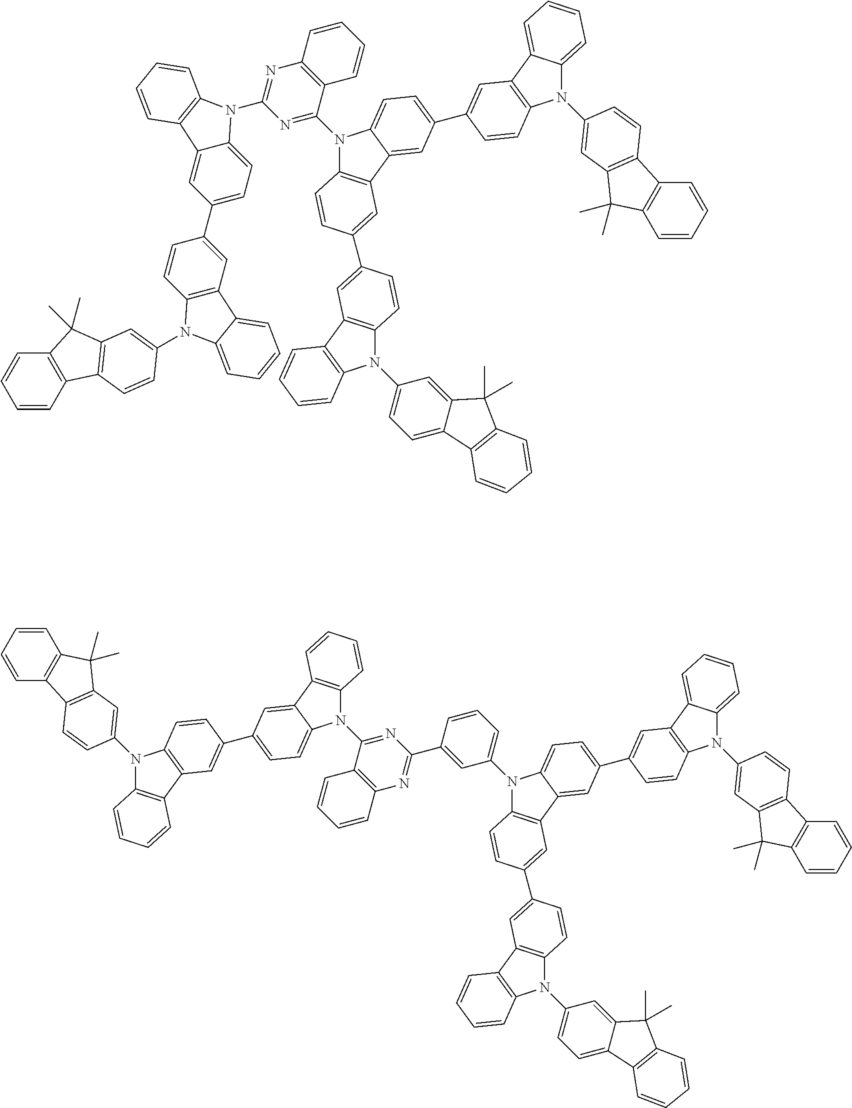 [SMILES: CC1(C)c2ccccc2-c2ccc(-n3c4ccccc4c4cc(-c5ccc6c(c5)c5ccccc5n6-c5nc(-n6c7ccc(-c8ccc9c(c8)c8ccccc8n9-c8ccc9c(c8)C(C)(C)c8ccccc8-9)cc7c7cc(-c8ccc9c(c8)c8ccccc8n9-c8ccc9c(c8)C(C)(C)c8ccccc8-9)ccc76)c6ccccc6n5)ccc43)cc21 ; CC1(C)c2ccccc2-c2ccc(-n3c4ccccc4c4cc(-c5ccc6c(c5)c5cc(-c7ccc8c(c7)c7ccccc7n8-c7ccc8c(c7)C(C)(C)c7ccccc7-8)ccc5n6-c5cccc(-c6nc(-n7c8ccccc8c8cc(-c9ccc%10c(c9)c9ccccc9n%10-c9ccc%10c(c9)C(C)(C)c9ccccc9-%10)ccc87)c7ccccc7n6)c5)ccc43)cc21]

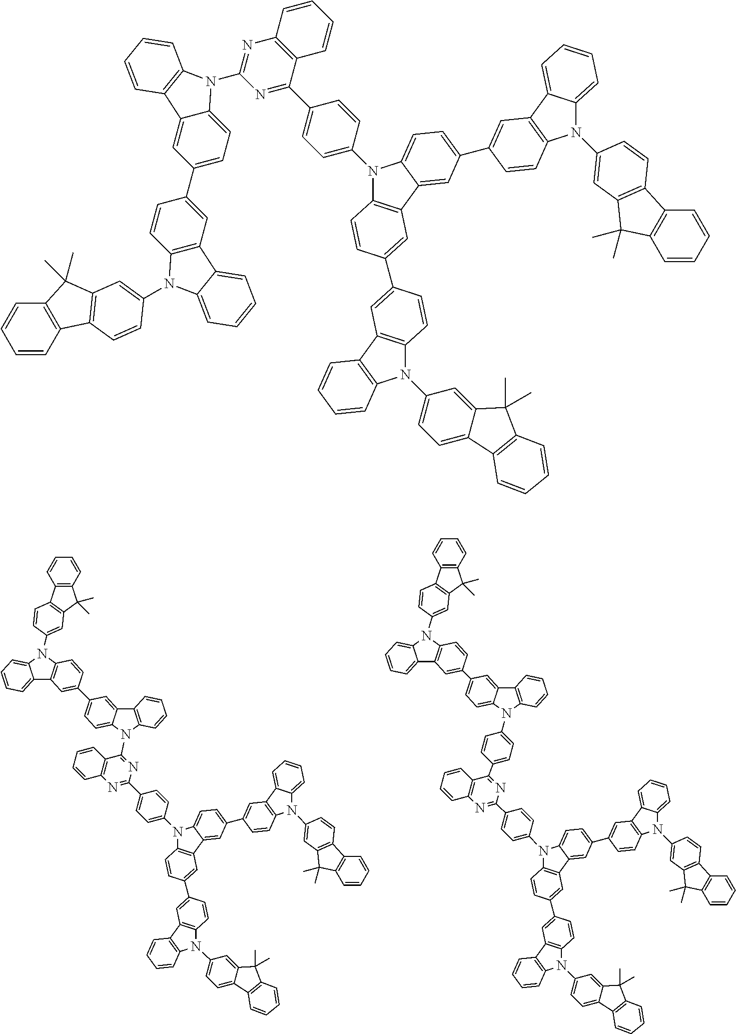

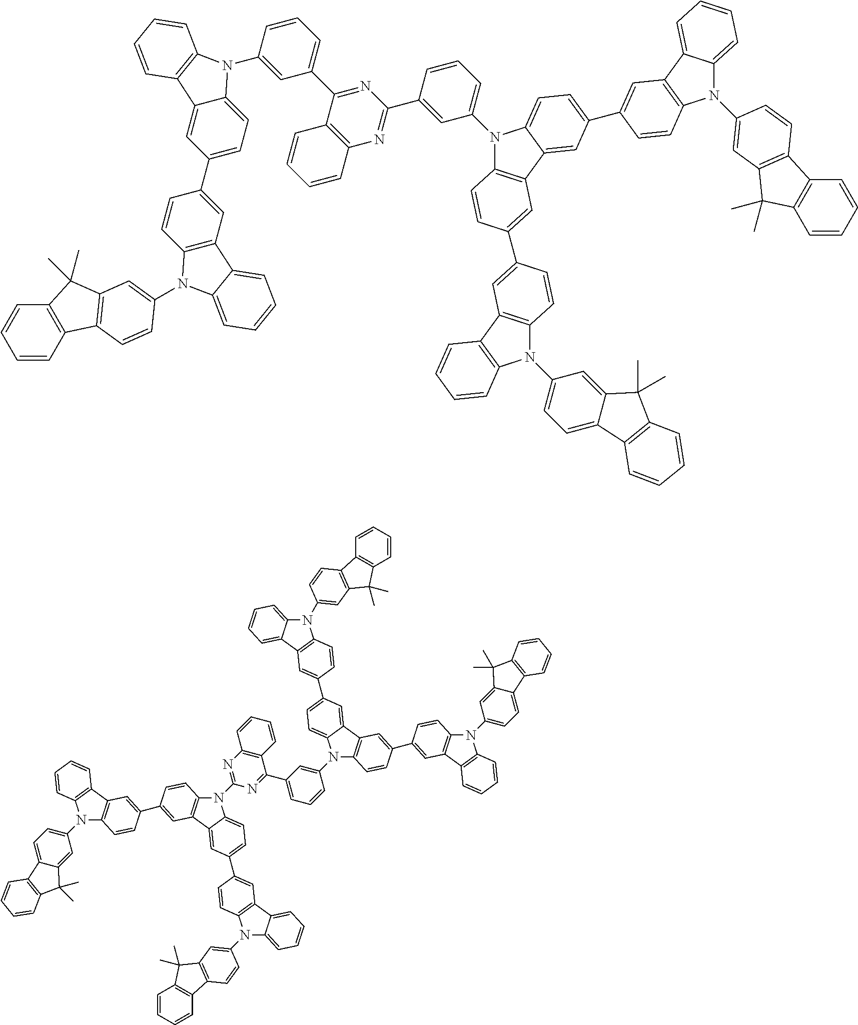

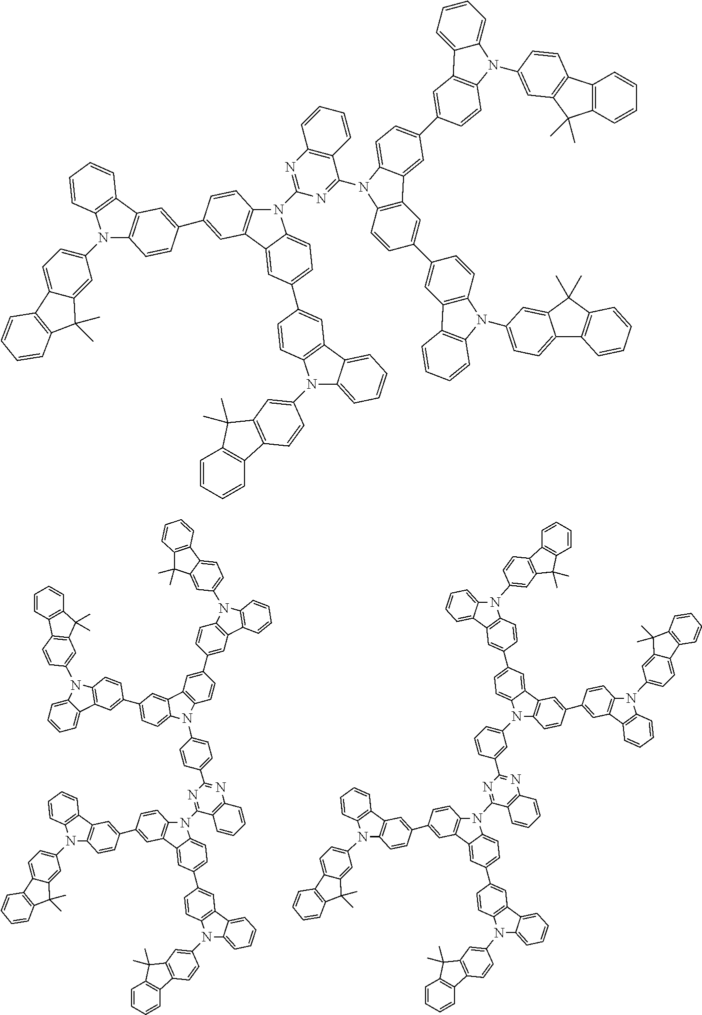

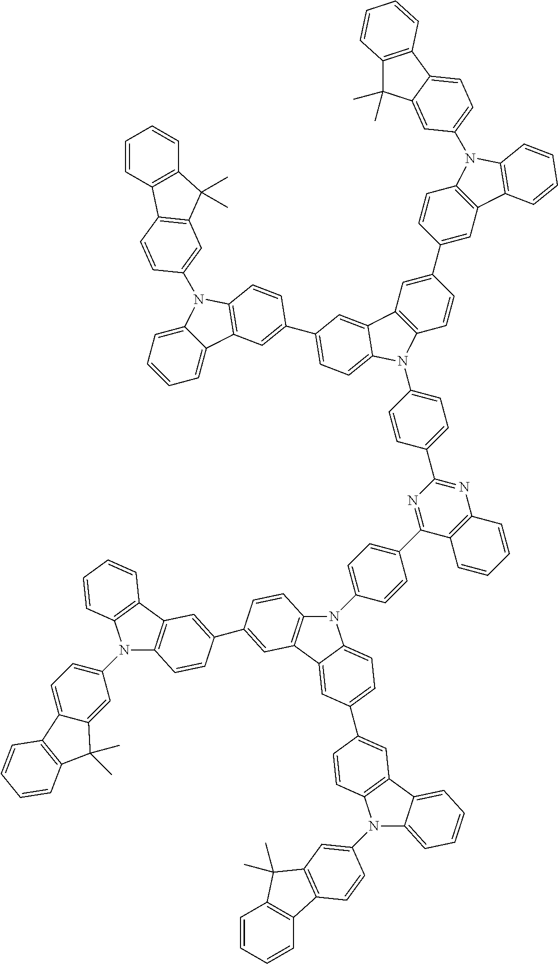 [SMILES: CC1(C)c2ccccc2-c2ccc(-n3c4ccccc4c4cc(-c5ccc6c(c5)c5cc(-c7ccc8c(c7)c7ccccc7n8-c7ccc8c(c7)C(C)(C)c7ccccc7-8)ccc5n6-c5ccc(-c6nc(-c7ccc(-n8c9ccc(-c%10ccc%11c(c%10)c%10ccccc%10n%11-c%10ccc%11c(c%10)C(C)(C)c%10ccccc%10-%11)cc9c9cc(-c%10ccc%11c(c%10)c%10ccccc%10n%11-c%10ccc%11c(c%10)C(C)(C)c%10ccccc%10-%11)ccc98)cc7)c7ccccc7n6)cc5)ccc43)cc21]

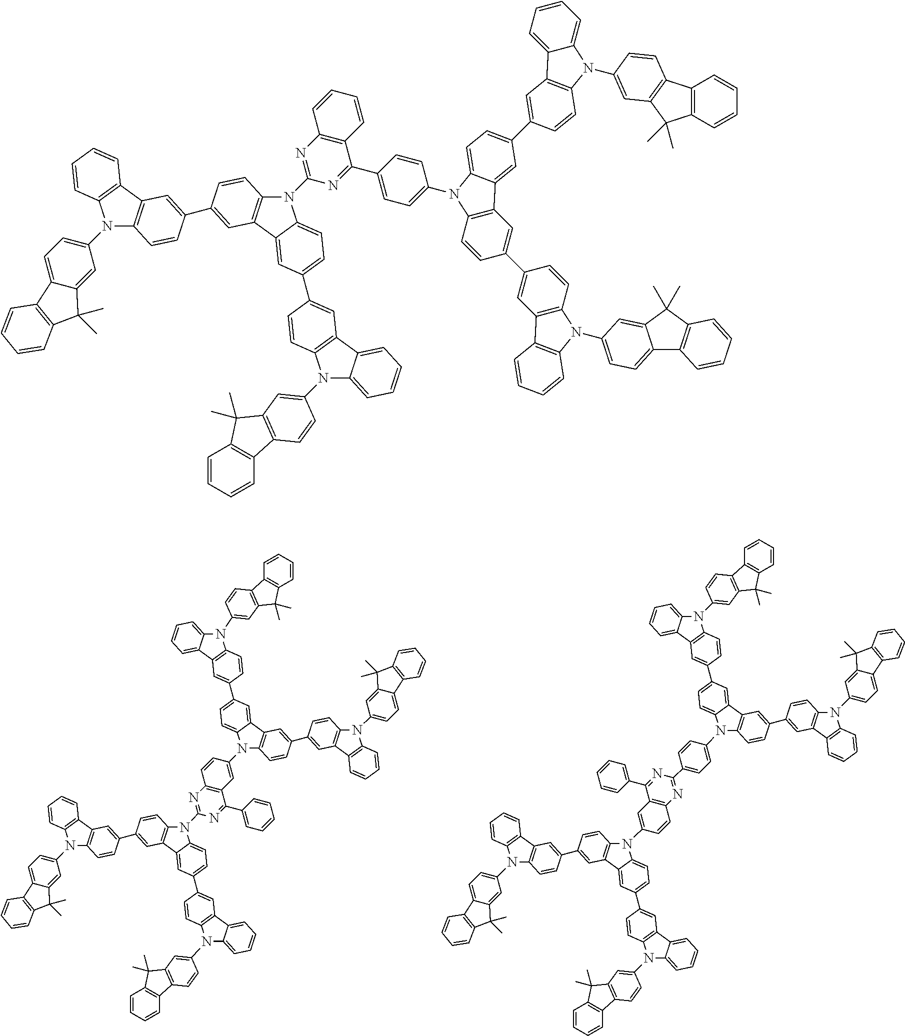

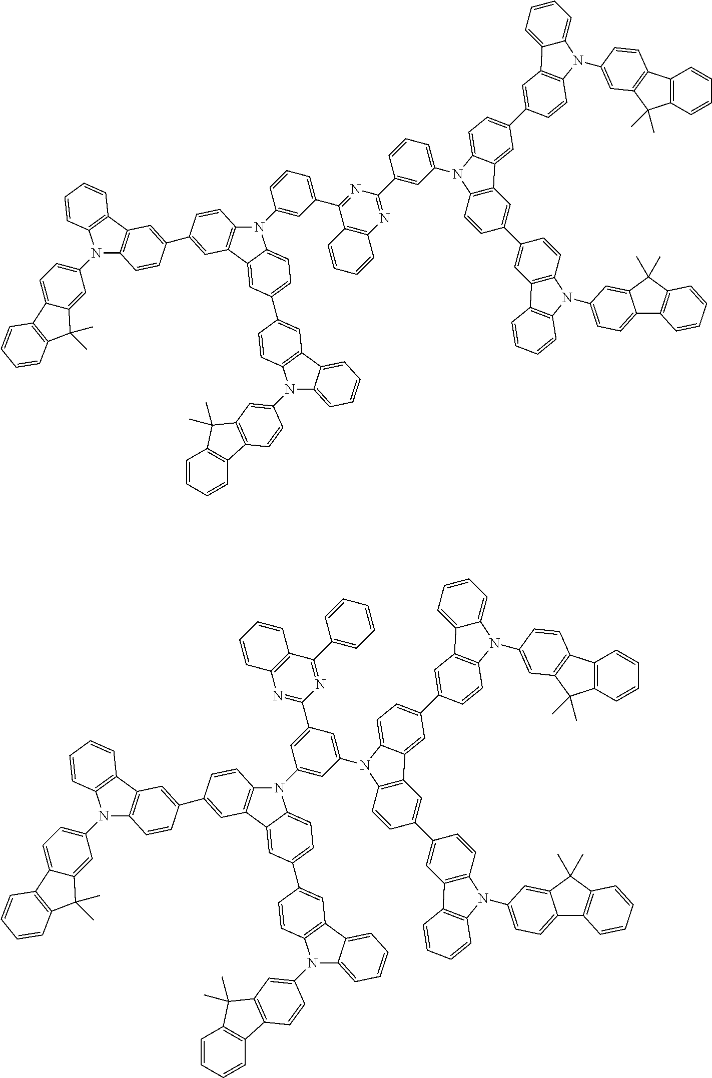

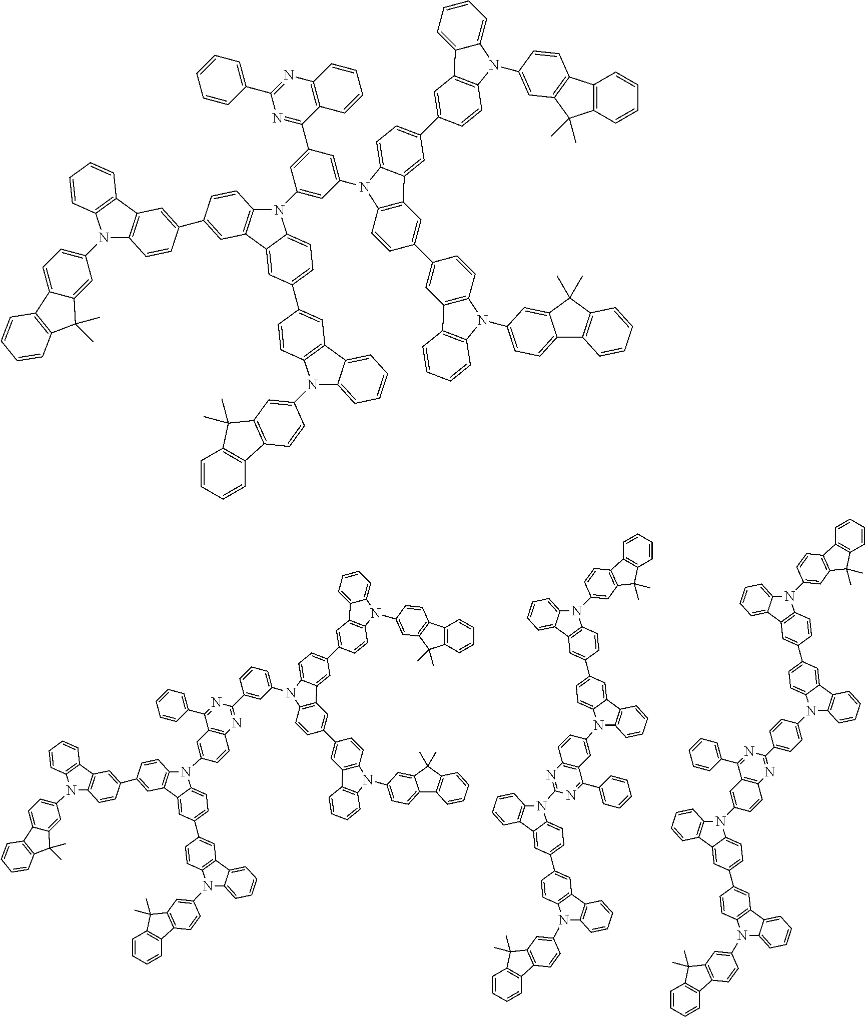

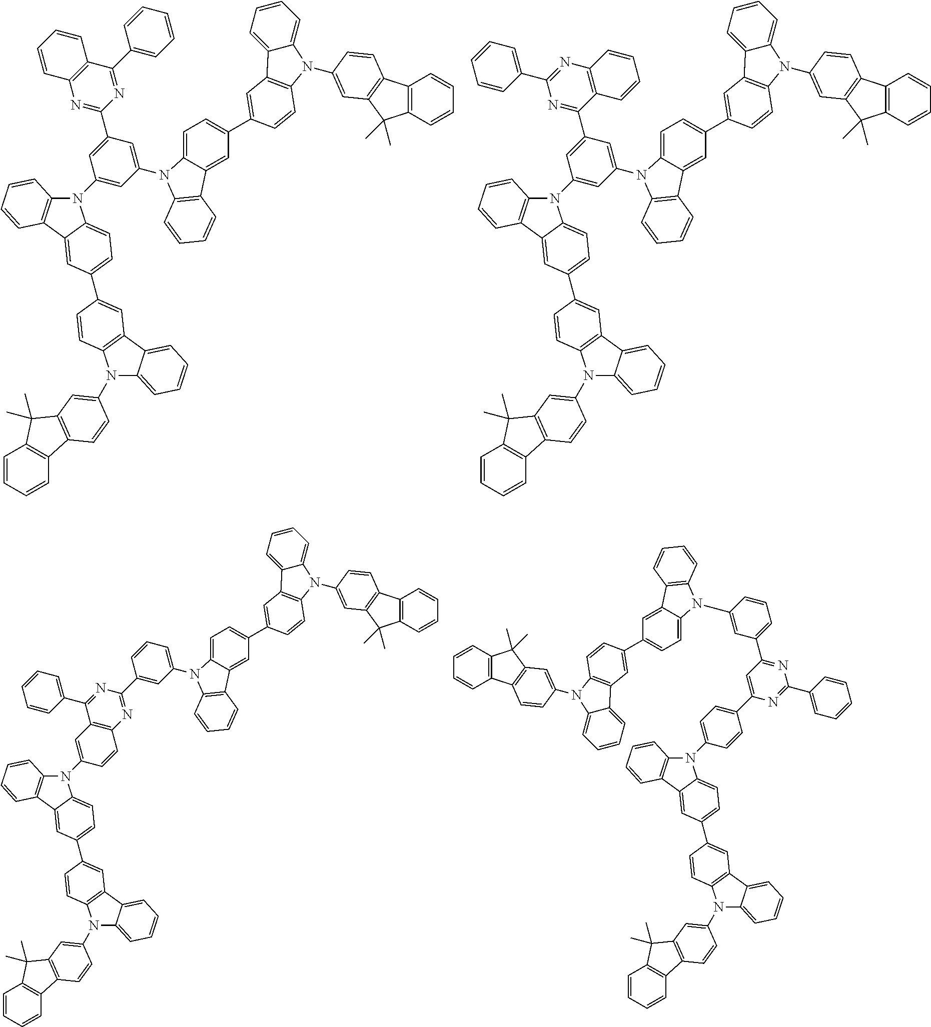

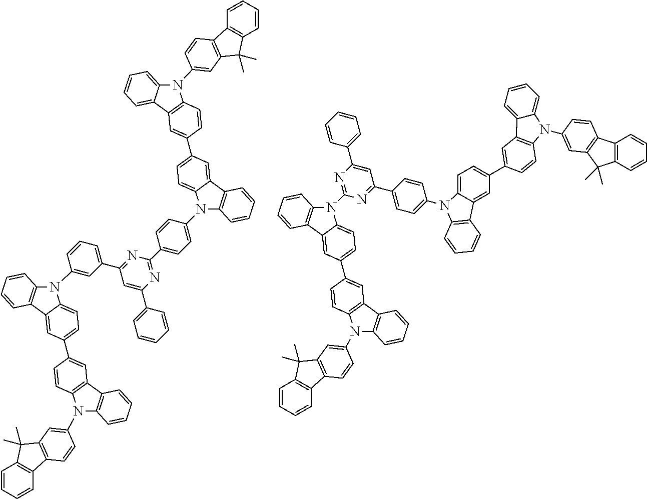 [SMILES: CC1(C)c2ccccc2-c2ccc(-n3c4ccccc4c4cc(-c5ccc6c(c5)c5ccccc5n6-c5ccc(-c6nc(-c7ccccc7)cc(-c7cccc(-n8c9ccccc9c9cc(-c%10ccc%11c(c%10)c%10ccccc%10n%11-c%10ccc%11c(c%10)C(C)(C)c%10ccccc%10-%11)ccc98)c7)n6)cc5)ccc43)cc21 ; CC1(C)c2ccccc2-c2ccc(-n3c4ccccc4c4cc(-c5ccc6c(c5)c5ccccc5n6-c5ccc(-c6cc(-c7ccccc7)nc(-n7c8ccccc8c8cc(-c9ccc%10c(c9)c9ccccc9n%10-c9ccc%10c(c9)C(C)(C)c9ccccc9-%10)ccc87)n6)cc5)ccc43)cc21]

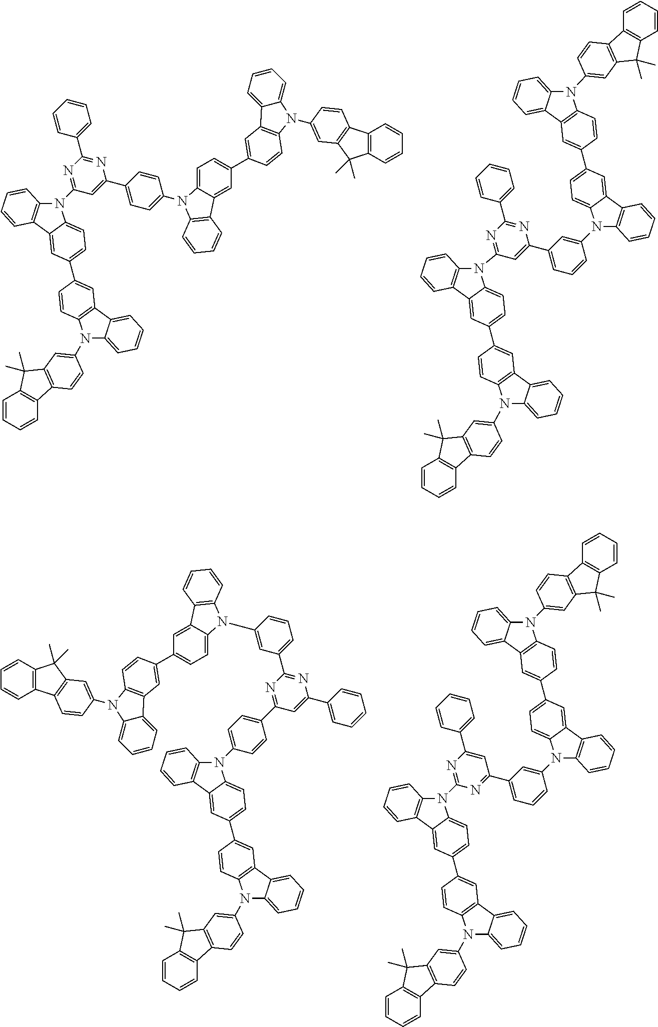

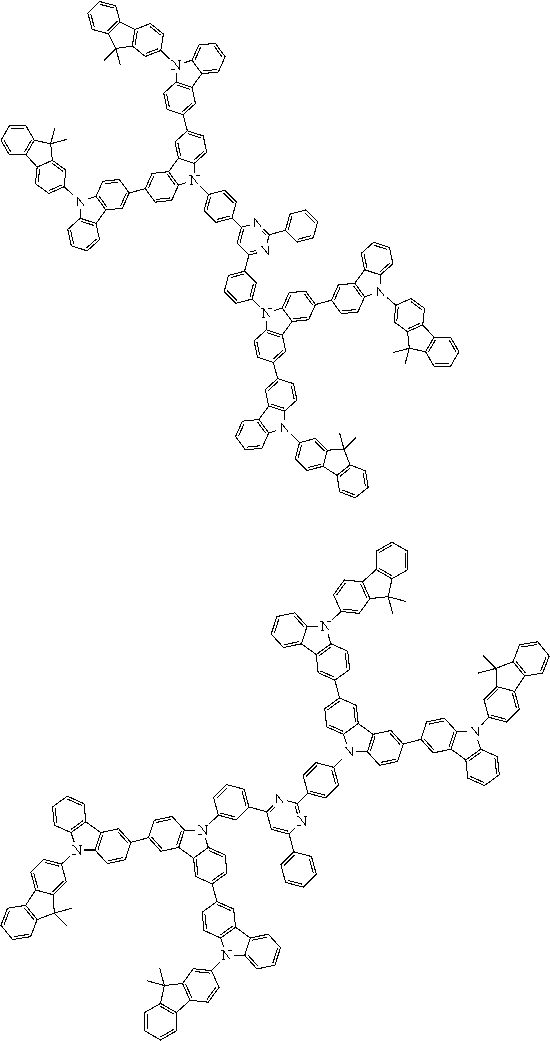

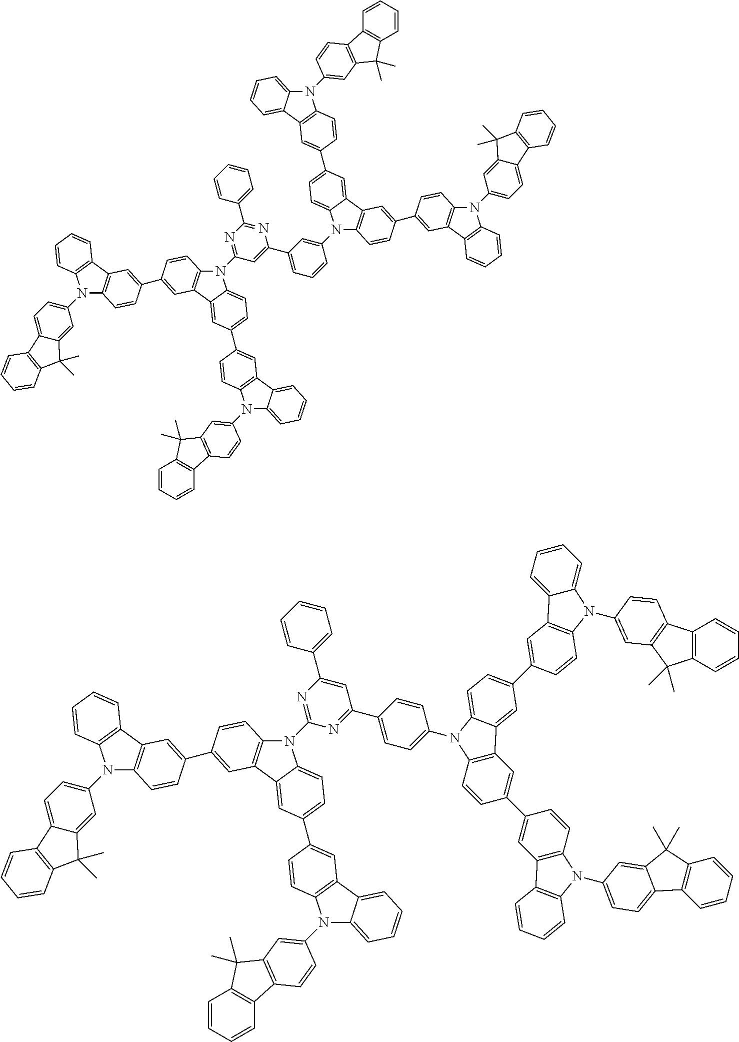

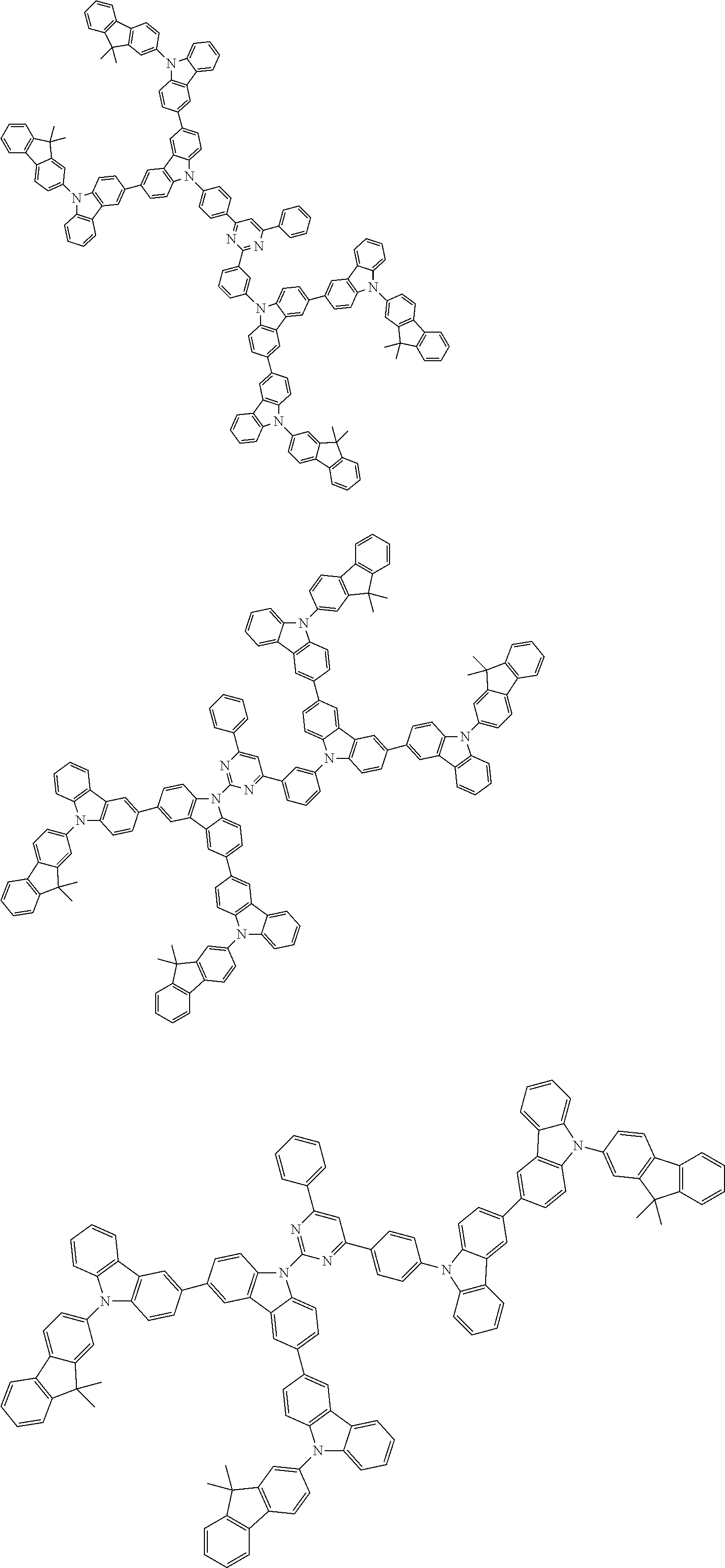

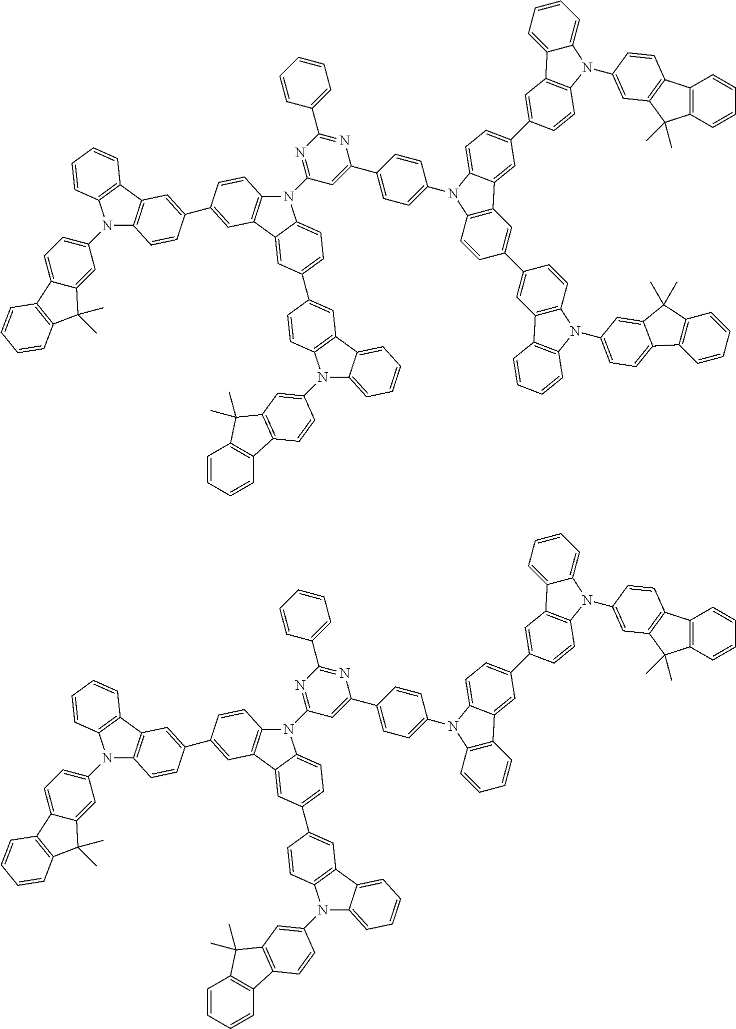

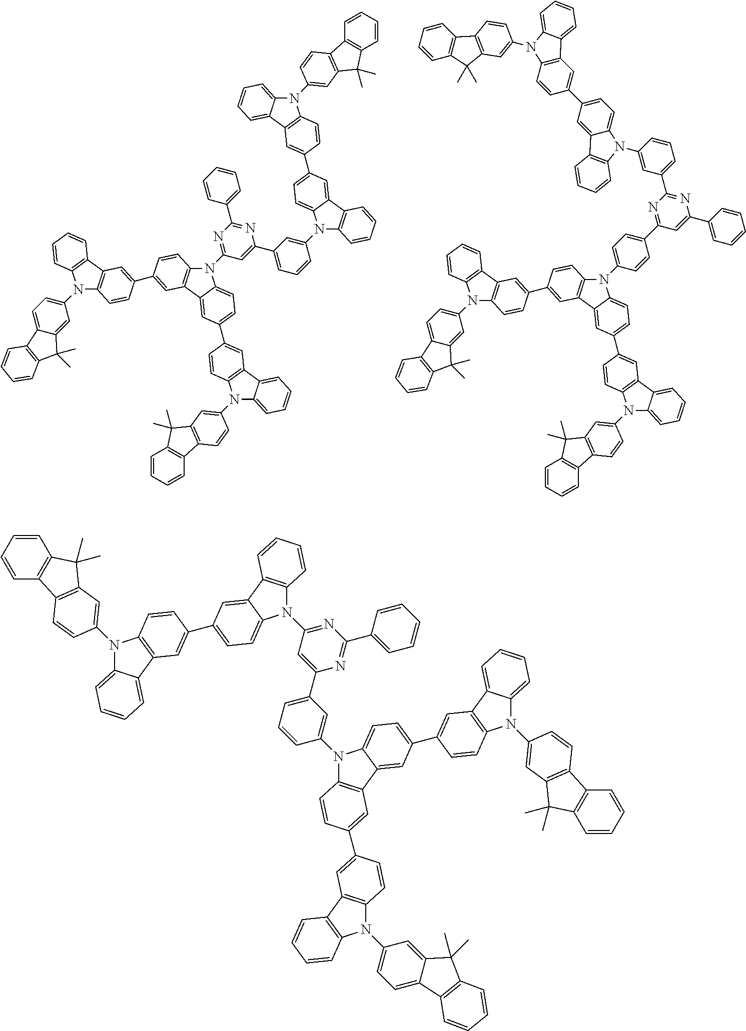

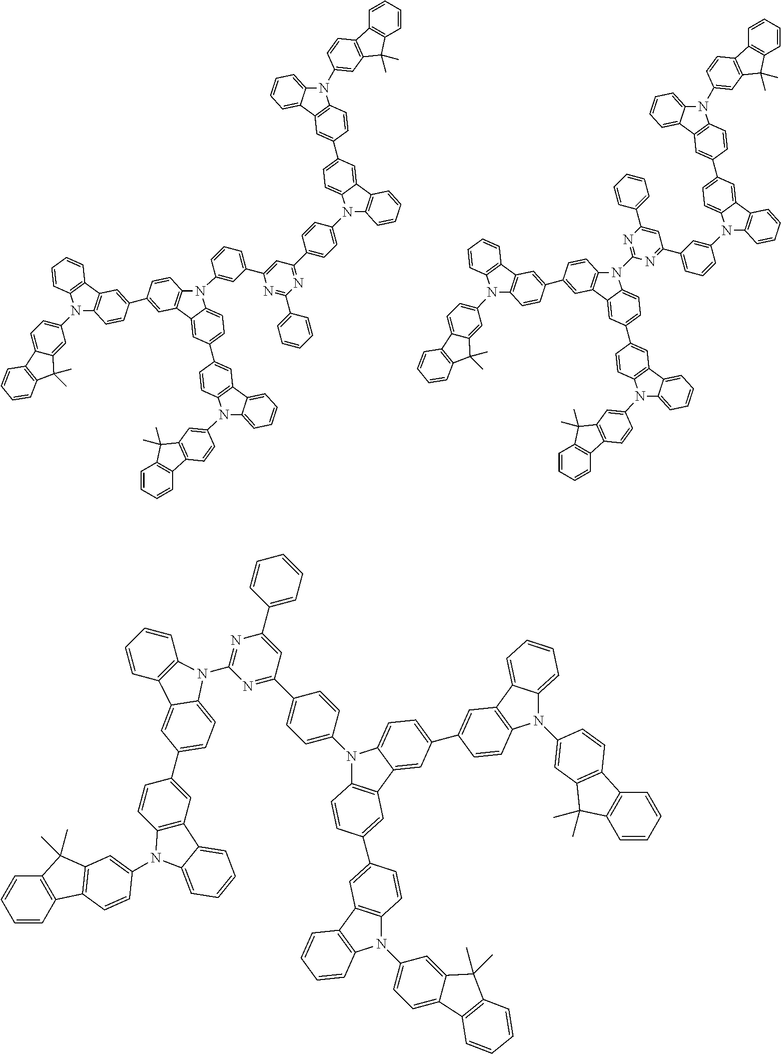

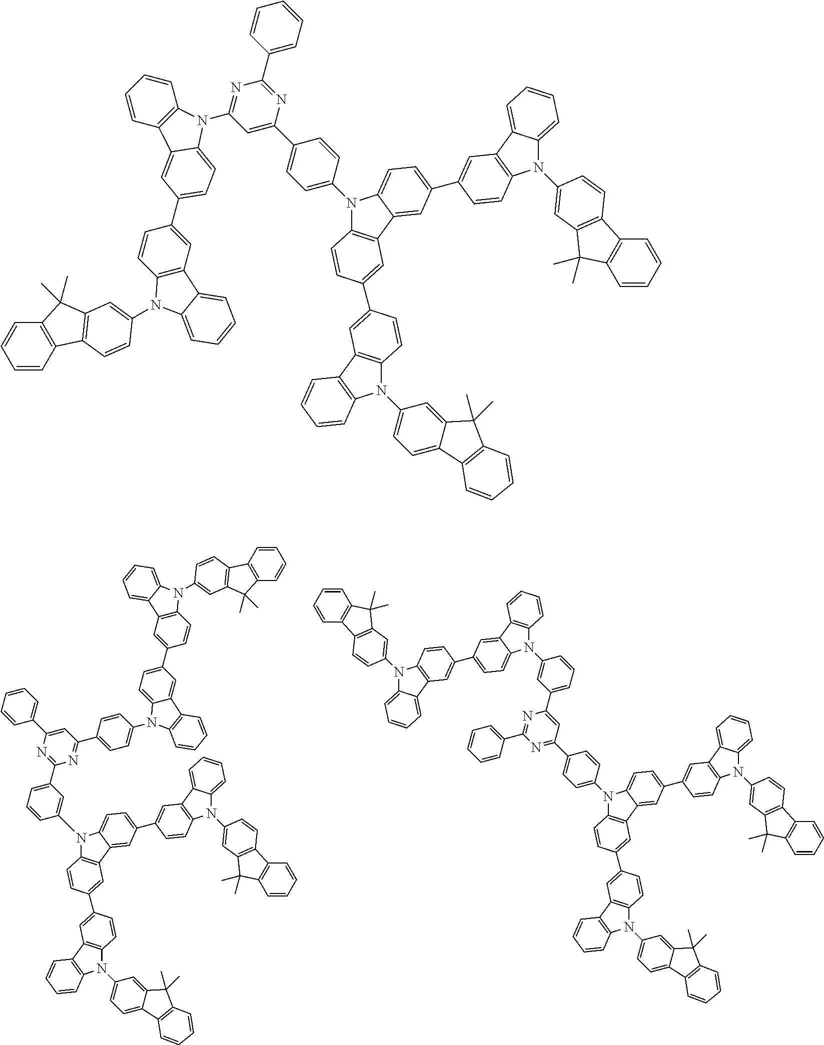

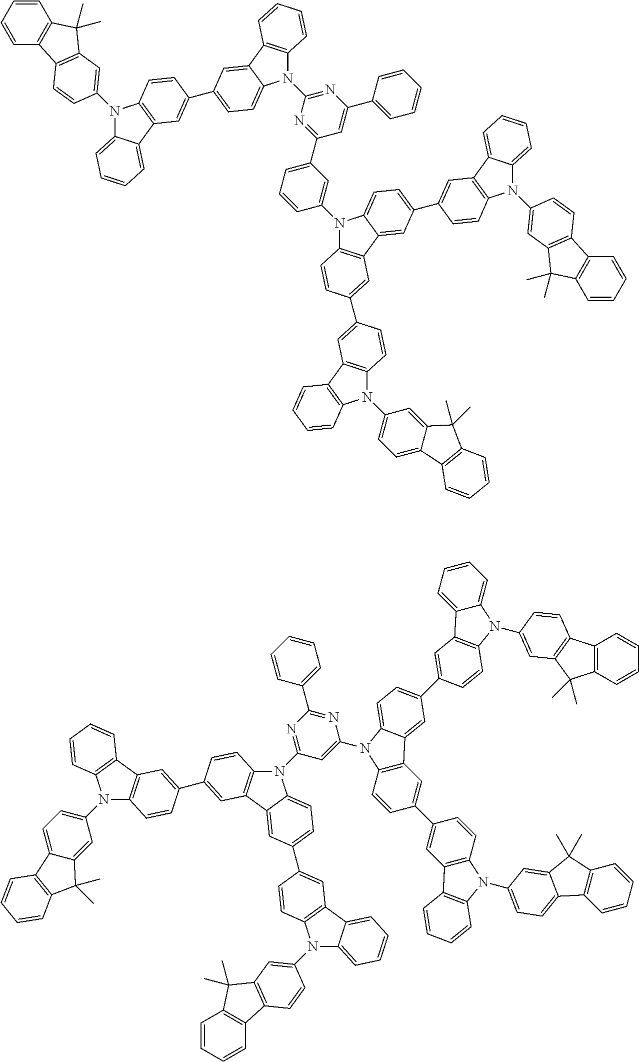

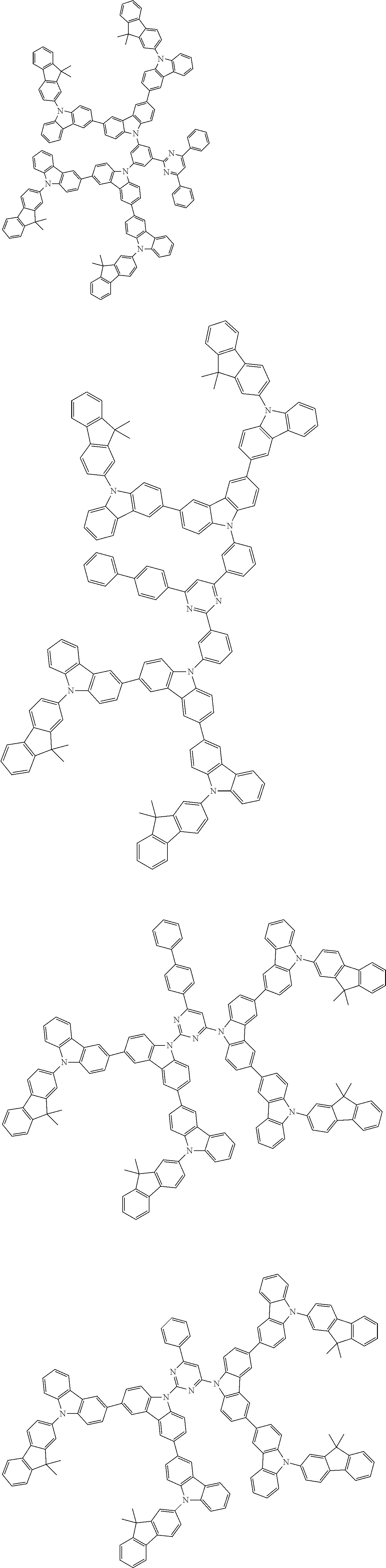

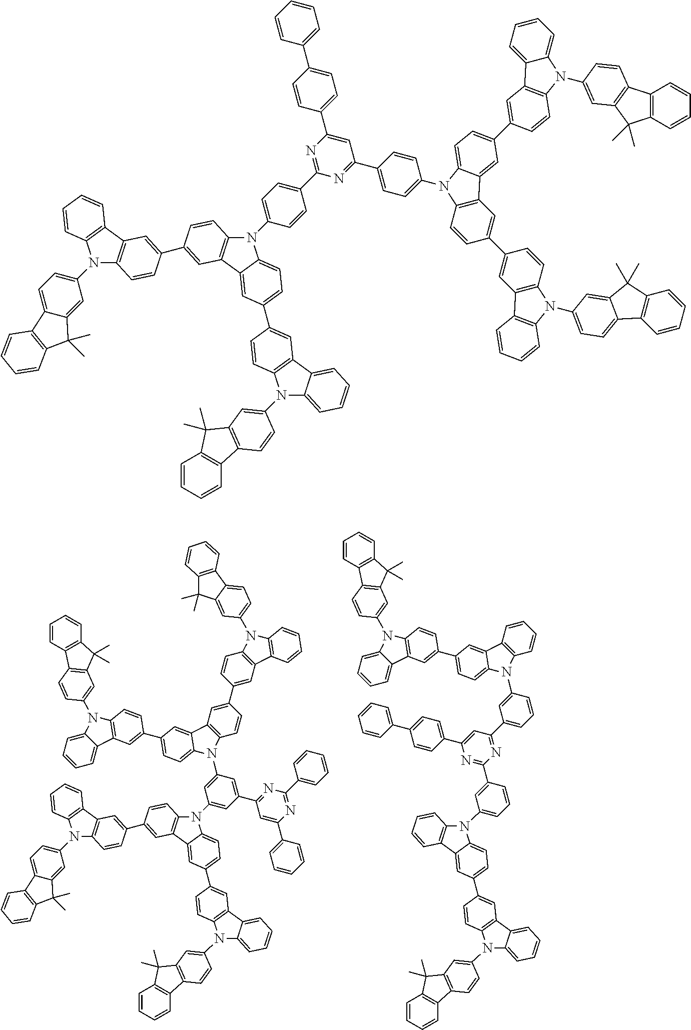

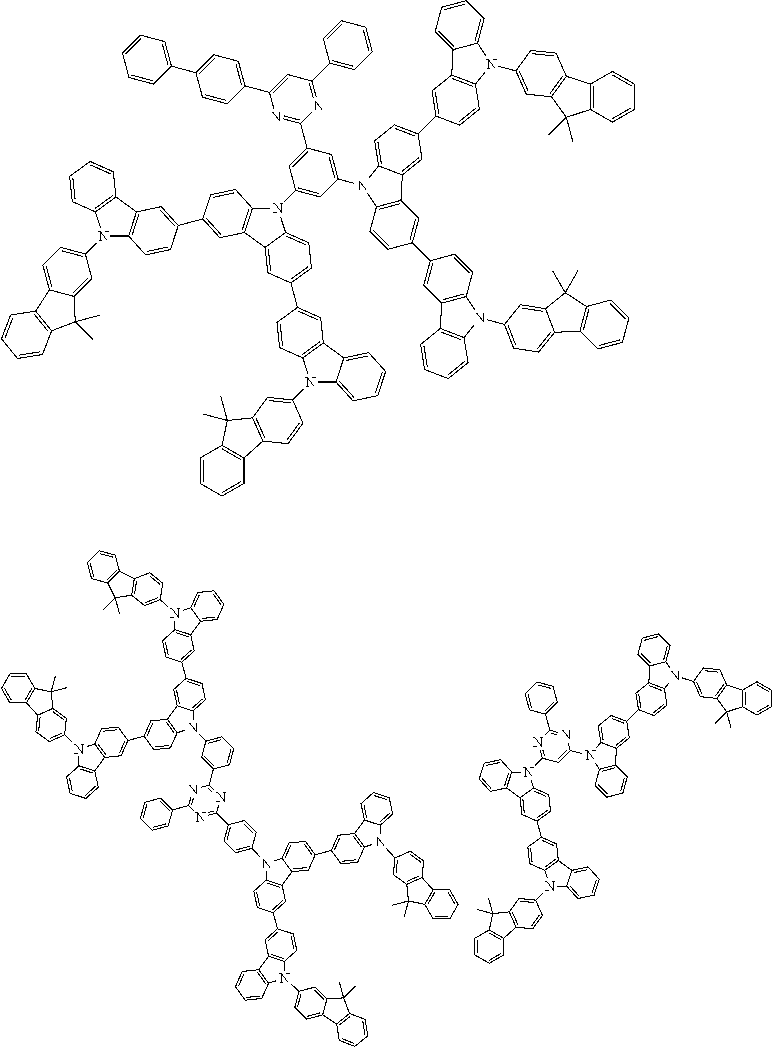

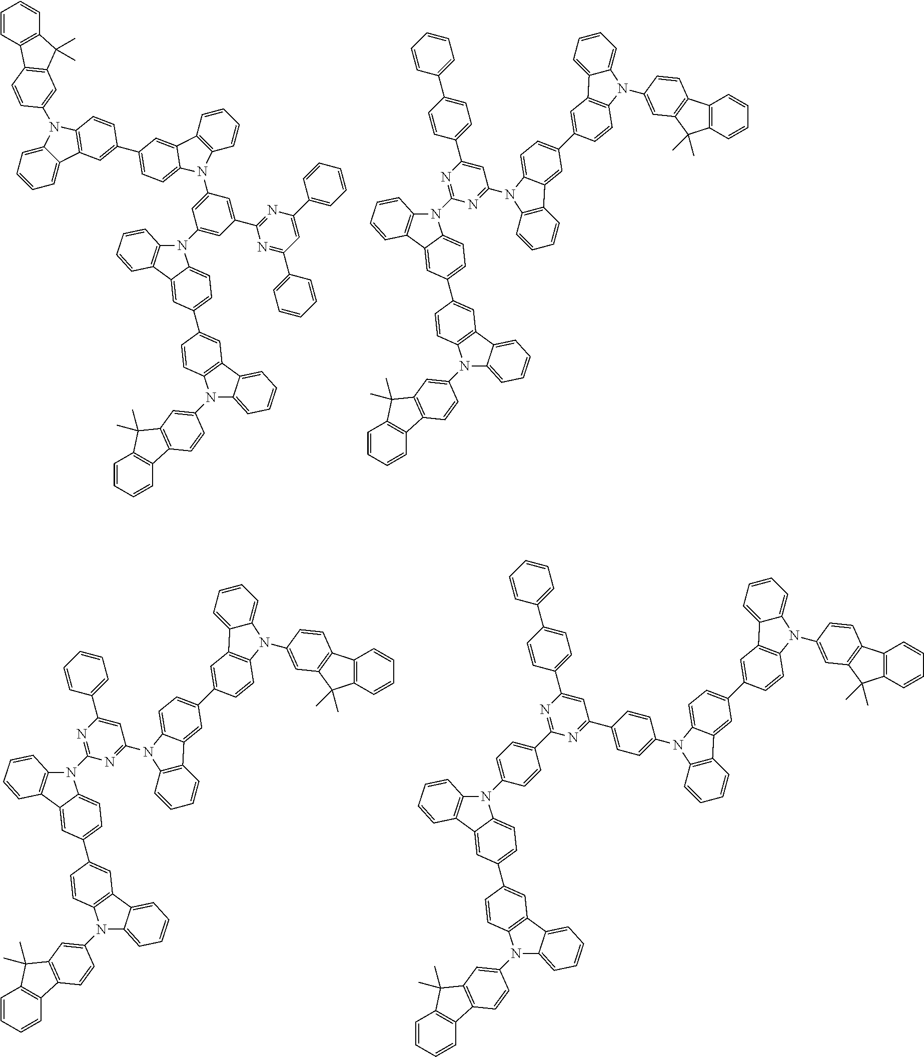

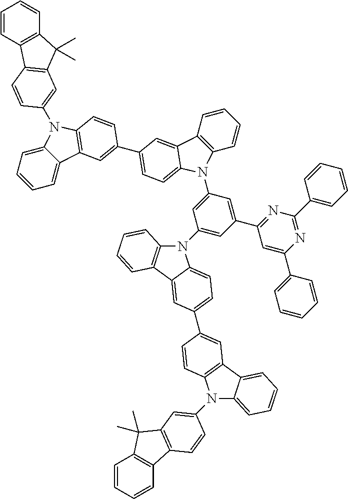 [SMILES: CC1(C)c2ccccc2-c2ccc(-n3c4ccccc4c4cc(-c5ccc6c(c5)c5ccccc5n6-c5cc(-c6cc(-c7ccccc7)nc(-c7ccccc7)n6)cc(-n6c7ccccc7c7cc(-c8ccc9c(c8)c8ccccc8n9-c8ccc9c(c8)C(C)(C)c8ccccc8-9)ccc76)c5)ccc43)cc21]

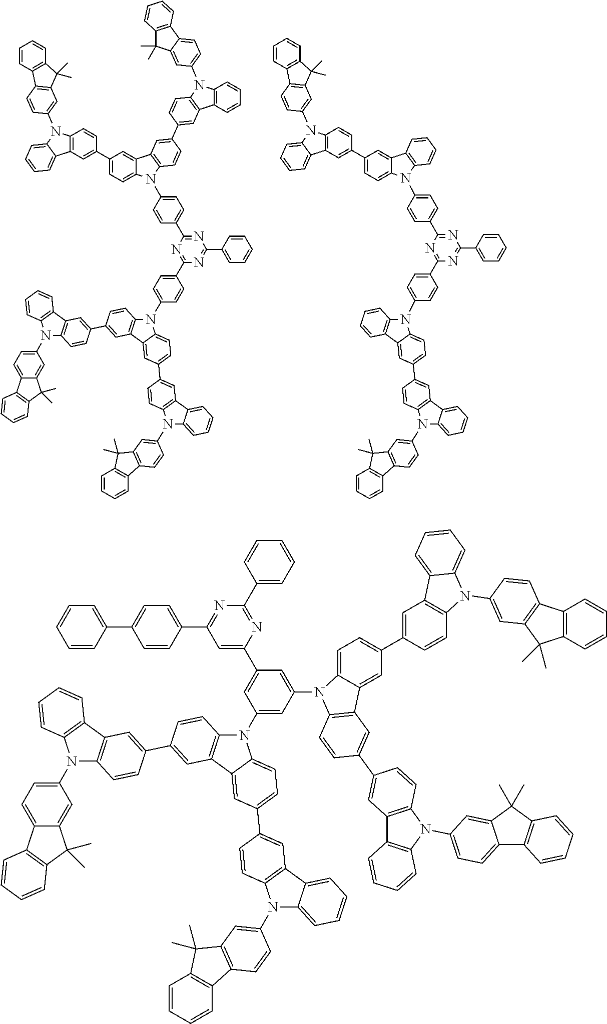

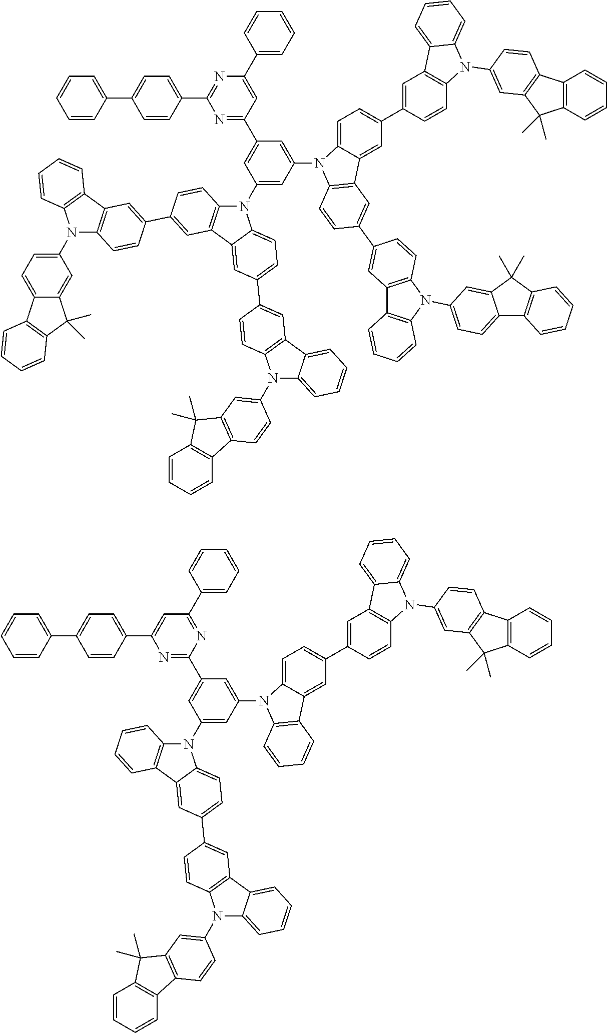

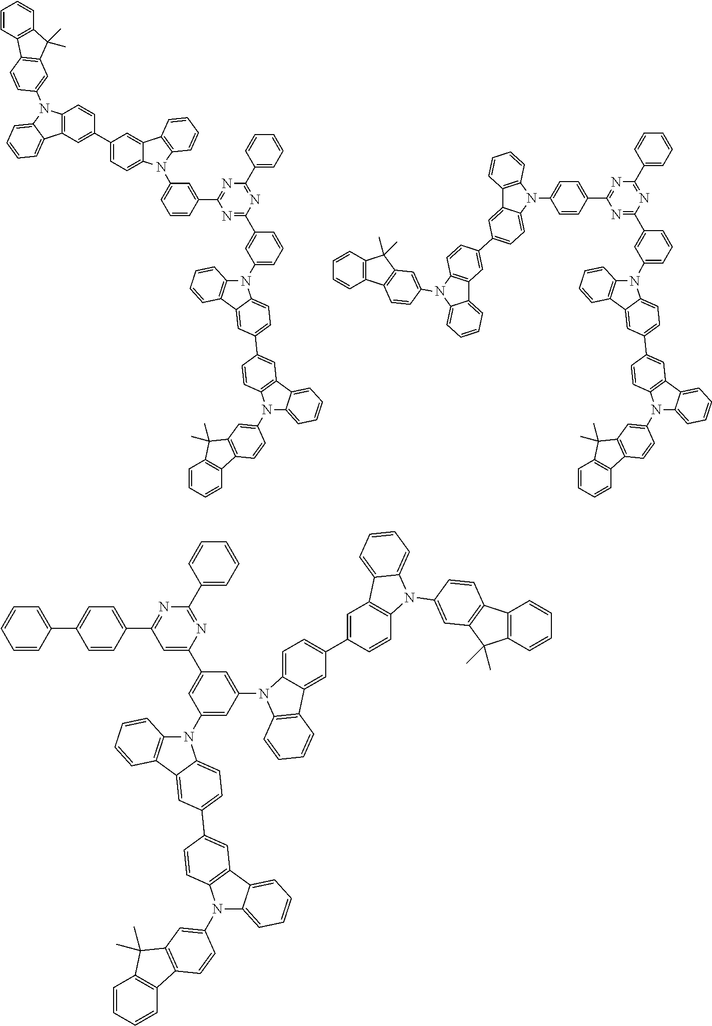

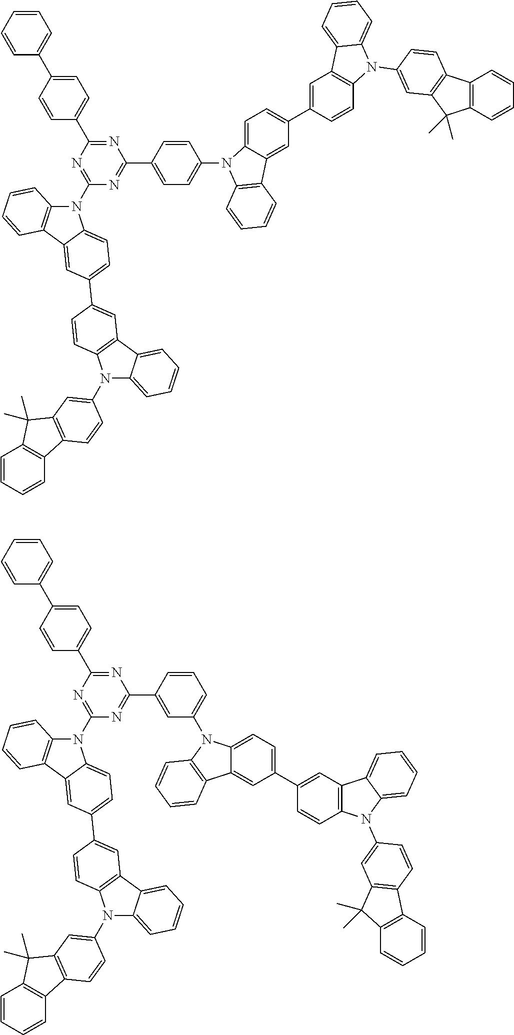 [SMILES: CC1(C)c2ccccc2-c2ccc(-n3c4ccccc4c4cc(-c5ccc6c(c5)c5ccccc5n6-c5ccc(-c6nc(-c7ccc(-c8ccccc8)cc7)nc(-n7c8ccccc8c8cc(-c9ccc%10c(c9)c9ccccc9n%10-c9ccc%10c(c9)C(C)(C)c9ccccc9-%10)ccc87)n6)cc5)ccc43)cc21 ; CC1(C)c2ccccc2-c2ccc(-n3c4ccccc4c4cc(-c5ccc6c(c5)c5ccccc5n6-c5cccc(-c6nc(-c7ccc(-c8ccccc8)cc7)nc(-n7c8ccccc8c8cc(-c9ccc%10c(c9)c9ccccc9n%10-c9ccc%10c(c9)C(C)(C)c9ccccc9-%10)ccc87)n6)c5)ccc43)cc21]

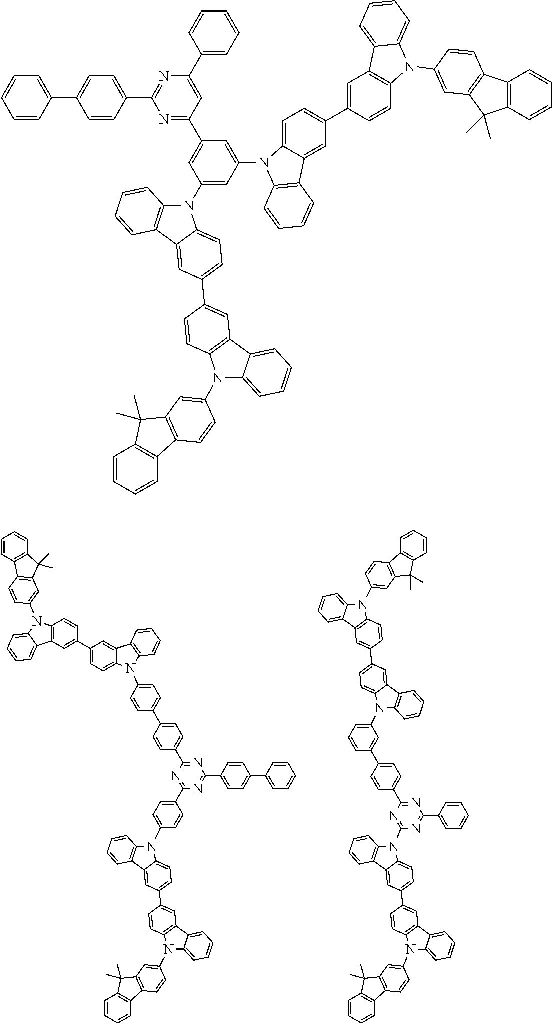

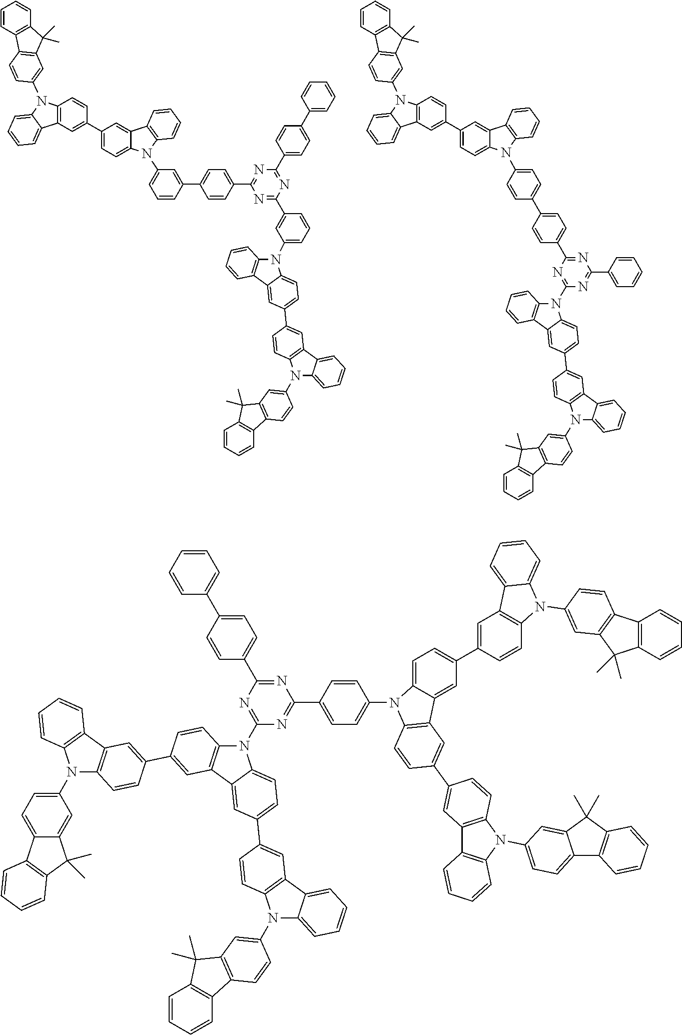

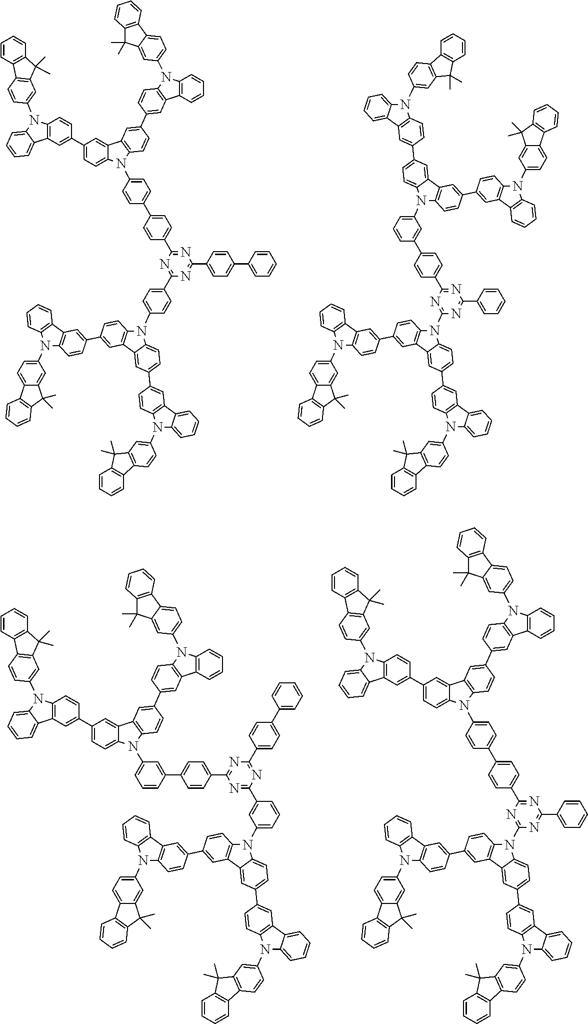

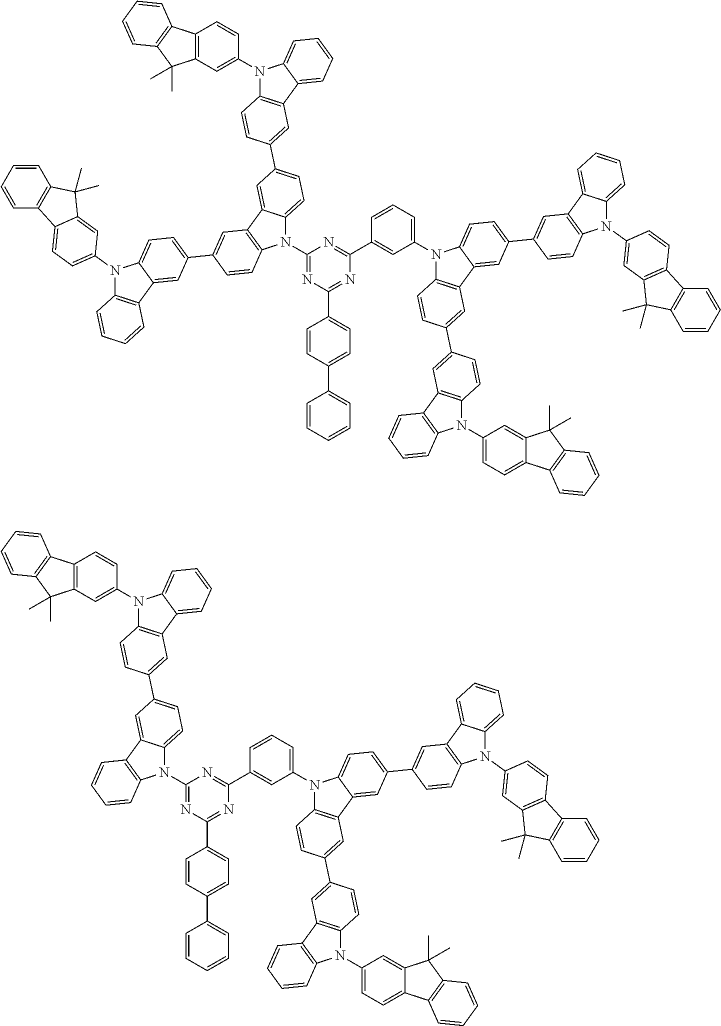

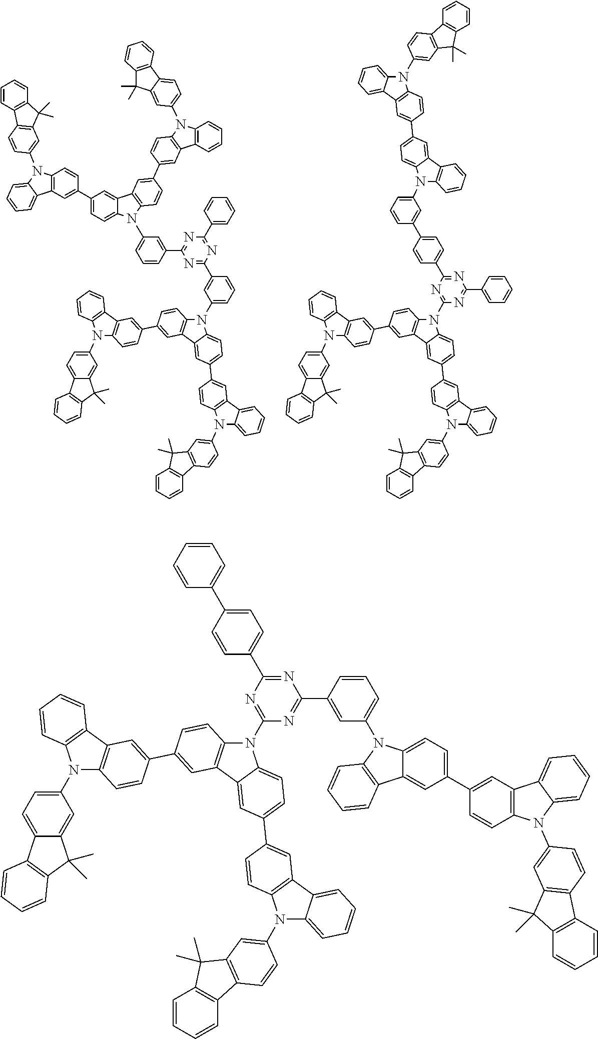

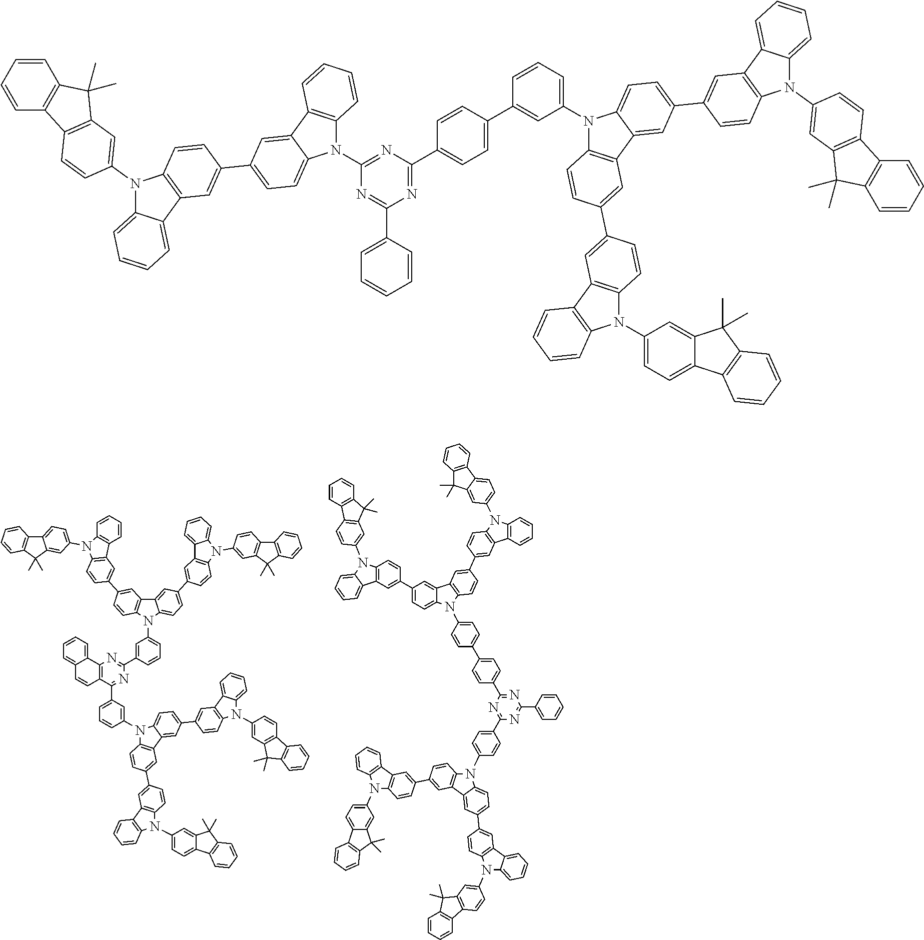

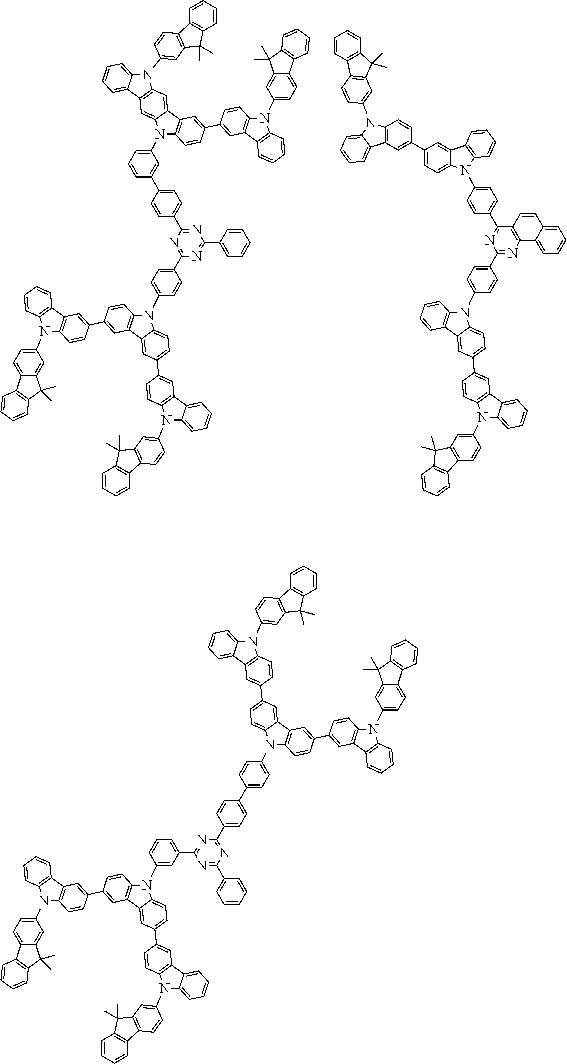

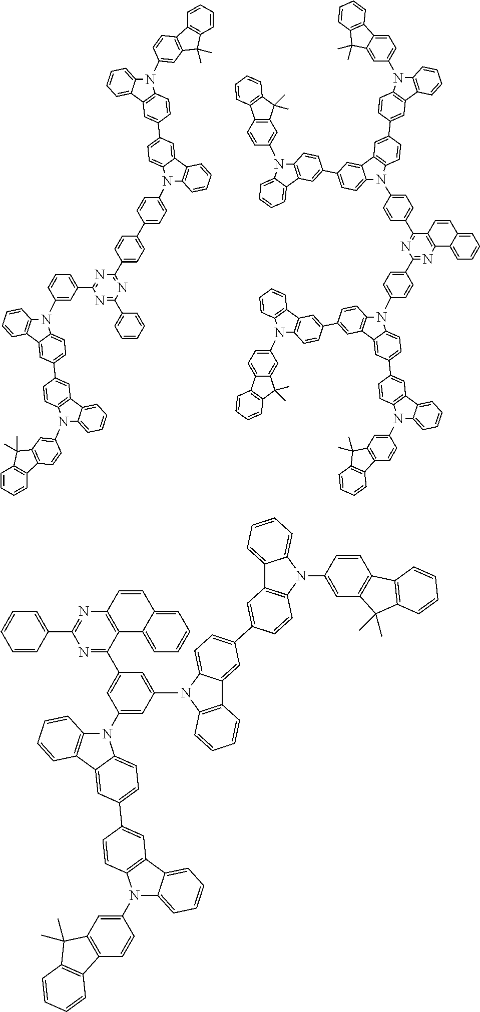

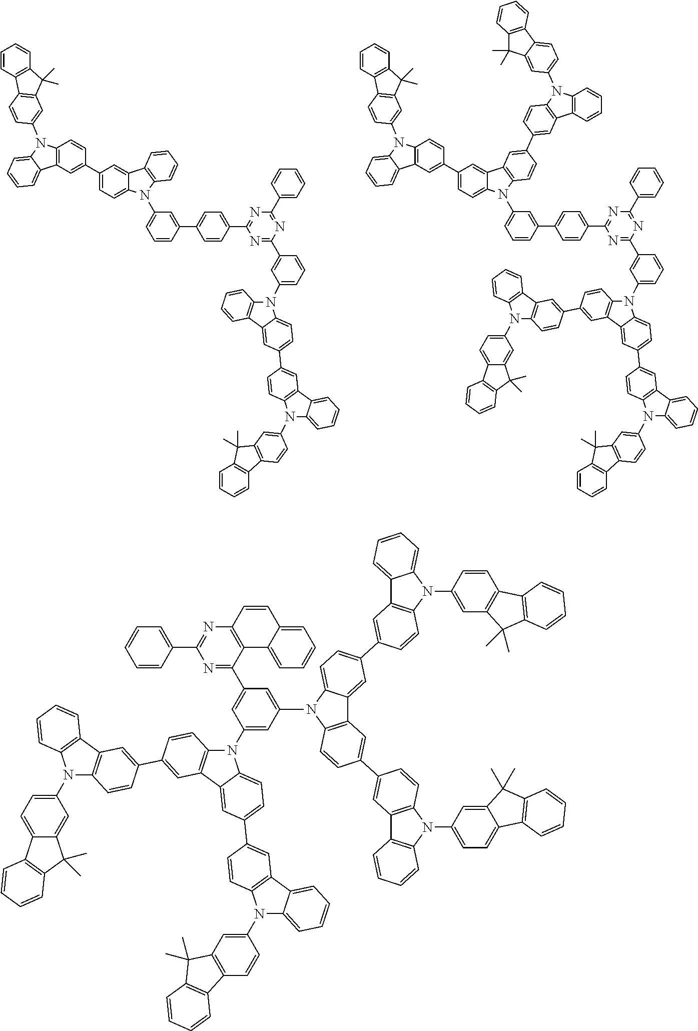

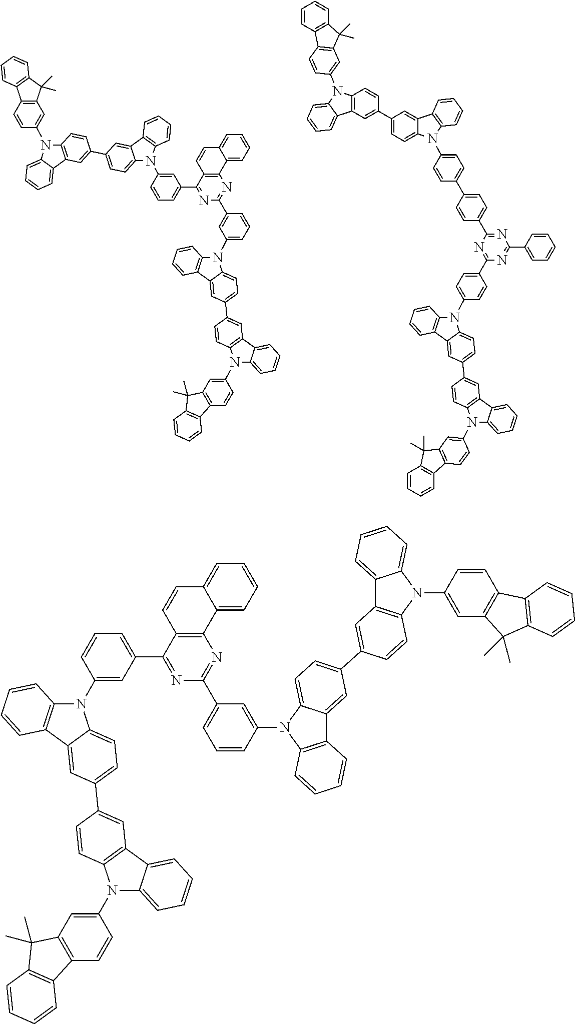

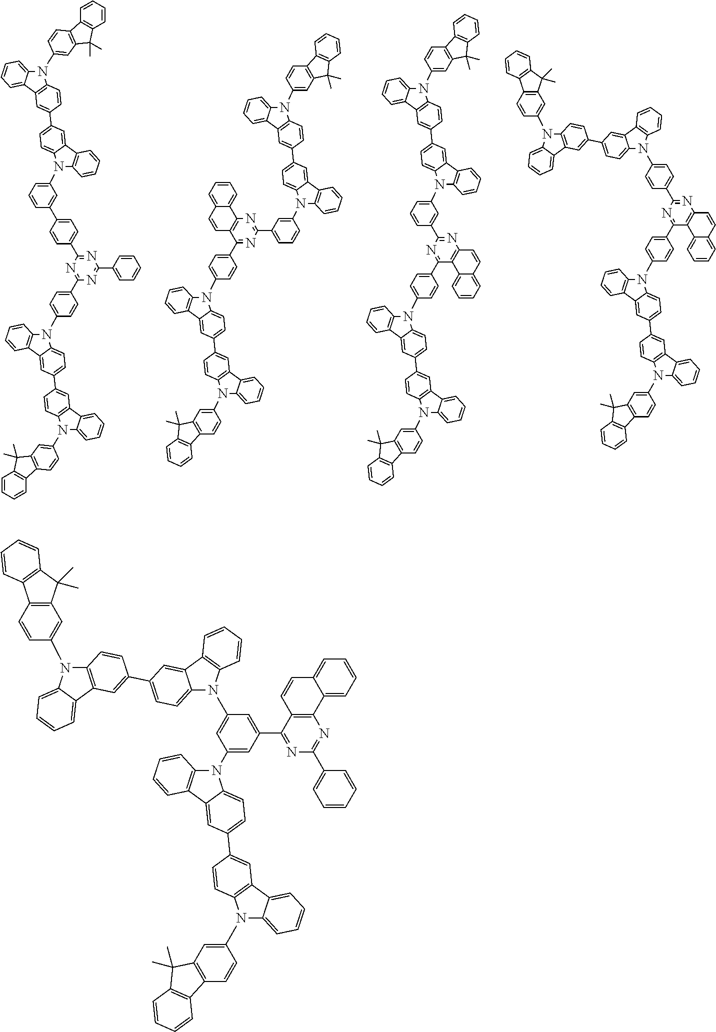

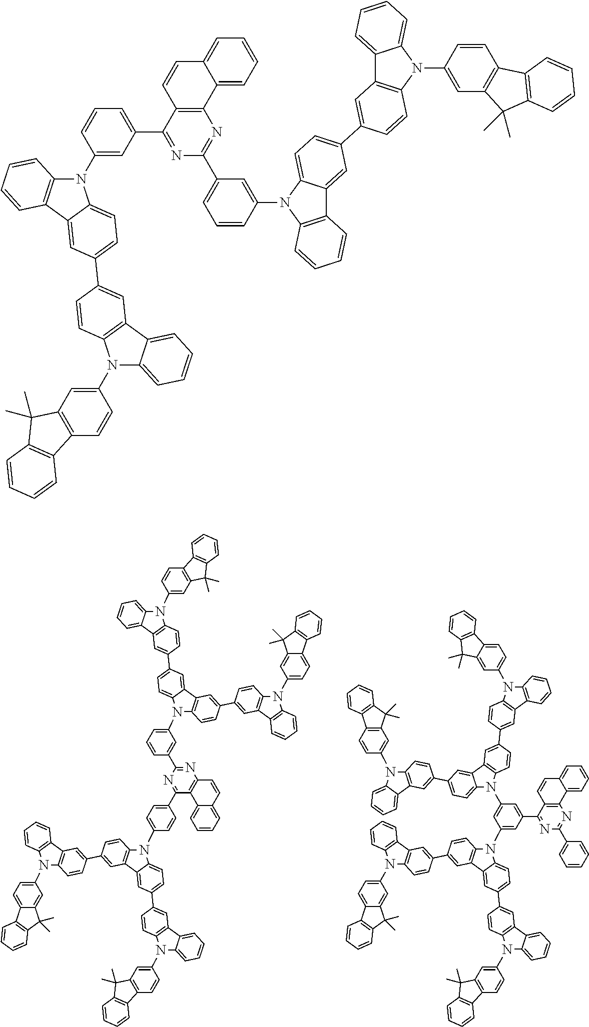

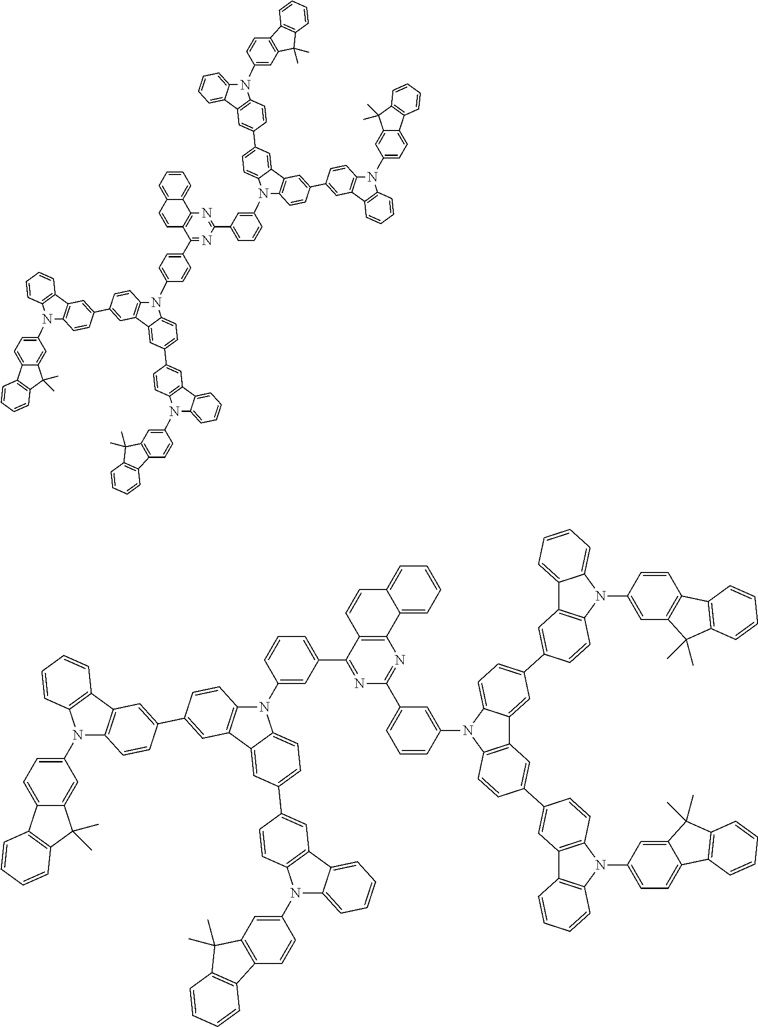

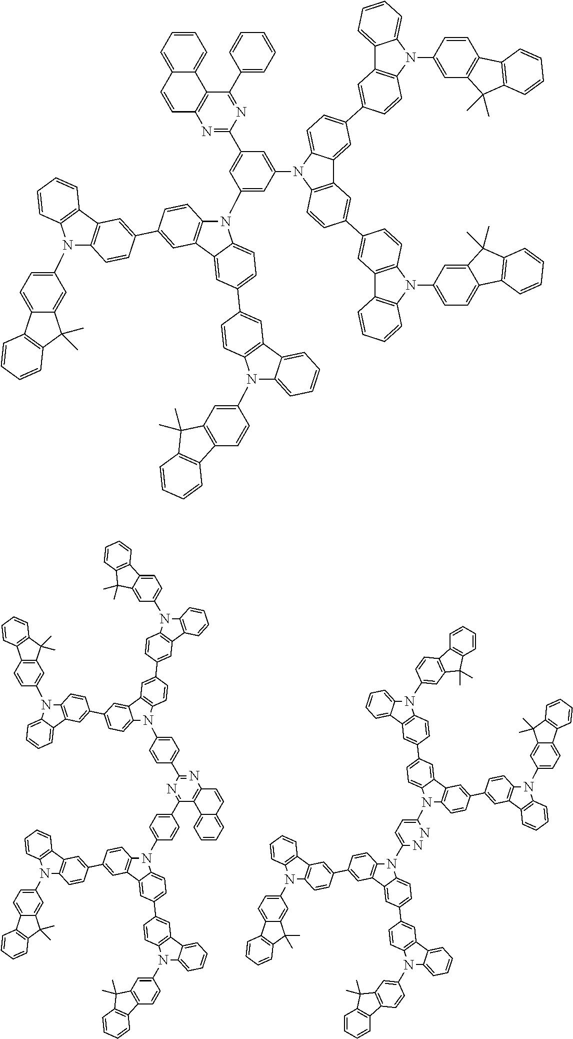

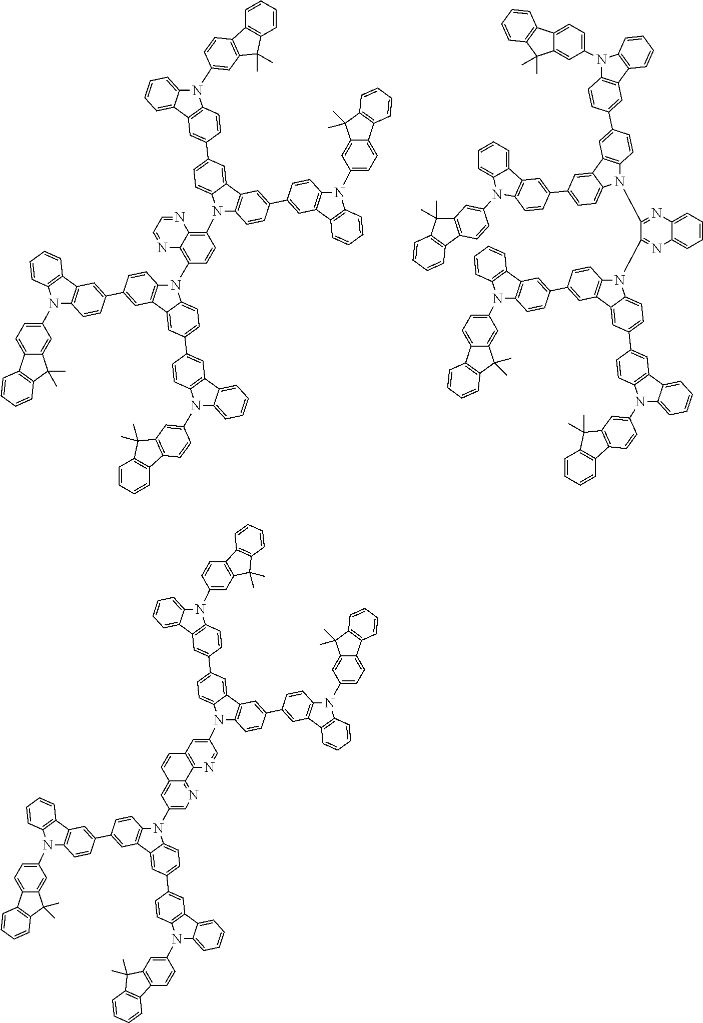

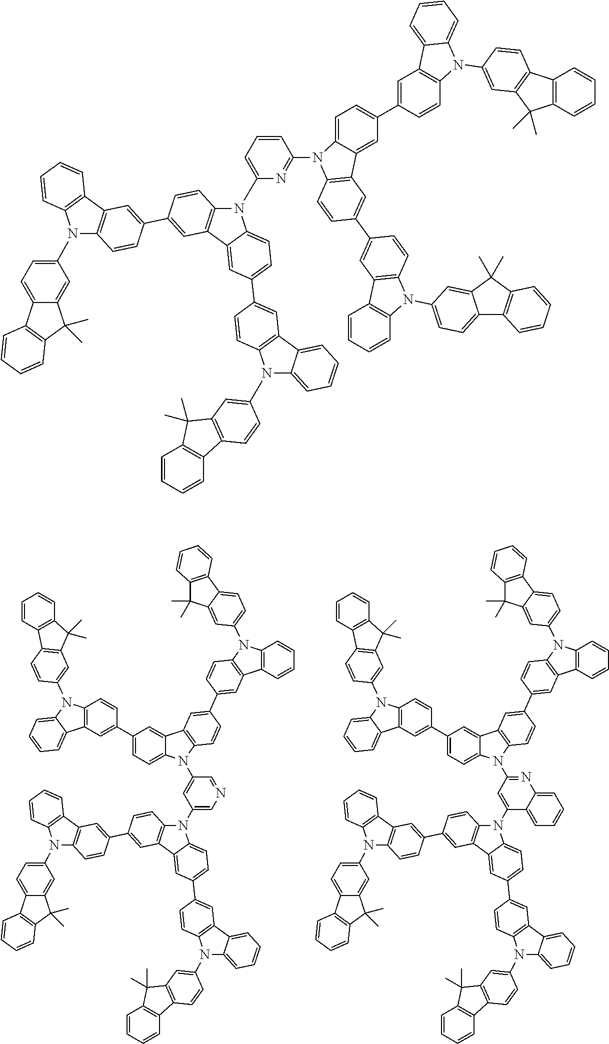

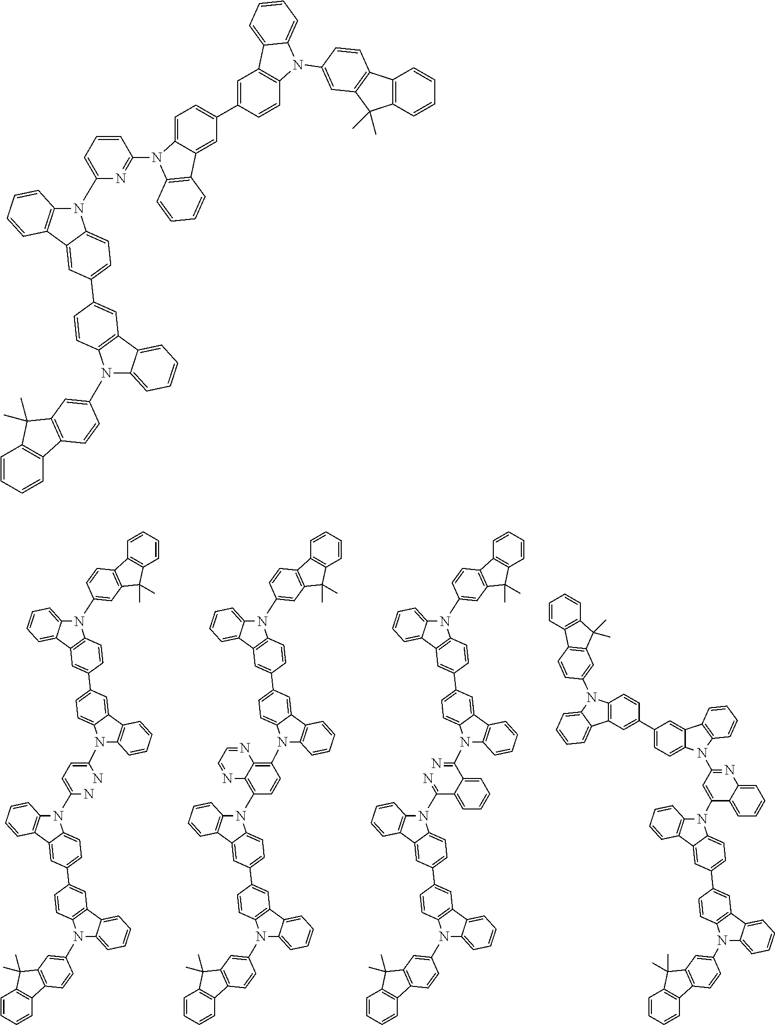

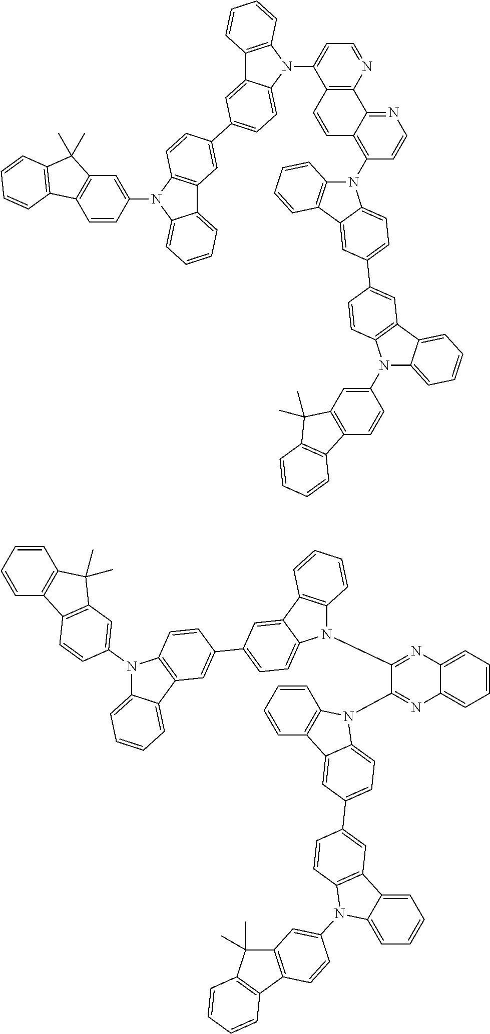 [SMILES: CC1(C)c2ccccc2-c2ccc(-n3c4ccccc4c4cc(-c5ccc6c(c5)c5ccccc5n6-c5ccnc6c5ccc5c(-n7c8ccccc8c8cc(-c9ccc%10c(c9)c9ccccc9n%10-c9ccc%10c(c9)C(C)(C)c9ccccc9-%10)ccc87)ccnc56)ccc43)cc21 ; CC1(C)c2ccccc2-c2ccc(-n3c4ccccc4c4cc(-c5ccc6c(c5)c5ccccc5n6-c5nc6ccccc6nc5-n5c6ccccc6c6cc(-c7ccc8c(c7)c7ccccc7n8-c7ccc8c(c7)C(C)(C)c7ccccc7-8)ccc65)ccc43)cc21]

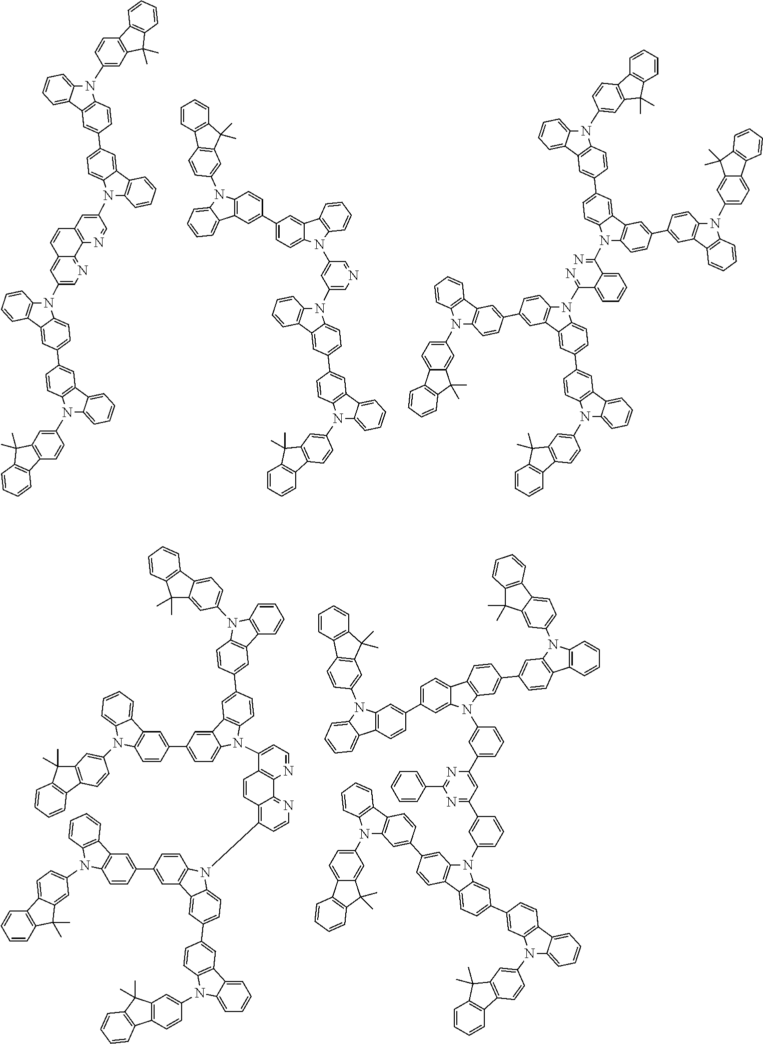

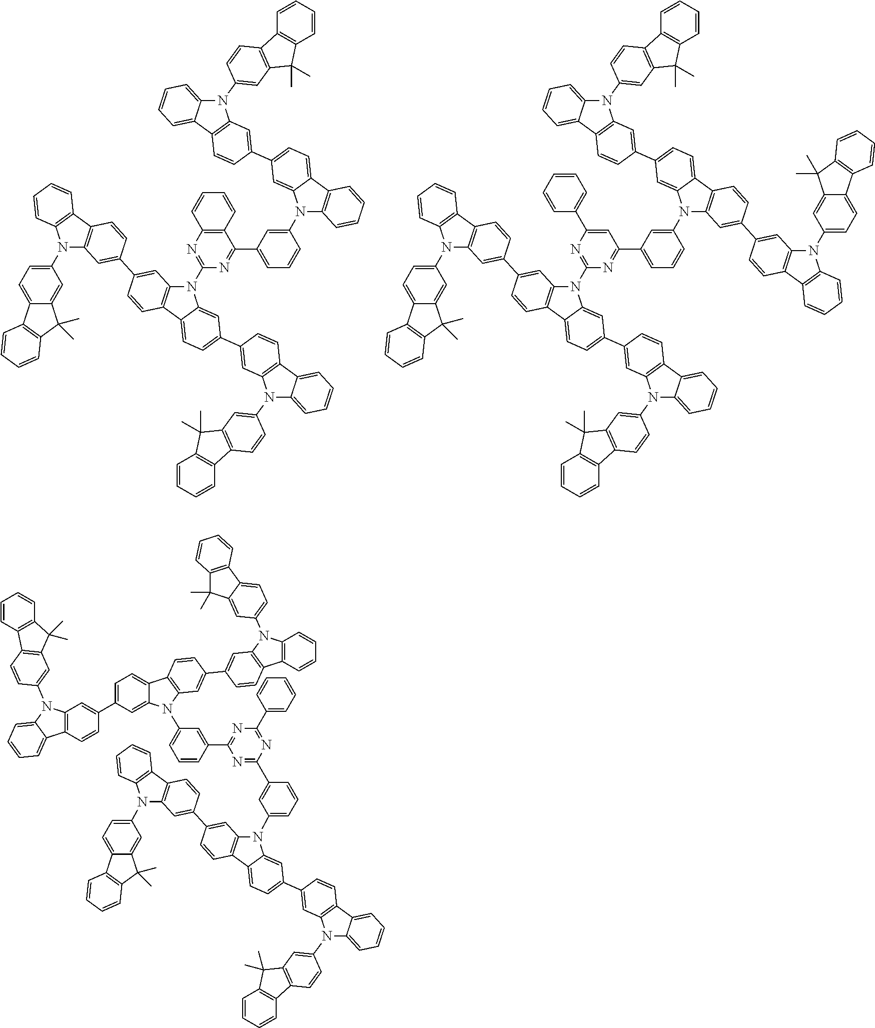

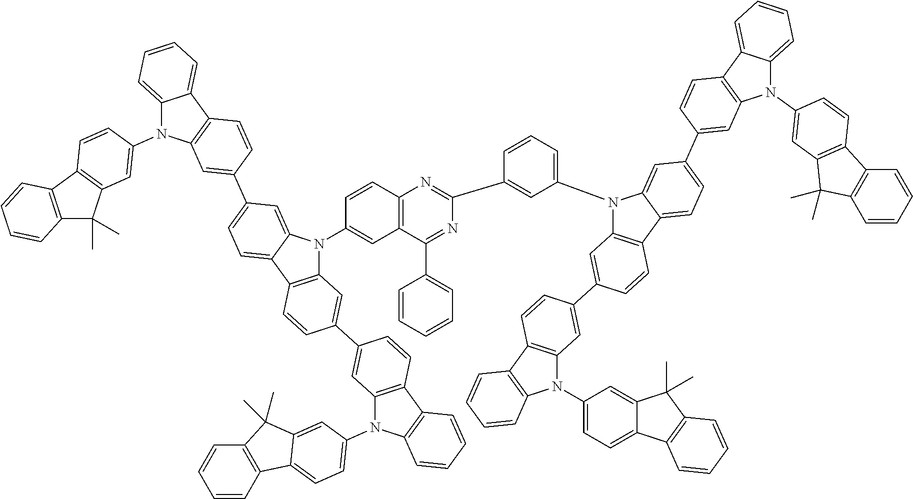 [SMILES: CC1(C)c2ccccc2-c2ccc(-n3c4ccccc4c4ccc(-c5ccc6c7ccc(-c8ccc9c%10ccccc%10n(-c%10ccc%11c(c%10)C(C)(C)c%10ccccc%10-%11)c9c8)cc7n(-c7cccc(-c8nc(-c9ccccc9)c9cc(-n%10c%11cc(-c%12ccc%13c%14ccccc%14n(-c%14ccc%15c(c%14)C(C)(C)c%14ccccc%14-%15)c%13c%12)ccc%11c%11ccc(-c%12ccc%13c%14ccccc%14n(-c%14ccc%15c(c%14)C(C)(C)c%14ccccc%14-%15)c%13c%12)cc%11%10)ccc9n8)c7)c6c5)cc43)cc21]

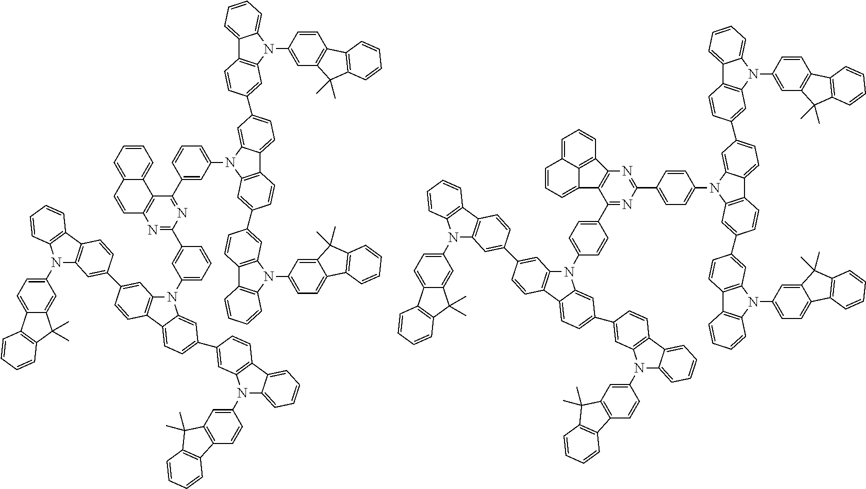

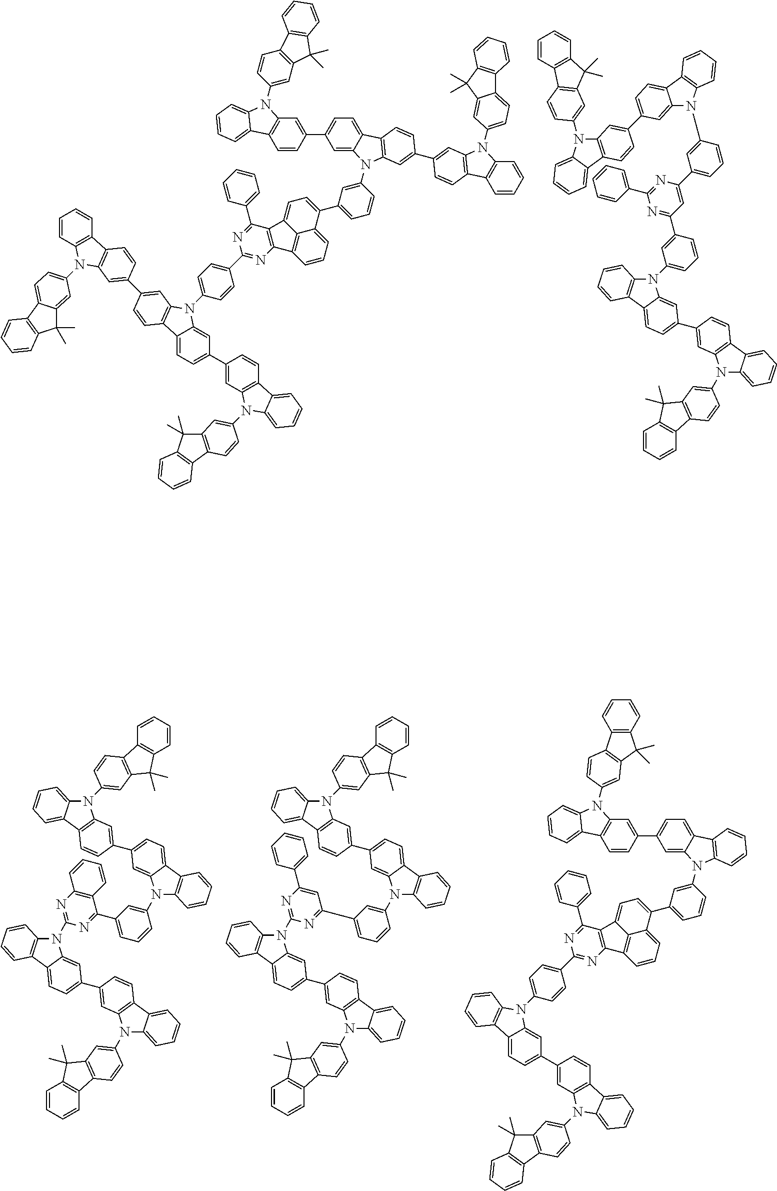

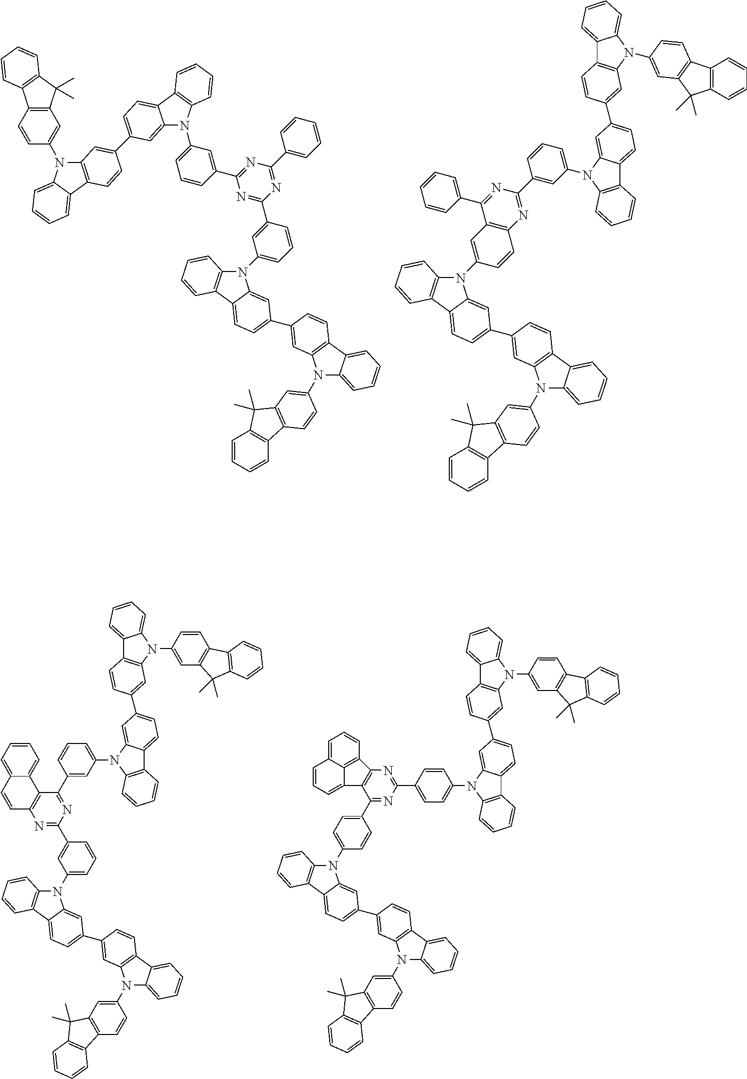

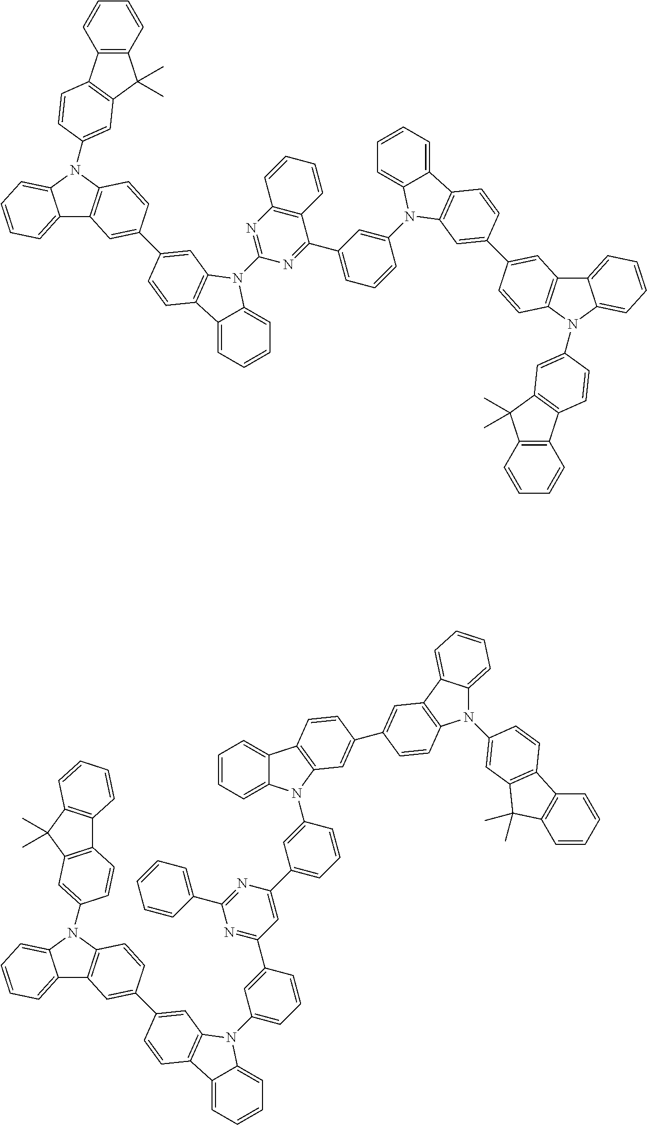 [SMILES: CC1(C)c2ccccc2-c2ccc(-n3c4ccccc4c4cc(-c5ccc6c7ccccc7n(-c7cccc(-c8nc(-n9c%10ccccc%10c%10ccc(-c%11ccc%12c(c%11)c%11ccccc%11n%12-c%11ccc%12c(c%11)C(C)(C)c%11ccccc%11-%12)cc%109)nc9ccccc89)c7)c6c5)ccc43)cc21 ; CC1(C)c2ccccc2-c2ccc(-n3c4ccccc4c4cc(-c5ccc6c7ccccc7n(-c7cccc(-c8cc(-c9cccc(-n%10c%11ccccc%11c%11ccc(-c%12ccc%13c(c%12)c%12ccccc%12n%13-c%12ccc%13c(c%12)C(C)(C)c%12ccccc%12-%13)cc%11%10)c9)nc(-c9ccccc9)n8)c7)c6c5)ccc43)cc21]

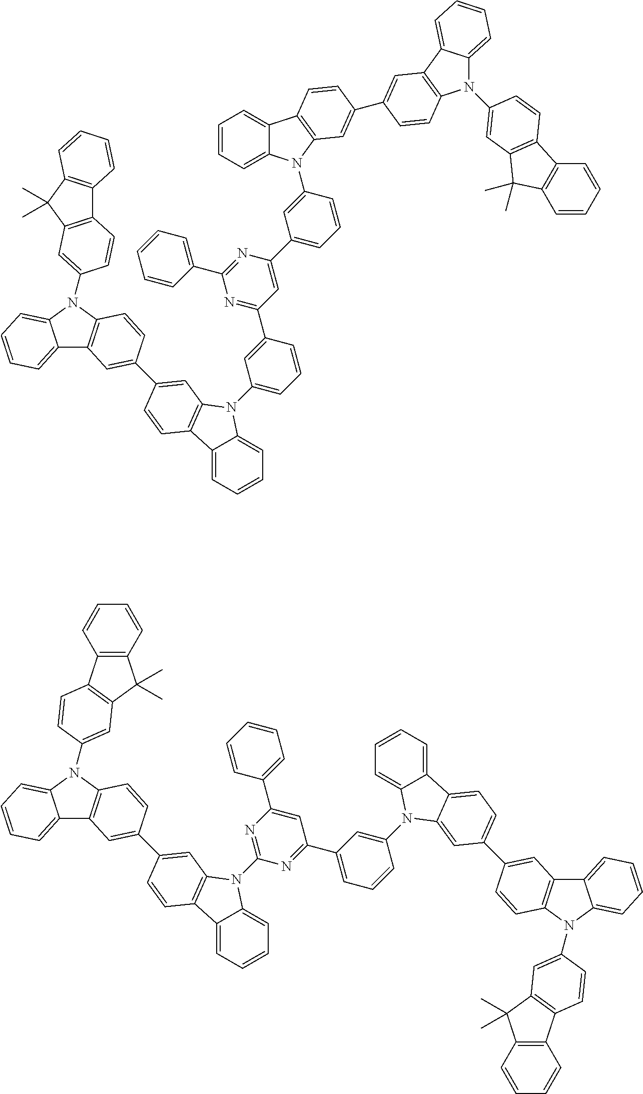 [SMILES: CC1(C)c2ccccc2-c2ccc(-n3c4ccccc4c4cc(-c5ccc6c7ccccc7n(-c7cccc(-c8cc(-c9cccc(-n%10c%11ccccc%11c%11ccc(-c%12ccc%13c(c%12)c%12ccccc%12n%13-c%12ccc%13c(c%12)C(C)(C)c%12ccccc%12-%13)cc%11%10)c9)nc(-c9ccccc9)n8)c7)c6c5)ccc43)cc21 ; CC1(C)c2ccccc2-c2ccc(-n3c4ccccc4c4cc(-c5ccc6c7ccccc7n(-c7cccc(-c8cc(-c9ccccc9)nc(-n9c%10ccccc%10c%10ccc(-c%11ccc%12c(c%11)c%11ccccc%11n%12-c%11ccc%12c(c%11)C(C)(C)c%11ccccc%11-%12)cc%109)n8)c7)c6c5)ccc43)cc21]

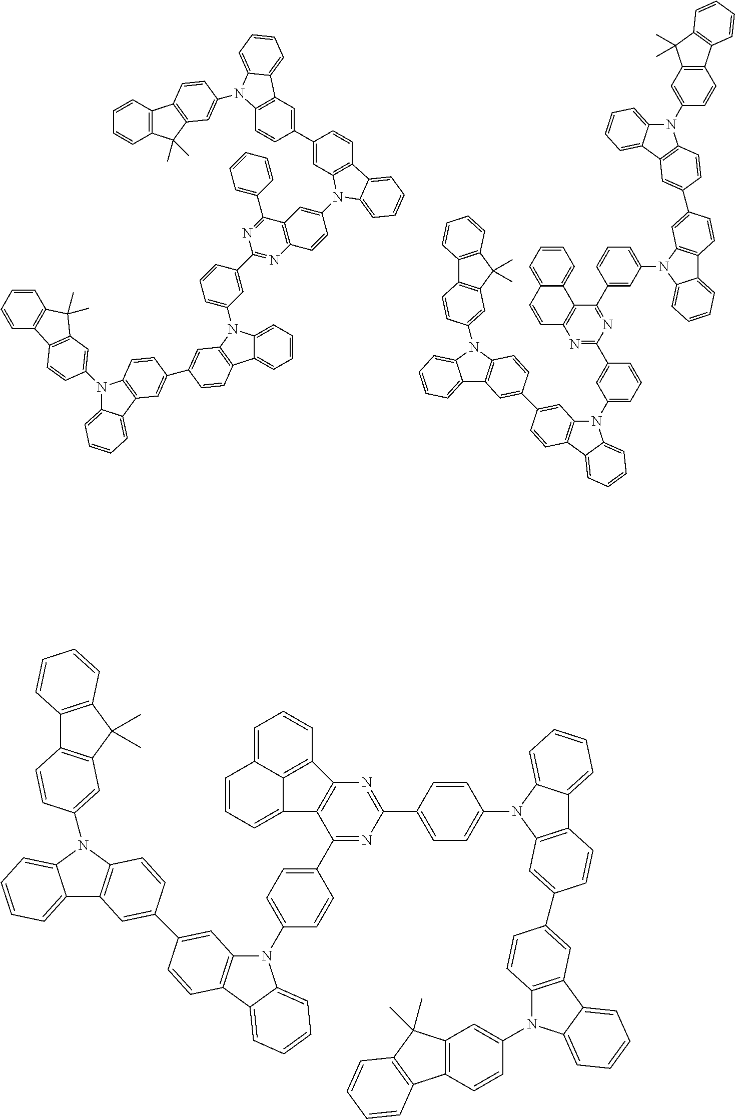

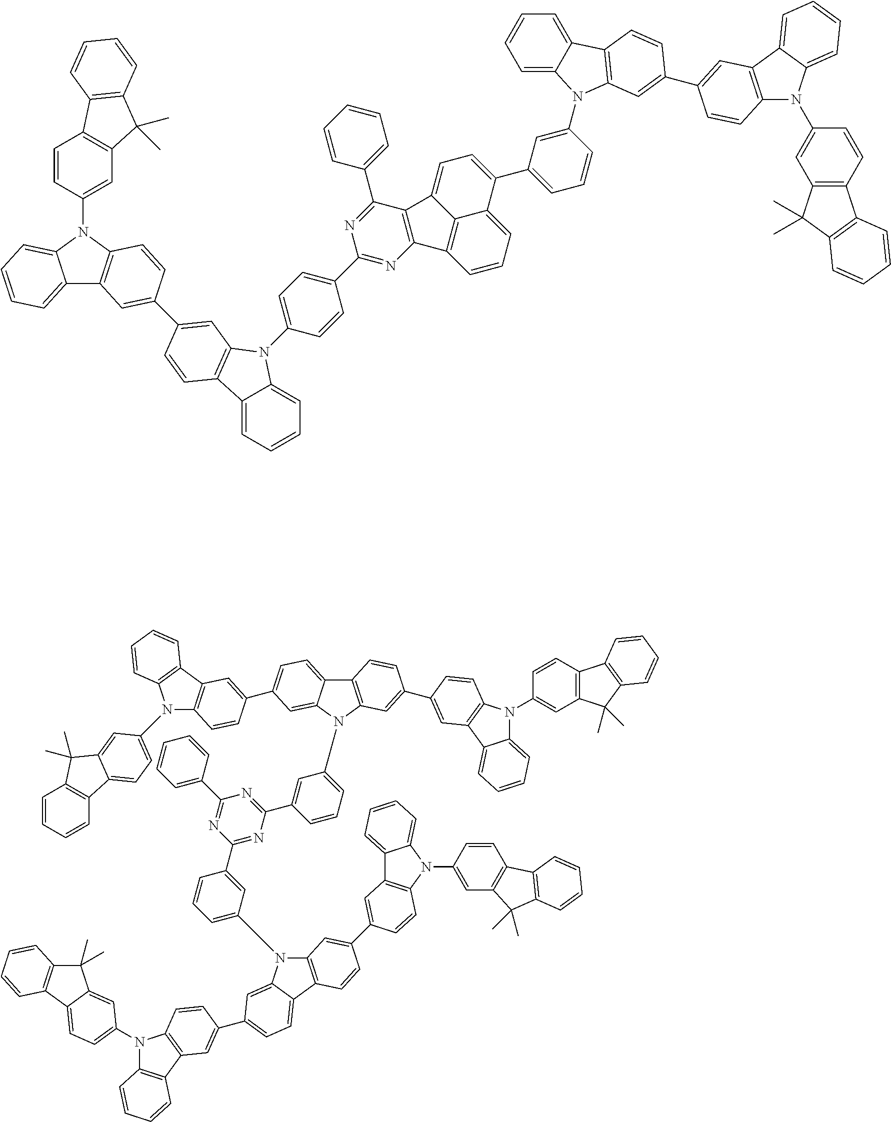

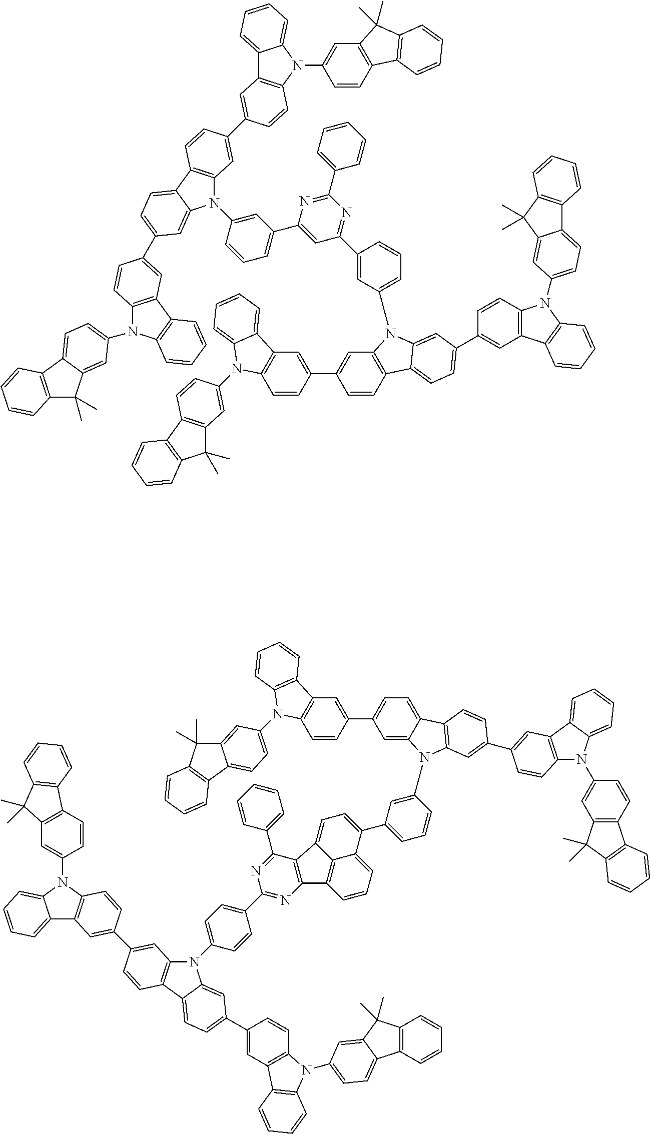

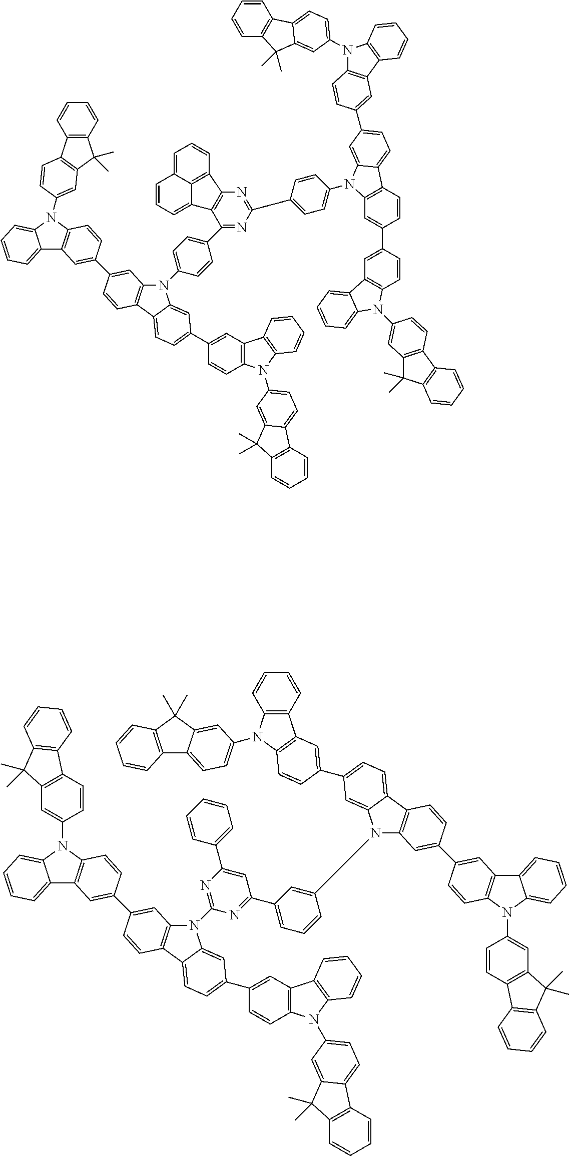

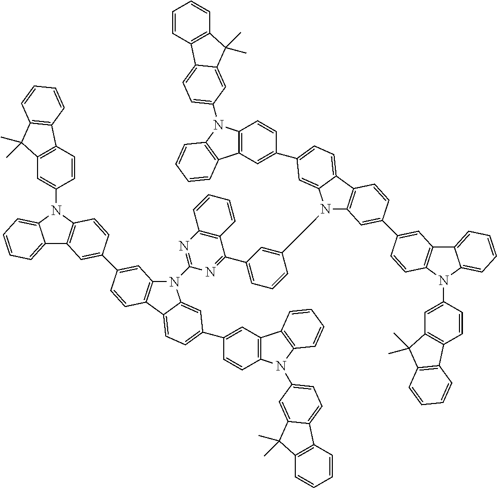 [SMILES: CC1(C)c2ccccc2-c2ccc(-n3c4ccccc4c4cc(-c5ccc6c7ccc(-c8ccc9c(c8)c8ccccc8n9-c8ccc9c(c8)C(C)(C)c8ccccc8-9)cc7n(-c7cccc(-c8nc(-n9c%10cc(-c%11ccc%12c(c%11)c%11ccccc%11n%12-c%11ccc%12c(c%11)C(C)(C)c%11ccccc%11-%12)ccc%10c%10ccc(-c%11ccc%12c(c%11)c%11ccccc%11n%12-c%11ccc%12c(c%11)C(C)(C)c%11ccccc%11-%12)cc%109)nc9ccccc89)c7)c6c5)ccc43)cc21]

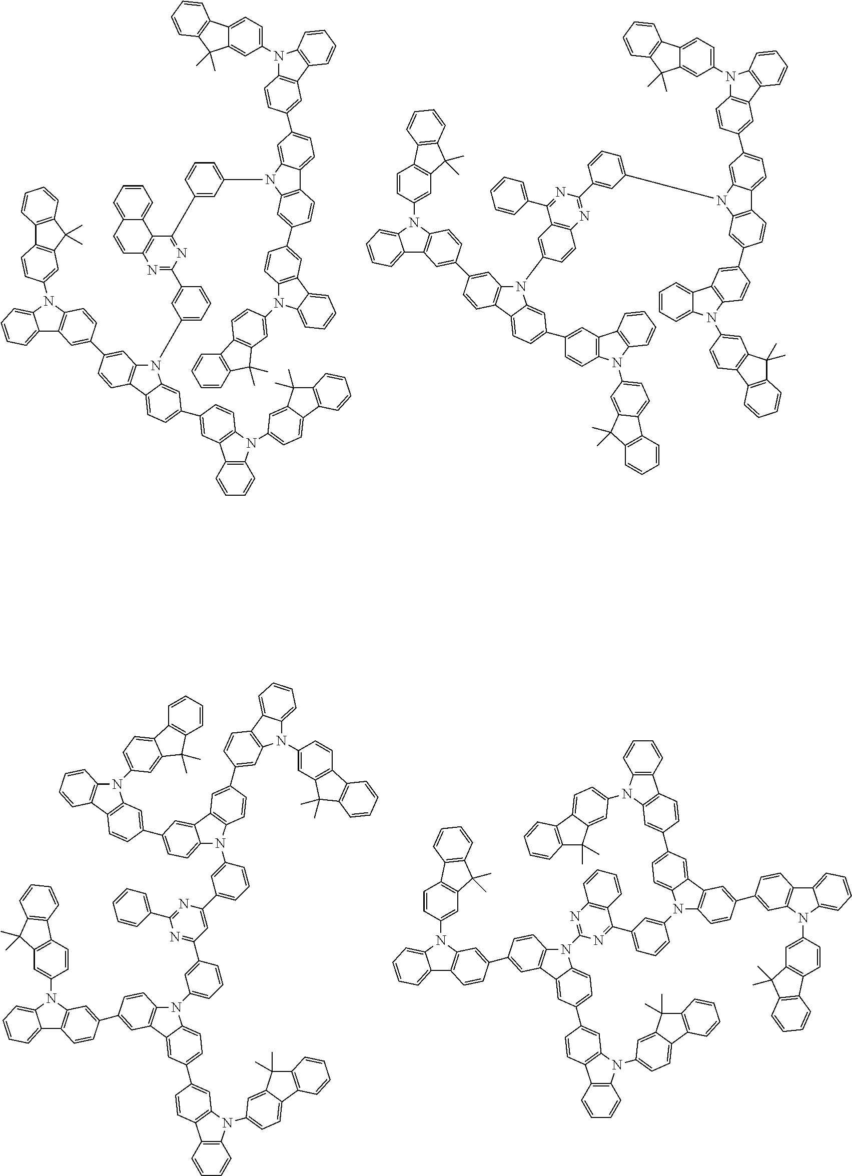

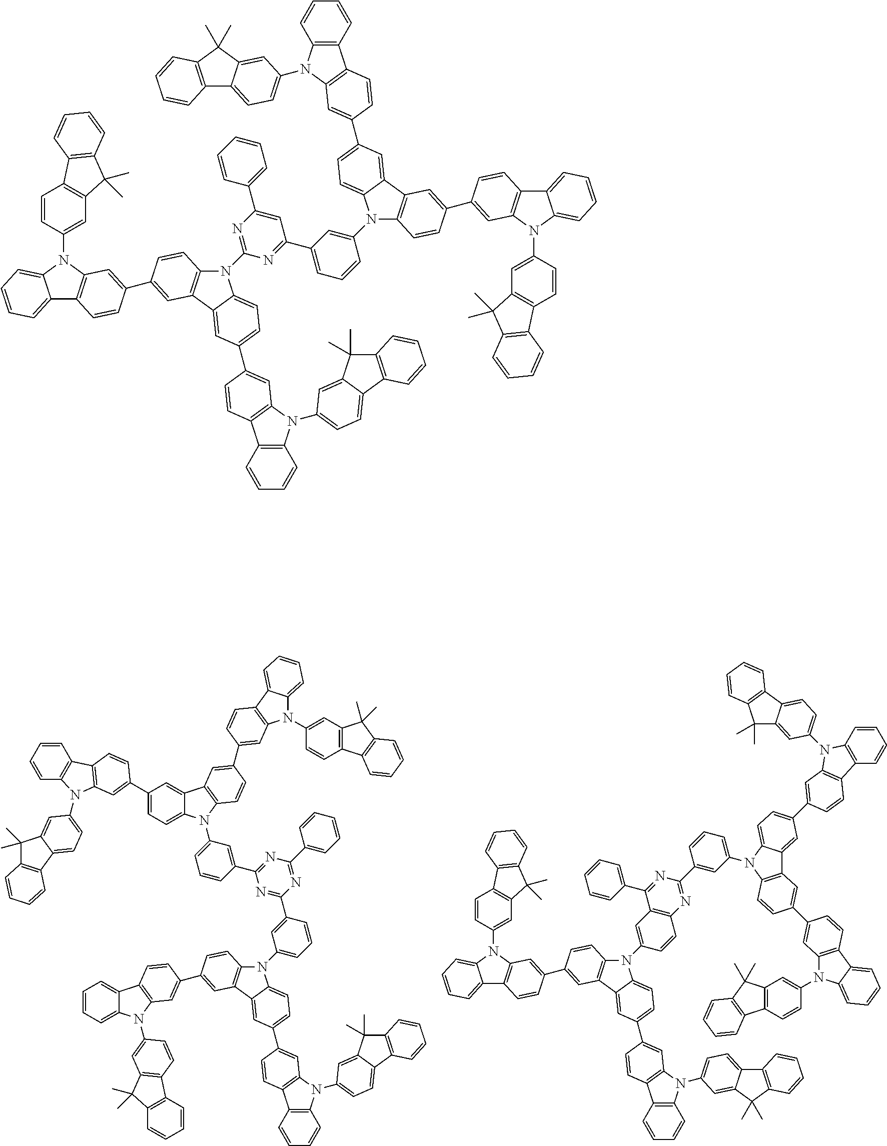

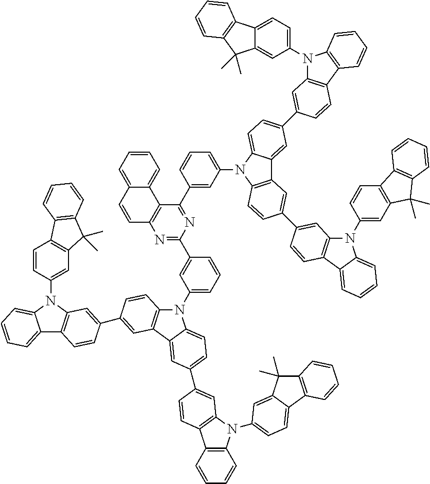 [SMILES: CC1(C)c2ccccc2-c2ccc(-n3c4ccccc4c4ccc(-c5ccc6c(c5)c5cc(-c7ccc8c9ccccc9n(-c9ccc%10c(c9)C(C)(C)c9ccccc9-%10)c8c7)ccc5n6-c5cccc(-c6nc(-c7cccc(-n8c9ccc(-c%10ccc%11c%12ccccc%12n(-c%12ccc%13c(c%12)C(C)(C)c%12ccccc%12-%13)c%11c%10)cc9c9cc(-c%10ccc%11c%12ccccc%12n(-c%12ccc%13c(c%12)C(C)(C)c%12ccccc%12-%13)c%11c%10)ccc98)c7)c7c(ccc8ccccc87)n6)c5)cc43)cc21]

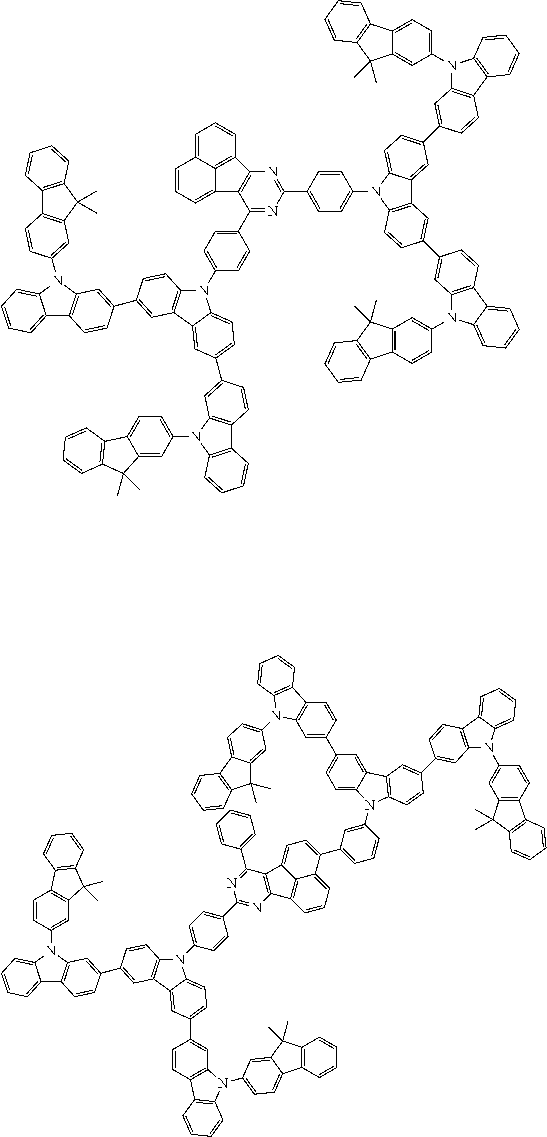

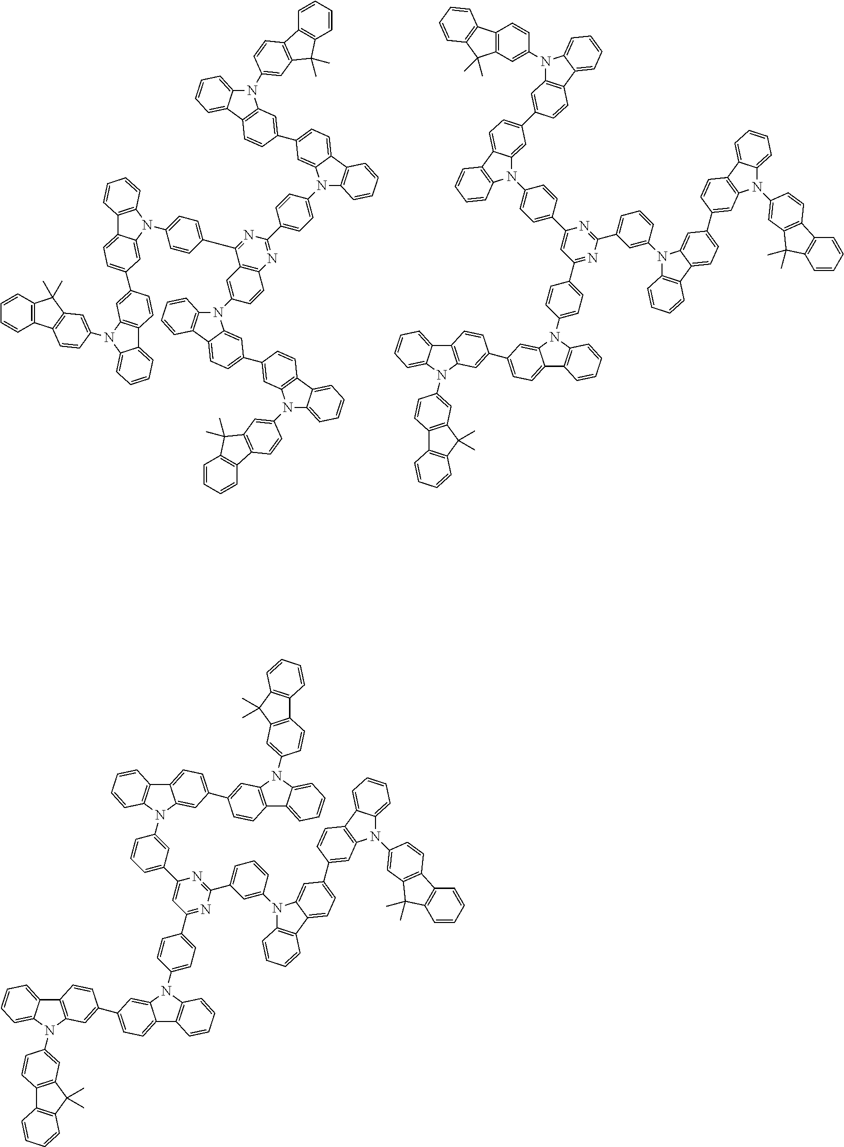

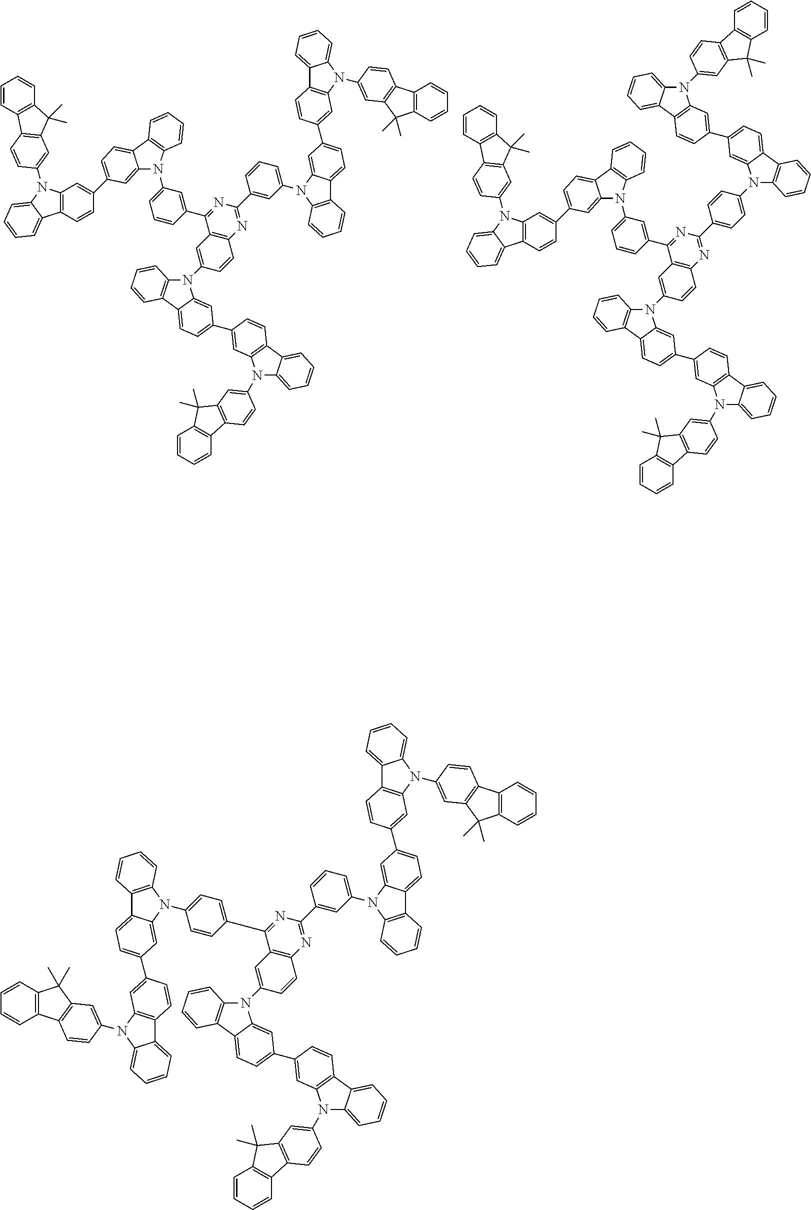

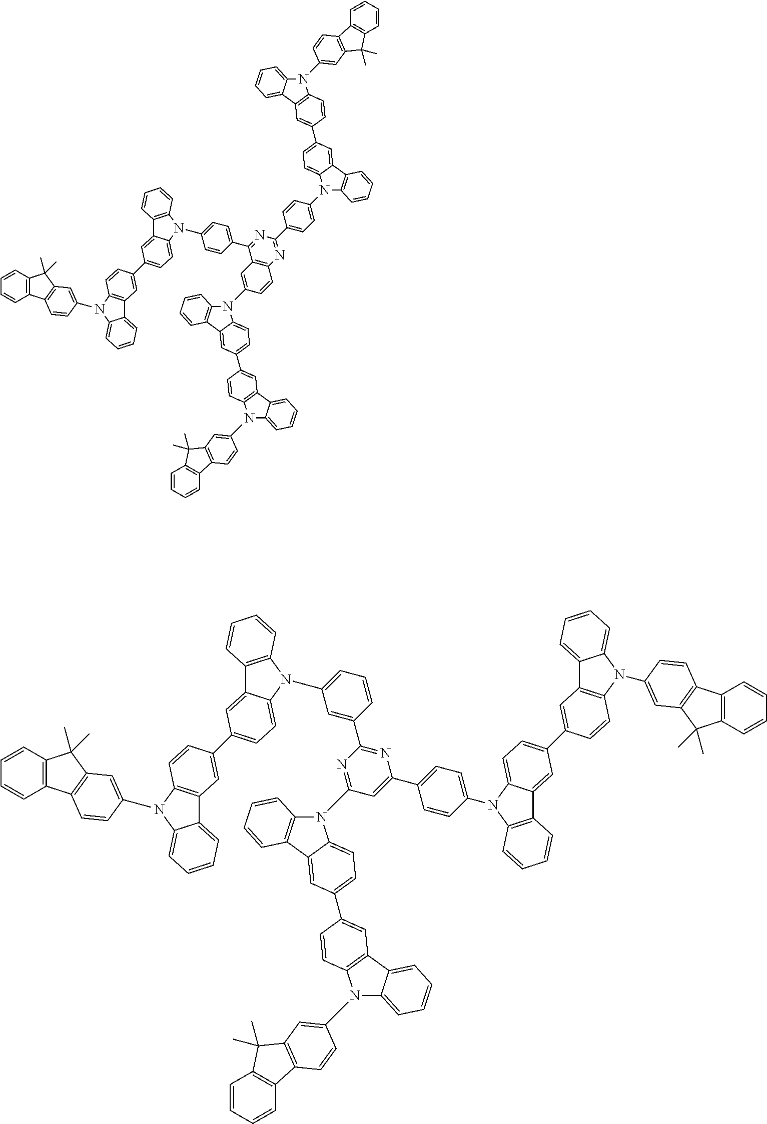

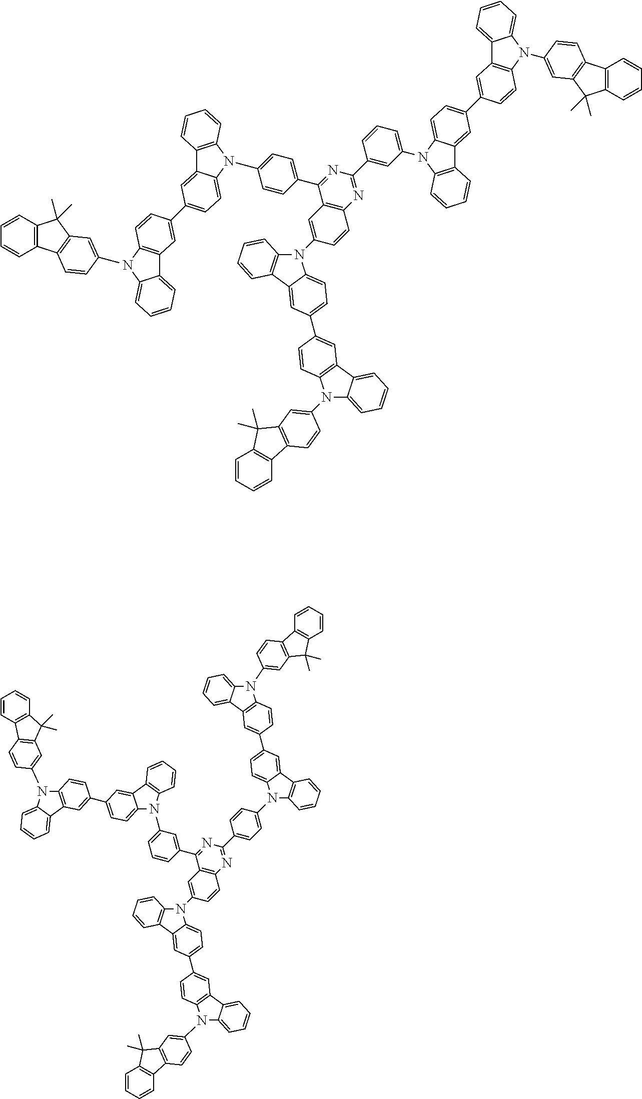

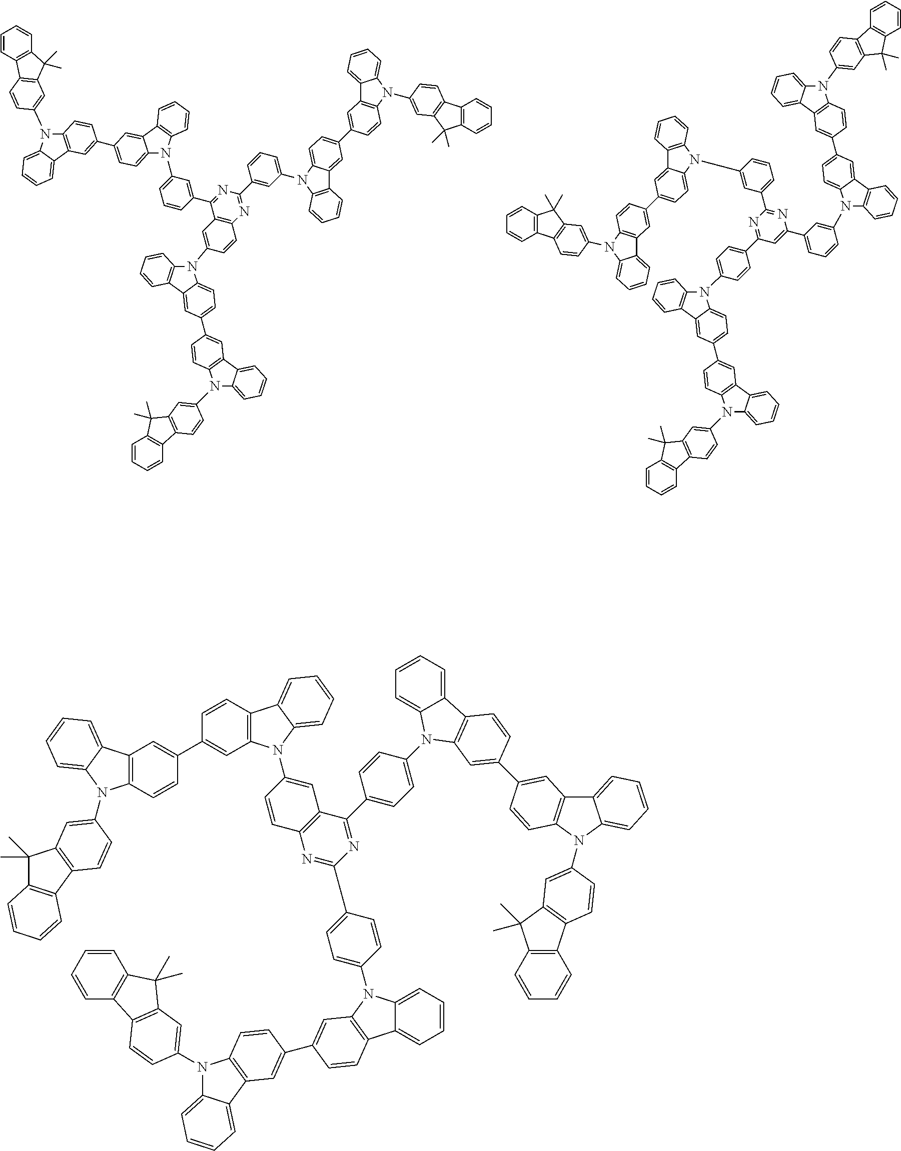

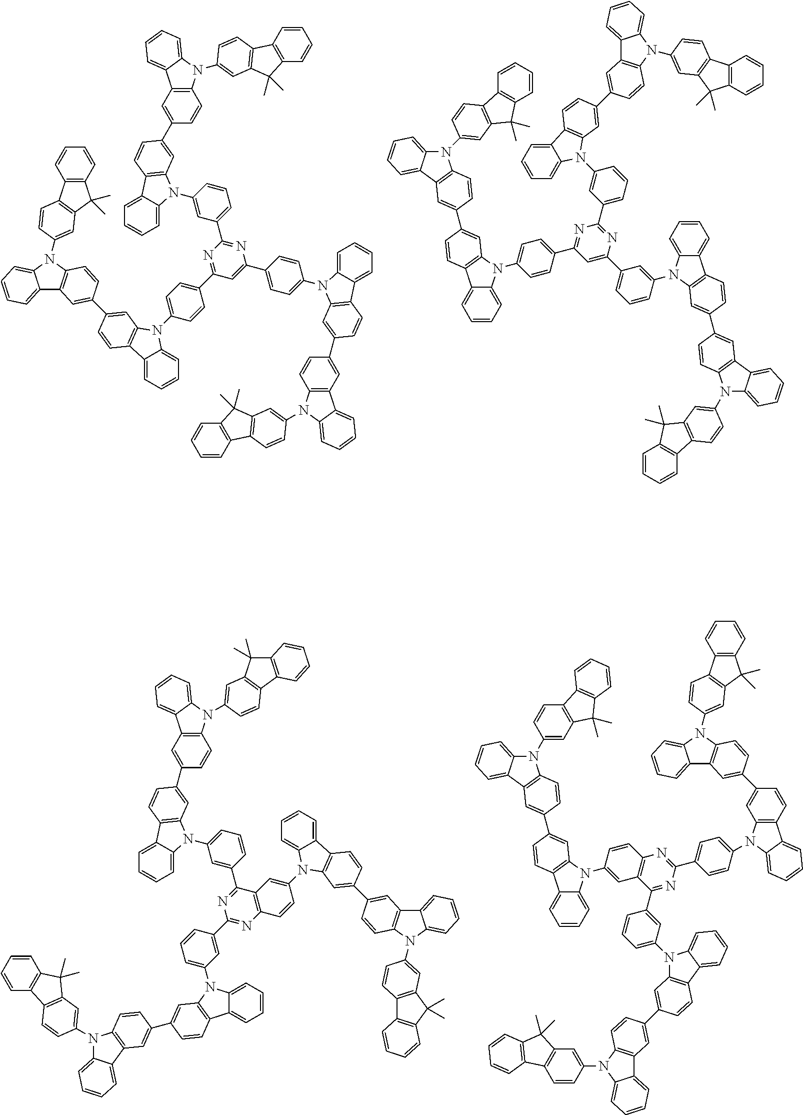

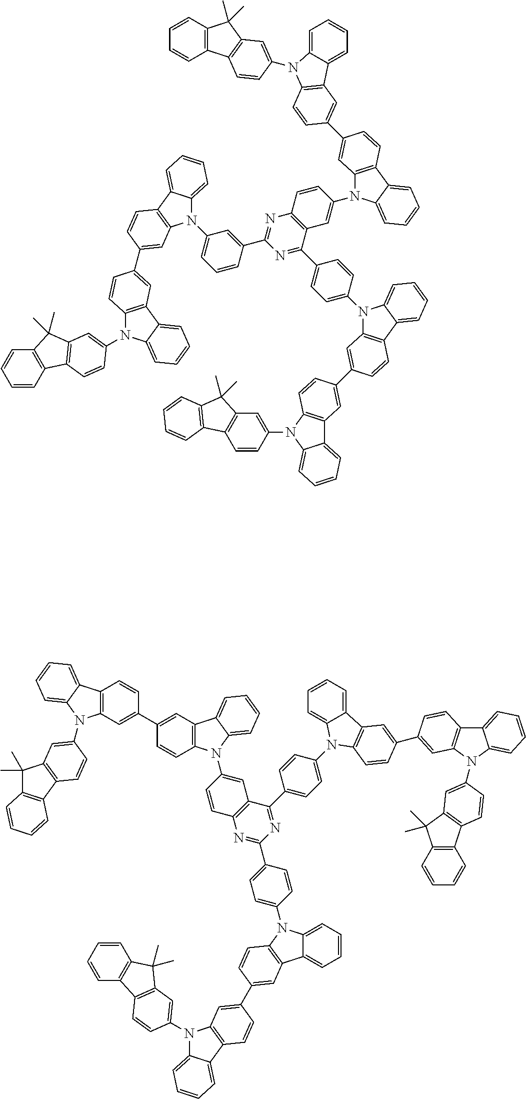

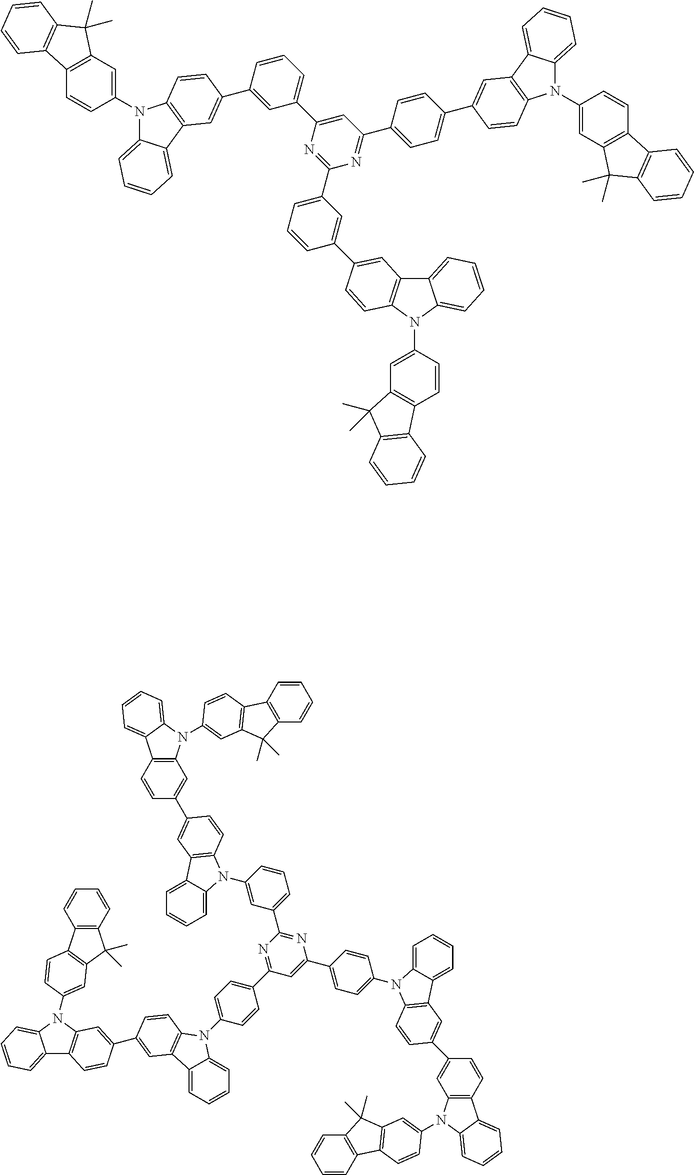

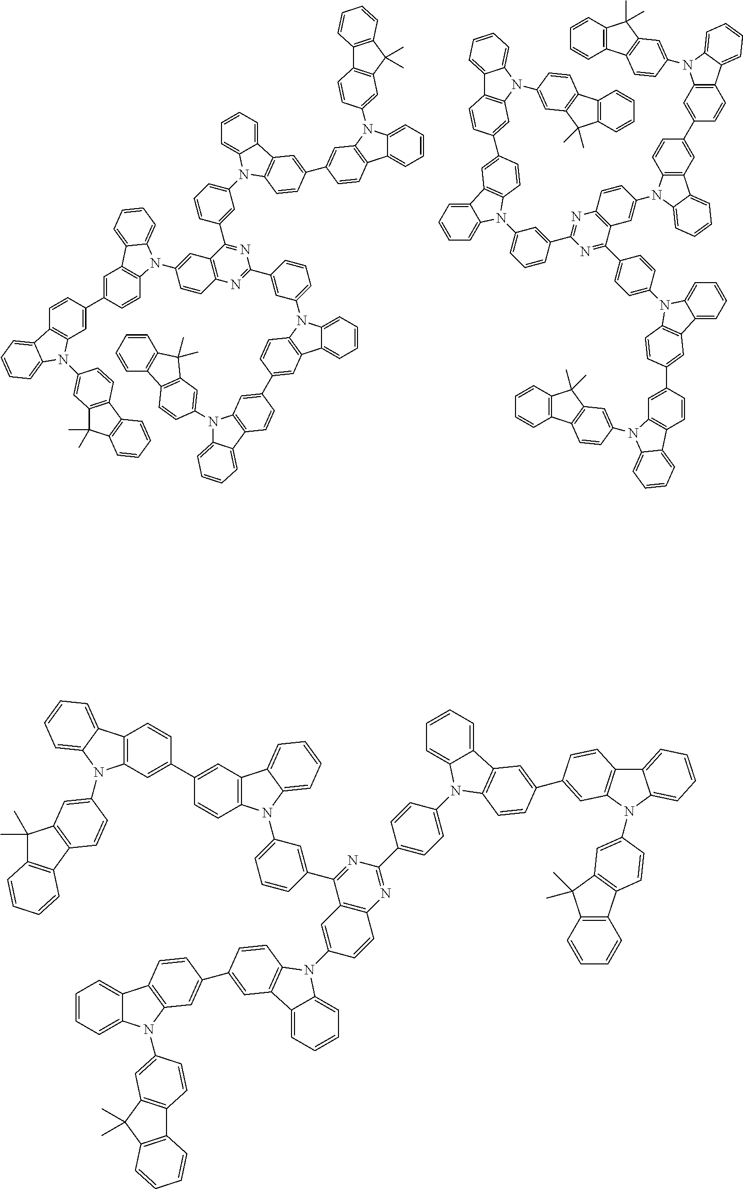

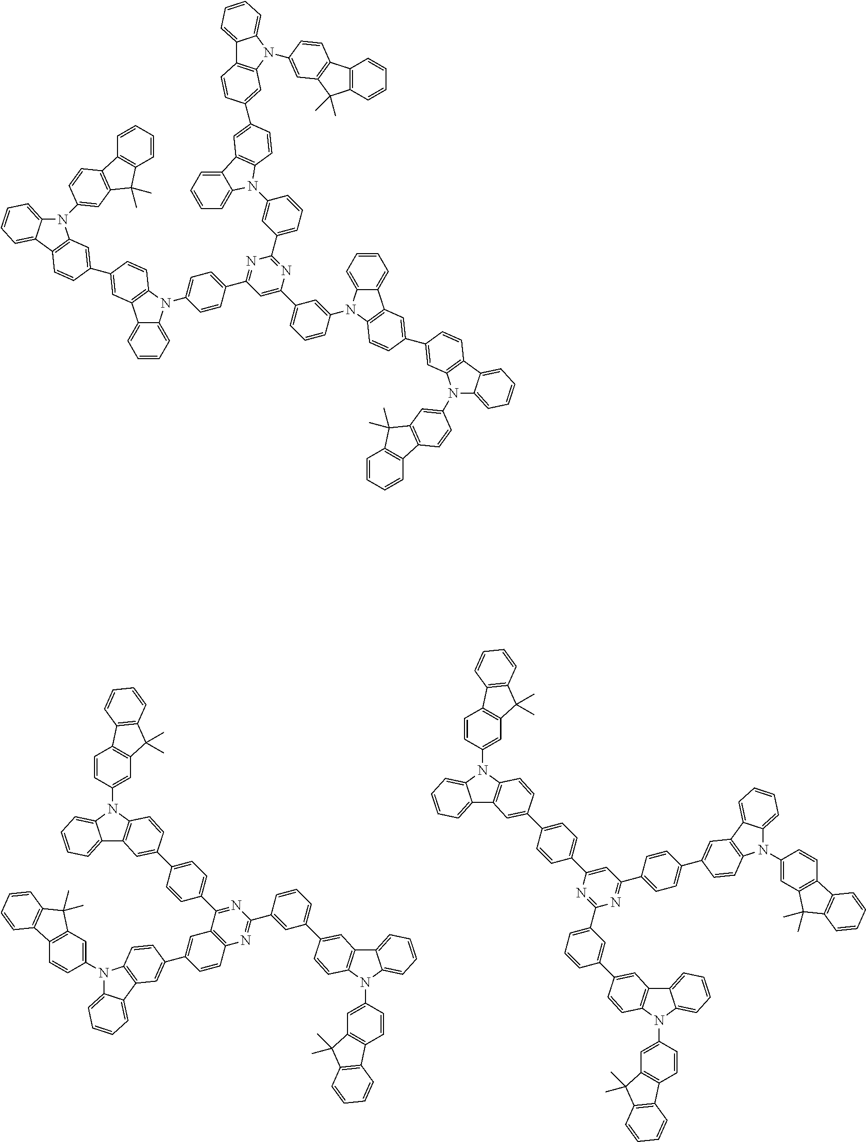

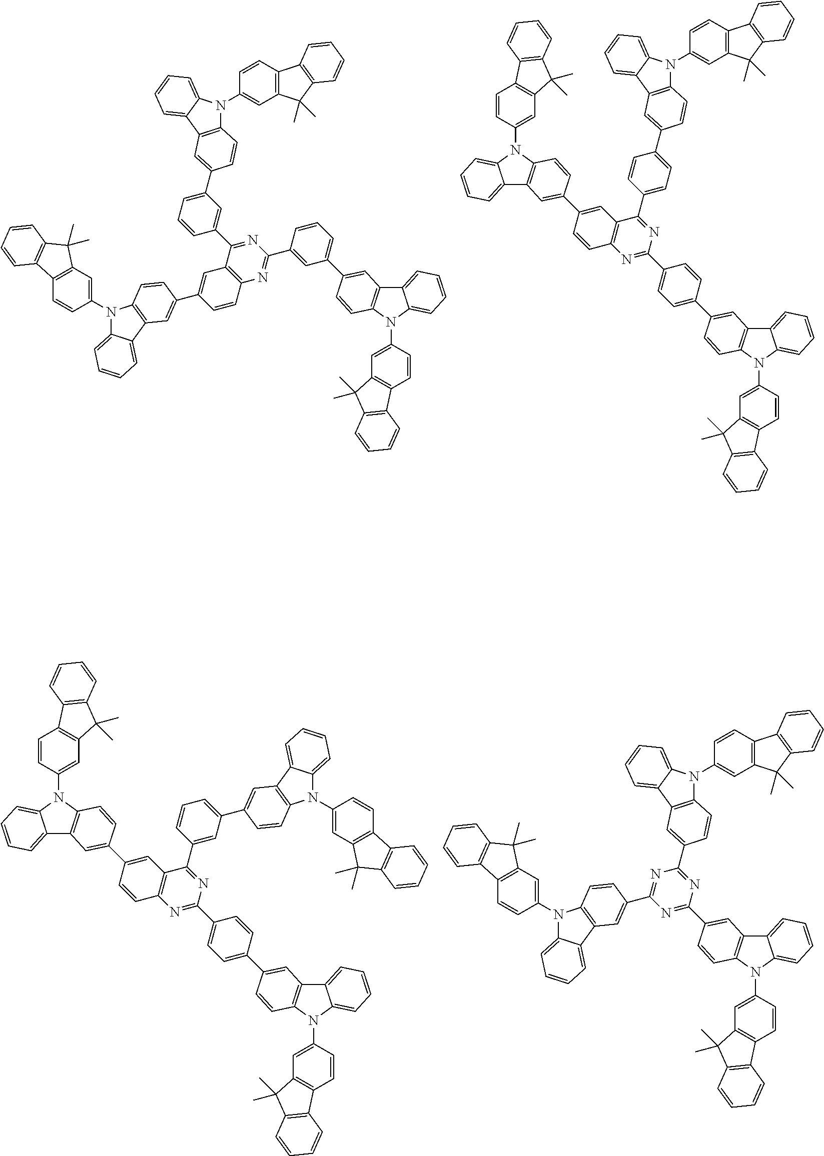

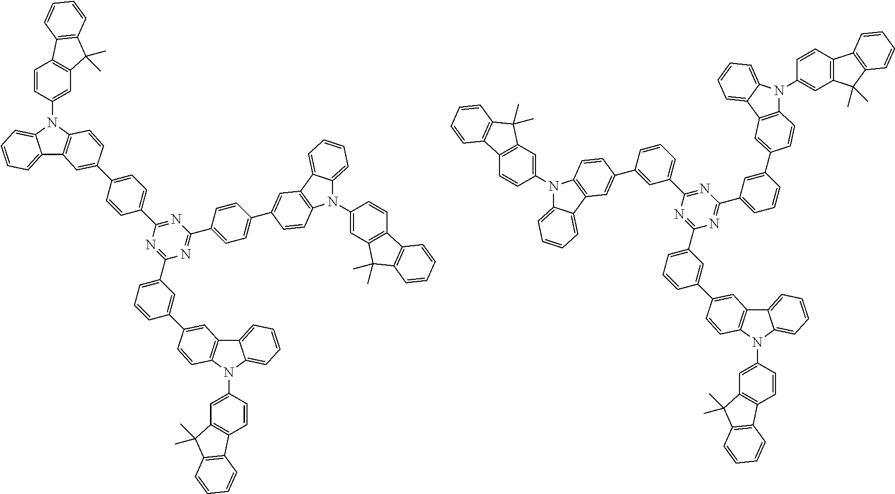 [SMILES: CC1(C)c2ccccc2-c2ccc(-n3c4ccccc4c4cc(-c5ccc(-c6nc(-c7ccc(-c8ccc9c(c8)c8ccccc8n9-c8ccc9c(c8)C(C)(C)c8ccccc8-9)cc7)nc(-c7cccc(-c8ccc9c(c8)c8ccccc8n9-c8ccc9c(c8)C(C)(C)c8ccccc8-9)c7)n6)cc5)ccc43)cc21 ; CC1(C)c2ccccc2-c2ccc(-n3c4ccccc4c4cc(-c5cccc(-c6nc(-c7cccc(-c8ccc9c(c8)c8ccccc8n9-c8ccc9c(c8)C(C)(C)c8ccccc8-9)c7)nc(-c7cccc(-c8ccc9c(c8)c8ccccc8n9-c8ccc9c(c8)C(C)(C)c8ccccc8-9)c7)n6)c5)ccc43)cc21]

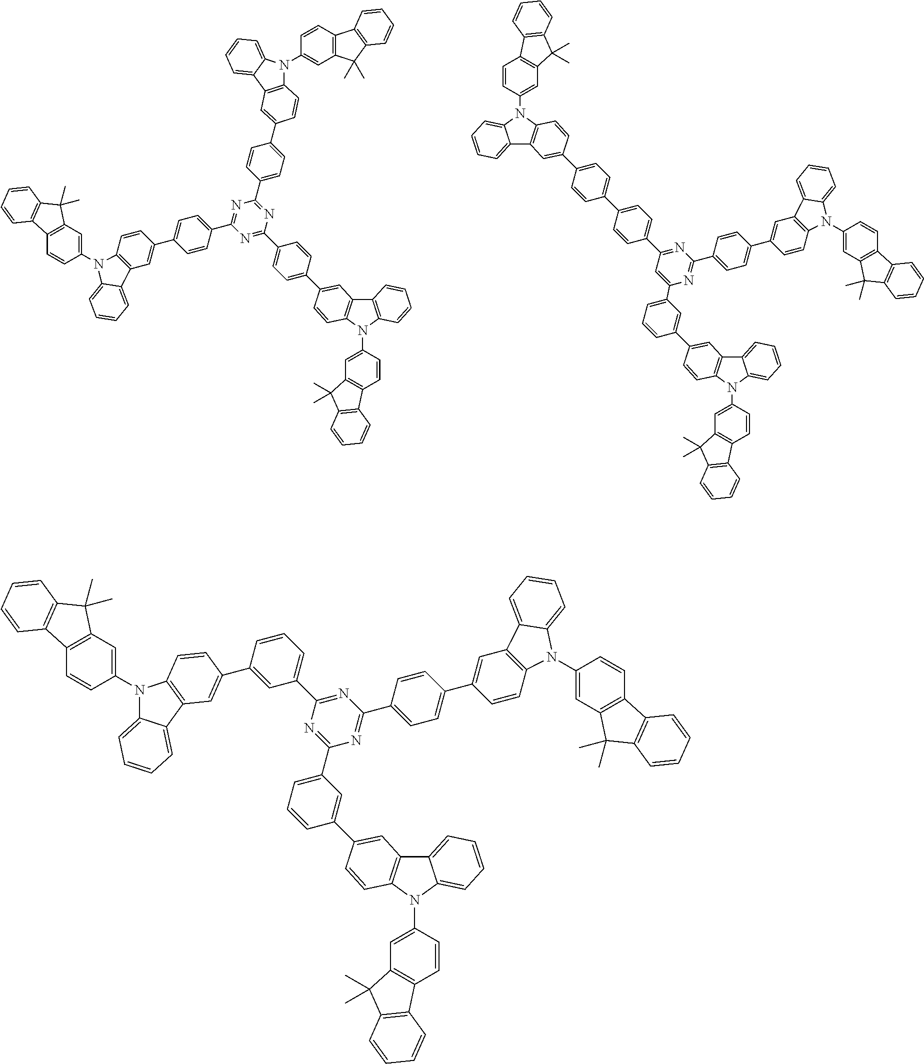

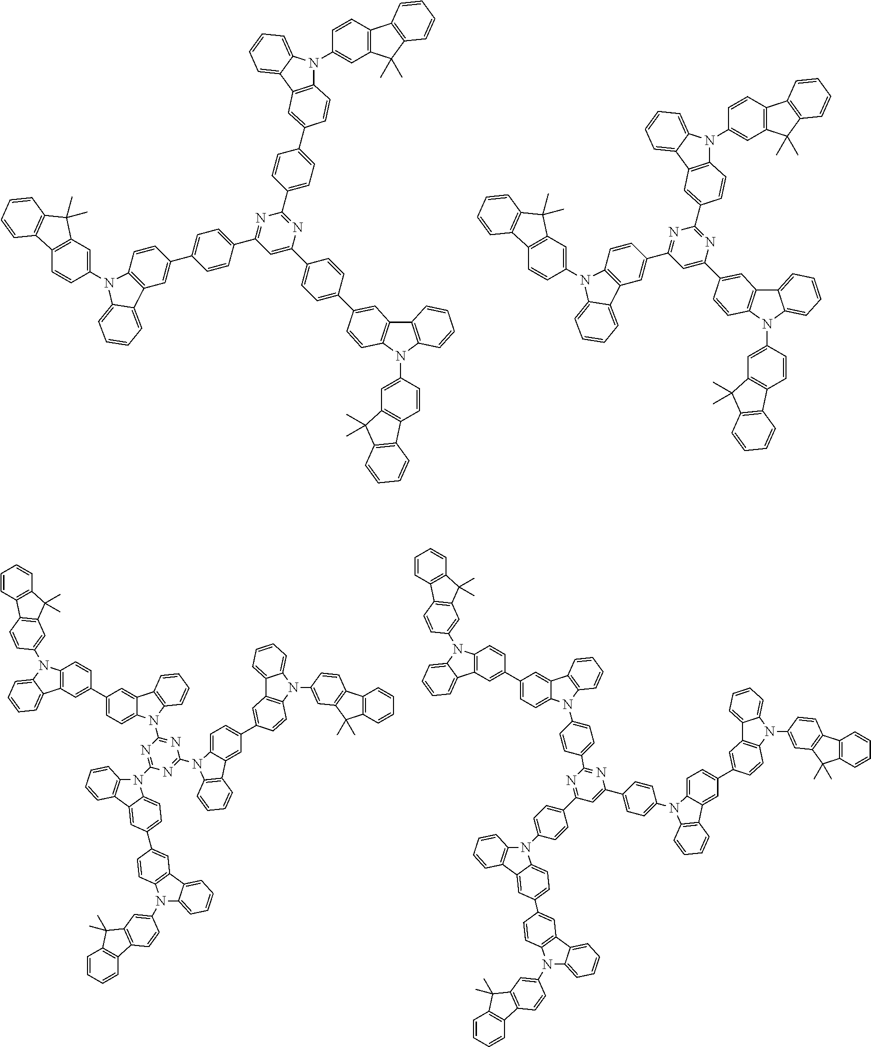

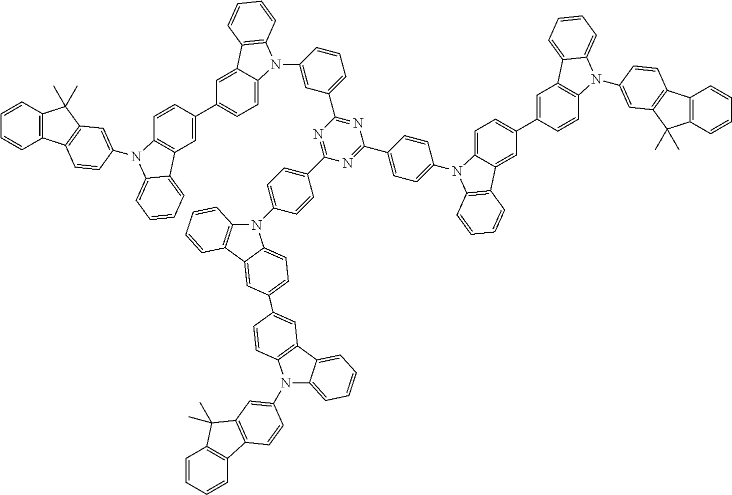 [SMILES: CC1(C)c2ccccc2-c2ccc(-n3c4ccccc4c4cc(-c5ccc6c(c5)c5ccccc5n6-c5ccc(-c6nc(-c7ccc(-n8c9ccccc9c9cc(-c%10ccc%11c(c%10)c%10ccccc%10n%11-c%10ccc%11c(c%10)C(C)(C)c%10ccccc%10-%11)ccc98)cc7)nc(-c7cccc(-n8c9ccccc9c9cc(-c%10ccc%11c(c%10)c%10ccccc%10n%11-c%10ccc%11c(c%10)C(C)(C)c%10ccccc%10-%11)ccc98)c7)n6)cc5)ccc43)cc21]

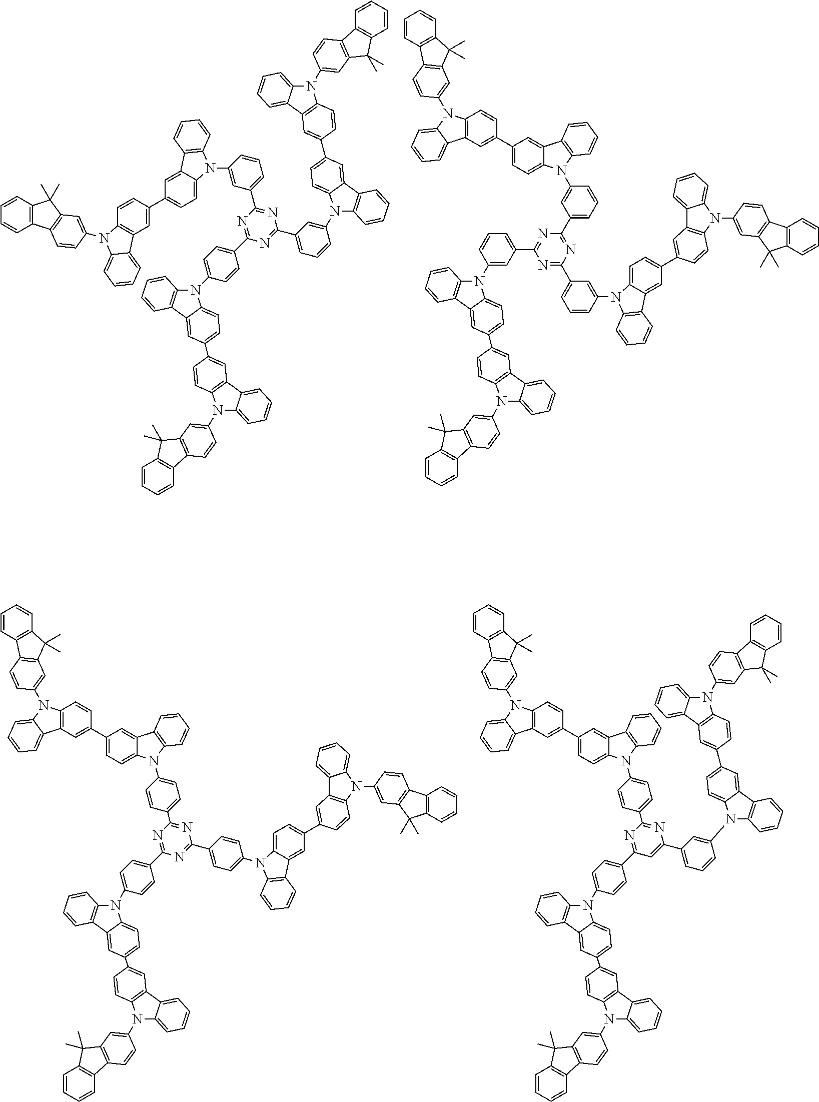

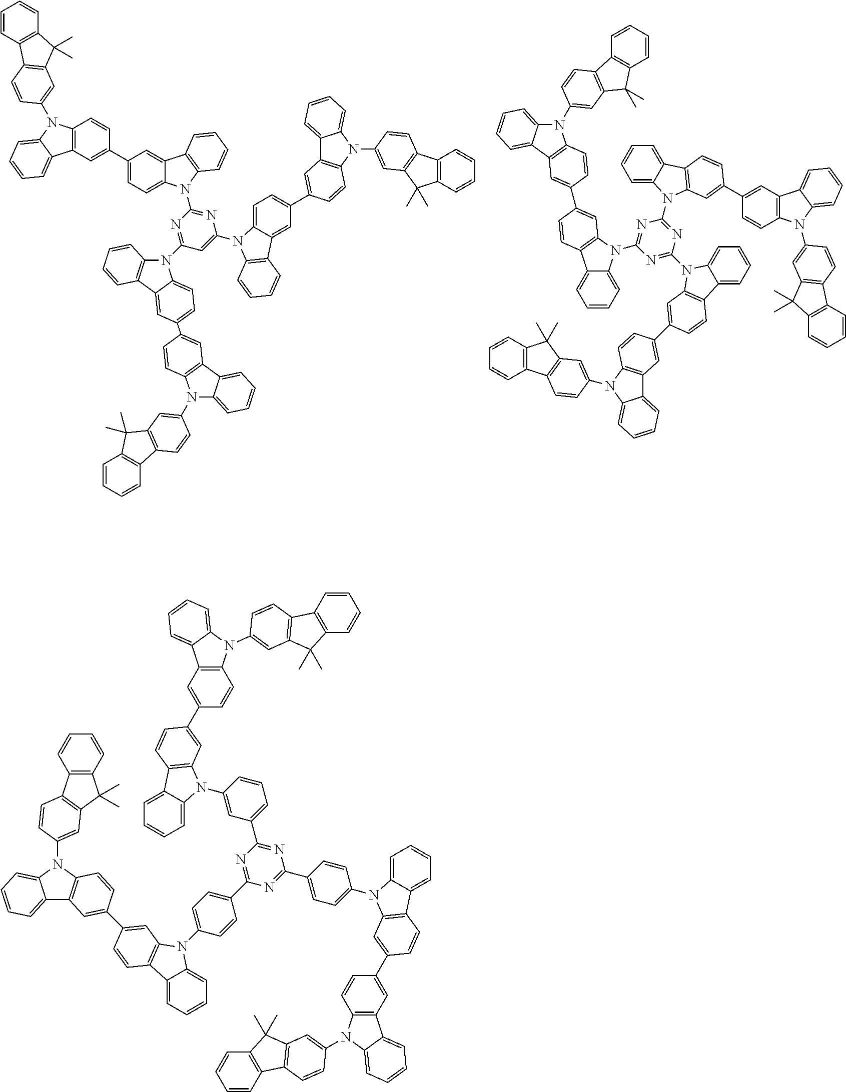

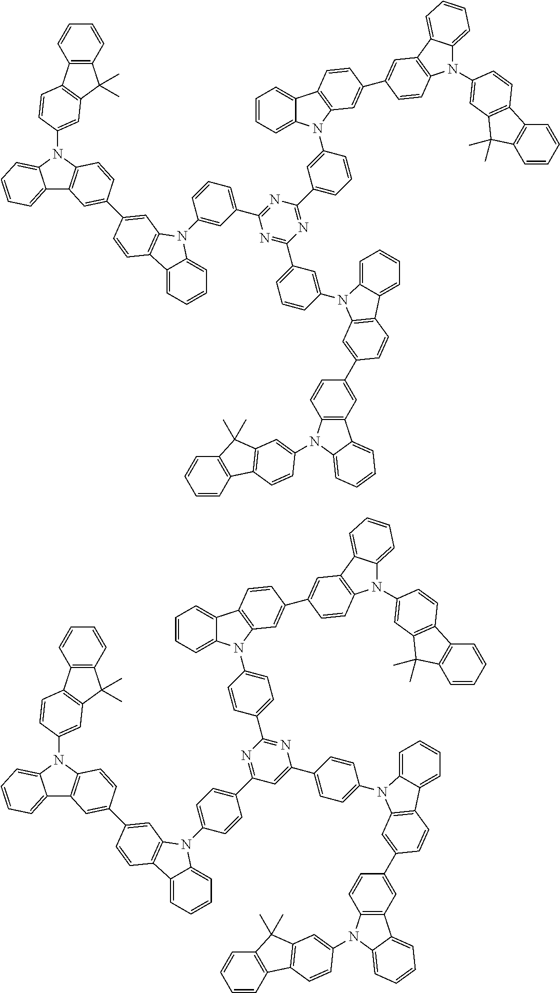

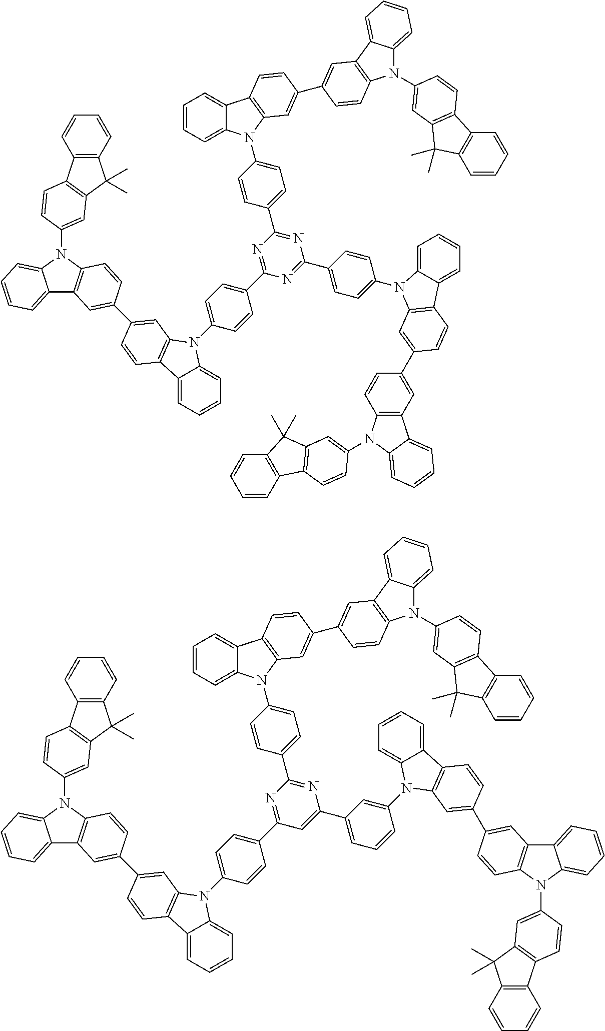

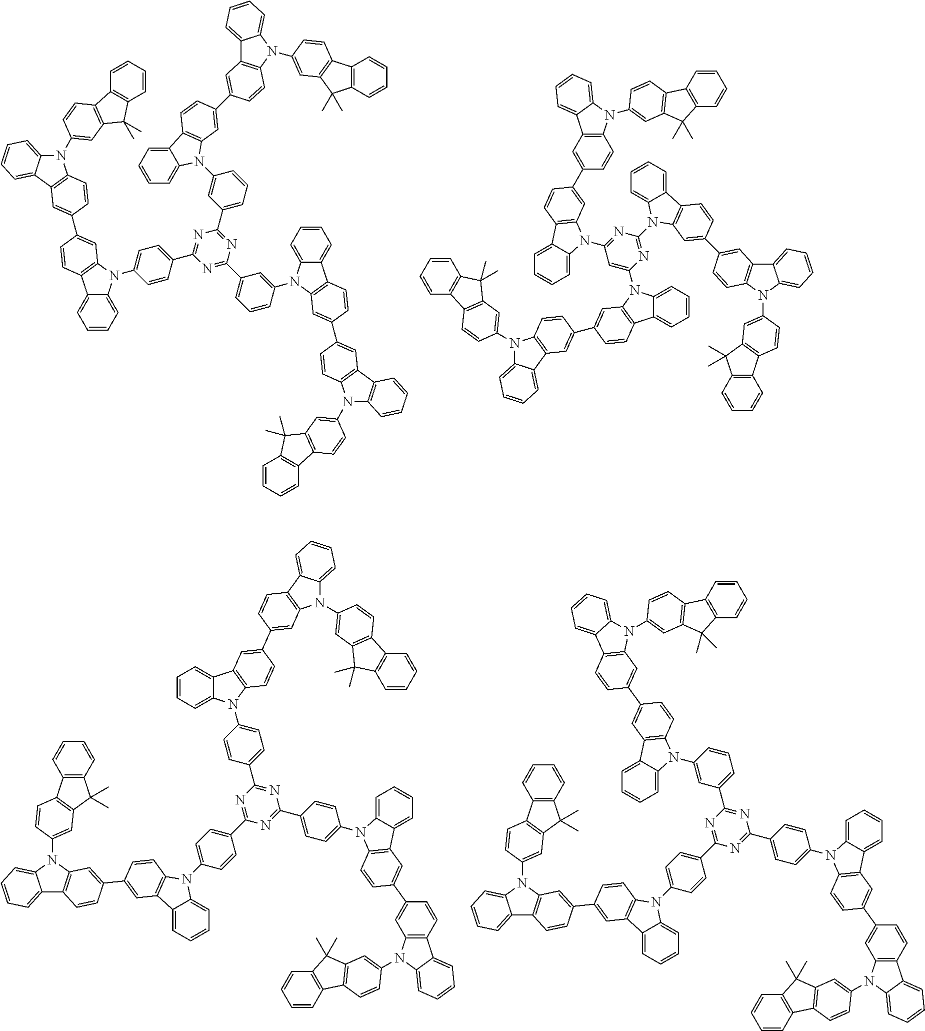

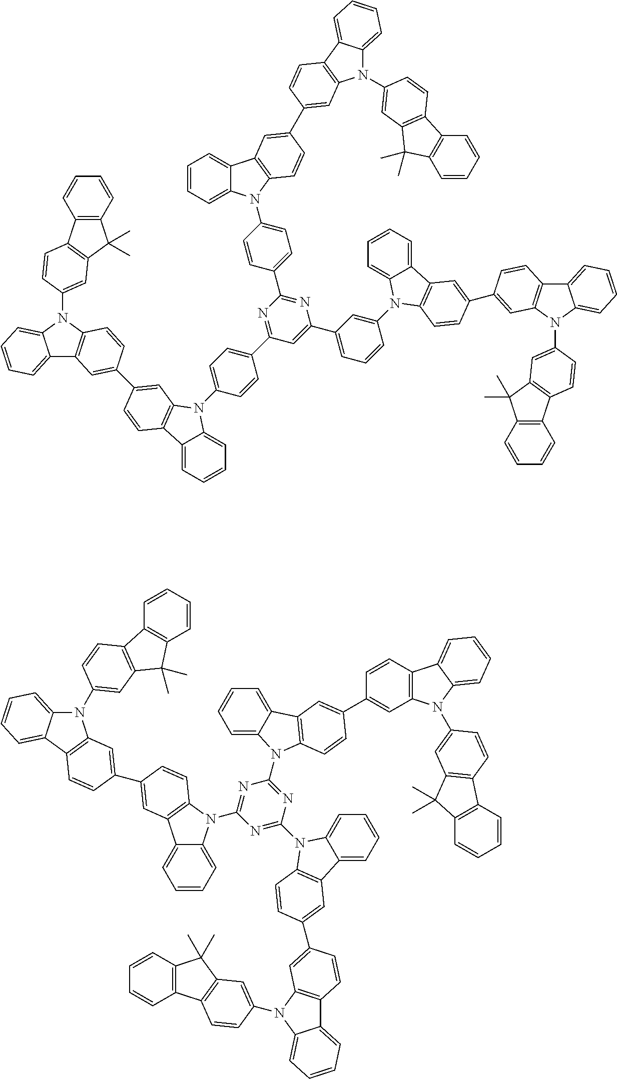

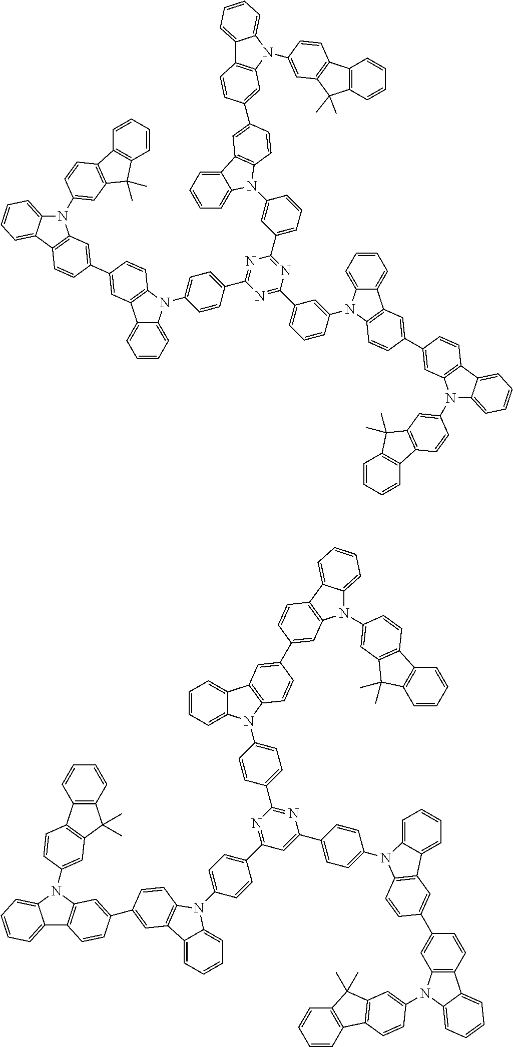

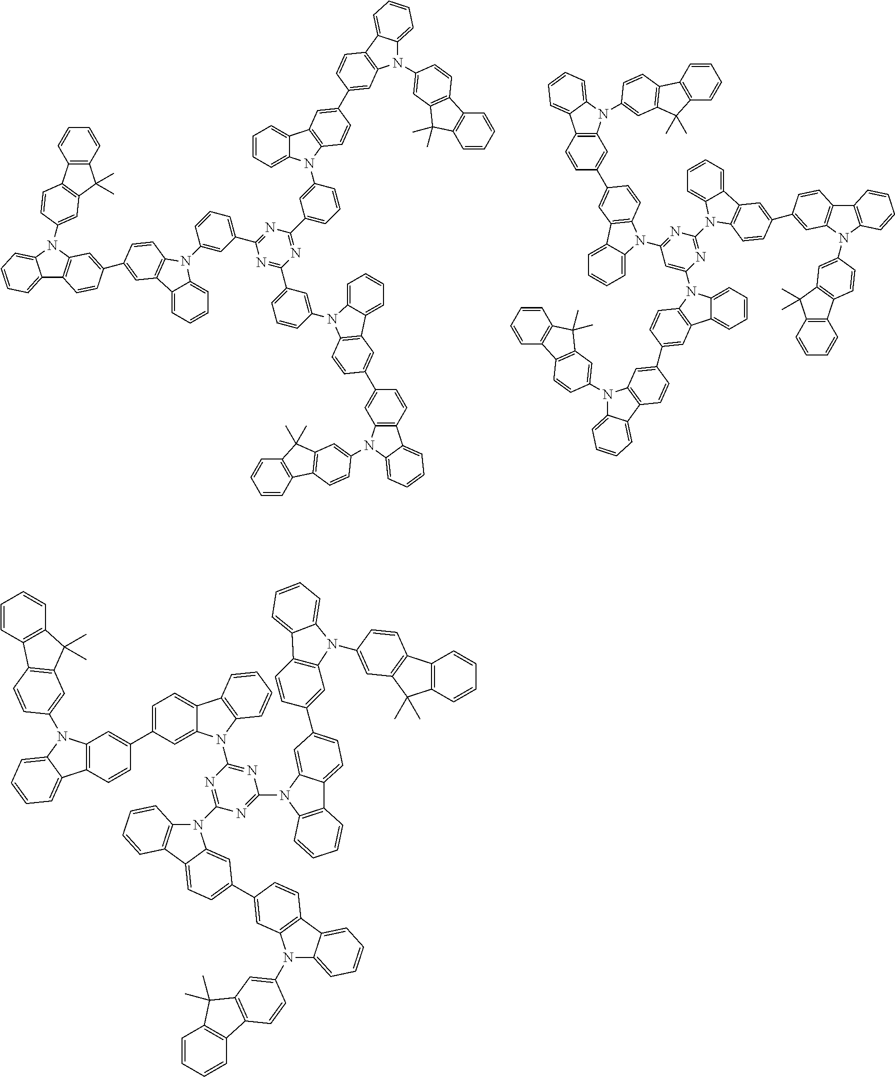

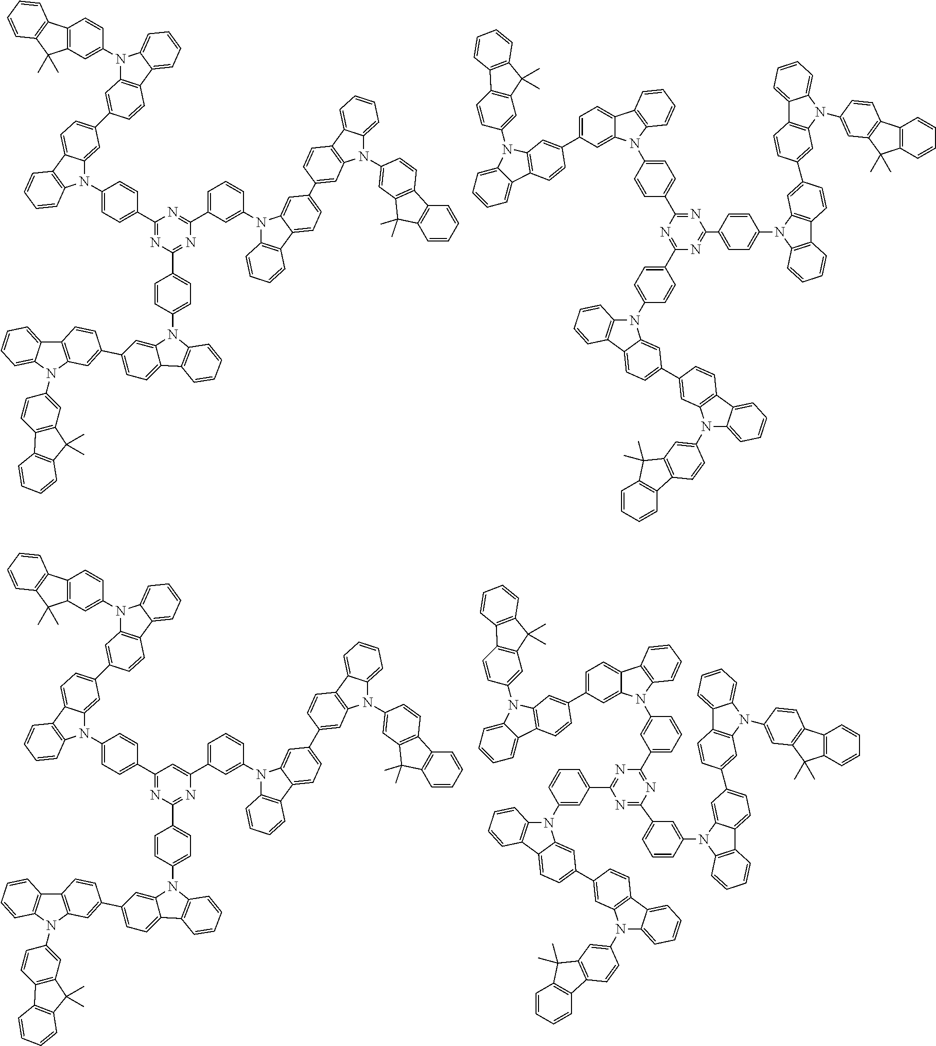

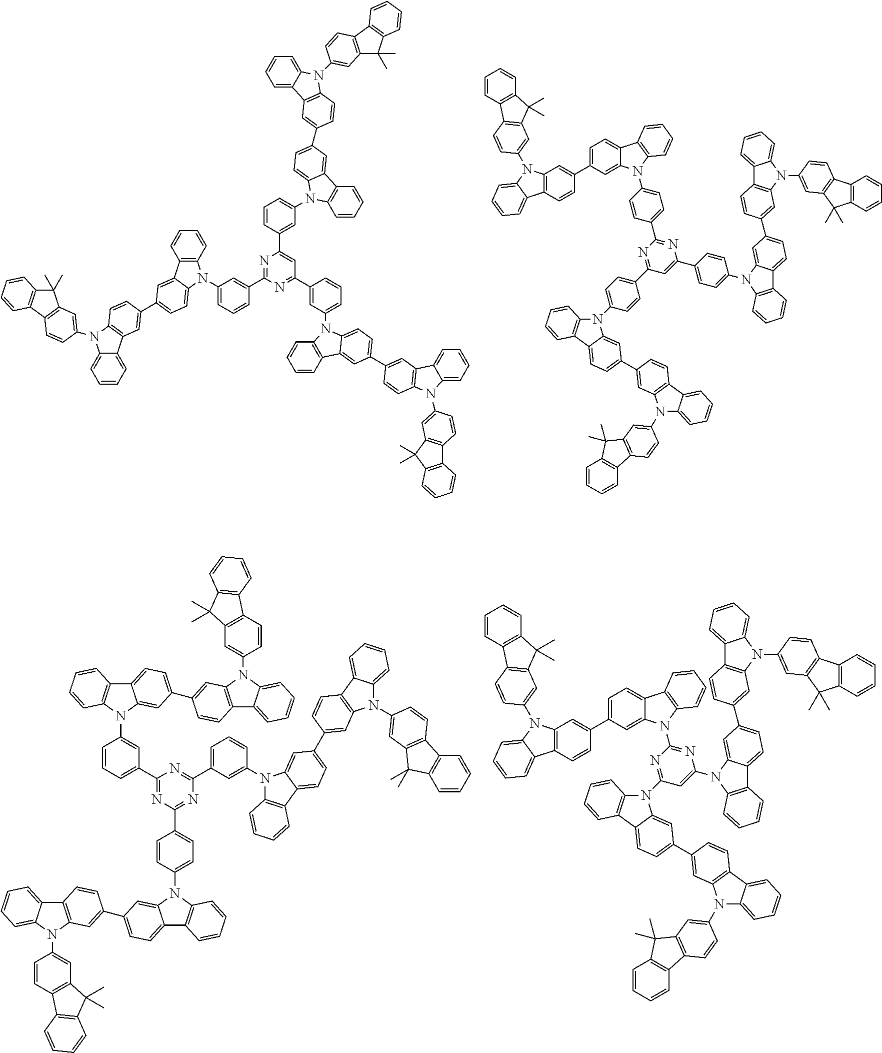

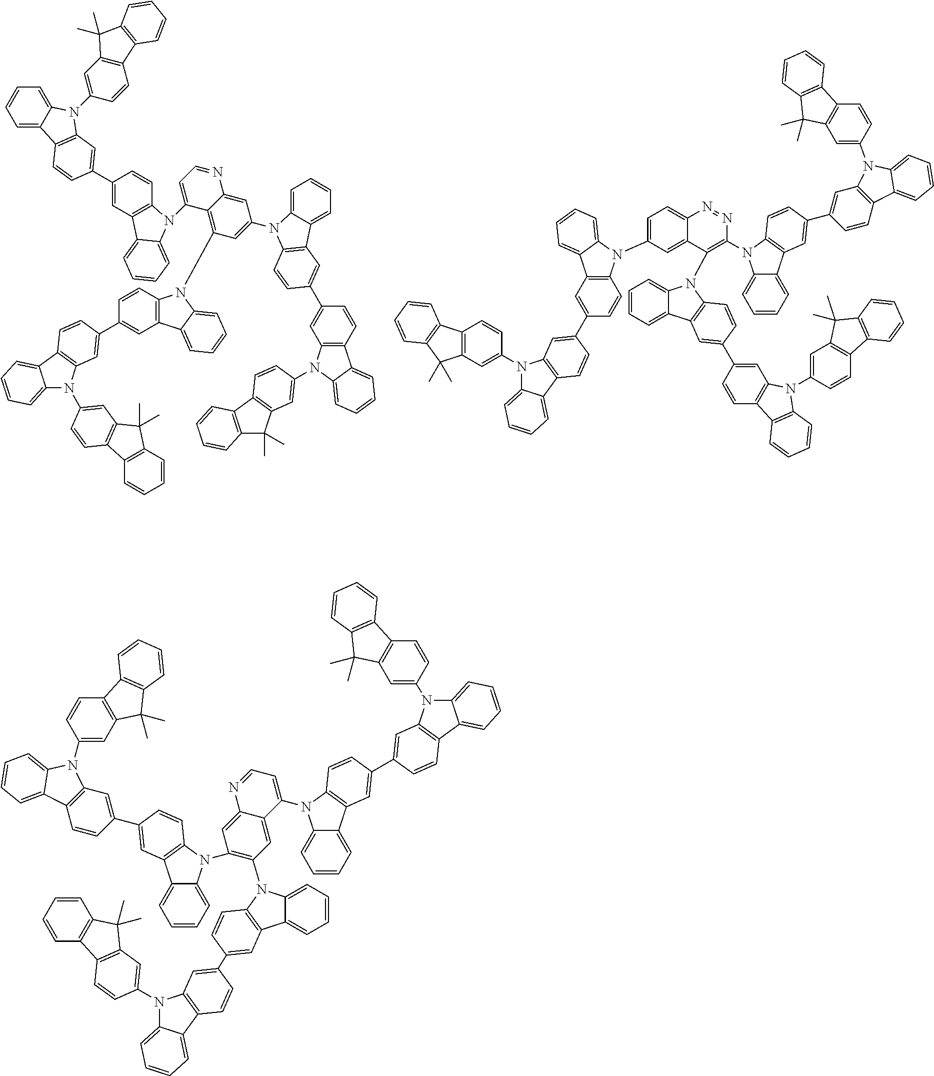

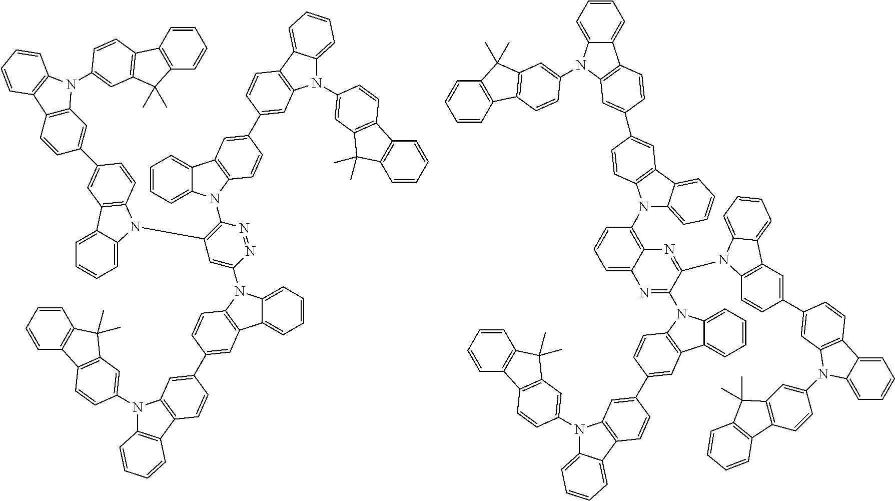

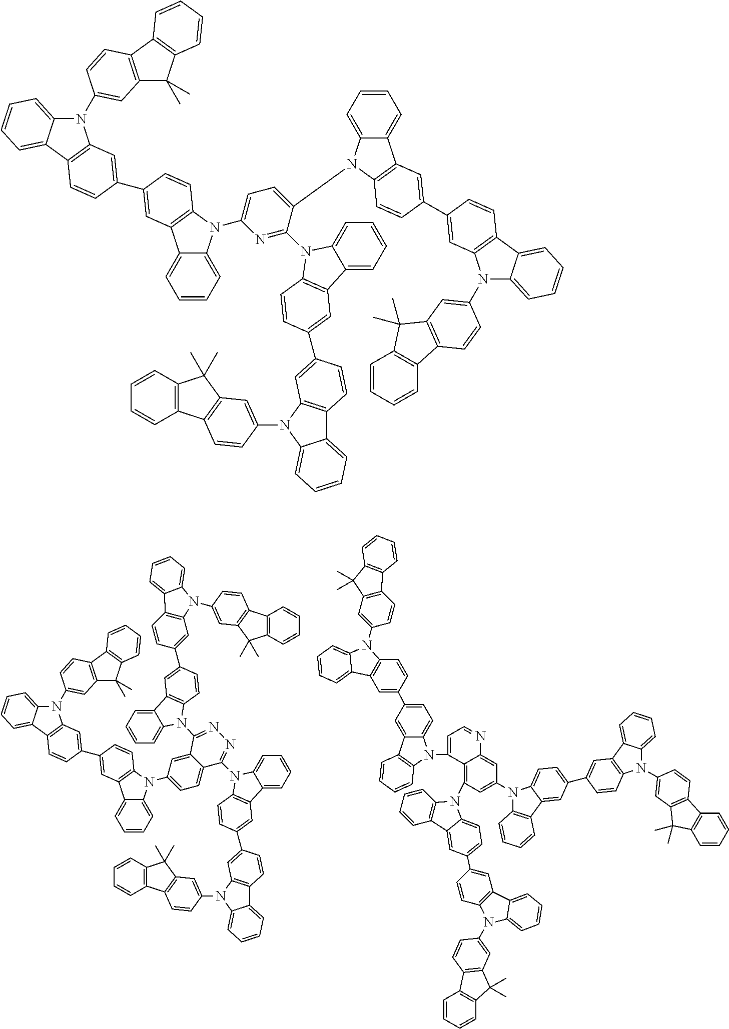

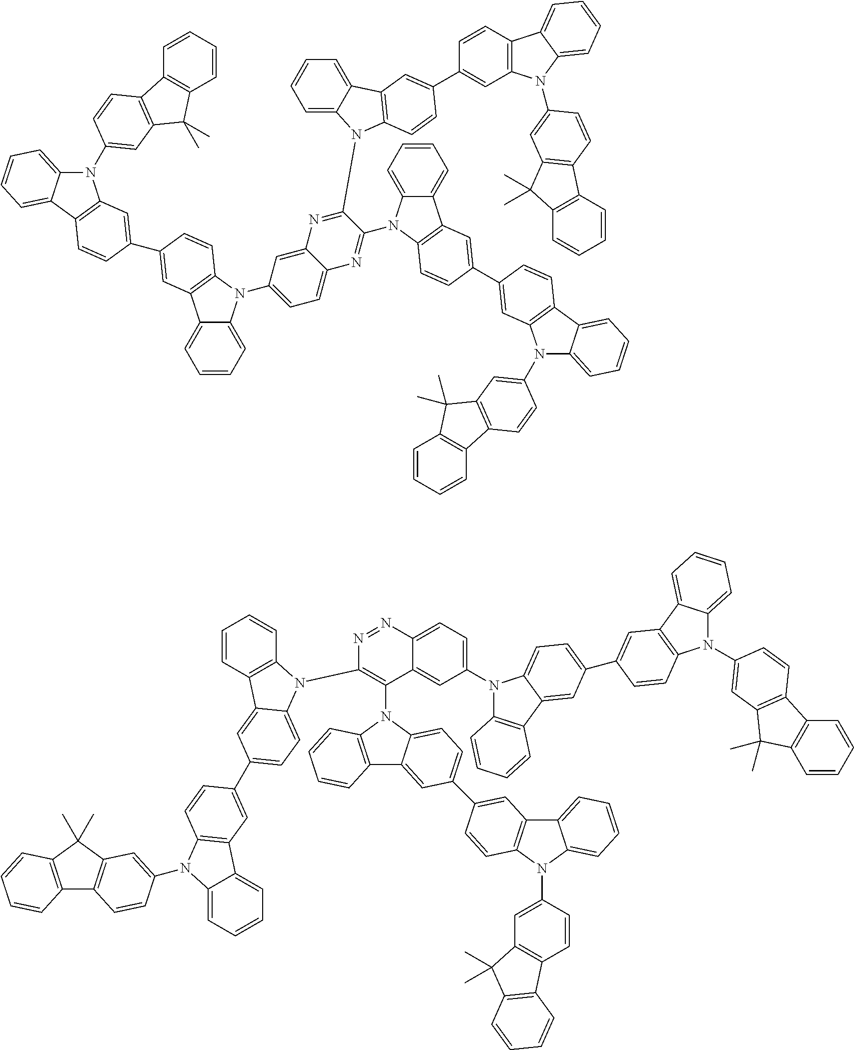

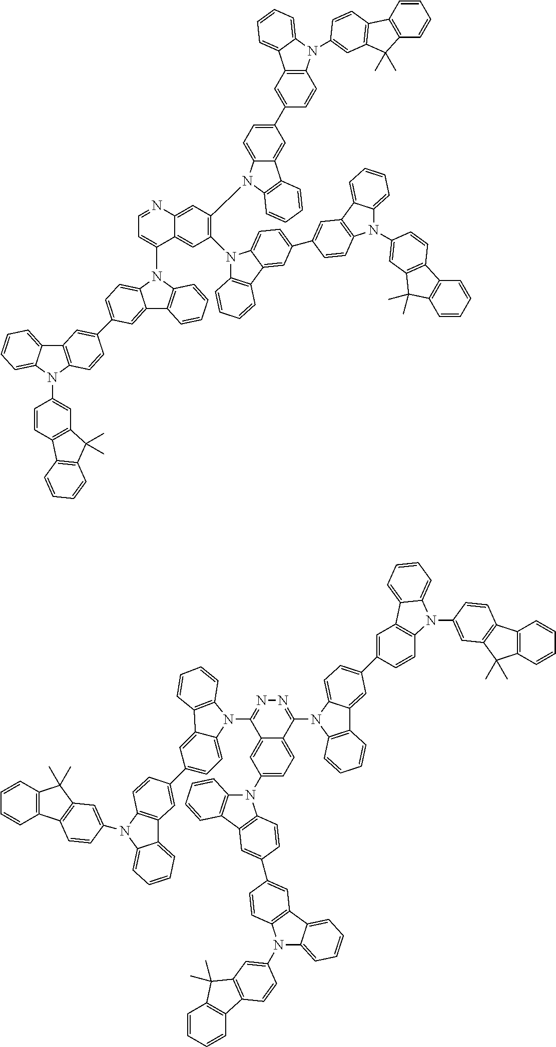 [SMILES: CC1(C)c2ccccc2-c2ccc(-n3c4ccccc4c4cc(-c5ccc6c(c5)c5ccccc5n6-c5cc6nccc(-n7c8ccccc8c8cc(-c9ccc%10c(c9)c9ccccc9n%10-c9ccc%10c(c9)C(C)(C)c9ccccc9-%10)ccc87)c6cc5-n5c6ccccc6c6cc(-c7ccc8c(c7)c7ccccc7n8-c7ccc8c(c7)C(C)(C)c7ccccc7-8)ccc65)ccc43)cc21 ; CC1(C)c2ccccc2-c2ccc(-n3c4ccccc4c4cc(-c5ccc6c(c5)c5ccccc5n6-c5ccc6c(-n7c8ccccc8c8cc(-c9ccc%10c(c9)c9ccccc9n%10-c9ccc%10c(c9)C(C)(C)c9ccccc9-%10)ccc87)nnc(-n7c8ccccc8c8cc(-c9ccc%10c(c9)c9ccccc9n%10-c9ccc%10c(c9)C(C)(C)c9ccccc9-%10)ccc87)c6c5)ccc43)cc21]

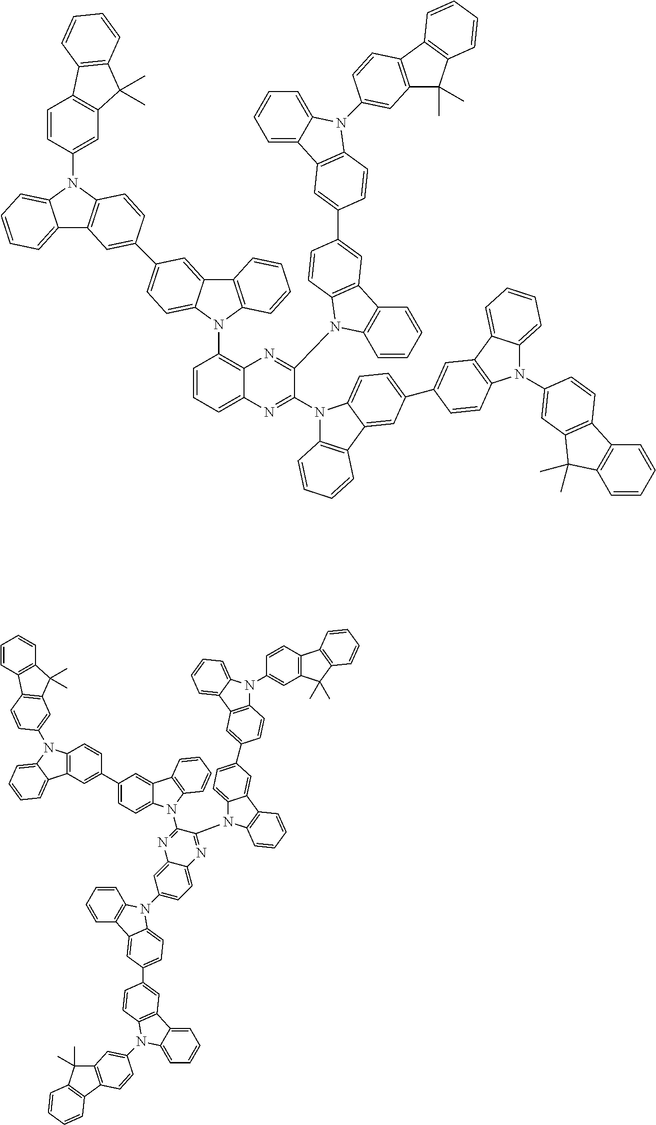

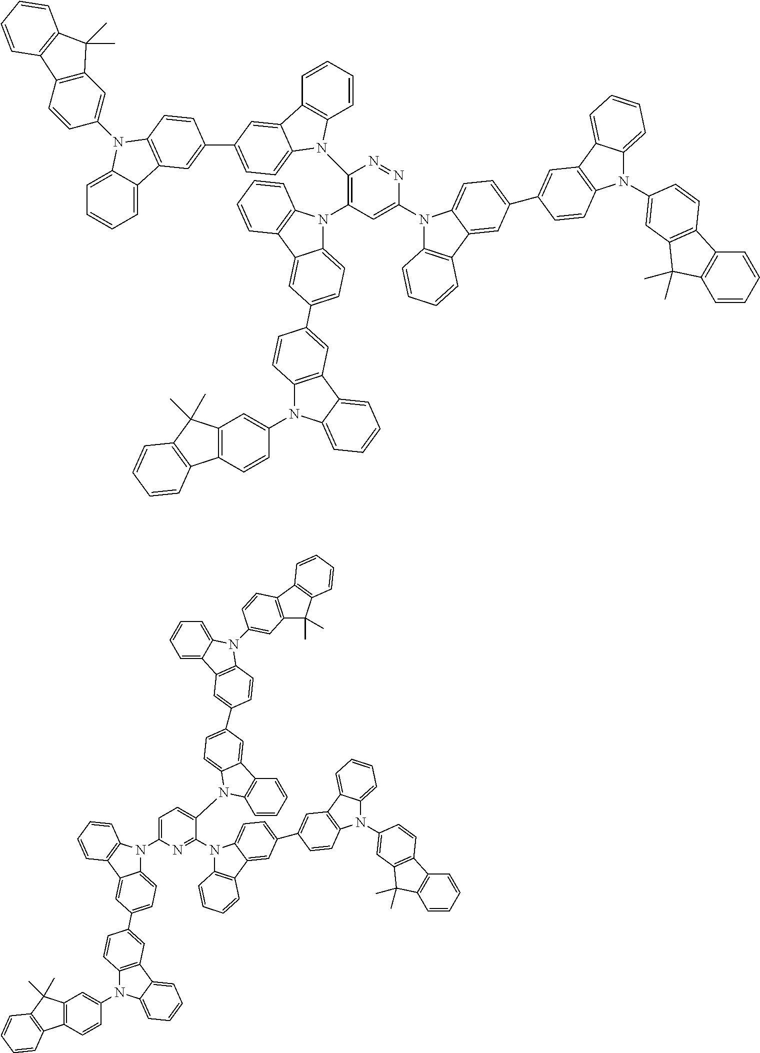

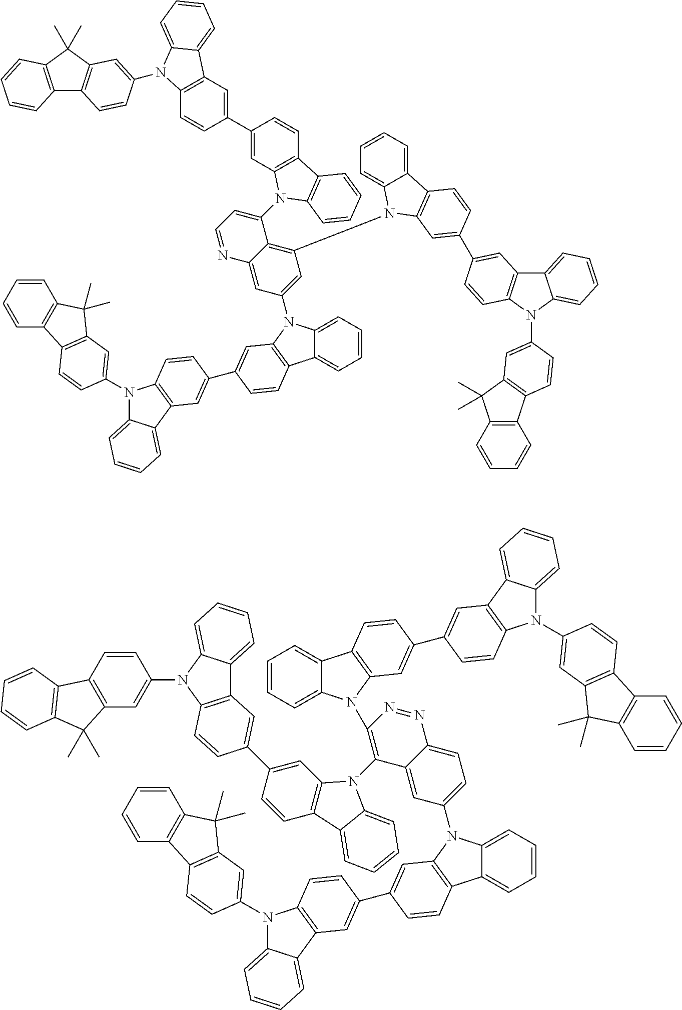

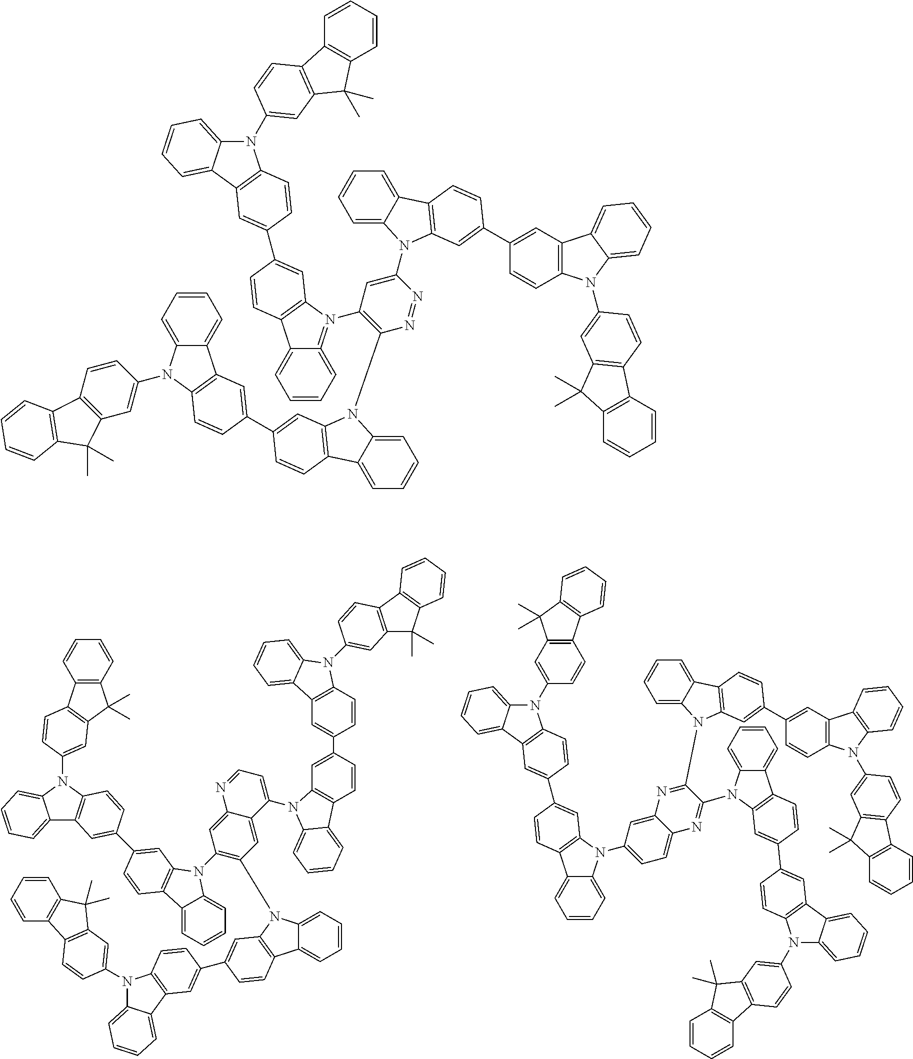

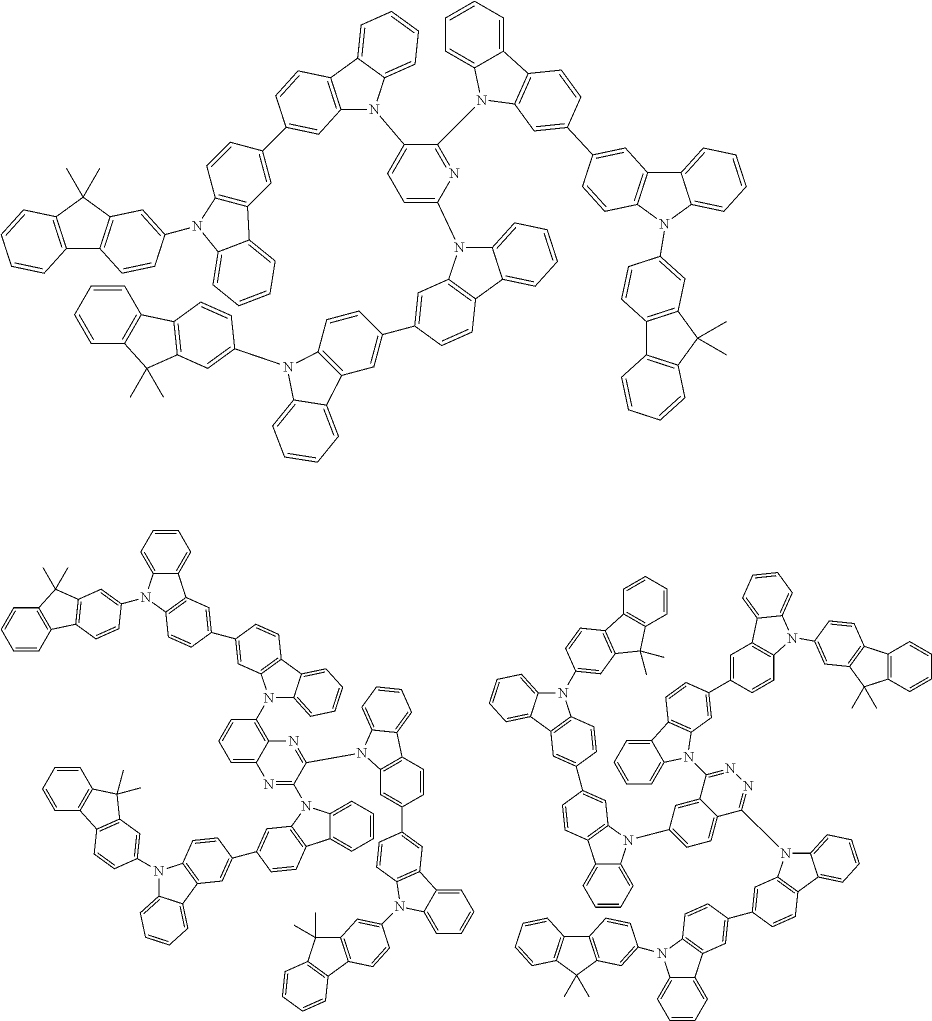

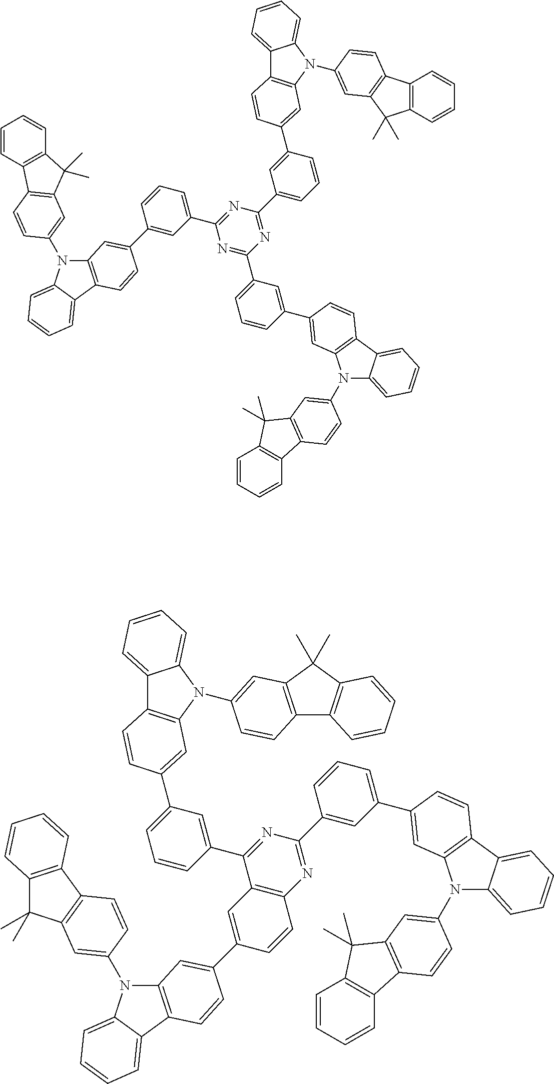 [SMILES: CC1(C)c2ccccc2-c2ccc(-n3c4ccccc4c4ccc(-c5cccc(-c6nc(-c7cccc(-c8ccc9c%10ccccc%10n(-c%10ccc%11c(c%10)C(C)(C)c%10ccccc%10-%11)c9c8)c7)nc(-c7cccc(-c8ccc9c%10ccccc%10n(-c%10ccc%11c(c%10)C(C)(C)c%10ccccc%10-%11)c9c8)c7)n6)c5)cc43)cc21 ; CC1(C)c2ccccc2-c2ccc(-n3c4ccccc4c4ccc(-c5cccc(-c6nc(-c7cccc(-c8ccc9c%10ccccc%10n(-c%10ccc%11c(c%10)C(C)(C)c%10ccccc%10-%11)c9c8)c7)c7cc(-c8ccc9c%10ccccc%10n(-c%10ccc%11c(c%10)C(C)(C)c%10ccccc%10-%11)c9c8)ccc7n6)c5)cc43)cc21]

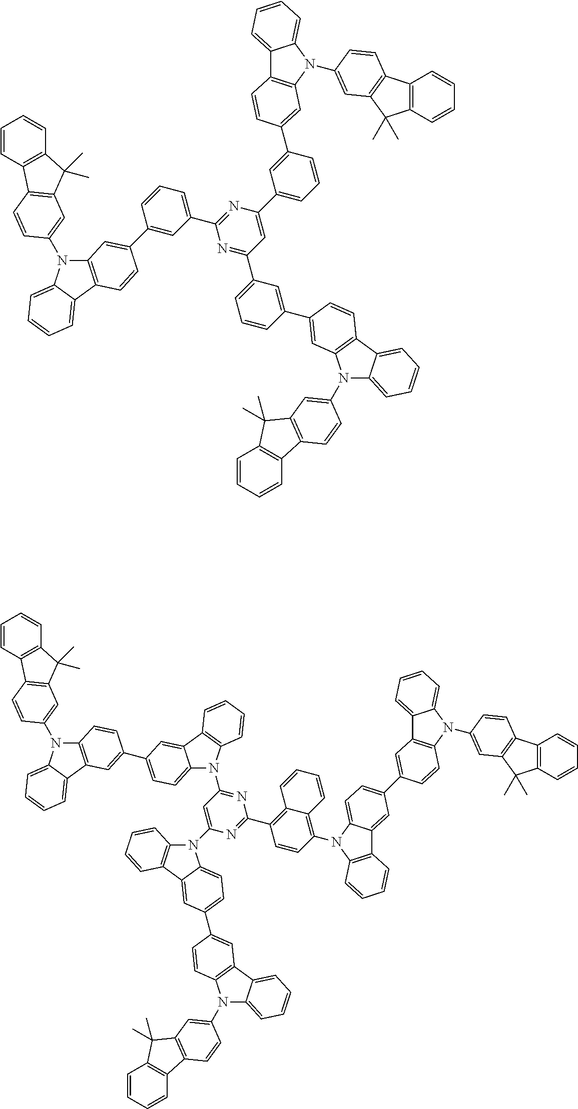 [SMILES: CC1(C)c2ccccc2-c2ccc(-n3c4ccccc4c4ccc(-c5cccc(-c6cc(-c7cccc(-c8ccc9c%10ccccc%10n(-c%10ccc%11c(c%10)C(C)(C)c%10ccccc%10-%11)c9c8)c7)nc(-c7cccc(-c8ccc9c%10ccccc%10n(-c%10ccc%11c(c%10)C(C)(C)c%10ccccc%10-%11)c9c8)c7)n6)c5)cc43)cc21 ; CC1(C)c2ccccc2-c2ccc(-n3c4ccccc4c4cc(-c5ccc6c(c5)c5ccccc5n6-c5cc(-n6c7ccccc7c7cc(-c8ccc9c(c8)c8ccccc8n9-c8ccc9c(c8)C(C)(C)c8ccccc8-9)ccc76)nc(-c6ccc(-n7c8ccccc8c8cc(-c9ccc%10c(c9)c9ccccc9n%10-c9ccc%10c(c9)C(C)(C)c9ccccc9-%10)ccc87)c7ccccc67)n5)ccc43)cc21]

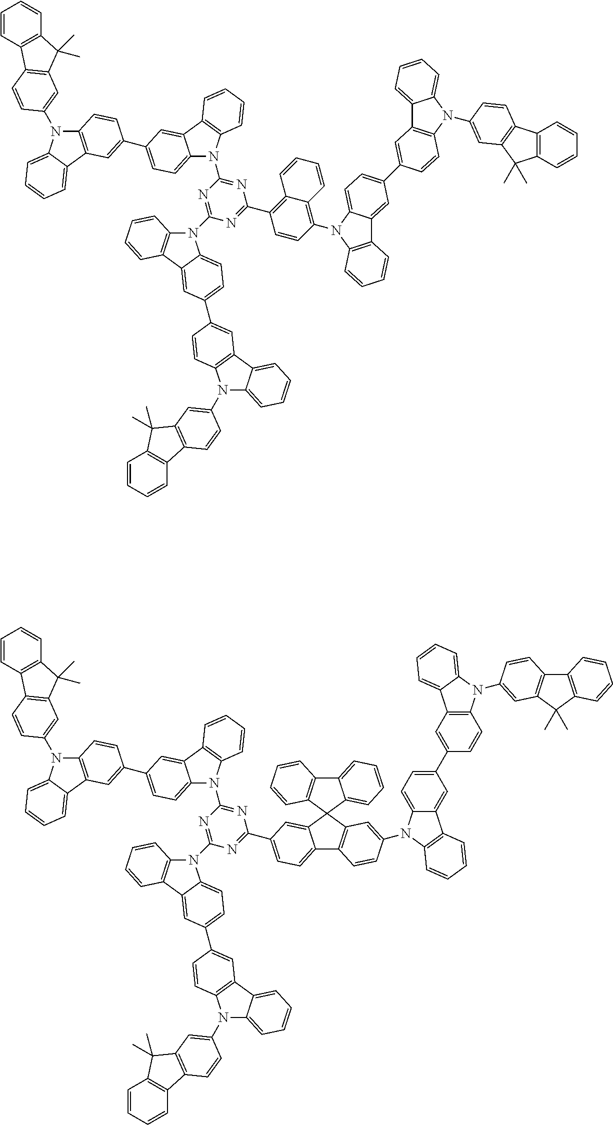

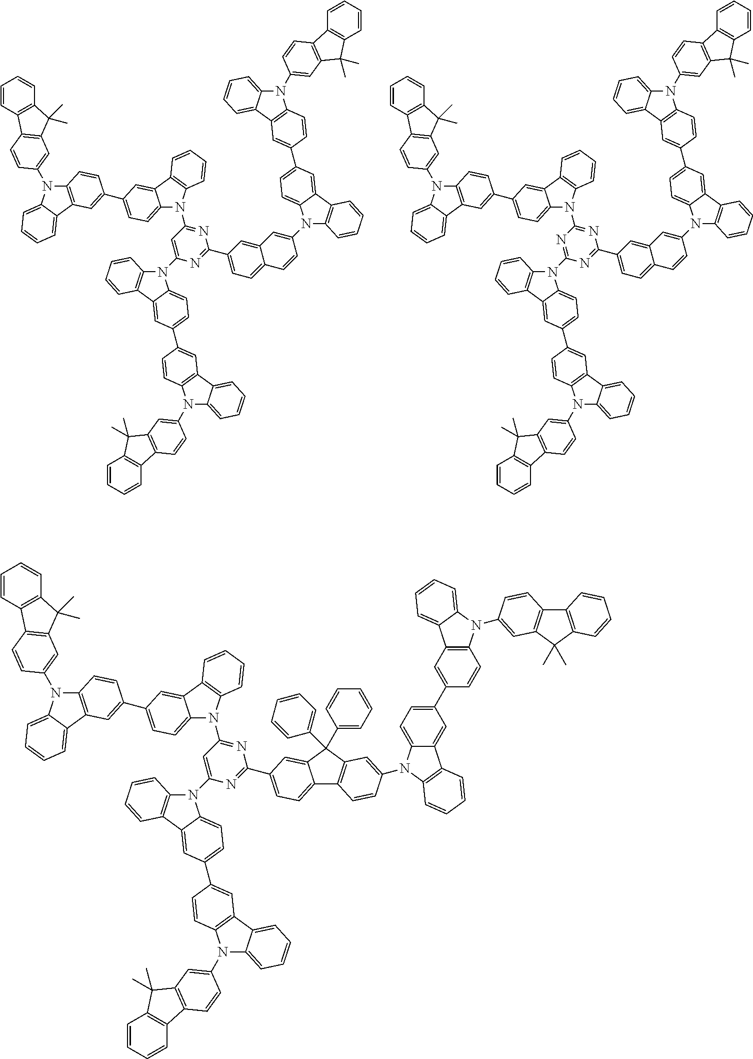

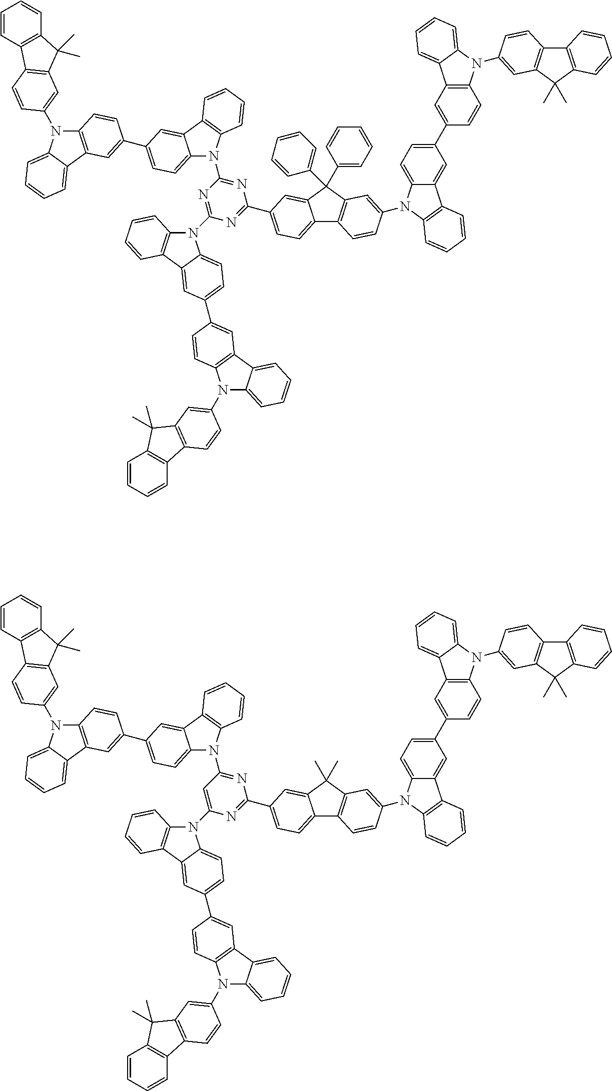

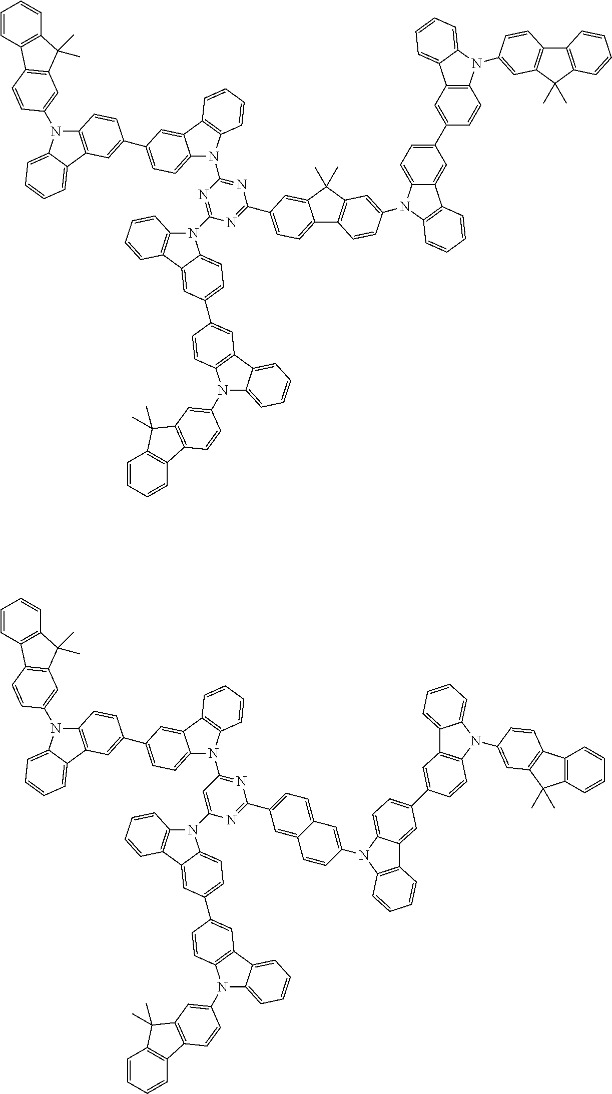

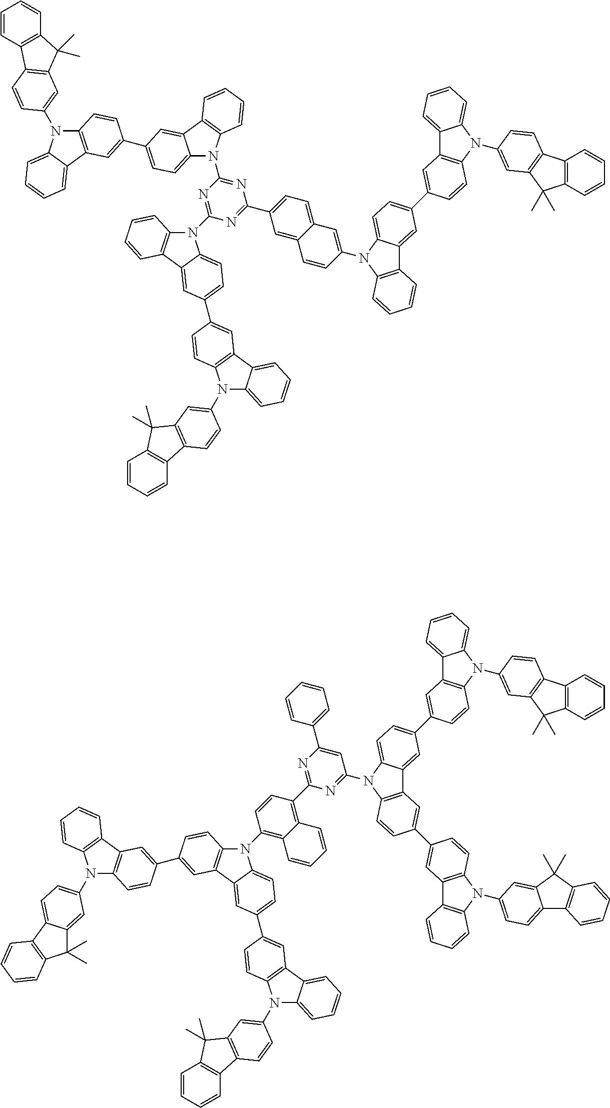

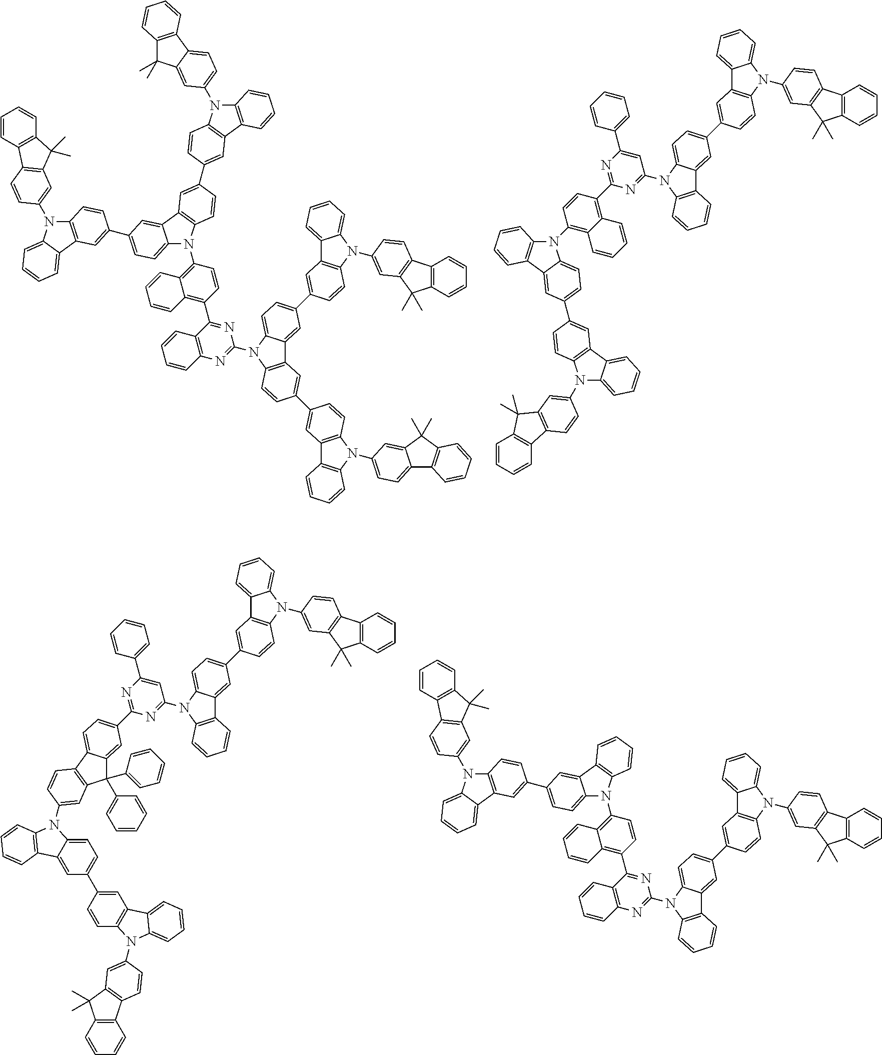

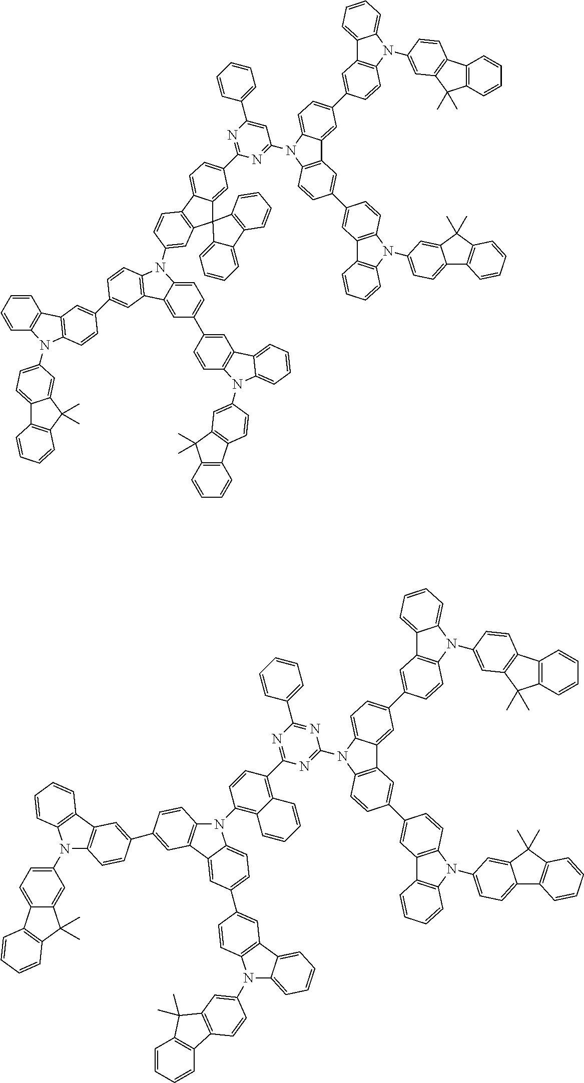

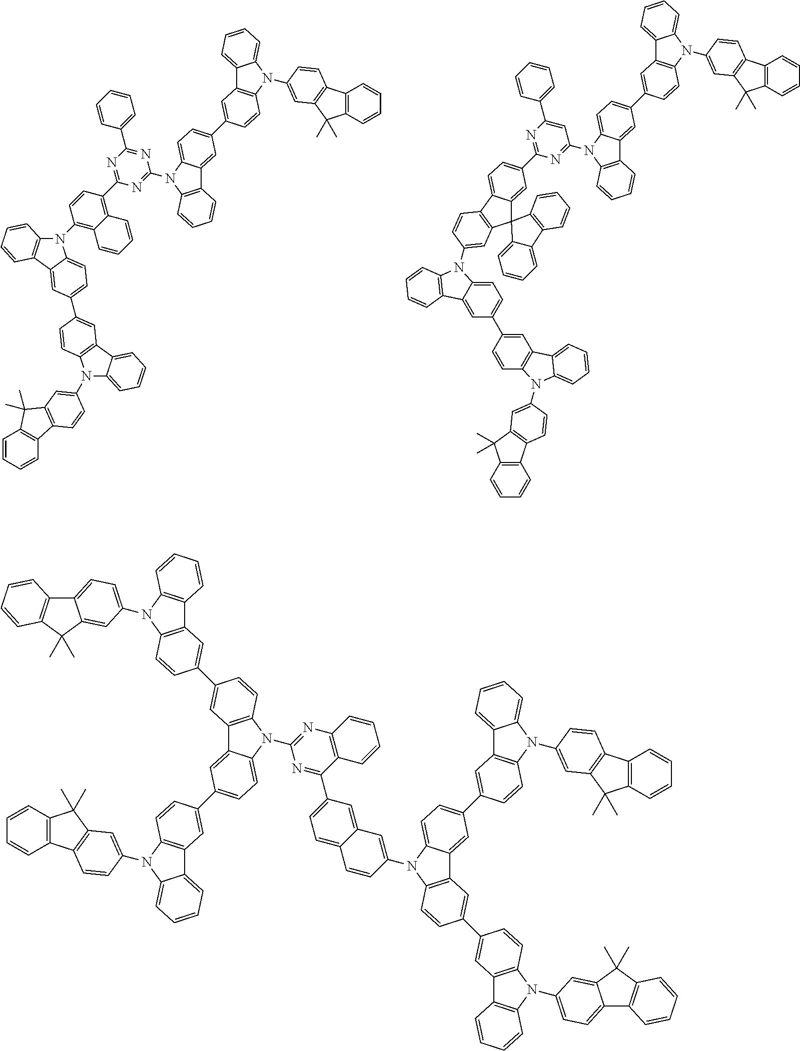

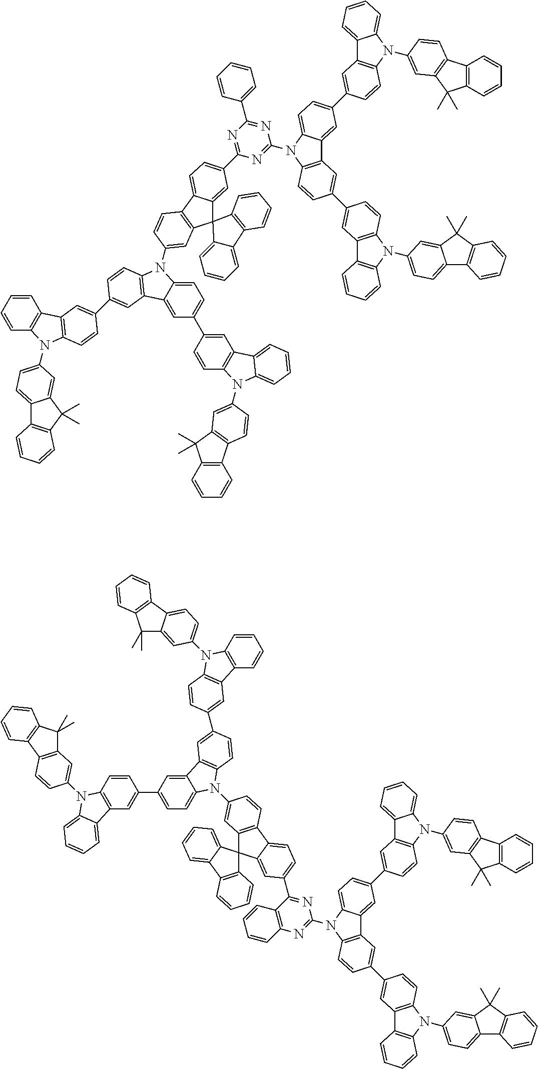

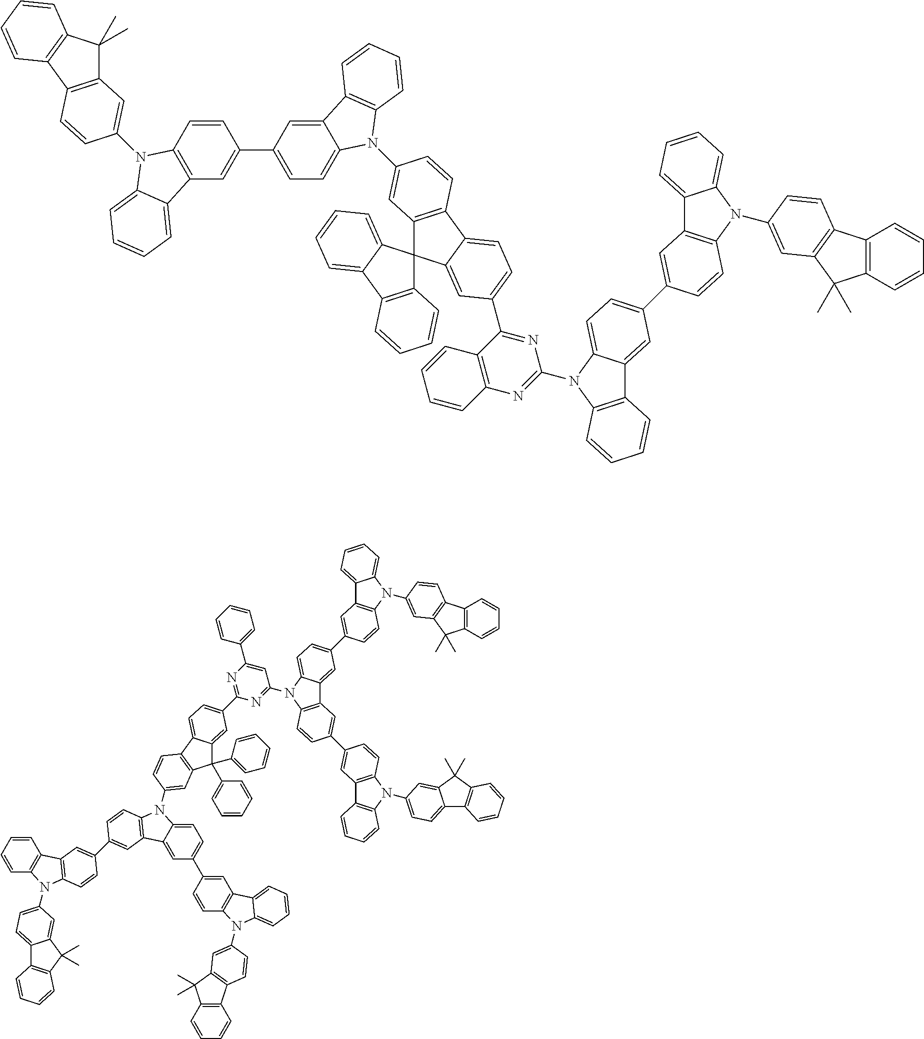

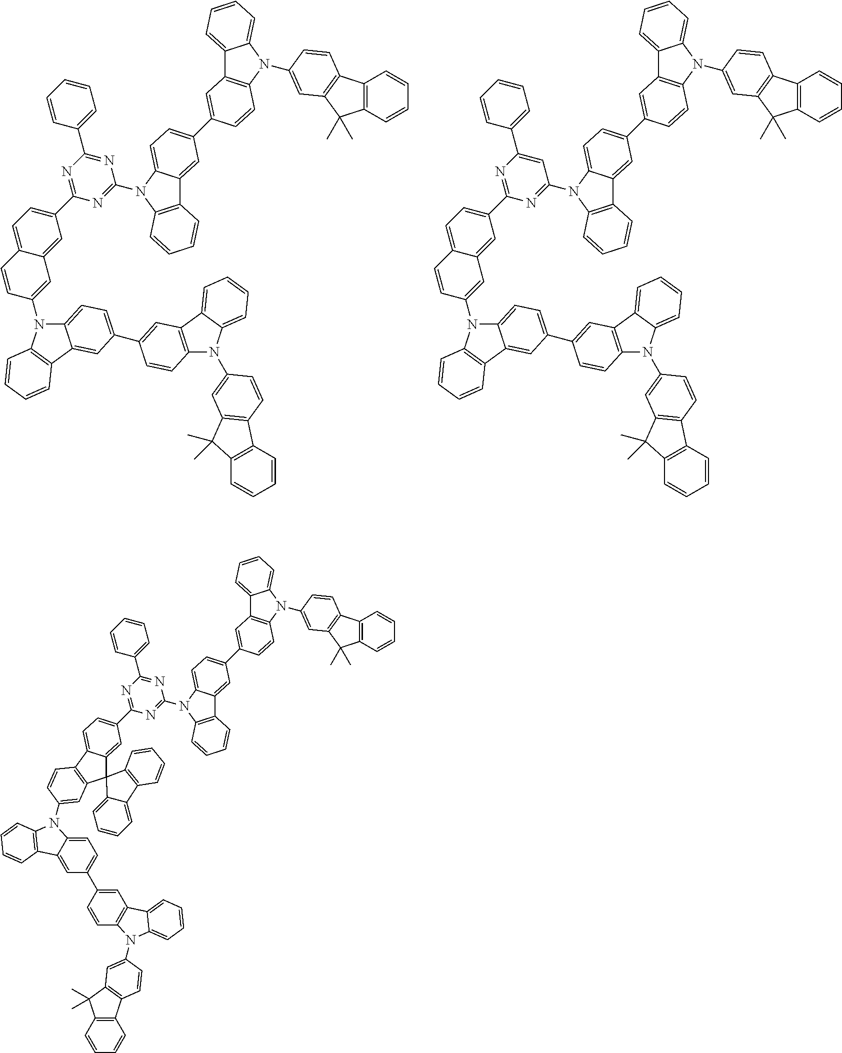

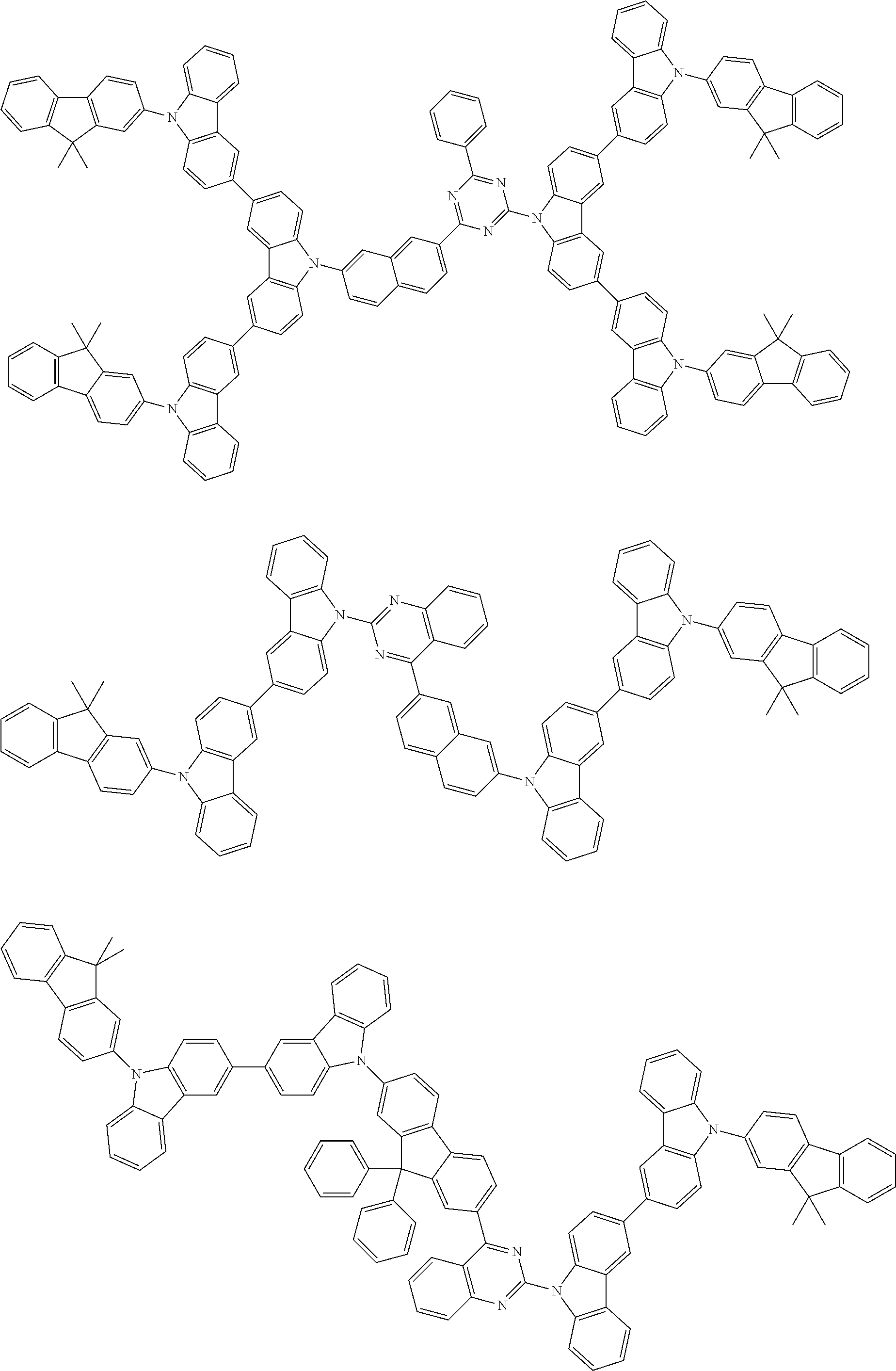

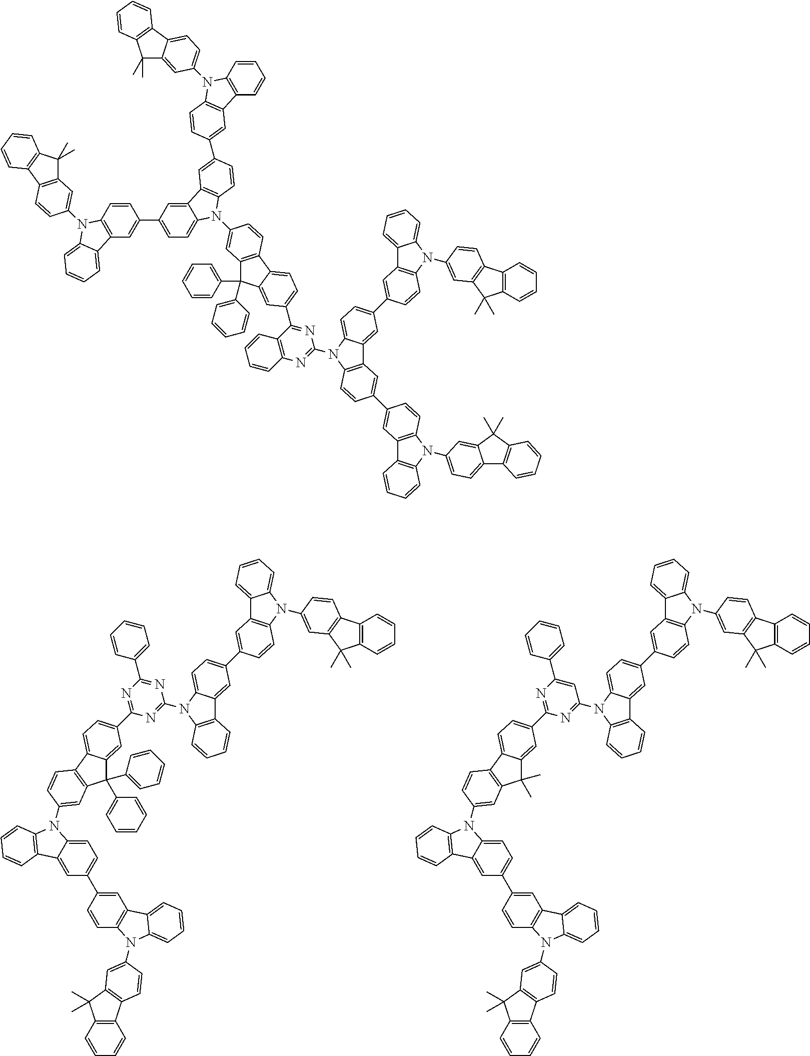

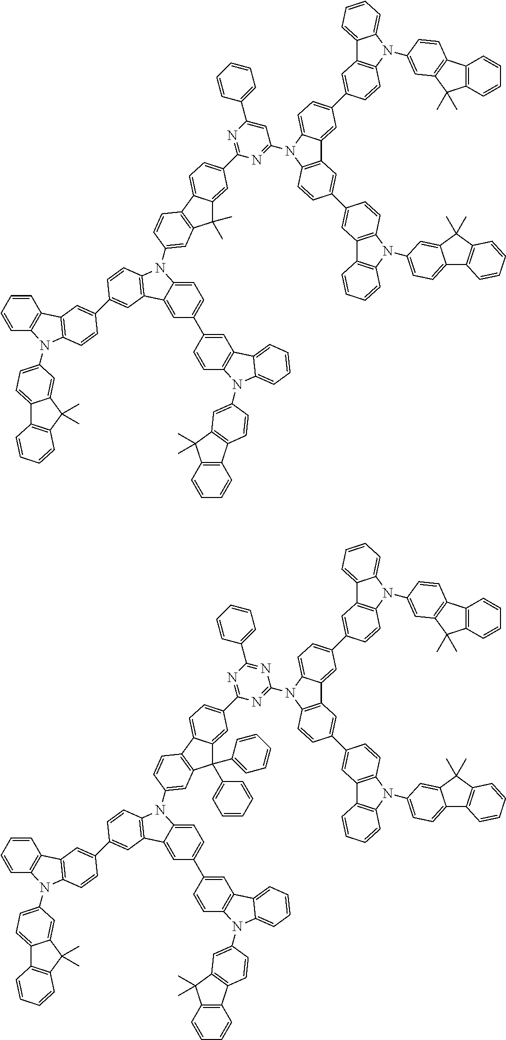

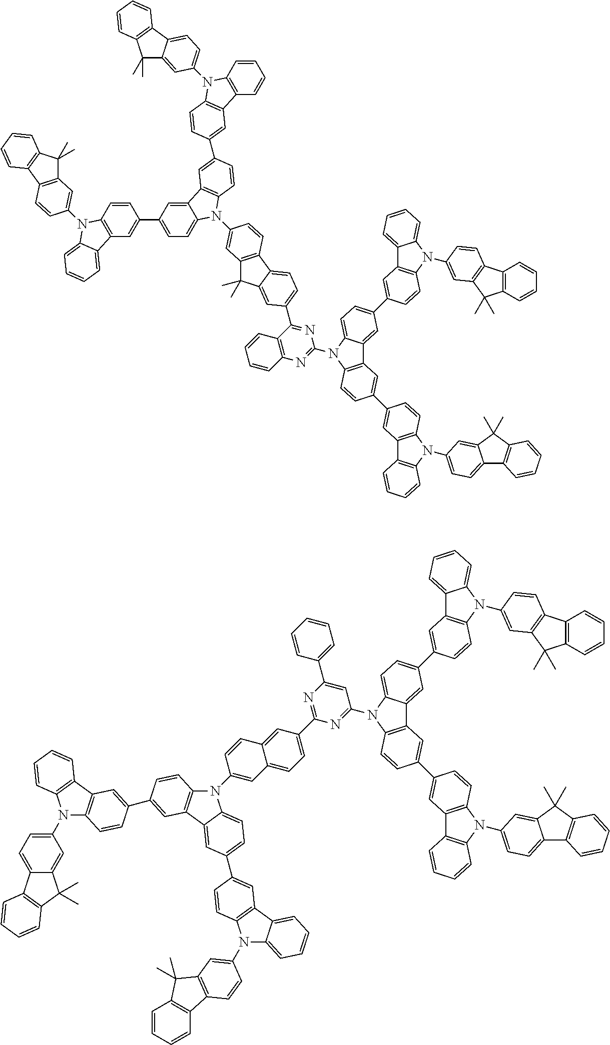

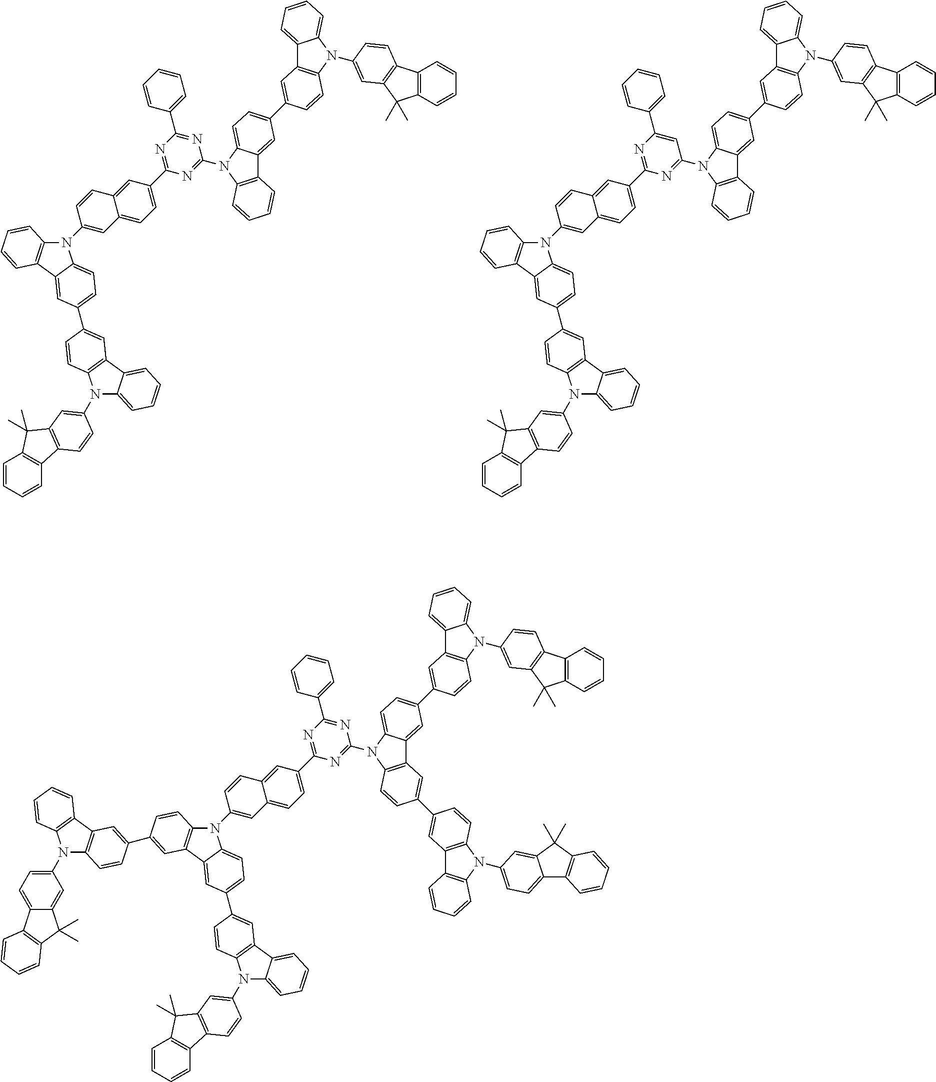

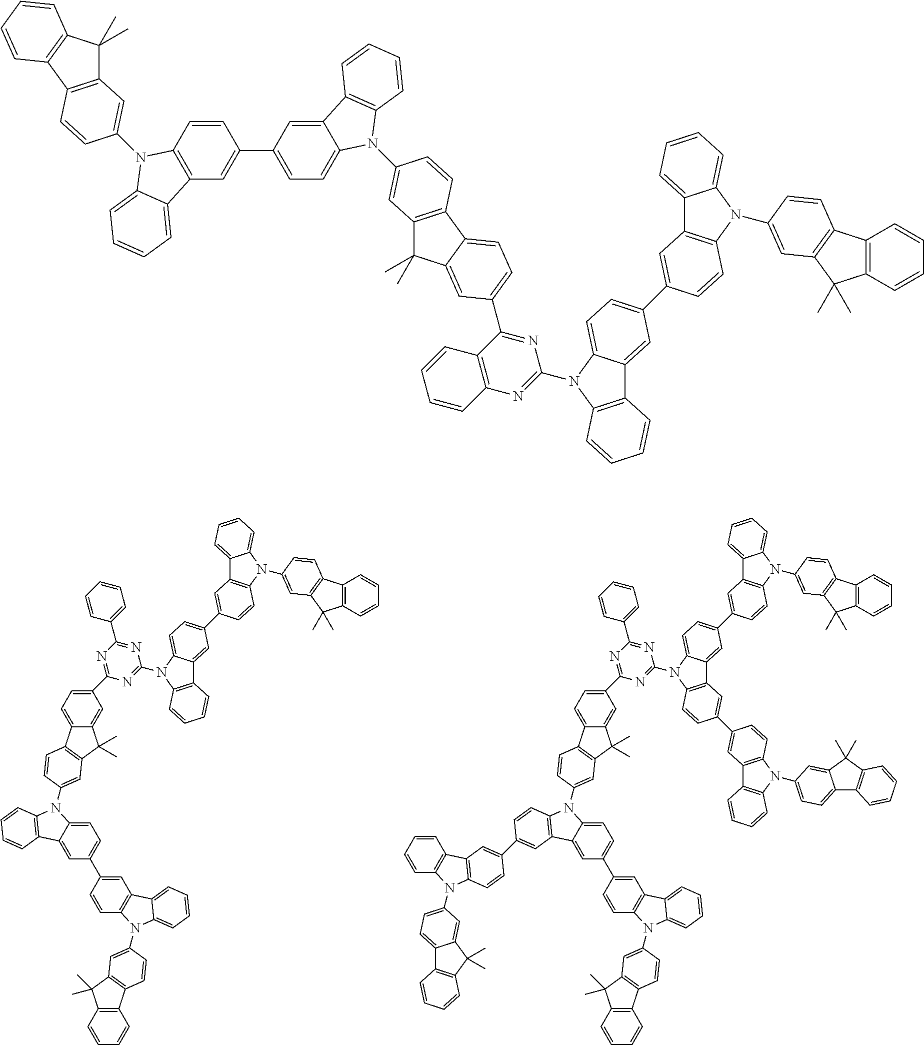

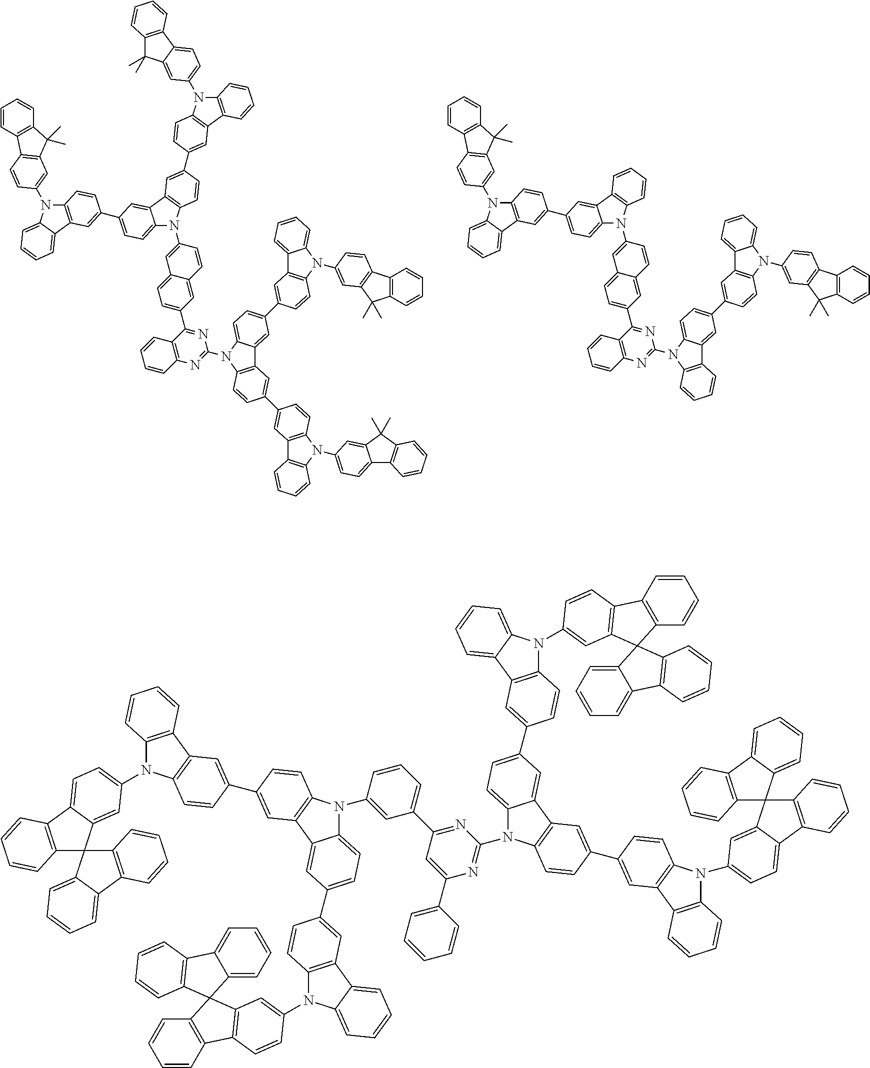

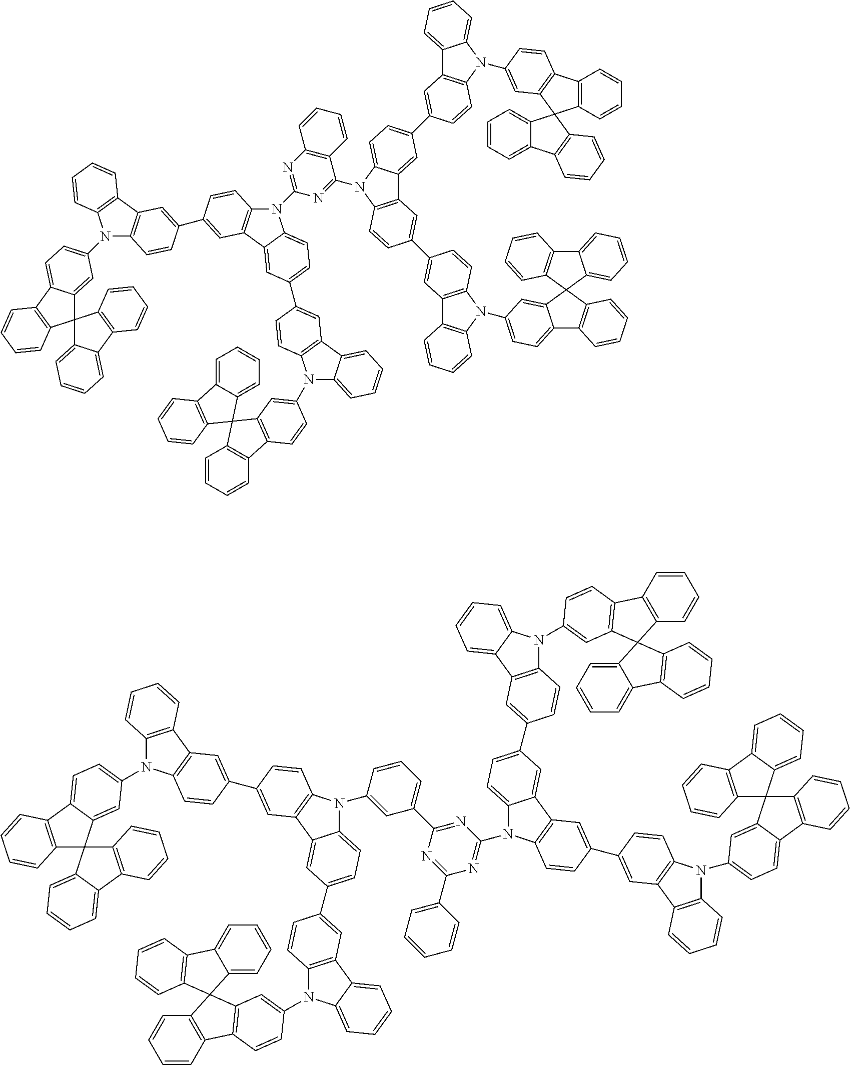

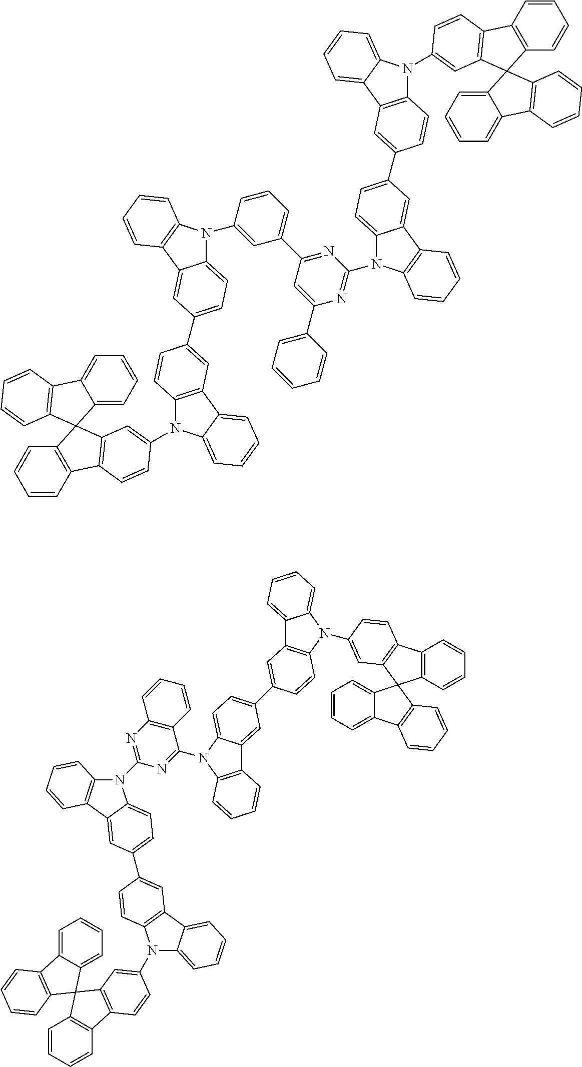 [SMILES: c1ccc(-c2cc(-c3cccc(-n4c5ccccc5c5cc(-c6ccc7c(c6)c6ccccc6n7-c6ccc7c(c6)C6(c8ccccc8-c8ccccc86)c6ccccc6-7)ccc54)c3)nc(-n3c4ccccc4c4cc(-c5ccc6c(c5)c5ccccc5n6-c5ccc6c(c5)C5(c7ccccc7-c7ccccc75)c5ccccc5-6)ccc43)n2)cc1 ; c1ccc2c(c1)-c1ccccc1C21c2ccccc2-c2ccc(-n3c4ccccc4c4cc(-c5ccc6c(c5)c5ccccc5n6-c5nc(-n6c7ccccc7c7cc(-c8ccc9c(c8)c8ccccc8n9-c8ccc9c(c8)C8(c%10ccccc%10-c%10ccccc%108)c8ccccc8-9)ccc76)c6ccccc6n5)ccc43)cc21]

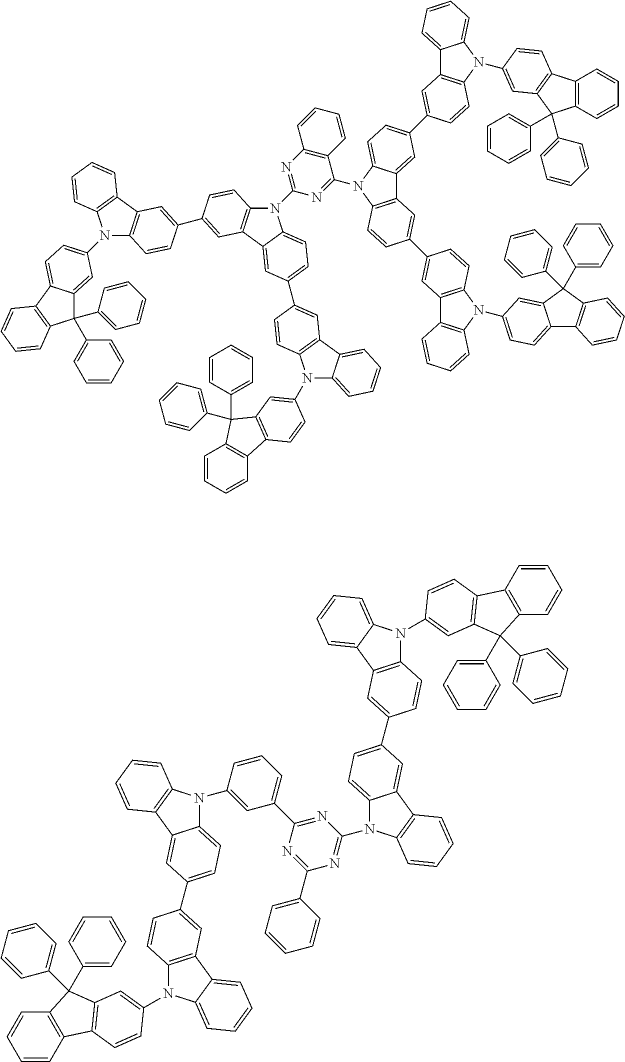

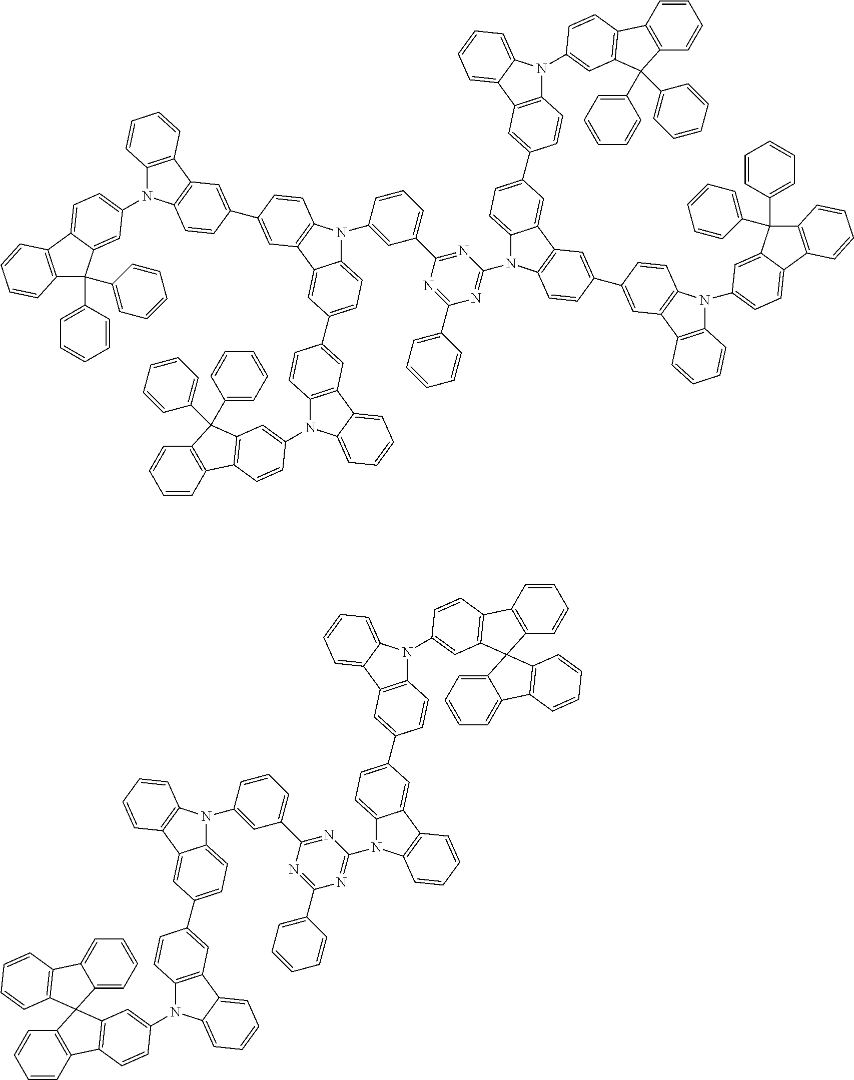

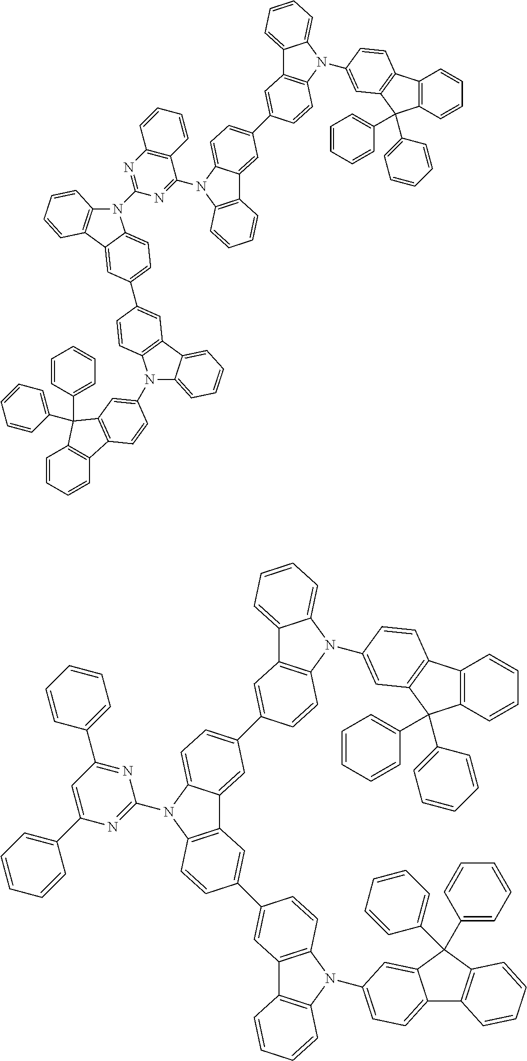 [SMILES: c1ccc(C2(c3ccccc3)c3ccccc3-c3ccc(-n4c5ccccc5c5cc(-c6ccc7c(c6)c6ccccc6n7-c6nc(-n7c8ccccc8c8cc(-c9ccc%10c(c9)c9ccccc9n%10-c9ccc%10c(c9)C(c9ccccc9)(c9ccccc9)c9ccccc9-%10)ccc87)c7ccccc7n6)ccc54)cc32)cc1 ; c1ccc(-c2cc(-c3ccccc3)nc(-n3c4ccc(-c5ccc6c(c5)c5ccccc5n6-c5ccc6c(c5)C(c5ccccc5)(c5ccccc5)c5ccccc5-6)cc4c4cc(-c5ccc6c(c5)c5ccccc5n6-c5ccc6c(c5)C(c5ccccc5)(c5ccccc5)c5ccccc5-6)ccc43)n2)cc1]

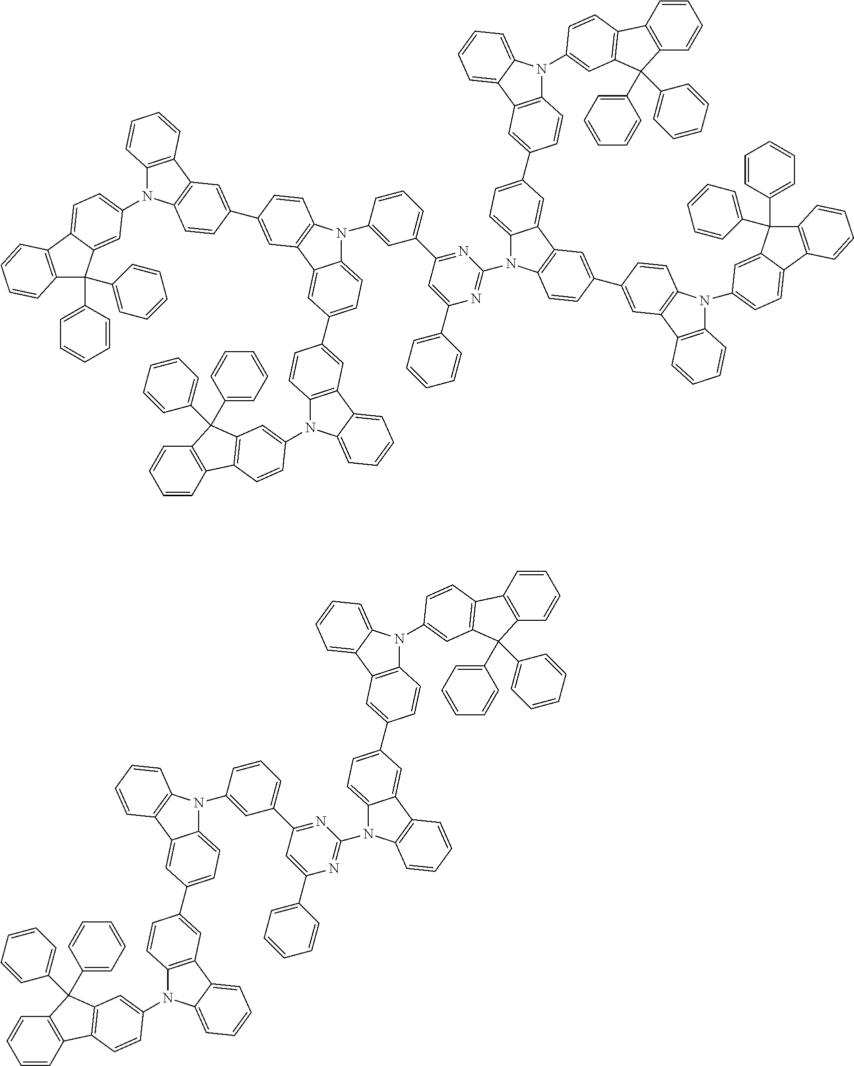

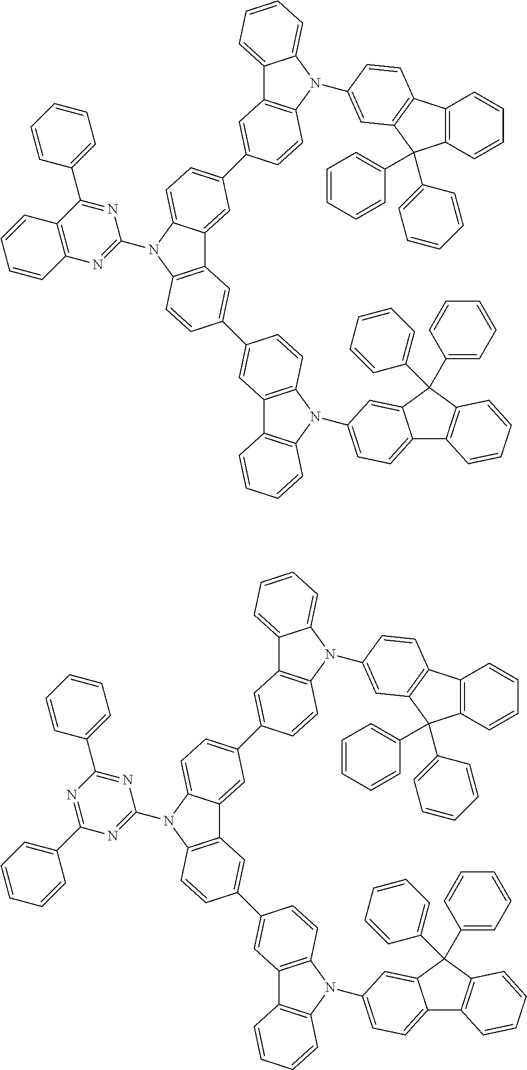 [SMILES: c1ccc(-c2nc(-n3c4ccc(-c5ccc6c(c5)c5ccccc5n6-c5ccc6c(c5)C(c5ccccc5)(c5ccccc5)c5ccccc5-6)cc4c4cc(-c5ccc6c(c5)c5ccccc5n6-c5ccc6c(c5)C(c5ccccc5)(c5ccccc5)c5ccccc5-6)ccc43)nc3ccccc23)cc1 ; c1ccc(-c2nc(-c3ccccc3)nc(-n3c4ccc(-c5ccc6c(c5)c5ccccc5n6-c5ccc6c(c5)C(c5ccccc5)(c5ccccc5)c5ccccc5-6)cc4c4cc(-c5ccc6c(c5)c5ccccc5n6-c5ccc6c(c5)C(c5ccccc5)(c5ccccc5)c5ccccc5-6)ccc43)n2)cc1]

### Organic Electroluminescence Device

An organic EL device as one embodiment of the present invention will be described.

The organic EL device contains a cathode, an anode, and one or more organic thin film layers between the cathode and the anode. The one or more organic thin film layers includes a light emitting layer, and at least one layer of the one or more organic thin film layers contains the compound of the present invention (which is the compounds (A) to (B) and compounds belonging to the subordinate concept that is encompassed thereby, which may be abbreviated simply as the compound).

Examples of the organic thin film layer that contains the compound of the present invention include the anode-side organic thin film layer (such as a hole transporting layer and a hole injecting layer) provided between the anode and the light emitting layer, the light emitting layer, the cathode-side organic thin film layer (such as an electron transporting layer and an electron injecting layer) provided between the cathode and the light emitting layer, a space layer, and a blocking layer, but the organic thin film layer is not limited to these layers. The compound may be contained in any of these layers, and may be used, for example, as a host material or a dopant material in a light emitting layer of a fluorescent light emitting unit, a host material in a light emitting layer of a phosphorescent light emitting unit, a hole transporting layer material or an electron transporting layer material of a light emitting unit, or the like. In particular, the compound is preferably contained in a light emitting layer, and in this case, the compound of the present invention is capable of functioning as a host material.

In one embodiment of the present invention, the organic EL device may be a fluorescent or phosphorescent light emission type monochromic light emitting device or a fluorescent-phosphorescent hybrid type white light emitting device, or may be a simple type having a sole light emitting unit or a tandem type having plural light emitting units, and among these, a phosphorescent light emission type one is preferred. The light emitting unit referred herein means a minimum unit that contains one or more organic layers, in which one of the layers is a light emitting layer capable of emitting light through recombination of holes and electrons injected thereto.

Accordingly, examples of the representative device structure of the simple type organic EL device include the following device structure.

(1) Anode/Light Emitting Unit/Cathode

The light emitting unit may be a laminated type having plural phosphorescent light emitting layers and fluorescent light emitting layers, and in this case, a space layer may be provided between the light emitting layers for preventing excitons formed in the phosphorescent light emitting layer from being diffused to the fluorescent light emitting layer. Representative examples of the layer structure of the light emitting unit are shown below.

(a) hole transporting layer/light emitting layer (/electron transporting layer)

(b) hole transporting layer/first phosphorescent light emitting layer/second phosphorescent light emitting layer (/electron transporting layer)

(c) hole transporting layer/phosphorescent light emitting layer/space layer/fluorescent light emitting layer (/electron transporting layer)

(d) hole transporting layer/first phosphorescent light emitting layer/second phosphorescent light emitting layer/space layer/fluorescent light emitting layer (/electron transporting layer)

(e) hole transporting layer/first phosphorescent light emitting layer/space layer/second phosphorescent light emitting layer/space layer/fluorescent light emitting layer (/electron transporting layer)

(f) hole transporting layer/phosphorescent light emitting layer/space layer/first fluorescent light emitting layer/second fluorescent light emitting layer (/electron transporting layer)

(g) hole transporting layer/electron blocking layer/light emitting layer (/electron transporting layer)

(h) hole transporting layer/light emitting layer/hole blocking layer (/electron transporting layer)

(i) hole transporting layer/fluorescent light emitting layer/triplet blocking layer (/electron transporting layer)

The phosphorescent or fluorescent light emitting layers may be ones that exhibit different light emission colors. Specifically, in the laminated light emitting unit (d), examples of the structure include a layer structure including hole transporting layer/first phosphorescent light emitting layer (red light emission)/second phosphorescent light emitting layer (green light emission)/space layer/fluorescent light emitting layer (blue light emission)/electron transporting layer.

An electron blocking layer may be appropriately provided between the light emitting layers and the hole transporting layer or the space layer. A hole blocking layer may be appropriately provided between the light emitting layers and the electron transporting layer. The electron blocking layer and the hole blocking layer provided may confine electrons or holes in the light emitting layer to increase the recombination probability of the charge in the light emitting layer, and thereby the light emission efficiency can be enhanced.

Examples of the representative device structure of the tandem type organic EL device include the following device structure.

(2) Anode/First Light Emitting Unit/Intermediate Layer/Second Light Emitting Unit/Cathode

The first light emitting unit and the second light emitting unit herein may be independently selected, for example, from ones that have the similar structure as the light emitting unit described above.

The intermediate layer may also be generally referred to as an intermediate electrode, an intermediate conductive layer, a charge generating layer, an electron withdrawing layer, a connecting layer, or an intermediate insulating layer, and may have known materials and structures capable of feeding electrons to the first light emitting unit and feeding holes to the second light emitting unit.

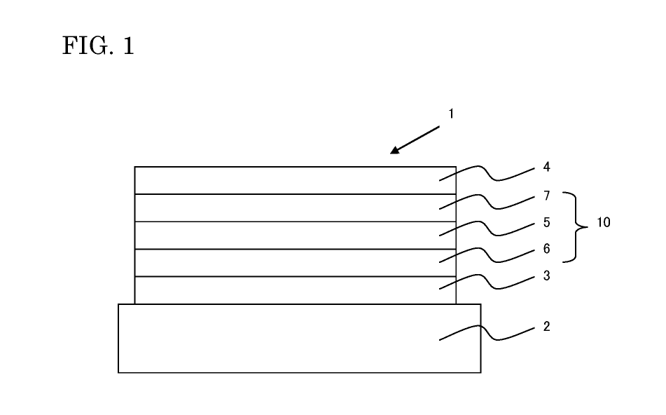

FIG. 1 shows a schematic structure of the organic EL device. The organic EL device 1 has a substrate 2, an anode 3, a cathode 4, and a light emitting unit 10 provided between the anode 3 and the cathode 4. The light emitting unit 10 has a light emitting layer 5 containing at least one phosphorescent light emitting layer containing a phosphorescent host material and a phosphorescent dopant (phosphorescent light emitting material). A hole injecting and transporting layer (anode-side organic thin film layer) 6 and the like may be provided between the light emitting layer 5 and the anode 3, and an electron injecting and transporting layer (cathode-side organic thin film layer) 7 and the like may be provided between the light emitting layer 5 and the cathode 4. An electron blocking layer (which is not shown in the FIGURE) may be provided on the side of the anode 3 of the light emitting layer 5, and a hole blocking layer (which is not shown in the FIGURE) may be provided on the side of the cathode 4 of the light emitting layer. According to the structure, electrons and holes can be confined in the light emitting layer 5, and thereby the formation probability of excitons in the light emitting layer 5 can be further enhanced.

In the present invention, a host that is combined with a fluorescent dopant (fluorescent material) is referred to as a fluorescent host, and a host that is combined with a phosphorescent dopant is referred to as a phosphorescent host. The fluorescent host and the phosphorescent host are not distinguished from each other not only from the molecular structures. Therefore, the phosphorescent host means a material that forms a phosphorescent light emitting layer containing a phosphorescent dopant, but does not mean that the phosphorescent host cannot be used as a material for forming a fluorescent light emitting layer. The same is applied to the fluorescent host.

### Substrate

The substrate is used as a support of the light emitting device. Examples of the substrate used include glass, quartz, and plastics. A flexible substrate may be used. The flexible substrate means a substrate that may be folded (flexible), and examples thereof include a plastic substrate formed of polycarbonate, polyvinyl chloride or the like.

### Anode

The anode formed on the substrate used is preferably a metal, an alloy, and an electroconductive compound that have a large work function (specifically 4.0 eV or more), a mixture thereof, and the like. Specific examples thereof include indium oxide-tin oxide (ITO: indium tin oxide), indium oxide-tin oxide containing silicon or silicon oxide, indium oxide-zinc oxide, indium oxide containing tungsten oxide and zinc oxide, and graphene. Examples thereof also include gold (Au), platinum (Pt), and a nitride of a metal material (such as titanium nitride).

### Cathode

The cathode used is preferably a metal, an alloy, and an electroconductive compound that have a small work function (specifically 3.8 eV or less), a mixture thereof, and the like. Specific examples of the cathode material include an element belonging to the group 1 and the group 2 of the periodic table, i.e., an alkali metal, such as lithium (Li) and cesium (Cs), an alkaline earth metal, such as magnesium (Mg), an alloy containing them (such as MgAg and AlLi), and a rare earth metal and an alloy containing a rare earth metal.

### Guest Material of Light Emitting Layer

The light emitting layer is a layer that contains a substance having a high light emitting capability, and various materials may be used. Examples of the substance having a high light emitting capability used include a fluorescent compound emitting fluorescent light and a phosphorescent compound emitting phosphorescent light. The fluorescent compound is a compound capable of emitting light from the singlet excited state, and the phosphorescent compound is a compound capable of emitting light from the triplet excited state.

Examples of the blue fluorescent light emitting material capable of being used in the light emitting layer include a pyrene derivative, a styrylamine derivative, a chrysene derivative, a fluoranthene derivative, a fluorene derivative, a diamine derivative, and a triarylamine derivative. Specific examples thereof include N,N′-bis[4-(9H-carbazol-9-yl)phenyl]-N,N′-diphenylstilbene-4,4′-diamine (abbreviation: YGA2S), 4-(9H-carbazol-9yl)-4′-(10-phenyl-9-anthryl)triphenylamine (abbreviation: YGAPA), and 4-(10-phenyl-9-anthryl)-4′-(9-phenyl-9H-carbazol-3-yl)triphenylamine (abbreviation: PCBAPA).

Examples of the green fluorescent light emitting material capable of being used in the light emitting layer include an aromatic amine derivative. Specific examples thereof include N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl)-N,N′,N′-triphenyl-1,4-phenylenediamine (abbreviation: 2 DPABPhA), N-[9,10-bis(1,1′-biphenyl-2-yl)]-N-[4-(9H-carbazol-9-yl)phenyl]-N-phenylanthracen-2-amine (abbreviation: 2YGABPhA), and N,N,9-triphenylanthracen-9-amine (abbreviation: DPhAPhA).

Examples of the red fluorescent light emitting material capable of being used in the light emitting layer include a tetracene derivative and a diamine derivative. Specific examples thereof include N,N,N′,N′-tetrakis(4-methylphenyl)tetracen-5,11-diamine (abbreviation: p-mPhTD) and 7,14-diphenyl-N, N,N′,N′-tetrakis(4-methylphenyl)acenaphtho[1,2-a]fluoranthen-3,10-diamine (abbreviation: p-mPhAFD).

Examples of the blue phosphorescent light emitting material capable of being used in the light emitting layer include a metal complex, such as an iridium complex, an osmium complex, and a platinum complex, and preferably an ortho-metalated complex of iridium, osmium, or platinum metal. Specific examples thereof include bis[2-(4′,6′-difluorophenyl)pyridinato-N,C2′]iridium(III) tetrakis(1-pyrazolyl)borate (abbreviation: Flr6), bis[2-(4′,6′-difluorophenyl)pyridinato-N,C2′]iridium(III) picolinate (abbreviation: Flrpic), bis[2-(3′,5′-bistrifluoromethylphenyl)pyridinato-N,C2′]iridium(III) picolinate (abbreviation: Ir(CF₃ppy)₂(pic)), and bis[2-(4′,6′-difluorophenyl)pyridinato-N,C2′]iridium(III) acetylacetonate (abbreviation: Flracac).

Examples of the green phosphorescent light emitting material capable of being used in the light emitting layer include an iridium complex. Examples thereof include tris(2-phenylpyridinato-N,C2′)iridium(III) (abbreviation: Ir(ppy)₃), bis(2-phenylpyridinato-N,C2′)iridium(III) acetylacetonate (abbreviation: Ir(ppy)₂(acac)), bis(1,2-diphenyl-1H-benzimidazolato)iridium(III) acetylacetonate (abbreviation: Ir(pbi)₂(acac)), and bis(benzo[h]quinolinato)iridium(III) acetylacetonate (abbreviation: Ir(bzq)₂(acac)).

Examples of the red phosphorescent light emitting material capable of being used in the light emitting layer include a metal complex, such as an iridium complex, a platinum complex, a terbium complex, and a europium complex. Specific examples thereof include an organic metal complex, such as bis[2-(2′-benzo[4,5-α]thienyl)pyridinato-N,C3′]iridium(III) acetylacetonate (abbreviation: Ir(btp)₂(acac)), bis(1-phenylisoquinolinato-N,C2′)iridium(III) acetylacetonate (abbreviation: Ir(piq)₂(acac)), (acetylacetonato) bis[2,3-bis(4-fluorophenyl)quinolinato)iridium(III) (abbreviation: Ir(Fdpq)₂(acac)), and 2,3,7,8,12,13,17,18-octaethyl-21H,23H-porphyrin platinum(II) (abbreviation: PtOEP).

Furthermore, a rare earth metal complex, such as tris(acetylacetonate) (monophenanthroline) terbium(III) (abbreviation: Tb(acac)₃(Phen)), tris(1,3-diphenyl-1,3-propanedioanto) (monophenanthroline)europium(III) (abbreviation: Eu(DBM)₃(Phen)), and tris[1-(2-thenoyl)-3,3,3-trifluoroacetonato] (monophenanthroline)europium(III) (abbreviation: Eu(TTA)₃(Phen)), may be used as a phosphorescent compound due to the light emission from a rare earth metal ion (electron transition between different multiplicities).

### Host Material of Light Emitting Layer

The light emitting layer may have a structure containing the aforementioned substance having a high light emitting capability (guest material) dispersed in another substance (host material). The substance used for dispersing the substance having a high light emitting capability may be various substances, and a substance that has a higher lowest unoccupied molecular orbital level (LUMO level) and a lower highest occupied molecular orbital level (HOMO level) than the substance having a high light emitting capability is preferably used.

The substance (host material) for dispersing the substance having a high light emitting capability is preferably the compound of the present invention. In addition to the compound of the present invention, for example, (1) a metal complex, such as an aluminum complex, a beryllium complex, and a zinc complex, (2) a heterocyclic compound, such as an oxadiazole derivative, a benzimidazole derivative, and a phenanthroline derivative, (3) a condensed aromatic compound, such as a carbazole derivative, an anthracene derivative, a phenanthrene derivative, a pyrene derivative, and a chrysene derivative, and (4) an aromatic amine compound, such as a triarylamine derivative and a condensed polycyclic aromatic amine derivative, may be used. More specific examples thereof include a metal complex, such as tris(8-quinolinolato)aluminum(III) (abbreviation: Alq), tris(4-methyl-8-quinolinolato)aluminum(III) (abbreviation: Almq3), bis(10-hydroxybenzo[h]quinolinato)beryllium(II) (abbreviation: BeBq2), bis(2-methyl-8-quinolinolato) (4-phenylphenolato)aluminum(III) (abbreviation: BAlq), bis(8-quinolinolato)zinc(II) (abbreviation: Znq), bis[2-(2-benzoxazolyl)phenolato] zinc(II) (abbreviation: ZnPBO), and bis[2-(2-benzothiazolyl)phenolato] zinc(II) (abbreviation: ZnBTZ), a heterocyclic compound, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1, 2, 4-triazole (abbreviation: TAZ), 2,2′,2″-(1,3,5-benzentriyl)-tris(1-phenyl-1H-benzimidazole) (abbreviation: TPBI), bathophenanthroline (abbreviation: BPhen), and bathocuproin (abbreviation: BCP), a condensed aromatic compound, such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilben-3,3′-diyl)diphenanthrene (abbreviation: DPNS), 9,9′-(stilben-4,4′-diyl)diphenanthrene (abbreviation: DPNS2), 3,3′,3″-(benzen-1,3,5-triyl)tripyrene (abbreviation: TPB3), 9,10-diphenylanthracene (abbreviation: DPAnth), and 6,12-dimethoxy-5,11-diphenylchrysene, and an aromatic amine compound, such as N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), N,9-diphenyl-N-[4-(10-phenyl-9-anthryl)phenyl]-9H-carbazol-3-amine (abbreviation: PCAPA), N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N, 9-diphenyl-9H-carbazol-3-amine (abbreviation: 2PCAPA), NPB (or α-NPD), TPD, DFLDPBi, and BSPB. Plural kinds of the substances (host materials) for dispersing the substance having a high light emitting capability (guest material) may be used.

### Electron Transporting Layer

The electron transporting layer is a layer that contains a substance having a high electron transporting capability. The electron transporting layer may contain (1) a metal complex, such as an aluminum complex, a beryllium complex, and a zinc complex, (2) a heteroaromatic compound, such as an imidazole derivative, a benzimidazole derivative, an azine derivative, a carbazole derivative, and a phenanthroline derivative, and (3) a polymer compound.

### Electron Injection Layer

The electron injecting layer is a layer that contains a substance having a high electron injecting capability. The electron injecting layer may contain an alkali metal, an alkaline earth metal, or compounds thereof, such as lithium (Li), lithium fluoride (LiF), cesium fluoride (CsF), calcium fluoride (CaF₂), a lithium oxide (LiOₓ) and the like.

### Hole Injection Layer

The hole injecting layer is a layer that contains a substance having a high hole injecting capability. Examples of the substance having a high hole injecting capability used include a molybdenum oxide, a titanium oxide, a vanadium oxide, a rhenium oxide, a ruthenium oxide, a chromium oxide, a zirconium oxide, a hafnium oxide, a tantalum oxide, a silver oxide, a tungsten oxide, a manganese oxide, an aromatic amine compound, and a polymer compound (such as an oligomer, a dendrimer, and a polymer).

### Hole Transporting Layer

The hole transporting layer is a layer that contains a substance having a high hole transporting capability. The hole transporting layer may contain a carbazole derivative, an anthracene derivative and the like. A polymer compound, such as poly(N-vinylcarbazole) (abbreviation: PVK) and poly(4-vinyltriphenylamine) (abbreviation: PVTPA), may also be used. Other materials that have a higher transporting capability for holes than that for electrons may be used. The layer containing the substance having a high hole transporting capability may be a single layer and may be in the form of two or more layers each formed of the substance laminated on each other.

In one embodiment of the present invention, the layers of the organic EL device may be formed by the ordinarily known method, such as a vapor deposition method and a spin coating method. For example, the layers may be formed by a known method, for example, a vapor deposition method, a molecular beam epitaxy method (MBE method), and a coating method, such as a dipping method, a spin coating method, a casting method, a bar coating method, and a roll coating method, which use a solution of a compound forming a layer.

The layers are not particularly limited in thickness, and in general, when the thickness is too small, defects, such as pinholes, are liable to form, and when the thickness is too large, a higher driving voltage may be required to deteriorate the efficiency. Accordingly, the thickness is generally preferably from several nm to 1 μm.

The layers (e.g., the light emitting layer, the hole transporting layer, and the electron transporting layer) containing the compound of the present invention are preferably formed by the coating method using a solution containing a solvent and the compound (i.e., an ink composition). The ink composition may contain other materials, such as a dopant, depending on necessity.

The coating method used is preferably a wet film forming method, and examples thereof capable of being applied include a relief printing method, an intaglio printing method, a planographic printing method, a stencil printing method, and a printing method combining an offset printing method with these printing methods, an ink-jet printing method, a dispenser coating method, a spin coating method, a bar coating method, a dip coating method, a spray coating method, a slit coating method, a roll coating method, a cap coating method, a gravure roll coating method, and a meniscus coating method. In the case where a particularly fine patterning is required, a relief printing method, an intaglio printing method, a planographic printing method, a stencil printing method, and a printing method combining an offset printing method with these printing methods, an ink-jet printing method, a dispenser coating method and the like are preferred. Furthermore, such a method may also be used that the polymer is formed into a film by the wet film forming method on a transfer substrate precursor, and then transferred to the target circuit board having an electrode with laser light or heat press.

The formation of the films by these methods may be performed under conditions having been known in the art, and the details thereof are omitted herein.

In the step of drying and removing an organic solvent after the coating film formation, there are cases where heat drying is performed. The heat drying temperature is preferably such a temperature that is capable of sufficiently removing the organic solvent used for coating and is lower than the temperature, at which the compound is heat decomposed.

In this point of view, the heat drying temperature range on drying after the coating film formation is preferably 50° C. or more, more preferably 80° C. or more, further preferably 100° C. or more, and still further preferably 140° C. or more, and is preferably 300° C. or less, more preferably 250° C. or less, and further preferably 240° C. or less.

The coating liquid used for the coating method (i.e., the ink composition) may contain at least one kind of the compound (1), which may be dissolved or dispersed in a solvent.

The content of the compound (1) in the coating liquid (ink composition) is preferably from 0.1 to 15% by mass, and more preferably from 0.5 to 10% by mass, based on the total solution for forming a film.

The solvent is preferably an organic solvent, and examples of the organic solvent include a chlorine solvent, such as chloroform, chlorobenzene, chlorotoluene, chloroxylene, chloroanisole, dichloromethane, dichlorobenzene, dichlorotoluene, dichloroethane, trichloroethane, trichlorobenzene, trichloromethylbenzene, bromobenzene, dibromobenzene, and bromoanisole, an ether solvent, such as tetrahydrofuran, dioxane, dioxolane, oxazole, methylbenzoxazole, benzisoxazole, furan, furazan, benzofurazan, and dihydrobenzofuran, an aromatic hydrocarbon solvent, such as ethylbenzene, diethylbenzene, triethylbenzene, trimethylbenzene, trimethoxybenzene, propylbenzene, isopropylbenzene, diisopropylbenzene, dibutylbenzene, amylbenzene, dihexylbenzene, cyclohexylbenzene, tetramethylbenzene, dodecylbenzene, benzonitrile, acetophenone, methylacetophenone, methoxyacetophenone, ethyl toluate, toluene, ethyltoluene, methoxytoluene, dimethoxytoluene, trimethoxytoluene, isopropyltoluene, xylene, butylxylene, isopropylxylene, anisole, ethylanisole, dimethylanisole, trimethylanisole, propylanisole, isopropylanisole, butylanisole, methylethylanisole, anethole, anisyl alcohol, methyl benzoate, ethyl benzoate, propyl benzoate, butyl benzoate, diphenyl ether, butyl phenyl ether, benzyl methyl ether, benzyl ethyl ether, methylenedioxybenzene, methylnaphthalene, tetrahydronaphthalene, aniline, methylaniline, ethylaniline, butylaniline, biphenyl, methylbiphenyl, and isopropylbiphenyl, an aliphatic hydrocarbon solvent, such as cyclohexane, methylcyclohexane, n-pentane, n-hexane, n-heptane, n-octane, n-nonane, n-decane, tetradecane, decalin, and isopropylcyclohexane, a ketone solvent, such as acetone, methyl ethyl ketone, cyclohexanone, and acetophenone, an ester solvent, such as ethyl acetate, butyl acetate, ethyl cellosolve acetate, methyl benzoate, and phenyl acetate, a polyhydric alcohol and a derivative thereof, such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerin, and 1,2-hexanediol, an alcohol solvent, such as methanol, ethanol, propanol, isopropanol, and cyclohexanol, a sulfoxide solvent, such as dimethylsulfoxide, and an amide solvent, such as N-methyl-2-pyrrolidone and N,N-dimethylformamide. The organic solvents may be used solely or as a combination of plural kinds thereof.

In the solvents described above, at least one of toluene, xylene, ethylbenzene, amylbenzene, anisole, 4-methoxytoluene, 2-methoxytoluene, 1,2-dimethoxybenzene, mesitylene, tetrahydronaphthalene, cyclohexylbenzene, 2,3-dihydrobenzofuran, cyclohexanone, and methylcyclohexanone is preferably contained from the standpoint of the solubility, the uniformity of film formation, the viscosity characteristics, and the like.

In the solvents described above, a solvent shown by the following general formula (5), which has a boiling point of 110° C. or more and a solubility in water at 20° C. of 1% by mass or less, is more preferably used with the compound.

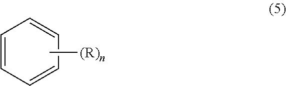

wherein in the general formula (5), R each independently represent a substituent having from 1 to 20 carbon atoms, and n represents an integer of from 0 to 6.

The coating liquid for film formation (ink composition) that contains the compound and the solvent shown by the general formula (5), which has a boiling point of 110° C. or more and a solubility in water at 20° C. of 1% by mass or less, is preferred.

The coating liquid for film formation (ink composition) may contain, depending on necessity, a viscosity modifier, a surface tension modifier, a crosslinking reaction initiator, and a crosslinking reaction catalyst. The viscosity modifier, the surface tension modifier, the crosslinking reaction initiator, and the crosslinking reaction catalyst are preferably ones that do not influence the device characteristics when they remain in the film or ones that are capable of being removed from the film in the film forming process.

In one embodiment of the present invention, the organic electroluminescence device may be used in an electronic equipment, for example, a display part, such as an organic EL panel module, a display device of a television set, a portable telephone, a personal computer or the like, and a light emitting device, such as an illumination and a vehicle lamp. Therefore, the present invention also provides an electronic equipment having the organic EL device mounted thereon.

### EXAMPLE

The present invention will be described in more detail with reference to examples and comparative examples below, but the present invention is not limited to the descriptions of the examples.

### Synthesis Example 1

### Synthesis of Compound H-1

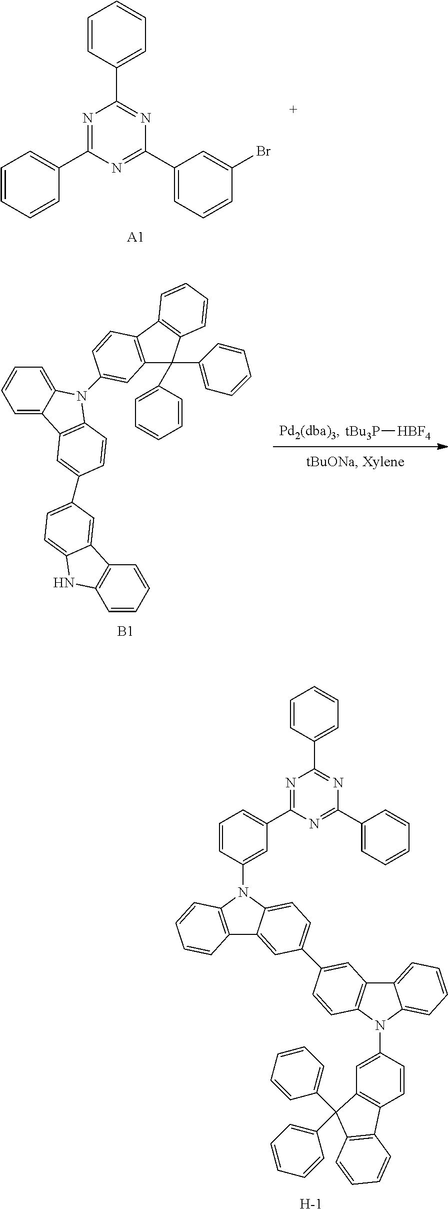

Under an argon atmosphere, a carbazolyl intermediate B1 (2.60 g, 4.00 mmol), a triazine intermediate A1 (1.55 g, 4.00 mmol), tris(dibenzylideneacetone)dipalladium (0.147 g, 0.160 mmol), tri-t-butylphosphonium tetrafluoroborate (0.186 g, 0.640 mmol), sodium t-butoxide (1.15 g, 12.0 mmol), and anhydrous xylene (80 mL) were added sequentially and refluxed under heating for 8 hours.

After cooling the reaction liquid to room temperature, the insoluble matters were removed by filtration, and the organic solvent was distilled off under reduced pressure. The resulting residue was purified by silica gel column chromatography, thereby providing the compound H-1 (3.21 g, yield: 84%).

The analysis results of HPLC (high performance liquid chromatography) and LC-MS (liquid chromatography-mass spectrometry) of the compound H-1 are shown below.

HPLC: purity 99.7%

LC-MS: calculated: C70H45N5=955.

found: m/z=955 (M+, 100).

### Synthesis Example 2

### Synthesis of Compound H-2

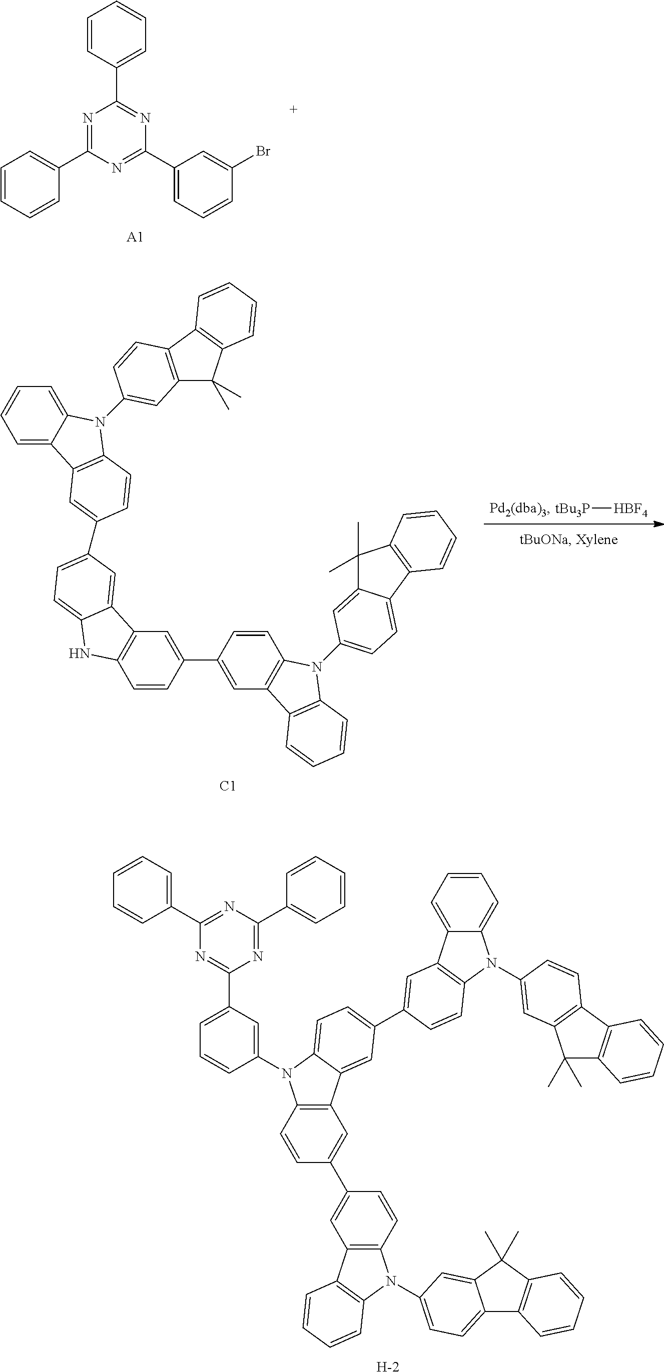

Under an argon atmosphere, a tricarbazolyl intermediate C1 (2.64 g, 2.99 mmol), a triazine intermediate A1 (1.16 g, 2.99 mmol), tris(dibenzylideneacetone)dipalladium (0.110 g, 0.120 mmol), tri-t-butylphosphonium tetrafluoroborate (0.139 g, 0.478 mmol), sodium t-butoxide (0.862 g, 0.897 mmol), and anhydrous xylene (60 mL) were added sequentially and refluxed under heating for 7.5 hours.

After cooling the reaction liquid to room temperature, the insoluble matters were removed by filtration, and the organic solvent was distilled off under reduced pressure. The resulting residue was purified by silica gel column chromatography, thereby providing the compound H-2 (2.32 g, yield: 65%).

The analysis results of HPLC (high performance liquid chromatography) and LC-MS (liquid chromatography-mass spectrometry) of the compound H-2 are shown below.

HPLC: purity 99.8%

LC-MS: calculated: C87H60N6=1,188.

found: m/z=1,188 (M+, 100).

### Synthesis Example 3

### Synthesis of Compound H-3

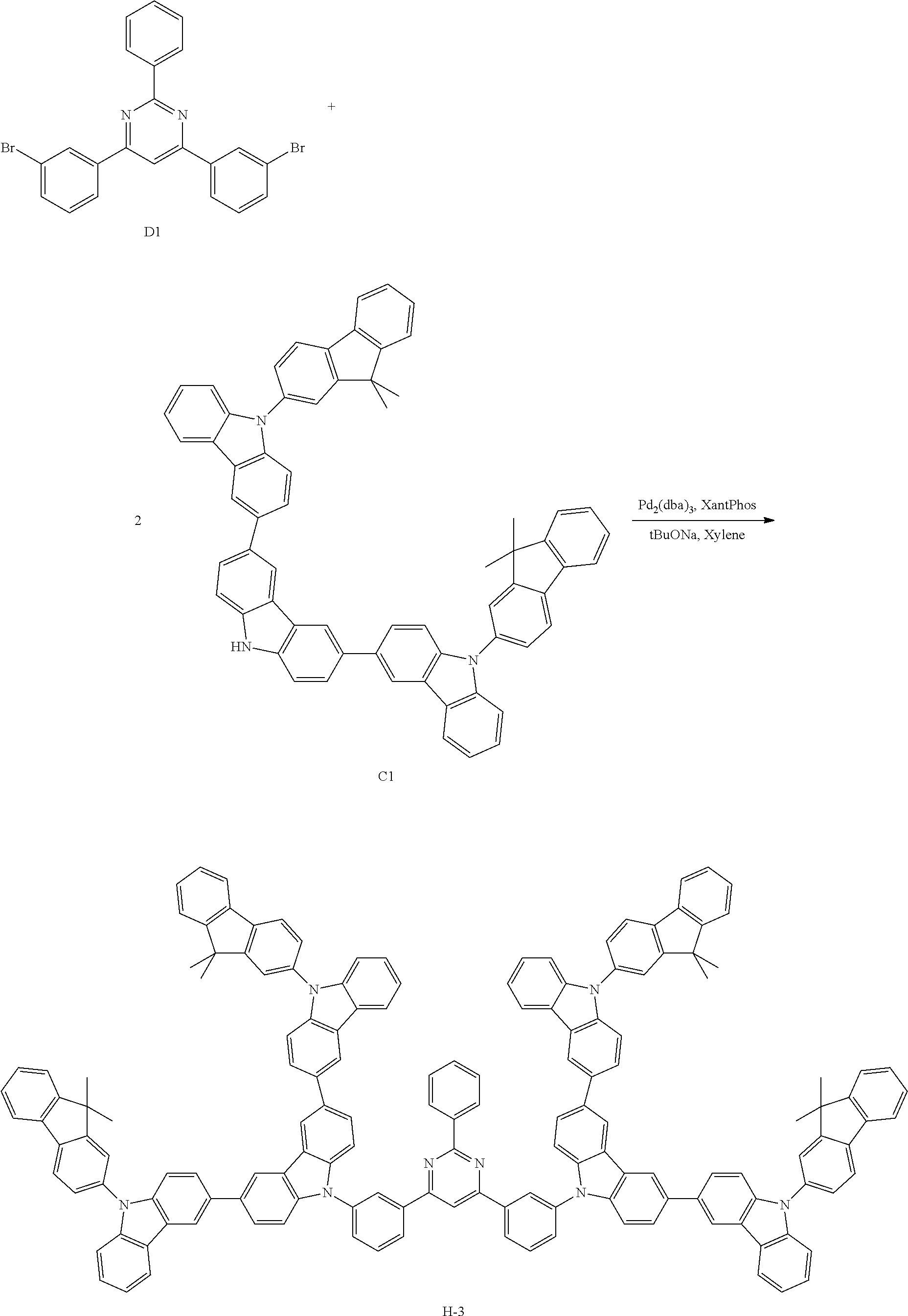

Under an argon atmosphere, a carbazolyl intermediate C1 (1.50 g, 1.70 mmol), a pyrimidine intermediate D1 (0.396 g, 0.850 mmol), tris(dibenzylideneacetone)dipalladium (0.0623 g, 0.0680 mmol), xantphos (0.0787 g, 0.136 mmol), sodium t-butoxide (0.245 g, 2.55 mmol), and anhydrous xylene (34 mL) were added sequentially and refluxed under heating for 8 hours.

After cooling the reaction liquid to room temperature, the insoluble matters were removed by filtration, and the organic solvent was distilled off under reduced pressure. The resulting residue was purified by silica gel column chromatography, thereby providing the compound H-3 (1.45 g, yield: 82%).

The analysis results of HPLC (high performance liquid chromatography) and LC-MS (liquid chromatography-mass spectrometry) of the compound H-3 are shown below.

HPLC: purity 98.5%

LC-MS: calculated: C154H106N8=2,066.

found: m/z=2,066 (M+, 100).

### Synthesis Example 4

### Synthesis of Compound H-4

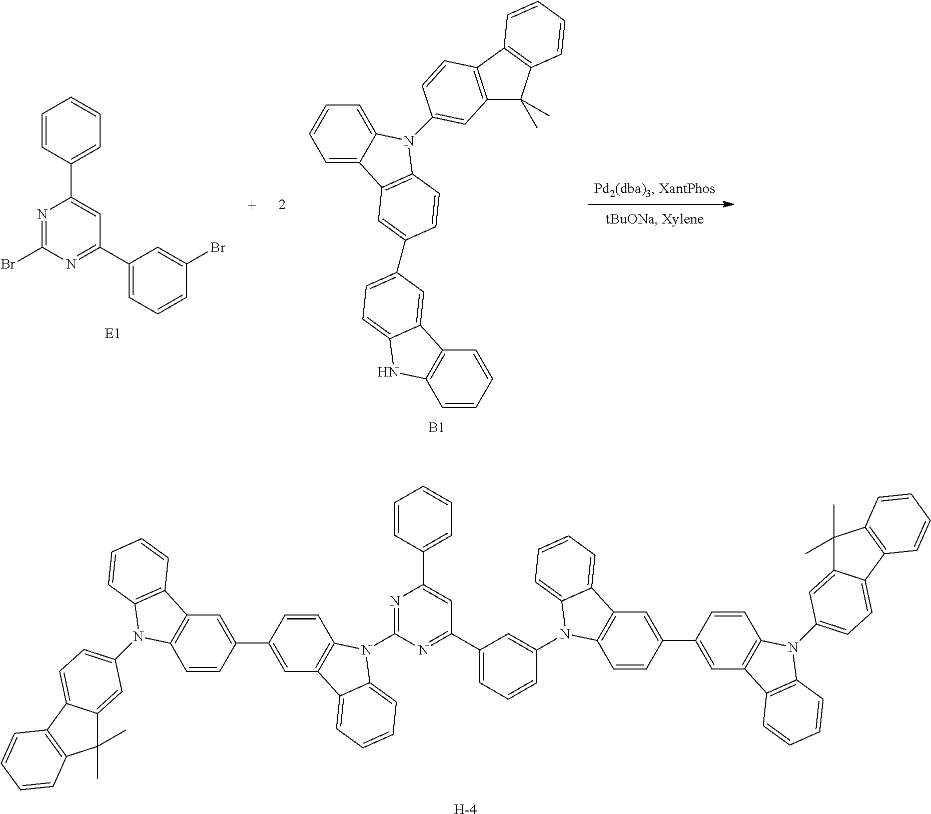

Under an argon atmosphere, a carbazolyl intermediate B1 (2.69 g, 5.13 mmol), a pyrimidine intermediate E1 (1.00 g, 2.56 mmol), tris(dibenzylideneacetone)dipalladium (0.0938 g, 0.102 mmol), xantphos (0.119 g, 0.205 mmol), sodium t-butoxide (0.738 g, 7.68 mmol), and anhydrous xylene (100 mL) were added sequentially and refluxed under heating for 8 hours.

After cooling the reaction liquid to room temperature, the insoluble matters were removed by filtration, and the organic solvent was distilled off under reduced pressure. The resulting residue was purified by silica gel column chromatography, thereby providing the compound H-4 (2.62 g, yield: 80%).

The analysis results of HPLC (high performance liquid chromatography) and LC-MS (liquid chromatography-mass spectrometry) of the compound H-4 are shown below.

HPLC: purity 99.0%

LC-MS: calculated: C94H64N6=1,276.

found: m/z=1,276 (M+, 100).

The compounds defined by the scope of claim can be synthesized by using known alternate reactions and raw materials corresponding to the target compounds with reference to the synthesis reactions shown above.

### Example 1

### Cleaning of Substrate

A glass substrate having a dimension of 25 mm×25 mm×1.1 mm in thickness having an ITO transparent electrode (produced by GEOMATEC Co., Ltd.) was subjected to ultrasonic cleaning in isopropyl alcohol for 5 minutes, and then subjected to UV ozone cleaning for 5 minutes.

### Formation of Underlayer

As a hole transporting material, Clevious AI 4083 (trade name), produced by Heraeus Deutschland GmbH & Co. KG, was formed into a film having a thickness of 30 nm on the ITO substrate by a spin coating method. After forming the film, the unnecessary portion thereof was removed with acetone, and the film was baked in the air with a hot-plate at 200° C. for 10 minutes, thereby producing an underlayer substrate.

### Formation of Light Emitting Layer

Using the compound H-1 obtained in the synthesis example as a host material, and the following compound D-1 as a dopant material, a toluene solution of 1.6% by weight thereof having a weight ratio (compound H-1)/(compound D-1) of 90/10 was prepared. The toluene solution was coated on the underlayer substrate by a spin coating method to a thickness of 50 nm. After coating the film, the unnecessary portion thereof was removed with toluene, and the film was heated and dried with a hot-plate at 150° C., thereby producing a coated and laminated substrate having a light emitting layer formed. The operations for forming the light emitting layer were all performed in a glove box under a nitrogen atmosphere.

### Vapor Deposition and Sealing

The coated and laminated substrate was transferred to a vapor deposition chamber, and the following compound ET-1 was vapor-deposited to 50 nm as an electron transporting layer. Furthermore, 1 nm of lithium fluoride and 80 nm of aluminum each were vapor-deposited and laminated. After completing all the vapor deposition processes, the substrate was sealed with counterbored glass in a glove box under a nitrogen atmosphere, thereby producing an organic EL device.

### Evaluation of Device

The resulting organic EL device was subjected to light emission by direct current driving, and the external quantum efficiency (EQE) at a current density of 10 mA/cm² was measured. The measurement result is shown in Table 1.

### Example 2

An organic EL device was produced in the same manner as in Example 1 except that the compound H-2 obtained in the synthesis example was used as the host material. The measurement result is shown in Table 1.

### Example 3

An organic EL device was produced in the same manner as in Example 1 except that the compound H-3 obtained in the synthesis example was used as the host material. The measurement result is shown in Table 1.

### Example 4

An organic EL device was produced in the same manner as in Example 1 except that the compound H-4 obtained in the synthesis example was used as the host material. The measurement result is shown in Table 1.

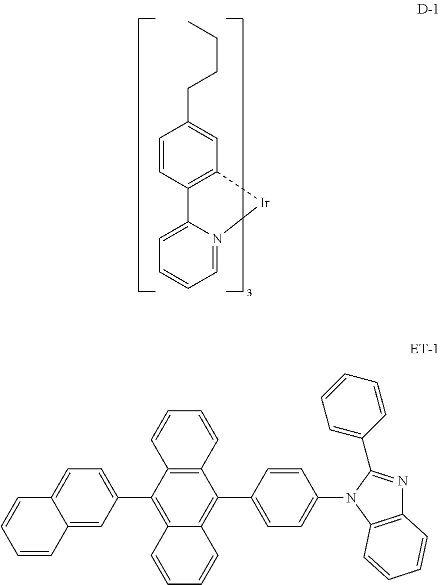

### Comparative Example 1

An organic EL device was tried to produce in the same manner as in Example 1 except that the following compound CEH-1 was used as the host material. However, on heating and drying the coated film, the film flowed to fail to provide a uniform film.

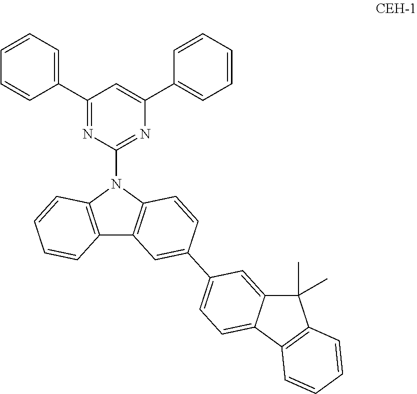 [SMILES: CC1(C)c2ccccc2-c2ccc(-c3ccc4c(c3)c3ccccc3n4-c3nc(-c4ccccc4)cc(-c4ccccc4)n3)cc21]

TABLE 1
Results of EQE Evaluation

| | Compound | EQE (%) |
|---|---|---|
| Example 1 | H-1 | 8.2% |
| Example 2 | H-2 | 9.1% |
| Example 3 | H-3 | 8.9% |
| Example 4 | H-4 | 8.7% |

It was confirmed from the examples that the compounds having the molecular structure according to the present invention had characteristics as an organic EL material.

### REFERENCE SIGNS LIST

1 organic EL device
2 substrate
3 anode
4 cathode
5 light emitting layer
6 anode-side organic thin film layer
7 cathode-side organic thin film layer
10 light emitting unit

1. A compound that is represented by the following general formulae (1) and (2), and contains 11 or more benzene rings in one molecule: 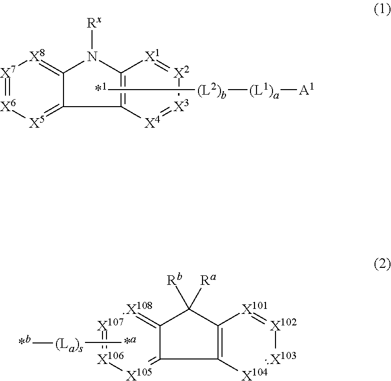
wherein
in the general formula (1),
A¹ represents a substituted or unsubstituted nitrogen-containing heteroaromatic group having from 5 to 30 ring atoms;
L¹ and L² each independently represent a substituted or unsubstituted aromatic hydrocarbon group having from 6 to 30 ring carbon atoms, a substituted or unsubstituted heterocyclic group having from 5 to 30 ring atoms, or a group containing from 2 to 4 of the groups bonded to each other;
a and b each independently represent 0 or 1, provided that when a is 0, (L¹)₀ represents a single bond, and when b is 0, (L²)₀ represents a single bond;
R^{x} represents a hydrogen atom or a substituent;
X¹ to X⁸ each represent C(R¹) to C(R⁸) or a nitrogen atom; and
R¹ to R⁸ each independently represent a hydrogen atom or a substituent, in which adjacent substituents may be bonded to each other to form a ring,
provided that any one of R¹ to R⁴ and R^{x} represents a single bond bonded to L² at the position of *¹,
in the general formula (2),
Lₐ represents a substituted or unsubstituted aromatic hydrocarbon group having from 6 to 30 ring carbon atoms or a substituted or unsubstituted heterocyclic group having from 5 to 30 ring atoms;
s represents 0 or 1, provided that when s is 0, (Lₐ)₀ represents a single bond;
X¹⁰¹ to X¹⁰⁸ each represent C(R¹⁰¹) to C(R¹⁰⁸) or a nitrogen atom, and R¹⁰¹ to R¹⁰⁸ each independently represent a hydrogen atom or a substituent, in which adjacent substituents may be bonded to each other to form a ring, provided that any one of R¹⁰⁵ to R¹⁰⁸ represents a single bond directly bonded to Lₐ at the position of *^{a}; and
R^{a} and R^{b} each independently represent a hydrogen atom, an alkyl group having from 1 to 10 carbon atoms, an aryl group having from 6 to 14 ring carbon atoms, or a heteroaryl group having from 5 to 14 ring atoms, in which R^{a} and R^{b} may be bonded to each other to form a ring, and R^{a} and R¹⁰¹, and R^{b} and R¹⁰⁸ each independently may be bonded to each other to form a ring,
the group represented by the formula (2) is bonded to at least one of L¹, L², R¹ to R⁸, and R^{x} at the position of *^{b}, in which any one of R¹ to R⁸, and R^{x} that is bonded to the group represented by the formula (2) represents a single bond, and
when plural groups each represented by the formula (2) are present, the groups may be the same as or different from each other.

2. The compound according to claim 1, wherein the compound is represented by the following general formulae (1a) and (2a): 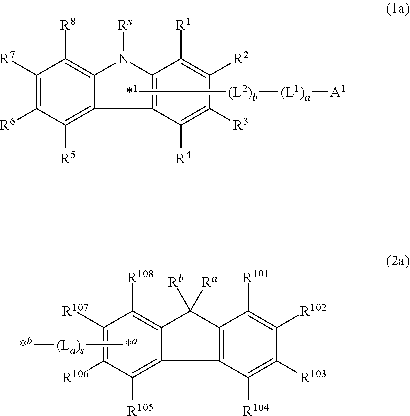
wherein
in the general formula (1a), A¹, L¹, L², a, b, R¹ to R⁸, and R^{x} have the same meanings as in claim 1, and
in the general formula (2a), Lₐ, s, R¹⁰¹ to R¹⁰⁸, R^{a}, and R^{b} have the same meanings as in claim 1.

3. The compound according to claim 2, wherein the compound is represented by the following general formulae (1a-1) and (2a): 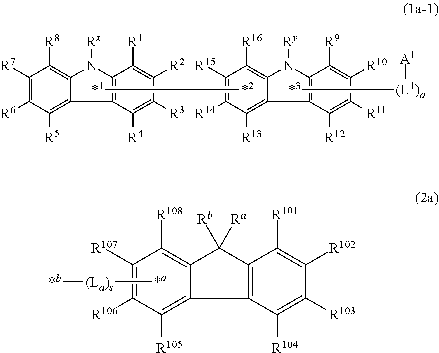
wherein
in the general formula (1a-1),
A¹, L¹, and a have the same meanings as in claim 2;
R^{x} and R^{y} each independently represent a hydrogen atom or a substituent; and
R¹ to R¹⁶ each independently represent a hydrogen atom or a substituent, in which adjacent substituents may be bonded to each other to form a ring,
provided that any one of R¹ to R⁴, and R^{x} represents a single bond bonded to *¹, any one of R¹³ to R¹⁶ represents a single bond bonded to *², and any one of R⁹ to R¹², and R^{y} represents a single bond bonded to L¹ at the position of *³, and
in the general formula (2a),
Lₐ, s, R¹⁰¹ to R¹⁰⁸, R^{a}, and R^{b} have the same meanings as in claim 2,
provided that the group represented by the formula (2a) is bonded to at least one of L¹, R¹ to R¹⁶, R^{x}, and R^{y} at the position of *^{b}.

4. The compound according to claim 3, wherein the compound is represented by the following general formula (1b-1): 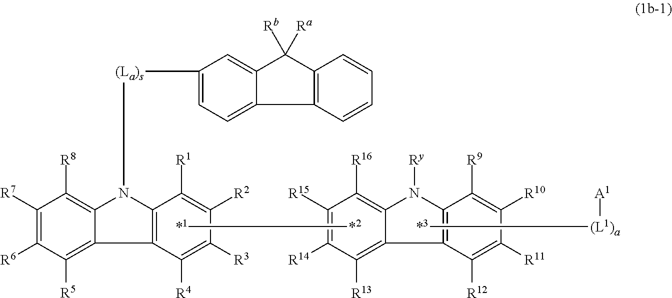
wherein in the general formula (1b-1), A¹, Lₐ, L¹, a, s, R¹ to R¹⁶, R^{y}, R^{a}, and R^{b} have the same meanings as in claim 3.

5. The compound according to claim 3, wherein the compound is represented by the following general formula (1b-2): 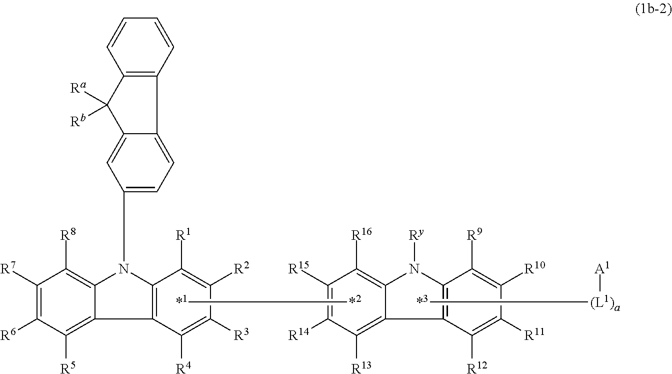
wherein in the general formula (1b-2), A¹, L¹, a, R¹ to R¹⁶, R^{y}, R^{a}, and R^{b} have the same meanings as in claim 3.

6. The compound according to claim 3, wherein the compound is represented by the following general formula (1b-3): 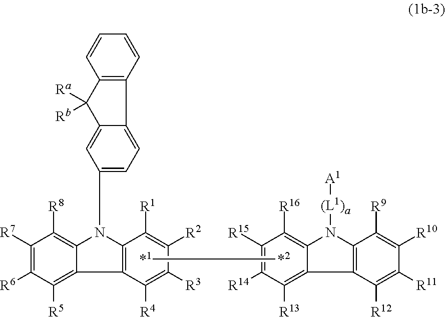
wherein in the general formula (1b-3), A¹, L¹, a, R¹ to R¹⁶, R^{a}, and R^{b} have the same meanings as in claim 3.

7. The compound according to claim 3, wherein the compound is represented by the following general formula (1b-4): 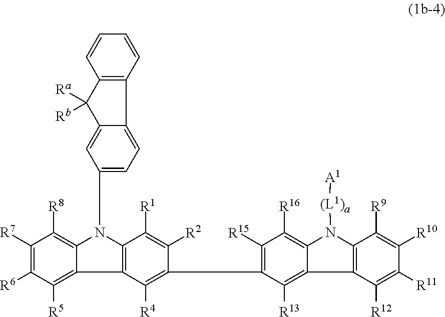
wherein in the general formula (1b-4), A¹, L¹, a, R², R⁴ to R¹³, R¹⁵, R¹⁶, R^{a}, and R^{b} have the same meanings as in claim 3.

8. The compound according to claim 3, wherein the compound is represented by the following general formulae (1a-a) and (2a): 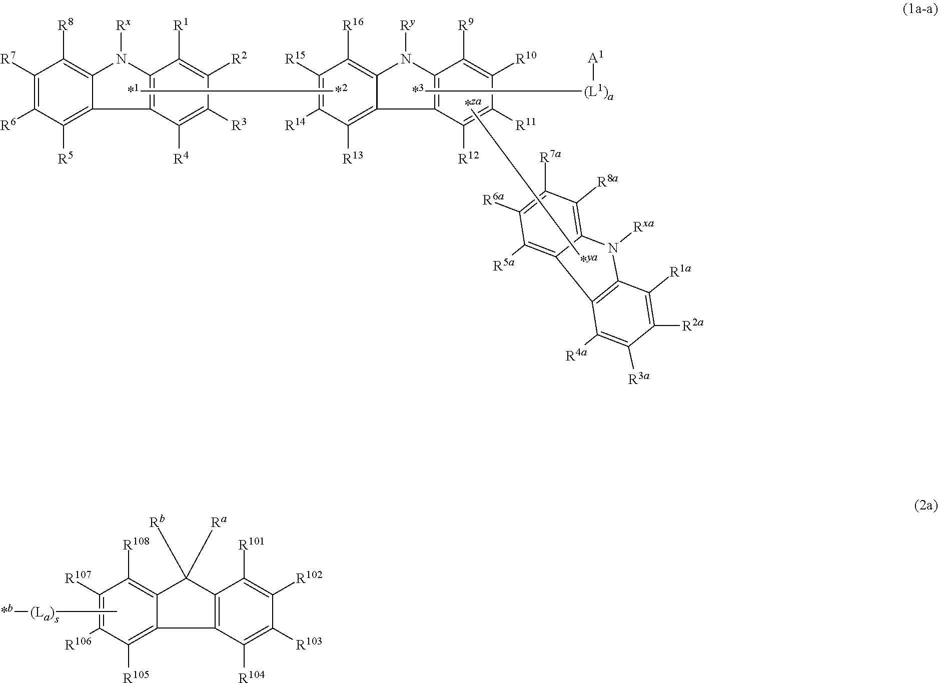
wherein
in the general formula (1a-a),
A¹, L¹, a, R¹ to R¹⁶, R^{x}, and R^{y} have the same meanings as in claim 3;
R^{xa} represents a hydrogen atom or a substituent; and
R^{1a} to R^{8a} each independently represent a hydrogen atom or a substituent, in which adjacent substituents may be bonded to each other to form a ring,
provided that any one of R^{5a} to R^{8a}, and R^{xa} represents a single bond bonded to *^{ya}, and any one of R⁹ to R¹² represents a single bond bonded to *^{za}, and
in the general formula (2a),
Lₐ, s, R¹⁰¹ to R¹⁰⁸, R^{a}, and R^{b} have the same meanings as in claim 3,
provided that the group represented by the formula (2a) is bonded to at least one of L¹, R¹ to R^{1b}, R^{1a} to R^{8a}, R^{x}, R^{y} and R^{xa} at the position of *^{b}.

9. The compound according to claim 3, wherein the compound is represented by the following general formulae (1-i) and (2a): 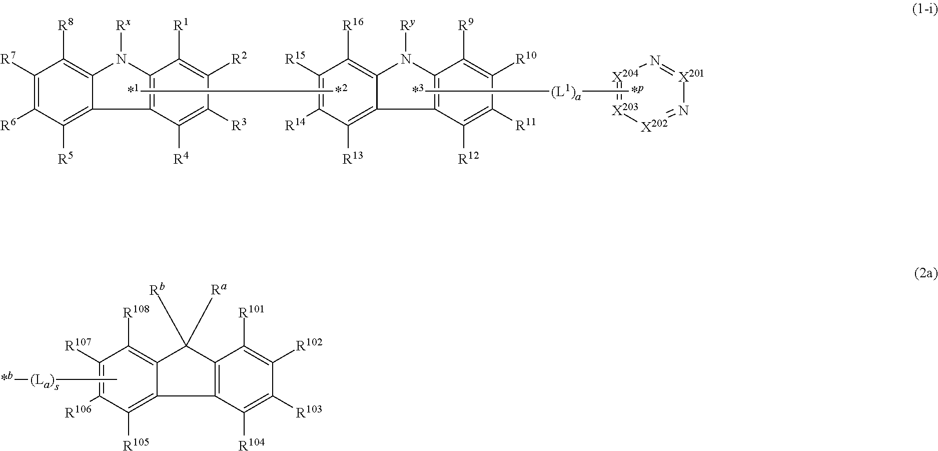
wherein
in the general formula (1-i),
L¹, a, R¹ to R¹⁶, R^{x}, and R^{y} have the same meanings as in claim 3; and
X²⁰¹ to X²⁰⁴ each represent C(R²⁰¹) to C(R²⁰⁴) or a nitrogen atom, and R²⁰¹ to R²⁰⁴ each independently represent a hydrogen atom or a substituent, in which adjacent substituents may be bonded to each other, provided that any one of R²⁰¹ to R²⁰⁴ represents a single bond directly bonded to L¹ at the position of *^{p}, and
in the general formula (2a),
Lₐ, s, R¹⁰¹ to R¹⁰⁸, R^{a}, and R^{b} have the same meanings as in claim 3.

10. The compound according to claim 3, wherein the compound is represented by the following general formulae (1-ii) and (2a): 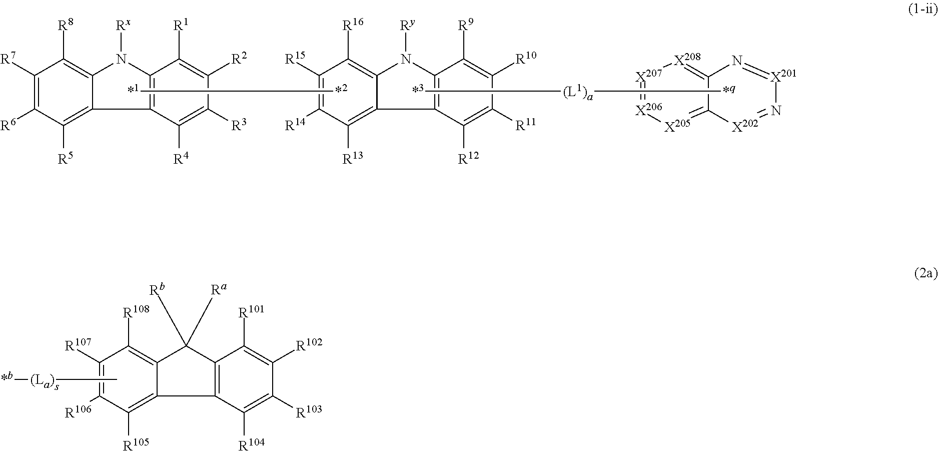
wherein
in the general formula (1-ii),
L¹, a, R¹ to R¹⁶, R^{x}, and R^{y} have the same meanings as in claim 3; and
X²⁰¹, X²⁰², and X²⁰⁵ to X²⁰⁸ each represent C(R²⁰¹), C(R²⁰²), and C(R²⁰⁵) to C(R²⁰⁸) or a nitrogen atom, and R²⁰¹, R²⁰², and R²⁰⁵ to R²⁰⁸ each independently represent a hydrogen atom or a substituent, in which adjacent substituents may be bonded to each other, provided that any one of R²⁰¹, R²⁰², and R²⁰⁵ to R²⁰⁸ represents a single bond directly bonded to L¹ at the position of *^{q}, and
in the general formula (2a),
Lₐ, s, R¹⁰¹ to R¹⁰⁸, R^{a}, and R^{b} have the same meanings as in claim 3.

11. The compound according to claim 3, wherein the compound is represented by the following general formulae (1-iii) and (2a): 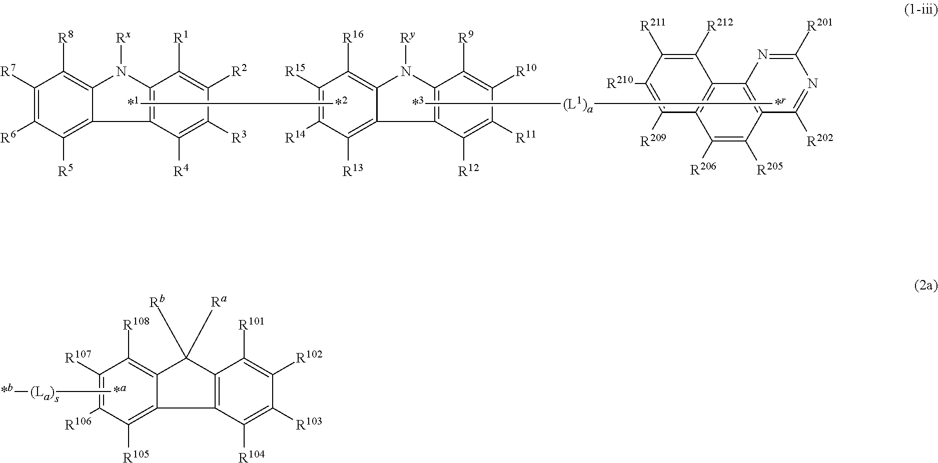
wherein
in the general formula (1-iii),
L¹, a, R¹ to R¹⁶, R^{x}, and R^{y} have the same meanings as in claim 3; and
R²⁰¹, R²⁰², R²⁰⁵, R²⁰⁶, and R²⁰⁹ to R²¹² each independently represent a hydrogen atom or a substituent, in which adjacent substituents may be bonded to each other, provided that any one of R²⁰¹, R²⁰², R²⁰⁵, R²⁰⁶, and R²⁰⁹ to R²¹² represents a single bond directly bonded to L¹ at the position of *^{r}, and
in the general formula (2a),
Lₐ, s, R¹⁰¹ to R¹⁰⁸, R^{a}, and R^{b} have the same meanings as in claim 3.

12. A compound that is represented by the following general formulae (3) and (4): 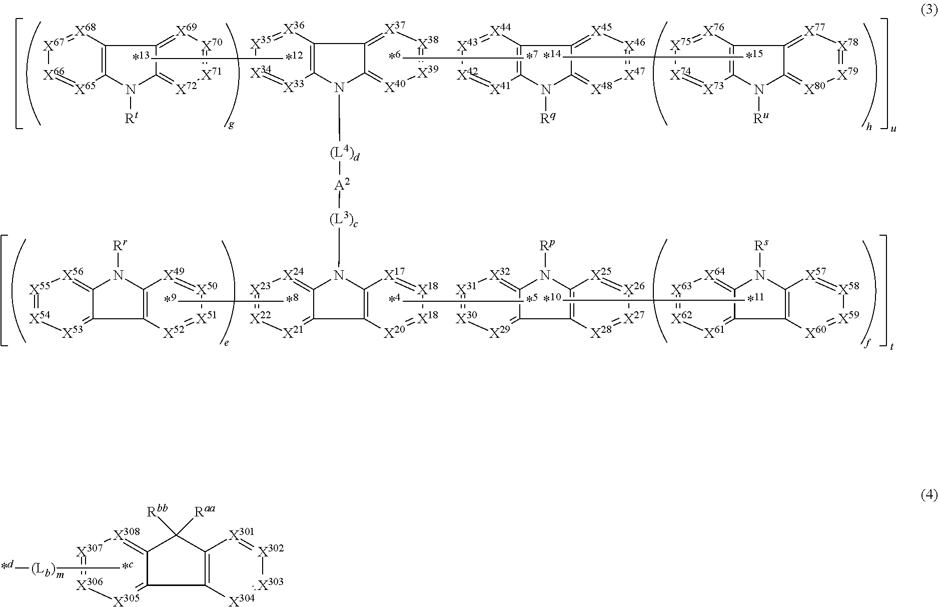
wherein
in the general formula (3),
A² represents a substituted or unsubstituted nitrogen-containing heteroaromatic group having from 5 to 30 ring atoms;
L³ and L⁴ each independently represent a substituted or unsubstituted aromatic hydrocarbon group having from 6 to 30 ring carbon atoms, a substituted or unsubstituted heterocyclic group having from 5 to 30 ring atoms, or a group containing from 2 to 4 of the groups bonded to each other;
c and d each independently represent 0 or 1, provided that when c is 0, (L³)₀ represents a single bond, and when d is 0, (L⁴)₀ represents a single bond;
R^{p} to R^{u} each independently represent a hydrogen atom or a substituent;
X¹⁷ to X⁸⁰ each represent C(R¹⁷) to C(R⁸⁰) or a nitrogen atom; and
R¹⁷ to R⁸⁰ each independently represent a hydrogen atom or a substituent, in which adjacent substituents may be bonded to each other to form a ring,
provided that any one of R¹⁷ to R²⁰ represents a single bond bonded to *⁴, any one of R^{p} and R²⁹ to R³² represents a single bond bonded to *⁵, any one of R³⁷ to R⁴⁰ represents a single bond bonded to *⁶, any one of R^{q} and R⁴¹ to R⁴⁴ represents a single bond bonded to *⁷, any one of R²¹ to R²⁴ represents a single bond bonded to *⁸, any one of R^{r} and R⁴⁹ to R⁵² represents a single bond bonded to *⁹, any one of R^{p} and R²⁵ to R²⁸ represents a single bond bonded to *¹⁰, any one of R^{s} and R⁶¹ to R⁶⁴ represents a single bond bonded to *¹¹, any one of R³³ to R³⁶ represents a single bond bonded to *¹², any one of R^{t} and R⁶⁹ to R⁷² represents a single bond bonded to *¹³, any one of R^{q} and R⁴⁵ to R⁴⁸ represents a single bond bonded to *¹⁴, and any one of R^{u} and R⁷³ to R⁷⁶ represents a single bond bonded to *¹⁵, and
when plural groups or atoms each represented by the same symbol are present for X¹⁷ to X⁸⁰, R¹⁷ to R⁸⁰, and R^{p} to R^{u}, the groups or the atoms may be the same as or different from each other;
e to h each independently represent an integer of 0 or 1, provided that when e to h are 0, the symbols in parentheses with a suffix of 0, (symbol)₀, each independently represent a hydrogen atom or a substituent; and
t and u each independently represent an integer of from 0 to 2, in which t+u=2, provided that the symbols in brackets with a suffix of 0, [symbol]₀, when t is 0, and the symbols in brackets with a suffix of 0, [symbol]₀, when u is 0 each represent a hydrogen atom,
in the general formula (4),
L_{b} represents a substituted or unsubstituted aromatic hydrocarbon group having from 6 to 30 ring carbon atoms or a substituted or unsubstituted heterocyclic group having from 5 to 30 ring atoms;
X³⁰¹ to X³⁰⁸ each represent C(R³⁰¹) to C(R³⁰⁸) or a nitrogen atom, and R³⁰¹ to R³⁰⁸ each independently represent a hydrogen atom or a substituent, in which adjacent substituents may be bonded to each other to form a ring, provided that any one of R³⁰⁵ to R³⁰⁸ represents a single bond directly bonded to L_{b} at the position of *^{c};
R^{aa} and R^{bb} each independently represent a hydrogen atom, an alkyl group having from 1 to 10 carbon atoms, an aryl group having from 6 to 14 ring carbon atoms, or a heteroaryl group having from 5 to 14 ring atoms, in which R^{aa} and R^{bb} may be bonded to each other to form a ring, and R^{aa} and R³⁰¹, and R^{bb} and R³⁰⁸ each independently may be bonded to each other to form a ring; and
m represents 0 or 1, provided that when m is 0, (L_{b})₀ represents a single bond;
the group represented by the formula (4) is bonded to at least one of L³, L⁴, X¹⁷ to X⁸⁰, and R^{p} to R^{u} at the position of *^{d}, in which any one of R¹⁷ to R⁸⁰ and R^{p} to R^{u} that is bonded to the group represented by the formula (4) represents a single bond, and
when plural groups each represented by the formula (4) are present, the groups may be the same as or different from each other.

13. The compound according to claim 12, wherein the compound is represented by the following general formulae (3a) and (4a): 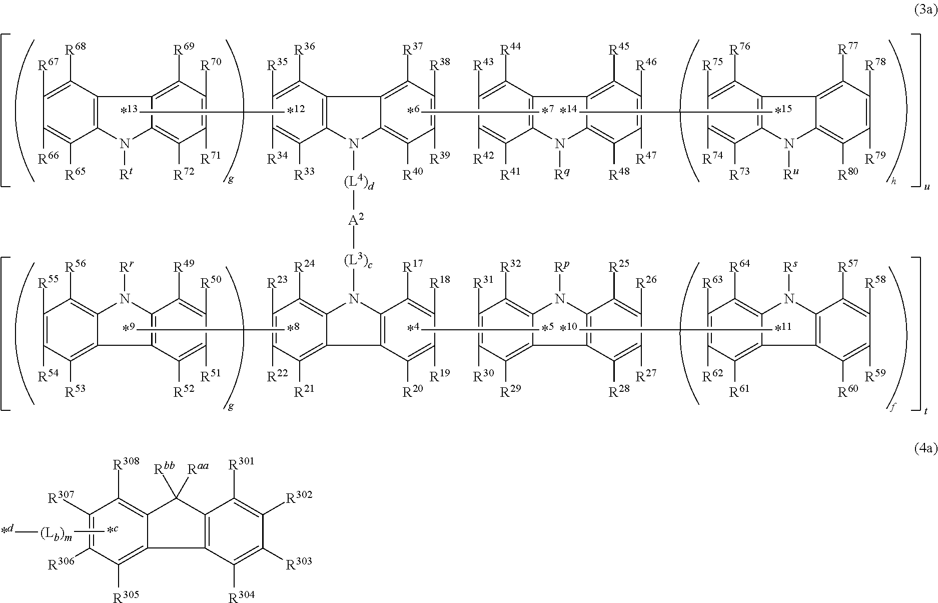
wherein
in the general formula (3a), A², L³, L⁴, c to h, t, u, R¹⁷ to R⁸⁰, and R^{p} to R^{u} have the same meanings as in claim 12, and
in the general formula (4a), L_{b}, m, R³⁰¹ to R³⁰⁸, R^{aa}, and R^{bb} have the same meanings as in claim 12.

14. The compound according to claim 12, wherein the compound is represented by the following general formula (3b): 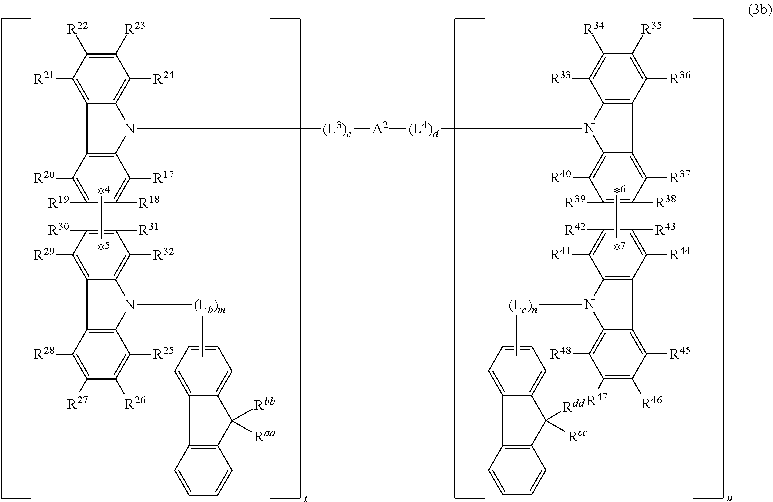
wherein
in the general formula (3b),
A², L³, L⁴, c, d, t, u, R¹⁷ to R⁴⁸, L_{b}, m, R^{aa} and R^{bb} have the same meanings as in claim 12;
L_{c} represents a substituted or unsubstituted aromatic hydrocarbon group having from 6 to 30 ring carbon atoms or a substituted or unsubstituted heterocyclic group having from 5 to 30 ring atoms;
n represents 0 or 1, provided that when n is 0, (L_{c})₀ represents a single bond; and
R^{cc} and R^{dd} each independently represent a hydrogen atom, an alkyl group having from 1 to 10 carbon atoms, an aryl group having from 6 to 14 ring carbon atoms, or a heteroaryl group having from 5 to 14 ring atoms, in which R^{cc} and R^{dd} may be bonded to each other to form a ring.

15. The compound according to claim 14, wherein the compound is represented by the following general formula (3b-1): 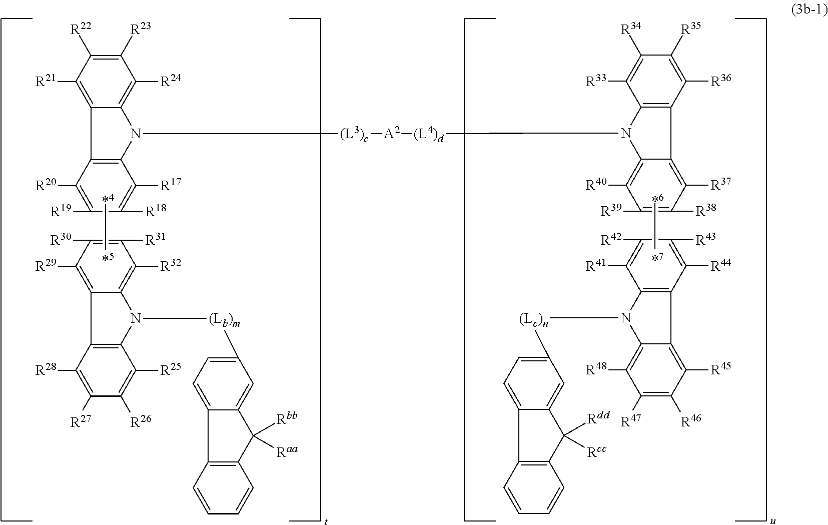
wherein in the general formula (3b-1), A², L³, L⁴, c, d, t, u, R¹⁷ to R⁴⁸, L_{b}, L, m, n, and R^{aa} to R^{dd} have the same meanings as in claim 14.

16. The compound according to claim 14, wherein the compound is represented by the following general formula (3b-2): 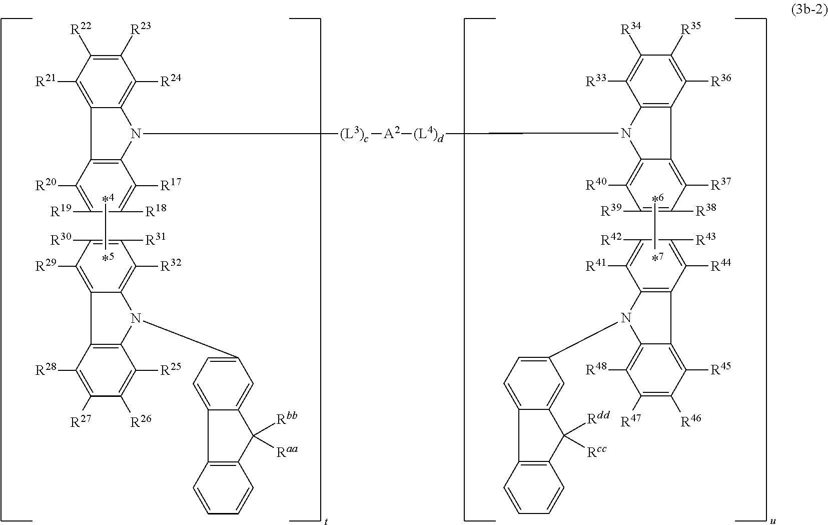
wherein in the general formula (3b-2), A², L³, L⁴, c, d, t, u, R¹⁷ to R⁴⁸, and R^{aa} to R^{dd} have the same meanings as in claim 14.

17. The compound according to claim 14, wherein the compound is represented by the following general formula (3b-3): 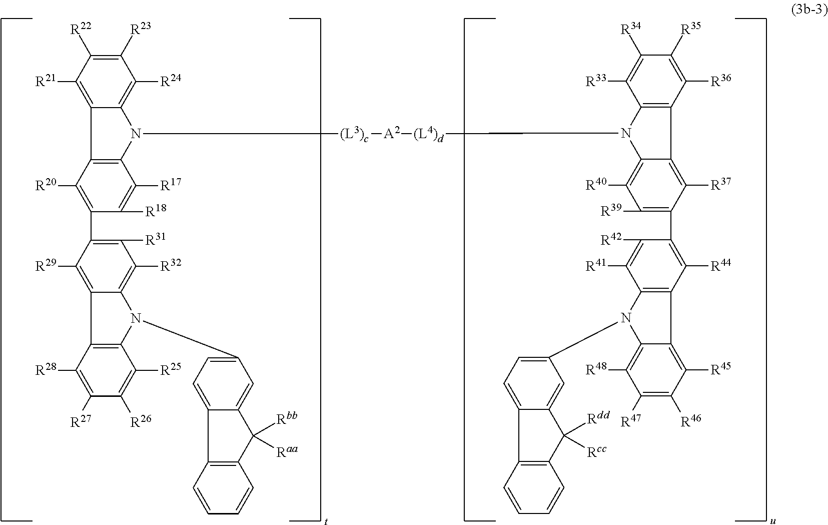
wherein in the general formula (3b-3), A², L³, L⁴, c, d, t, u, R¹⁷, R¹⁸, R²⁰ to R²⁹, R³¹ to R³⁷, R³⁹ to R⁴², R⁴⁴ to R⁴⁸, and R^{aa} to R^{dd} have the same meanings as in claim 14.

18. The compound according to claim 12, wherein the compound is represented by the following general formulae (3c) and (4a): 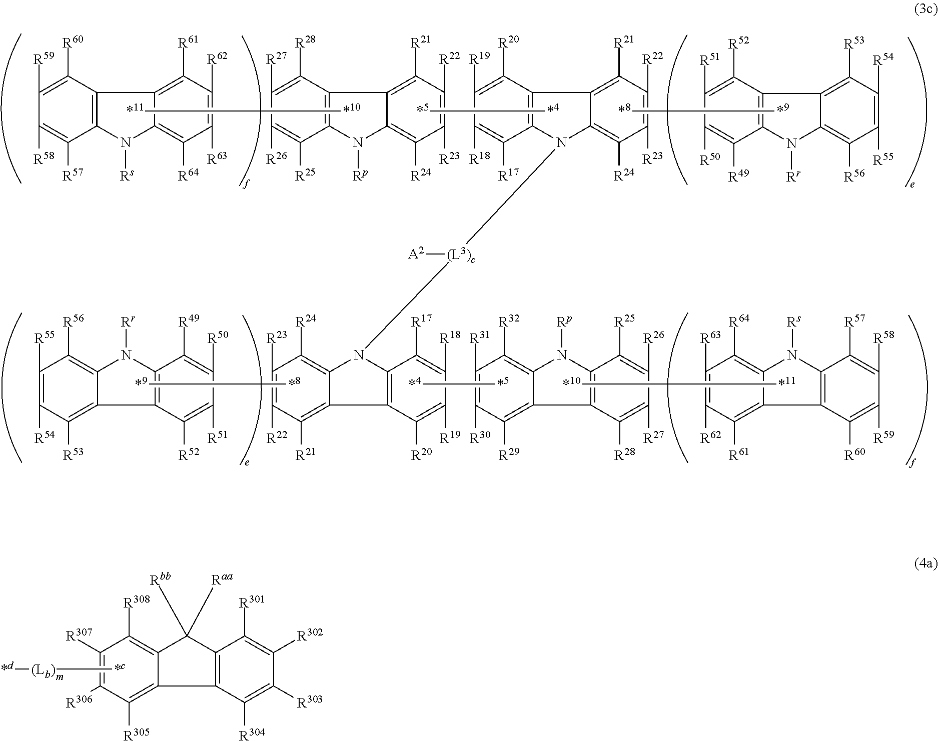
wherein
in the general formula (3c), A², L³, c, e, f, R¹⁷ to R³², R⁴⁹ to R⁶⁴, R^{p}, R^{r}, and R^{s} have the same meanings as in claim 12, and
in the general formula (4a), L_{b}, m, R³⁰⁰ to R³⁰⁸, R^{aa}, and R^{bb} have the same meanings as in claim 12.

19. The compound according to claim 12, wherein the compound is represented by the following general formula (3e): 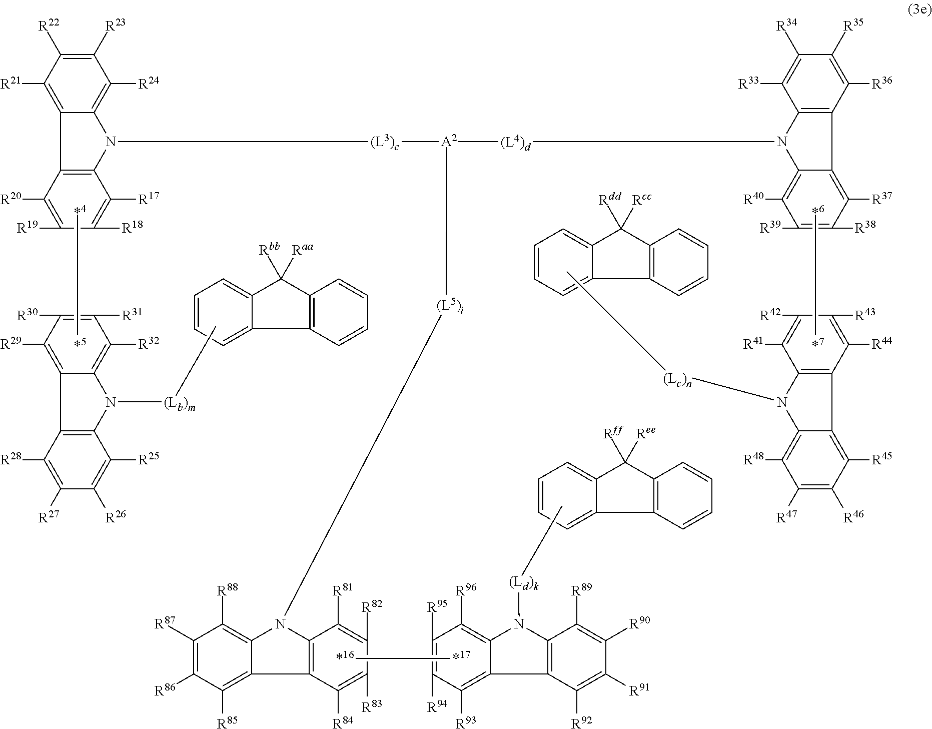
wherein
in the general formula (3e) A², L³, L⁴, c, d, R¹⁷ to R⁴⁸, L_{b}, m, R^{aa}, and R^{bb} have the same meanings as in claim 12;
L⁵ represents a substituted or unsubstituted aromatic hydrocarbon group having from 6 to 30 ring carbon atoms, a substituted or unsubstituted heterocyclic group having from 5 to 30 ring atoms, or a group containing from 2 to 4 of the groups bonded to each other;
i represents 0 or 1, provided that when i is 0, (L⁵)₀ represents a single bond;
R⁸¹ to R⁹⁶ each independently represent a hydrogen atom or a substituent, in which adjacent substituents may be bonded to each other to form a ring,
provided that any one of R⁸¹ to R⁸⁴ represents a single bond bonded to *¹⁶, and any one of R⁹³ to R⁹⁶ represents a single bond bonded to *¹⁷;
L_{c} and L_{d} each independently represent a substituted or unsubstituted aromatic hydrocarbon group having from 6 to 30 ring carbon atoms or a substituted or unsubstituted heterocyclic group having from 5 to 30 ring atoms;
k and n each independently represent 0 or 1, provided that when k is 0, (L_{d})₀ represents a single bond, and when n is 0, (L_{c})₀ represents a single bond; and
R^{cc} to R^{ff} each independently represent a hydrogen atom, an alkyl group having from 1 to 10 carbon atoms, an aryl group having from 6 to 14 ring carbon atoms, or a heteroaryl group having from 5 to 14 ring atoms, in which R^{cc} and R^{dd}, and R^{ee} and R^{ff} each independently may be bonded to each other to form a ring.

20. The compound according to claim 12, wherein in the general formula (3), at least one of L³ and L⁴ is any one of groups represented by the following formulae: 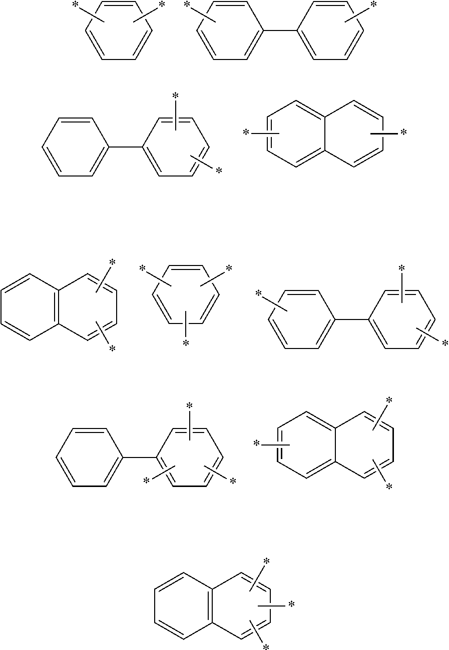
wherein in the formulae, * represents the bonding position, and the carbon atoms at the positions other than the bonding positions may have a substituent.

21. The compound according to claim 12, wherein R^{aa} to R^{ff} each independently represent a methyl group or a phenyl group.

22. The compound according to claim 12, wherein the substituent in, or the substituent in the expression “substituted or unsubstituted” in A², L³, L⁴, L⁵, L_{b}, L_{c}, L_{d}, X¹⁷ to X⁸⁰, X³⁰¹ to X³⁰⁸, and R^{p} to R^{u} each are selected from the group consisting of an alkyl group having from 1 to 50 carbon atoms, a cycloalkyl group having from 3 to 50 ring carbon atoms, an aryl group having from 6 to 50 ring carbon atoms, an aralkyl group having from 7 to 51 carbon atoms having an aryl group having from 6 to 50 ring carbon atoms, an amino group, a monosubstituted or disubstituted amino group having a substituent selected from an alkyl group having from 1 to 50 carbon atoms and an aryl group having from 6 to 50 ring carbon atoms, an alkoxy group having an alkyl group having from 1 to 50 carbon atoms, an aryloxy group having an aryl group having from 6 to 50 ring carbon atoms, a monosubstituted, disubstituted or trisubstituted silyl group having a substituent selected from an alkyl group having from 1 to 50 carbon atoms and an aryl group having from 6 to 50 ring carbon atoms, a heteroaryl group having from 5 to 50 ring atoms, a haloalkyl group having from 1 to 50 carbon atoms, a halogen atom, a cyano group, a nitro group, a sulfonyl group having a substituent selected from an alkyl group having from 1 to 50 carbon atoms and an aryl group having from 6 to 50 ring carbon atoms, a disubstituted phosphoryl group having a substituent selected from an alkyl group having from 1 to 50 carbon atoms and an aryl group having from 6 to 50 ring carbon atoms, an alkylsulfonyloxy group, an arylsulfonyloxy group, an alkylcarbonyloxy group, an arylcarbonyloxy group, a boron-containing group, a zinc-containing group, a tin-containing group, a silicon-containing group, a magnesium-containing group, a lithium-containing group, a hydroxyl group, an alkyl-substituted or aryl-substituted carbonyl group, a carboxyl group, a vinyl group, a (meth)acryloyl group, an epoxy group, and an oxetanyl group.

23. An ink composition comprising a solvent and the compound according to claim 1.

24. The ink composition according to claim 23, which further comprises a metal complex.

25. A material for an organic electroluminescence device, comprising the compound according to claim 1.

26. An organic electroluminescence device comprising a cathode, an anode, and one or more organic thin film layers between the cathode and the anode,
wherein the one or more organic thin film layers includes a light emitting layer, and at least one layer of the one or more organic thin film layers comprises the compound according to claim 1.

27. The organic electroluminescence device according to claim 26, wherein the light emitting layer comprises the compound according to claim 1.

28. An electronic equipment having the organic electroluminescence device according to claim 26 mounted thereon.

29. An ink composition comprising a solvent and the compound according to claim 12.

30. A material for an organic electroluminescence device, comprising the compound according to claim 12.

31. An organic electroluminescence device comprising a cathode, an anode, and one or more organic thin film layers between the cathode and the anode,
wherein the one or more organic thin film layers includes a light emitting layer, and at least one layer of the one or more organic thin film layers comprises the compound according to claim 12.

32. The organic electroluminescence device according to claim 26, wherein the light emitting layer comprises the compound according to claim 12.

33. The ink composition according to claim 29, which further comprises a metal complex.

34. An electronic equipment having the organic electroluminescence device according to claim 31 mounted thereon.
